SEQUENCE LISTING (451 sequences)

SEQ ID NO: 1 - protein, 548 aa, herpes simplex virus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPPPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 2 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASLFVLGVALSANGTFVYNGSDYGSCDPAQLPFSAPRLGPSSVYTPGASRPTPPRTTTSPSSPRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 3 - protein, 550 aa, herpes simplex virus 1
MDRGAVVGFLLGVCVVSCLAGTPKTSWRRVSVGEDVSLLPAPGPTGRGPTQKLLWAVEPLDGCGPLHPSWVSLMPPKQVPETVVDAACMRAPVPLAMAYAPPAPSATGGLRTDFVWQERAAVVNRSLVIHGVRETDSGLYTLSVGDIKDPARQVASVVLVVQPAPVPTPPPTPADYDEDDNDEGEDESLAGTPASGTPRLPPPPAPPRSWPSAPEVSHVRGVTVRMETPEAILFSPGETFSTNVSIHAIAHDDQTYSMDVVWLRFDVPTSCAEMRIYESCLYHPQLPECLSPADAPCAASTWTSRLAVRSYAGCSRTNPPPRCSAEAHMEPVPGLAWQAASVNLEFRDASPQHSGLYLCVVYVNDHIHAWGHITISTAAQYRNAVVEQPLPQRGADLAEPTHPHVGAPPHAPPTHGALRLGAVMGAALLLSALGLSVWACMTCWRRRAWRAVKSRASGKGPTYIRVADSELYADWSSDSEGERDQVPWLAPPERPDSPSTNGSGFEILSPTAPSVYPRSDGHQSRRQLTTFGSGRPDRRYSQASDSSVFW

SEQ ID NO: 4 - protein, 390 aa, herpes simplex virus 1
MPCRPLQGLVLVGLWVCATSLVVRGPTVSLVSNSFVDAGALGPDGVVEEDLLILGELRFVGDQVPHTTYYDGGVELWHYPMGHKCPRVVHVVTVTACPRRPAVAFALCRATDSTHSPAYPTLELNLAQQPLLRVQRATRDYAGVYVLRVWVGDAPNASLFVLGMAIAAEGTLAYNGSAYGSCDPKLLPSSAPRLAPASVYQPAPNQASTPSTTTSTPSTTIPAPSTTIPAPQASTTPFPTGDPKPQPPGVNHEPPSNATRATRDSRYALTVTQIIQIAIPASIIALVFLGSCICFIHRCQRRYRRSRRPIYSPQMPTGISCAVNEAAMARLGAELKSHPSTPPKSRRRSSRTPMPSLTAIAEESEPAGAAGLPTPPVDPTTPTPTPPLLV

SEQ ID NO: 5 - protein, 419 aa, Artificial Sequence
  HSV2 gE ectodomain
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPPPAPSARGPLRLG

SEQ ID NO: 6 - protein, 256 aa, Artificial Sequence
  HSV2 gI ectodomain
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASLFVLGVALSANGTFVYNGSDYGSCDPAQLPFSAPRLGPSSVYTPGASRPTPPRTTTSPSSPRDPTPAPGDTGTPAPASGERAPPNSTRSASESRHRLT

SEQ ID NO: 7 - protein, 420 aa, Artificial Sequence
  HSV1 gE ectodomain
MDRGAVVGFLLGVCVVSCLAGTPKTSWRRVSVGEDVSLLPAPGPTGRGPTQKLLWAVEPLDGCGPLHPSWVSLMPPKQVPETVVDAACMRAPVPLAMAYAPPAPSATGGLRTDFVWQERAAVVNRSLVIHGVRETDSGLYTLSVGDIKDPARQVASVVLVVQPAPVPTPPPTPADYDEDDNDEGEDESLAGTPASGTPRLPPPPAPPRSWPSAPEVSHVRGVTVRMETPEAILFSPGETFSTNVSIHAIAHDDQTYSMDVVWLRFDVPTSCAEMRIYESCLYHPQLPECLSPADAPCAASTWTSRLAVRSYAGCSRTNPPPRCSAEAHMEPVPGLAWQAASVNLEFRDASPQHSGLYLCVVYVNDHIHAWGHITISTAAQYRNAVVEQPLPQRGADLAEPTHPHVGAPPHAPPTHGALRL

SEQ ID NO: 8 - protein, 270 aa, Artificial Sequence
  HSV1 gI ectodomain
MPCRPLQGLVLVGLWVCATSLVVRGPTVSLVSNSFVDAGALGPDGVVEEDLLILGELRFVGDQVPHTTYYDGGVELWHYPMGHKCPRVVHVVTVTACPRRPAVAFALCRATDSTHSPAYPTLELNLAQQPLLRVQRATRDYAGVYVLRVWVGDAPNASLFVLGMAIAAEGTLAYNGSAYGSCDPKLLPSSAPRLAPASVYQPAPNQASTPSTTTSTPSTTIPAPSTTIPAPQASTTPFPTGDPKPQPPGVNHEPPSNATRATRDSRYALT

SEQ ID NO: 9 - protein, 119 aa, Artificial Sequence
  mAb 2 VH
QVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARSRGYPYAMDYWGQGTSVTVSS

SEQ ID NO: 10 - protein, 5 aa, Artificial Sequence
  mAb 2 HCDR1
NYLIE

SEQ ID NO: 11 - protein, 17 aa, Artificial Sequence
  mAb 2 HCDR2
VINPGSGGTNYNEKFKG

SEQ ID NO: 12 - protein, 10 aa, Artificial Sequence
  mAb 2 HCDR3
SRGYPYAMDY

SEQ ID NO: 13 - protein, 118 aa, Artificial Sequence
  mAb 3,21, VH
EVQLQQSGPELVKPGASVKISCKTSGYTFTEYTMHWVKQSHGKSLEWIGGINPNNGGTSYNQKFKGKATLTVDKSSSTAYMELRSLTSEDSAVYYCARSLVEGDFAYWGQGTLVTVSA

SEQ ID NO: 14 - protein, 5 aa, Artificial Sequence
  mAb 3,21 HCDR1
EYTMH

SEQ ID NO: 15 - protein, 17 aa, Artificial Sequence
  mAb 3,21 HCDR2
GINPNNGGTSYNQKFKG

SEQ ID NO: 16 - protein, 9 aa, Artificial Sequence
  mAb 3,21 HCDR3
SLVEGDFAY

SEQ ID NO: 17 - protein, 124 aa, Artificial Sequence
  mAb 5 VH
EVKLVESGGGLVQPGGSLRLSCATSGFTFSDFYMEWVRQPPGKRLEWIAASRNKANDYTTEYSASVKGRFIVSRDTSQSILYLQMNALRAEDTAIYYCARDFLPYYGSPWFAYWGQGTLVTVSA

SEQ ID NO: 18 - protein, 5 aa, Artificial Sequence
  mAb 5 HCDR1
DFYME

SEQ ID NO: 19 - protein, 19 aa, Artificial Sequence
  mAb 5 HCDR2
ASRNKANDYTTEYSASVKG

SEQ ID NO: 20 - protein, 13 aa, Artificial Sequence
  mAb 5 HCDR3
DFLPYYGSPWFAY

SEQ ID NO: 21 - protein, 126 aa, Artificial Sequence
  mAb 9 VH
EVQLQQSGPELEKPGASVKISCKASGYSFTGYNMNWVKQSNGKSLEWIGNIDPYYGGTSYNQKFKGKATLTLDKSSSTAYMQLKSLTSEDSAVYYCARSRFITTVVATDYYAMDYWGQGTSVTVSS

SEQ ID NO: 22 - protein, 5 aa, Artificial Sequence
  mAb 9 HCDR1
GYNMN

SEQ ID NO: 23 - protein, 17 aa, Artificial Sequence
  mAb 9 HCDR2
NIDPYYGGTSYNQKFKG

SEQ ID NO: 24 - protein, 17 aa, Artificial Sequence
  mAb 9 HCDR3
SRFITTVVATDYYAMDY

SEQ ID NO: 25 - protein, 121 aa, Artificial Sequence
  mAb 10 VH
EVKLVESGGGLVQPGGSLRLSCATSGFTFSDFYMEWVRQPPGKRLEWIAASRNKANDYTTEYSASVKGRFIVSRDTSQSILYLQMNALRAEDTAIYYCARENYGNPAMDYWGQGTSVTVSS

SEQ ID NO: 26 - protein, 5 aa, Artificial Sequence
  mAb 10 HCDR1
DFYME

SEQ ID NO: 27 - protein, 19 aa, Artificial Sequence
  mAb 10 HCDR2
ASRNKANDYTTEYSASVKG

SEQ ID NO: 28 - protein, 10 aa, Artificial Sequence
  mAb 10 HCDR3
ENYGNPAMDY

SEQ ID NO: 29 - protein, 121 aa, Artificial Sequence
  mAb 12,13 VH
QVQLKESGPGLVAPSQSLSITCTVSGFSLTSYGVHWVRQPPGKGLEWLGVIWAGGSTNYNSALMSRLSISKDNSKSQVFLKMNSLQTDDTAMYFCARGGITSLHNYVMDYWGQGTSVTVSS

SEQ ID NO: 30 - protein, 5 aa, Artificial Sequence
  mAb 12,13 HCDR1
SYGVH

SEQ ID NO: 31 - protein, 16 aa, Artificial Sequence
  mAb 12,13 HCDR2
VIWAGGSTNYNSALMS

SEQ ID NO: 32 - protein, 13 aa, Artificial Sequence
  mAb 12,13 HCDR3
GGITSLHNYVMDY

SEQ ID NO: 33 - protein, 125 aa, Artificial Sequence
  mAb 14 VH
QVQLQQSGAELMKPGASVKISCKATGYTFSSYWIEWVKQRPGHGLEWIGEILPGSGSTNYNEKFKGKATFTADTSSNTAYMQLSSLTSEDSAVYYCASGSAGNYYGSSYEAMDYWGQGTSVTVSS

SEQ ID NO: 34 - protein, 5 aa, Artificial Sequence
  mAb 14 HCDR1
SYWIE

SEQ ID NO: 35 - protein, 17 aa, Artificial Sequence
  mAb 14 HCDR2
EILPGSGSTNYNEKFKG

SEQ ID NO: 36 - protein, 16 aa, Artificial Sequence
  mAb 14 HCDR3
GSAGNYYGSSYEAMDY

SEQ ID NO: 37 - protein, 121 aa, Artificial Sequence
  mAb 15 VH
QVQLKESGPGLVAPSQSLSITCTVSGFSLTSYGVSWVRQPPGKGLEWLGVIWGDGSTNYHSALISRLSISKDNSKSQVFLKLNSLQTDDTATYYCAKPDLYYDSPYAMDYWGQGTSVTVSS

SEQ ID NO: 38 - protein, 5 aa, Artificial Sequence
  mAb 15 HCDR1
SYGVS

SEQ ID NO: 39 - protein, 16 aa, Artificial Sequence
  mAb 15 HCDR2
VIWGDGSTNYHSALIS

SEQ ID NO: 40 - protein, 13 aa, Artificial Sequence
  mAb 15 HCDR3
PDLYYDSPYAMDY

SEQ ID NO: 41 - protein, 114 aa, Artificial Sequence
  mAb 16 VH
EVKLLESGGGLVQPGGSLKLSCAASGFDFSRYWMSWVRQAPGKGLEWIGEINPDSSTINYTPSLKDKFIISRDNAKNTLYLQMSKVRSEDTALYYCARPLMDYWGQGTSVTVSS

SEQ ID NO: 42 - protein, 5 aa, Artificial Sequence
  mAb 16 HCDR1
RYWMS

SEQ ID NO: 43 - protein, 17 aa, Artificial Sequence
  mAb 16 HCDR2
EINPDSSTINYTPSLKD

SEQ ID NO: 44 - protein, 5 aa, Artificial Sequence
  mAb 16 HCDR3
PLMDY

SEQ ID NO: 45 - protein, 120 aa, Artificial Sequence
  mAb 17 VH
QVQLKESGPGLVAPSQSLSITCTVSGFSLTSYGVHWVRQPPGKGLEWLGVIWAGGSSNYNSALMSRLSISKDNSKSQVFLKMNSLQTDDTAMYYCARDGYGYVYYAMDYWGQGTSVTVSS

SEQ ID NO: 46 - protein, 5 aa, Artificial Sequence
  mAb 17 HCDR1
SYGVH

SEQ ID NO: 47 - protein, 16 aa, Artificial Sequence
  mAb 17 HCDR2
VIWAGGSSNYNSALMS

SEQ ID NO: 48 - protein, 12 aa, Artificial Sequence
  mAb 17 HCDR3
DGYGYVYYAMDY

SEQ ID NO: 49 - protein, 115 aa, Artificial Sequence
  mAb 18 VH
QVQLQQSGAELVRPGTSVKMSCKAAGYTFTNYWIGWVKQRPGHGLEWIGDIYPGGGYTKYNEKFKGKATLTADTSSSTAYMQLSSLTSEDSAIYYCARDGYSPYWGQGTSLTVSS

SEQ ID NO: 50 - protein, 5 aa, Artificial Sequence
  mAb 18 HCDR1
NYWIG

SEQ ID NO: 51 - protein, 17 aa, Artificial Sequence
  mAb 18 HCDR2
DIYPGGGYTKYNEKFKG

SEQ ID NO: 52 - protein, 6 aa, Artificial Sequence
  mAb 18 HCDR3
DGYSPY

SEQ ID NO: 53 - protein, 122 aa, Artificial Sequence
  mAb 19 VH
QVQLQQSGAELVRPGVSVKISCKGSGYTFTDYAMHWVKQSHAKSLEWIGVISTYYGDARYNQKFKGKATMTVDKSSSTAYMELARLTSEDSAIYYCARRGITVVADYAMDYWGQGTSVTVSS

SEQ ID NO: 54 - protein, 5 aa, Artificial Sequence
  mAb 19 HCDR1
DYAMH

SEQ ID NO: 55 - protein, 17 aa, Artificial Sequence
  mAb 19 HCDR2
VISTYYGDARYNQKFKG

SEQ ID NO: 56 - protein, 13 aa, Artificial Sequence
  mAb 19 HCDR3
RGITVVADYAMDY

SEQ ID NO: 57 - protein, 120 aa, Artificial Sequence
  mAb 20 VH
QVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARSHYGNYYAMDYWGQGTSVTVSS

SEQ ID NO: 58 - protein, 5 aa, Artificial Sequence
  mAb 20 HCDR1
NYLIE

SEQ ID NO: 59 - protein, 17 aa, Artificial Sequence
  mAb 20 HCDR2
VINPGSGGTNYNEKFKG

SEQ ID NO: 60 - protein, 11 aa, Artificial Sequence
  mAb 20 HCDR3
SHYGNYYAMDY

SEQ ID NO: 61 - protein, 124 aa, Artificial Sequence
  mAb 24 VH
EVQLQQSGPELVKPGASVKMSCKASGYTFTDYYMKWVKQSHGKSLEWIGDINPNNGDTFYNQKFKGKATLTVDKSSSTAYMQLNSLTSEDSAVYYCARRGFYYYGSRGGIMDYWGQGTSVTVSS

SEQ ID NO: 62 - protein, 5 aa, Artificial Sequence
  mAb 24 HCDR1
DYYMK

SEQ ID NO: 63 - protein, 17 aa, Artificial Sequence
  mAb 24 HCDR2
DINPNNGDTFYNQKFKG

SEQ ID NO: 64 - protein, 15 aa, Artificial Sequence
  mAb 24 HCDR3
RGFYYYGSRGGIMDY

SEQ ID NO: 65 - protein, 118 aa, Artificial Sequence
  mAb 34 VH
EVKLVESGGGLVQPGGSLKLSCATSGFTFSDYYMYWVRQTPEKRLEWVAYISNGGDSTYYPDTVKGRFTISRDNAKNTLYLQMSRLKSEDTAMYYCARSYYGNPFPYWGQGTLVTVSA

SEQ ID NO: 66 - protein, 5 aa, Artificial Sequence
  mAb 34 HCDR1
DYYMY

SEQ ID NO: 67 - protein, 17 aa, Artificial Sequence
  mAb 34 HCDR2
YISNGGDSTYYPDTVKG

SEQ ID NO: 68 - protein, 9 aa, Artificial Sequence
  mAb 34 HCDR3
SYYGNPFPY

SEQ ID NO: 69 - protein, 120 aa, Artificial Sequence
  mAb 35, 36 VH
QVQLKESGPGLVAPSQSLSITCTVSGFSLTSYGVHWVRQPPGKGLEWLGVIWAGGSTNYNSALMSRLSISKDNSKSQVFLKMNSLQTDDTAMYYCATDGYKFFYYAMDYWGQGTSVTVSS

SEQ ID NO: 70 - protein, 5 aa, Artificial Sequence
  mAb 35, 36 HCDR1
SYGVH

SEQ ID NO: 71 - protein, 16 aa, Artificial Sequence
  mAb 35, 36 HCDR2
VIWAGGSTNYNSALMS

SEQ ID NO: 72 - protein, 12 aa, Artificial Sequence
  mAb 35, 36 HCDR3
DGYKFFYYAMDY

SEQ ID NO: 73 - protein, 118 aa, Artificial Sequence
  mAb 37 VH
EVQLQQSGPELVKPGASVKISCKASGYTFTDYNMHWVKQSHGKSLEWIGYIYPYNGGTGYNQKFKTKATLTVDNSSSTAYMELRSLTSEDSAVYYCAIPGYYYAMDYWGQGTSVTVSS

SEQ ID NO: 74 - protein, 5 aa, Artificial Sequence
  mAb 37 HCDR1
DYNMH

SEQ ID NO: 75 - protein, 17 aa, Artificial Sequence
  mAb 37 HCDR2
YIYPYNGGTGYNQKFKT

SEQ ID NO: 76 - protein, 9 aa, Artificial Sequence
  mAb 37 HCDR3
PGYYYAMDY

SEQ ID NO: 77 - protein, 122 aa, Artificial Sequence
  mAb 39 VH
EVKLVESGGGLVQPGGSLKLSCAASGFTFSSYTMSWVRQTPEKRLEWVAYISNGGGSTYYPDTVKGRFTISRDNAKNTLYLQMSSLKSEDTAMYYCARGVSYYGDLYYFDYWGQGTTLTVSS

SEQ ID NO: 78 - protein, 5 aa, Artificial Sequence
  mAb 39 HCDR1
SYTMS

SEQ ID NO: 79 - protein, 17 aa, Artificial Sequence
  mAb 39 HCDR2
YISNGGGSTYYPDTVKG

SEQ ID NO: 80 - protein, 13 aa, Artificial Sequence
  mAb 39 HCDR3
GVSYYGDLYYFDY

SEQ ID NO: 81 - protein, 121 aa, Artificial Sequence
  mAb 40 VH
QIQLVQSGPELKKPGETVKISCKASGYTFTNYGMNWVKQAPGKGLKWMGWINTYTGEPTYADDFKGRFAFSLETSASTAYLQINNLKNEDTATYFCARSLYGYDEGYFDVWGAGTTVTVSS

SEQ ID NO: 82 - protein, 5 aa, Artificial Sequence
  mAb 40 HCDR1
NYGMN

SEQ ID NO: 83 - protein, 17 aa, Artificial Sequence
  mAb 40 HCDR2
WINTYTGEPTYADDFKG

SEQ ID NO: 84 - protein, 12 aa, Artificial Sequence
  mAb 40 HCDR3
SLYGYDEGYFDV

SEQ ID NO: 85 - protein, 121 aa, Artificial Sequence
  mAb 41 VH
QVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARSGLRRDYYAMDYWGQGTSVTVSS

SEQ ID NO: 86 - protein, 5 aa, Artificial Sequence
  mAb 41 HCDR1
NYLIE

SEQ ID NO: 87 - protein, 17 aa, Artificial Sequence
  mAb 41 HCDR2
VINPGSGGTNYNEKFKG

SEQ ID NO: 88 - protein, 12 aa, Artificial Sequence
  mAb 41 HCDR3
SGLRRDYYAMDY

SEQ ID NO: 89 - protein, 117 aa, Artificial Sequence
  mAb 42 VH
QVQLKESGPGLVAPSQSLSITCTVSGFSLTSYGVHWVRQPPGKGLEWLGVIWAGGSTNYNSALMSRLSISKDNSKSQVFLKMNSLQTDDTAMYYCAREGNWDDFDYWGQGTTLTVSS

SEQ ID NO: 90 - protein, 5 aa, Artificial Sequence
  mAb 42 HCDR1
SYGVH

SEQ ID NO: 91 - protein, 16 aa, Artificial Sequence
  mAb 42 HCDR2
VIWAGGSTNYNSALMS

SEQ ID NO: 92 - protein, 9 aa, Artificial Sequence
  mAb 42 HCDR3
EGNWDDFDY

SEQ ID NO: 93 - protein, 122 aa, Artificial Sequence
  mAb 43 VH
DVKLVESGGGLVKPGGSLKLSCAASGFTFSSYTMSWVRQTPEKRLEWVATISSGGSYTYYPDSVKGRFTISRDNAKNTLYLQMSSLKAEDTAMYYCTRGGYGSSFENYFDYWGQGTTLTVSS

SEQ ID NO: 94 - protein, 5 aa, Artificial Sequence
  mAb 43 HCDR1
SYTMS

SEQ ID NO: 95 - protein, 17 aa, Artificial Sequence
  mAb 43 HCDR2
TISSGGSYTYYPDSVKG

SEQ ID NO: 96 - protein, 13 aa, Artificial Sequence
  mAb 43 HCDR3
GGYGSSFENYFDY

SEQ ID NO: 97 - protein, 122 aa, Artificial Sequence
  mAb 44 VH
EVQLQQSGPELEKPGASVKISCKASGYSFTGYNMNWVKQSNGKSLEWIGNIDPYYGGIRYNQKFKGKATLTVDKSSSTAYMQLKSLTSEDSAVYYCAREDRYLVHYYAMDYWGQGTSVTVSS

SEQ ID NO: 98 - protein, 5 aa, Artificial Sequence
  mAb 44 HCDR1
GYNMN

SEQ ID NO: 99 - protein, 18 aa, Artificial Sequence
  mAb 44 HCDR2
GNIDPYYGGIRYNQKFKG

SEQ ID NO: 100 - protein, 13 aa, Artificial Sequence
  mAb 44 HCDR3
EDRYLVHYYAMDY

SEQ ID NO: 101 - protein, 122 aa, Artificial Sequence
  mAb 45 VH
DVKLVESGGGLVKPGGSLKLSCAASGFTFSSYTMSWVRQTPEKRLEWVATISSGGSYTYYPDSVKGRFTISRDNAKNTLYLQMSSLKSEDTAMYYCTRNYGSRPPYYAMDYWGQGTSVTVSS

SEQ ID NO: 102 - protein, 5 aa, Artificial Sequence
  mAb 45 HCDR1
SYTMS

SEQ ID NO: 103 - protein, 17 aa, Artificial Sequence
  mAb 45 HCDR2
TISSGGSYTYYPDSVKG

SEQ ID NO: 104 - protein, 13 aa, Artificial Sequence
  mAb 45 HCDR3
NYGSRPPYYAMDY

SEQ ID NO: 105 - protein, 118 aa, Artificial Sequence
  mAb 47 VH
EVKLVESGGGLVQPGGSLRLSCATSGFTFTDYYMSWVRQPPGKALEWLGFIRNKANGYTTEYSASVKGRFTISRDNSQSILYLQMNTLRAEDSATYYCARDRPGFDYWGQGTTLTVSS

SEQ ID NO: 106 - protein, 5 aa, Artificial Sequence
  mAb 47 HCDR1
DYYMS

SEQ ID NO: 107 - protein, 19 aa, Artificial Sequence
  mAb 47 HCDR2
FIRNKANGYTTEYSASVKG

SEQ ID NO: 108 - protein, 7 aa, Artificial Sequence
  mAb 47 HCDR3
DRPGFDY

SEQ ID NO: 109 - protein, 129 aa, Artificial Sequence
  mAb 48 VH
QVTLKESGPGILQPSQTLSLTCSFSGFSLSTSGMGVSWIRQPSGKGLEWLAHIYWDDDKRYNPSLKSRLTISKDTSSNQVFLKITSVDTADTATYYCARRGFYYRYDGGRRYYYAMDYWGQGTSVTVSS

SEQ ID NO: 110 - protein, 7 aa, Artificial Sequence
  mAb 48 HCDR1
TSGMGVS

SEQ ID NO: 111 - protein, 16 aa, Artificial Sequence
  mAb 48 HCDR2
HIYWDDDKRYNPSLKS

SEQ ID NO: 112 - protein, 19 aa, Artificial Sequence
  mAb 48 HCDR3
RGFYYRYDGGRRYYYAMDY

SEQ ID NO: 113 - protein, 111 aa, Artificial Sequence
  mAb 2 VL
DIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIK

SEQ ID NO: 114 - protein, 15 aa, Artificial Sequence
  mAb 2 LCDR1
KASQSVDYDGDSYMN

SEQ ID NO: 115 - protein, 7 aa, Artificial Sequence
  mAb 2 LCDR2
AASNLES

SEQ ID NO: 116 - protein, 9 aa, Artificial Sequence
  mAb 2 LCDR3
QQSNEDPYT

SEQ ID NO: 117 - protein, 111 aa, Artificial Sequence
  mAb 3, 21 VL
DIVLTQSPASLAVSLGQRATISCRASQSVSTSSYSYMHWYQQKPGQPPKLLIKYASNLESGVPARFSGSGSGTDFTLNIHPVEEEDTATYYCQHSWEIPYTFGGGTKLEIK

SEQ ID NO: 118 - protein, 15 aa, Artificial Sequence
  mAb 3,21 LCDR1
RASQSVSTSSYSYMH

SEQ ID NO: 119 - protein, 7 aa, Artificial Sequence
  mAb 3, 21 LCDR2
YASNLES

SEQ ID NO: 120 - protein, 9 aa, Artificial Sequence
  mAb 3, 21 LCDR3
QHSWEIPYT

SEQ ID NO: 121 - protein, 107 aa, Artificial Sequence
  mAb 5 VL
DIVMTQSPATLSVTPGDRVSLSCRASQSISDYLHWYQQKSHESPRLLIKYASQSISGIPSRFSGSGSGSDFTLSINSVEPEDVGVYYCQNGHSFPYTFGGGTKLEIK

SEQ ID NO: 122 - protein, 11 aa, Artificial Sequence
  mAb 5 LCDR1
RASQSISDYLH

SEQ ID NO: 123 - protein, 7 aa, Artificial Sequence
  mAb 5 LCDR2
YASQSIS

SEQ ID NO: 124 - protein, 9 aa, Artificial Sequence
  mAb 5 LCDR3
QNGHSFPYT

SEQ ID NO: 125 - protein, 113 aa, Artificial Sequence
  mAb 9 VL
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYPPTFGGGTKLEIK

SEQ ID NO: 126 - protein, 17 aa, Artificial Sequence
  mAb 9 LCDR1
KSSQSLLYSSNQKNYLA

SEQ ID NO: 127 - protein, 7 aa, Artificial Sequence
  mAb 9 LCDR2
WASTRES

SEQ ID NO: 128 - protein, 9 aa, Artificial Sequence
  mAb 9 LCDR3
QQYYSYPPT

SEQ ID NO: 129 - protein, 111 aa, Artificial Sequence; identical to SEQ ID NO: 113
  mAb 10 VL

SEQ ID NO: 130 - protein, 15 aa, Artificial Sequence
  mAb 10 LCDR1
KASQSVDYDGDSYMN

SEQ ID NO: 131 - protein, 7 aa, Artificial Sequence
  mAb 10 LCDR2
AASNLES

SEQ ID NO: 132 - protein, 9 aa, Artificial Sequence
  mAb 10 LCDR3
QQSNEDPYT

SEQ ID NO: 133 - protein, 111 aa, Artificial Sequence
  mAb 12, 13 LV
DIVLTQSPASLAVSLGQRATISCRASESVEYYGTSLMQWYQQKPGQPPKLLIYAASNVESGVPARFSGSGSGTHFSLNIHPVEEDDIAMYFCQQSRKVPSTFGGGTKLEIK

SEQ ID NO: 134 - protein, 15 aa, Artificial Sequence
  mAb 12, 13 LCDR1
RASESVEYYGTSLMQ

SEQ ID NO: 135 - protein, 7 aa, Artificial Sequence
  mAb 12,13 LCDR2
AASNVES

SEQ ID NO: 136 - protein, 9 aa, Artificial Sequence
  mAb 12, 13 LCDR3
QQSRKVPST

SEQ ID NO: 137 - protein, 111 aa, Artificial Sequence
  mAb 14 VL
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPLTFGAGTKLELK

SEQ ID NO: 138 - protein, 15 aa, Artificial Sequence
  mAb 14 LCDR1
RASKSVSTSGYSYMH

SEQ ID NO: 139 - protein, 7 aa, Artificial Sequence
  mAb 14 LCDR2
LASNLES

SEQ ID NO: 140 - protein, 9 aa, Artificial Sequence
  mAb 14 LCDR3
QHSRELPLT

SEQ ID NO: 141 - protein, 107 aa, Artificial Sequence
  mAb 15 VL
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 142 - protein, 11 aa, Artificial Sequence
  mAb 15 LCDR1
RASGNIHNYLA

SEQ ID NO: 143 - protein, 7 aa, Artificial Sequence
  mAb 15 LCDR2
NAKTLAD

SEQ ID NO: 144 - protein, 9 aa, Artificial Sequence
  mAb 15 LCDR3
QHFWSTPWT

SEQ ID NO: 145 - protein, 112 aa, Artificial Sequence
  mAb 16 VL
DVVMTQTPLTLSVTIGQPASISCKSSQSLLDSDGKTYLKWLLQRPGQSPKRLIYLVSKLDSGVPDRFTGSGSGTDFTLKISRVEAEDLGVYYCWQGTHFPWTFGGGTKLEIK

SEQ ID NO: 146 - protein, 16 aa, Artificial Sequence
  mAb 16 LCDR1
KSSQSLLDSDGKTYLK

SEQ ID NO: 147 - protein, 7 aa, Artificial Sequence
  mAb 16 LCDR2
LVSKLDS

SEQ ID NO: 148 - protein, 9 aa, Artificial Sequence
  mAb 16 LCDR3
WQGTHFPWT

SEQ ID NO: 149 - protein, 112 aa, Artificial Sequence
  mAb 17 VL
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGAGTKLELK

SEQ ID NO: 150 - protein, 16 aa, Artificial Sequence
  mAb 17 LCDR1
RSSKSLLHSNGNTYLY

SEQ ID NO: 151 - protein, 7 aa, Artificial Sequence
  mAb 17 LCDR2
RMSNLAS

SEQ ID NO: 152 - protein, 9 aa, Artificial Sequence
  mAb 17 LCDR3
MQHLEYPFT

SEQ ID NO: 153 - protein, 107 aa, Artificial Sequence
  mAb 18 VL
TIVMTQTPKFLLVSAGDRVTITCKASQSVSNAVAWYQQKPGQSPKLLIYSASNRYTGVPDRFTGSGYGTDFTFTISSVQAEDLAVYFCQQDYTSPFTFGSGTKLEIK

SEQ ID NO: 154 - protein, 11 aa, Artificial Sequence
  mAb 18 LCDR1
KASQSVSNAVA

SEQ ID NO: 155 - protein, 7 aa, Artificial Sequence
  mAb 18 LCDR2
SASNRYT

SEQ ID NO: 156 - protein, 9 aa, Artificial Sequence
  mAb 18 LCDR3
QQDYTSPFT

SEQ ID NO: 157 - protein, 106 aa, Artificial Sequence
  mAb 19 VL
QIVLTQSPAIMSASPGEKVTMSCSASSSVTYMHWYQQKSGTSPKRWIYDTSKLASGVPARFSGSGSGTSYSLTISSMEAEDAATYYCQQWSSNPPTFGGGTKLEIK

SEQ ID NO: 158 - protein, 10 aa, Artificial Sequence
  mAb 19 LCDR1
SASSSVTYMH

SEQ ID NO: 159 - protein, 7 aa, Artificial Sequence
  mAb 19 LCDR2
DTSKLAS

SEQ ID NO: 160 - protein, 9 aa, Artificial Sequence
  mAb 19 LCDR3
QQWSSNPPT

SEQ ID NO: 161 - protein, 111 aa, Artificial Sequence
  mAb 20 VL
NIVLTQSPASLAVSLGQRATISCRASESVDSYGNSFMHWYQQKPGQPPKLLIYLASNLESGVPARFSGSGSRTDFTLTIDPVEADDAATYYCQQNNEDPWTFGGGTKLEIK

SEQ ID NO: 162 - protein, 15 aa, Artificial Sequence
  mAb 20 LCDR1
RASESVDSYGNSFMH

SEQ ID NO: 163 - protein, 7 aa, Artificial Sequence
  mAb 20 LCDR2
LASNLES

SEQ ID NO: 164 - protein, 9 aa, Artificial Sequence
  mAb 20 LCDR3
QQNNEDPWT

SEQ ID NO: 165 - protein, 112 aa, Artificial Sequence
  mAb 24 VL
NIMMTQSPSSLAVSAGEKVTMSCKSSQSVLYSSNQKNYLAWYQQKPGQSPKLLIYWASTRESGVPDRFTGSGSGTDFTLTISSVQAEDLAVYYCHQYLSSYTFGGGTKLEIK

SEQ ID NO: 166 - protein, 17 aa, Artificial Sequence
  mAb 24 LCDR1
KSSQSVLYSSNQKNYLA

SEQ ID NO: 167 - protein, 7 aa, Artificial Sequence
  mAb 24 LCDR2
WASTRES

SEQ ID NO: 168 - protein, 7 aa, Artificial Sequence
  mAb 24 LCDR3
HQYLSSY

SEQ ID NO: 169 - protein, 108 aa, Artificial Sequence
  mAb 34 VL
ENVLTQSPAIMSASPGEKVTMTCRASSSVSSSYLHWYQQKSGASPKLWIYSTSNLASGVPARFSGSGSGTSYSLTISSVEAEDAATYYCQQYSGYPLTFGAGTKLELK

SEQ ID NO: 170 - protein, 12 aa, Artificial Sequence
  mAb 34 LCDR1
RASSSVSSSYLH

SEQ ID NO: 171 - protein, 7 aa, Artificial Sequence
  mAb 34 LCDR2
STSNLAS

SEQ ID NO: 172 - protein, 9 aa, Artificial Sequence
  mAb 34 LCDR3
QQYSGYPLT

SEQ ID NO: 173 - protein, 112 aa, Artificial Sequence
  mAb 35, 36 VL
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGSGTKLEIK

SEQ ID NO: 174 - protein, 16 aa, Artificial Sequence
  mAb 35, 36 LCDR1
RSSKSLLHSNGNTYLY

SEQ ID NO: 175 - protein, 7 aa, Artificial Sequence
  mAb 35, 36 LCDR2
RMSNLAS

SEQ ID NO: 176 - protein, 9 aa, Artificial Sequence
  mAb 35, 36 LCDR3
MQHLEYPFT

SEQ ID NO: 177 - protein, 111 aa, Artificial Sequence
  mAb 37 VL
DIVLTQSPASLAVSLGQRATISCRASKSVSTSGYSYMHWYQQKPGQPPKLLIYLASNLESGVPARFSGSGSGTDFTLNIHPVEEEDASTYYCHHSRELPRTFGGGTKLEIK

SEQ ID NO: 178 - protein, 15 aa, Artificial Sequence
  mAb 37 LCDR1
RASKSVSTSGYSYMH

SEQ ID NO: 179 - protein, 7 aa, Artificial Sequence
  mAb 37 LCDR2
LASNLES

SEQ ID NO: 180 - protein, 9 aa, Artificial Sequence
  mAb 37 LCDR3
HHSRELPRT

SEQ ID NO: 181 - protein, 112 aa, Artificial Sequence
  mAb 39 VL
DVVVTQTPLSLPVSFGDQVSISCRSSQSLANSYGNTYLSWYLHKPGQSPQLLIYGISNRFSGVPDRFSGSGSGTDFTLKISTIKPEDLGMYYCLQGTHQPWTFGGGTKLEIK

SEQ ID NO: 182 - protein, 16 aa, Artificial Sequence
  mAb 39 LCDR1
RSSQSLANSYGNTYLS

SEQ ID NO: 183 - protein, 7 aa, Artificial Sequence
  mAb 39 LCDR2
GISNRFS

SEQ ID NO: 184 - protein, 9 aa, Artificial Sequence
  mAb 39 LCDR3
LQGTHQPWT

SEQ ID NO: 185 - protein, 107 aa, Artificial Sequence
  mAb 40 VL
DIQMTQSPASLSASVGETVTITCRASGNIHNYLAWYQQKQGKSPQLLVYNAKTLADGVPSRFSGSGSGTQYSLKINSLQPEDFGSYYCQHFWSTPYTFGGGTKLEIK

SEQ ID NO: 186 - protein, 11 aa, Artificial Sequence
  mAb 40 LCDR1
RASGNIHNYLA

SEQ ID NO: 187 - protein, 7 aa, Artificial Sequence
  mAb 40 LCDR2
NAKTLAD

SEQ ID NO: 188 - protein, 9 aa, Artificial Sequence
  mAb 40 LCDR3
QHFWSTPYT

SEQ ID NO: 189 - protein, 111 aa, Artificial Sequence
  mAb 41 VL
DIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPWTFGGGTKLEIK

SEQ ID NO: 190 - protein, 15 aa, Artificial Sequence
  mAb 41 LCDR1
KASQSVDYDGDSYMN

SEQ ID NO: 191 - protein, 7 aa, Artificial Sequence
  mAb 41 LCDR2
AASNLES

SEQ ID NO: 192 - protein, 9 aa, Artificial Sequence
  mAb 41 LCDR3
QQSNEDPWT

SEQ ID NO: 193 - protein, 112 aa, Artificial Sequence
  mAb 42 - L1 VL
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPWTFGGGTKLEIK

SEQ ID NO: 194 - protein, 16 aa, Artificial Sequence
  mAb 42 - L1 LCDR1
KSSQSLLDSDGKTYLN

SEQ ID NO: 195 - protein, 7 aa, Artificial Sequence
  mAb 42 - L1 LCDR2
LVSKLDS

SEQ ID NO: 196 - protein, 9 aa, Artificial Sequence
  mAb 42 - L1 LCDR3
WQGTHFPWT

SEQ ID NO: 197 - protein, 107 aa, Artificial Sequence
  mAb 42 - L2 VL
DIVLTQSPATLSVTPGDRVSLSCRASQSISNYLHWYQQKSHESPRLLIKYASQSISGIPSRFSGSGSGTDFTLSINSVETEDFGMYFCQQSNSWPFTFGSGTKLEIK

SEQ ID NO: 198 - protein, 11 aa, Artificial Sequence
  mAb 42 - L2 LCDR1
RASQSISNYLH

SEQ ID NO: 199 - protein, 7 aa, Artificial Sequence
  mAb 42 - L2 LCDR2
YASQSIS

SEQ ID NO: 200 - protein, 9 aa, Artificial Sequence
  mAb 42 - L2 LCDR3
QQSNSWPFT

SEQ ID NO: 201 - protein, 113 aa, Artificial Sequence
  mAb 43 VL
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYSCKQSYNLPLTFGAGTKLELK

SEQ ID NO: 202 - protein, 17 aa, Artificial Sequence
  mAb 43 LCDR1
KSSQSLLNSRTRKNYLA

SEQ ID NO: 203 - protein, 7 aa, Artificial Sequence
  mAb 43 LCDR2
WASTRES

SEQ ID NO: 204 - protein, 9 aa, Artificial Sequence
  mAb 43 LCDR3
KQSYNLPLT

SEQ ID NO: 205 - protein, 106 aa, Artificial Sequence
  mAb 44 VL
QIVLTQSPAIMSASPGEKVTMTCSASSSISYMHWYQQKPGTSPKRWIYDTSKLASGVPARFSGSGSGTSYSLTISSMEAEDAATYYCHQRSSYPYTFGGGTKLEIK

SEQ ID NO: 206 - protein, 10 aa, Artificial Sequence
  mAb 44 LCDR1
SASSSISYMH

SEQ ID NO: 207 - protein, 7 aa, Artificial Sequence
  mAb 44 LCDR2
DTSKLAS

SEQ ID NO: 208 - protein, 9 aa, Artificial Sequence
  mAb 44 LCDR3
HQRSSYPYT

SEQ ID NO: 209 - protein, 111 aa, Artificial Sequence
  mAb 45 VL
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPYTFGGGTKLEIK

SEQ ID NO: 210 - protein, 15 aa, Artificial Sequence
  mAb 45 LCDR1
RASKSVSTSGYSYMH

SEQ ID NO: 211 - protein, 7 aa, Artificial Sequence
  mAb 45 LCDR2
LASNLES

SEQ ID NO: 212 - protein, 9 aa, Artificial Sequence
  mAb 45 LCDR3
QHSRELPYT

SEQ ID NO: 213 - protein, 112 aa, Artificial Sequence
  mAb 47 VL
NIVLTQSPASLAVSLGQRATISCRASESVDSFGNSFMHWYQQKPGQPPKLLIYLASNLESGVPARFSGSGSRTDFTLTIDPVEADDAATYYCQQNNEDPPWTFGGGTKLEIK

SEQ ID NO: 214 - protein, 15 aa, Artificial Sequence
  mAb 47 LCDR1
RASESVDSFGNSFMH

SEQ ID NO: 215 - protein, 7 aa, Artificial Sequence
  mAb 47 LCDR2
LASNLES

SEQ ID NO: 216 - protein, 10 aa, Artificial Sequence
  mAb 47 LCDR3
QQNNEDPPWT

SEQ ID NO: 217 - protein, 107 aa, Artificial Sequence
  mAb 48 VL
DILLTQSPAILSVSPGERVSFSCRASQSIGTSIHWYQQGTNGSPRLLIKFASESISGIPSRFSGSGSGTDFTLSINSVESEDIADYYCQQSNSWPLTFGAGTKLELK

SEQ ID NO: 218 - protein, 11 aa, Artificial Sequence
  mAb 48 LCDR1
RASQSIGTSIH

SEQ ID NO: 219 - protein, 7 aa, Artificial Sequence
  mAb 48 LCDR2
FASESIS

SEQ ID NO: 220 - protein, 9 aa, Artificial Sequence
  mAb 48 LCDR3
QQSNSWPLT

SEQ ID NO: 221 - DNA, 357 bp, Artificial Sequence
  mAb 2 VH
caggtccagctgcagcagtctggagctgagctggtaaggcctgggacttcagtgaaggtgtcctgcaaggcttctggatacgccttcactaattacttgatagagtgggtaaagcagaggcctggacagggccttgagtggattggagtgattaatcctggaagtggtggtactaactacaatgagaagttcaagggcaaggcaacactgactgcagacaaatcctccagcactgcctacatgcagctcagcagcctgacatctgatgactctgcggtctatttctgtgcgagatcacggggttacccctatgctatggactactggggtcaaggaacctcagtcaccgtctcctca

SEQ ID NO: 222 - DNA, 15 bp, Artificial Sequence
  mAb 2 HCDR1
aattacttgatagag

SEQ ID NO: 223 - DNA, 51 bp, Artificial Sequence
  mAb 2 HCDR2
gtgattaatcctggaagtggtggtactaactacaatgagaagttcaagggc

SEQ ID NO: 224 - DNA, 30 bp, Artificial Sequence
  mAb 2 HCDR3
tcacggggttacccctatgctatggactac

SEQ ID NO: 225 - DNA, 354 bp, Artificial Sequence
  mAb 3, 21 VH
gaggtccagctgcaacagtctggacctgagctggtgaagcctggggcttcagtgaagatatcctgcaagacctctggatacacattcactgaatacaccatgcactgggtgaagcagagccatggaaagagccttgagtggattggaggtattaatcctaacaatggtggtactagctacaaccagaagttcaagggcaaggccacattgactgtagacaagtcctccagcacagcctacatggagctccgcagcctgacatctgaggattctgcagtctattactgtgcaagatcgttagtagagggggactttgcttactggggccaagggactctggtcactgtctctgca

SEQ ID NO: 226 - DNA, 15 bp, Artificial Sequence
  mAb 3, 21 HCDR1
gaatacaccatgcac

SEQ ID NO: 227 - DNA, 51 bp, Artificial Sequence
  mAb 3, 21 HCDR2
ggtattaatcctaacaatggtggtactagctacaaccagaagttcaagggc

SEQ ID NO: 228 - DNA, 27 bp, Artificial Sequence
  mAb 3, 21 HCDR3
tcgttagtagagggggactttgcttac

SEQ ID NO: 229 - DNA, 372 bp, Artificial Sequence
  mAb 5 VH
gaggtgaagctggtggaatctggaggaggcttggtacagcctgggggttctctgagactctcctgtgcaacttctgggttcaccttcagtgatttctacatggagtgggtccgccagcctccagggaagagactggagtggattgctgcaagtagaaacaaagctaatgattatacaacagagtacagtgcatctgtgaagggtcggttcatcgtctccagagacacttcccaaagcatcctctaccttcagatgaatgccctgagagctgaggacactgccatttattactgtgcaagagatttcctcccttactacggtagtccctggtttgcttactggggccaagggactctggtcactgtctctgca

SEQ ID NO: 230 - DNA, 15 bp, Artificial Sequence
  mAb 5 HCDR1
gatttctacatggag

SEQ ID NO: 231 - DNA, 57 bp, Artificial Sequence
  mAb 5 HCDR2
gcaagtagaaacaaagctaatgattatacaacagagtacagtgcatctgtgaagggt

SEQ ID NO: 232 - DNA, 39 bp, Artificial Sequence
  mAb 5 HCDR3
gatttcctcccttactacggtagtccctggtttgcttac

SEQ ID NO: 233 - DNA, 378 bp, Artificial Sequence
  mAb 9 VH
gaggtccagctgcagcagtctggacctgagctggagaagcctggcgcttcagtgaagatatcctgcaaggcttctggttactcattcactggctacaacatgaactgggtgaagcagagcaatggaaagagccttgagtggattggaaatattgatccttactatggtggtactagctacaaccagaagttcaagggcaaggccacattgactctagacaaatcctccagcacagcctacatgcagctcaagagcctgacatctgaggactctgcagtctattactgtgcaagatcgcggtttattactacggtagtagctaccgattactatgctatggactactggggtcaaggaacctcagtcaccgtctcctca

SEQ ID NO: 234 - DNA, 15 bp, Artificial Sequence
  mAb 9 HCDR1
ggctacaacatgaac

SEQ ID NO: 235 - DNA, 51 bp, Artificial Sequence
  mAb 9 HCDR2
aatattgatccttactatggtggtactagctacaaccagaagttcaagggc

SEQ ID NO: 236 - DNA, 51 bp, Artificial Sequence
  mAb 9 HCDR3
tcgcggtttattactacggtagtagctaccgattactatgctatggactac

SEQ ID NO: 237 - DNA, 363 bp, Artificial Sequence
  mAb 10 VH
gaggtgaagctggtggaatctggaggaggcttggtacagcctgggggttctctgagactctcctgtgcaacttctgggttcaccttcagtgatttctacatggagtgggtccgccagcctccagggaagagactggagtggattgctgcaagtagaaacaaagctaatgattatacaacagagtacagtgcatctgtgaagggtcggttcatcgtctccagagacacttcccaaagcatcctctaccttcagatgaatgccctgagagctgaggacactgccatttattactgtgcaagagagaactatggtaaccccgctatggactactggggtcaaggaacctcagtcaccgtctcctca

SEQ ID NO: 238 - DNA, 15 bp, Artificial Sequence
  mAb 10 HCDR1
gatttctacatggag

SEQ ID NO: 239 - DNA, 57 bp, Artificial Sequence; identical to SEQ ID NO: 231
  mAb 10 HCDR2

SEQ ID NO: 240 - DNA, 30 bp, Artificial Sequence
  mAb 10 HCDR3
gagaactatggtaaccccgctatggactac

SEQ ID NO: 241 - DNA, 363 bp, Artificial Sequence
  mAb 12, 13 VH
caggtgcagctgaaggagtcaggacctggcctggtggcgccctcacagagcctgtccatcacttgcactgtctctgggttttcattaaccagctatggtgtacactgggttcgccagcctccaggaaagggtctggagtggctgggagtaatatgggctggtggaagcacaaattataattcggctctcatgtccagactgagcatcagcaaagacaactccaagagccaagttttcttaaaaatgaacagtctgcaaactgatgacacagccatgtacttctgtgccagaggggggattacgtcccttcataactatgttatggactactggggtcaaggaacctcagtcaccgtctcctca

SEQ ID NO: 242 - DNA, 15 bp, Artificial Sequence
  mAb 12, 13 HCDR1
agctatggtgtacac

SEQ ID NO: 243 - DNA, 48 bp, Artificial Sequence
  mAb 12, 13 HCDR2
gtaatatgggctggtggaagcacaaattataattcggctctcatgtcc

SEQ ID NO: 244 - DNA, 39 bp, Artificial Sequence
  mAb 12, 13 HCDR3
ggggggattacgtcccttcataactatgttatggactac

SEQ ID NO: 245 - DNA, 375 bp, Artificial Sequence
  mAb 14 VH
caggttcagctgcagcagtctggagctgagctgatgaagcctggggcctcagtgaagatatcctgcaaggctactggctacacattcagtagctactggatagagtgggtaaagcagaggcctggacatggccttgagtggattggagagattttacctggaagtggtagtactaactacaatgagaagttcaagggcaaggccacattcactgcagatacatcctccaacacagcctacatgcaactcagcagcctgacatctgaggactctgccgtctattactgtgcaagtgggagtgcaggtaattactacggtagtagctacgaggctatggactactggggtcaaggaacctcagtcaccgtctcctca

SEQ ID NO: 246 - DNA, 15 bp, Artificial Sequence
  mAb 14 HCDR1
agctactggatagag

SEQ ID NO: 247 - DNA, 51 bp, Artificial Sequence
  mAb 14 HCDR2
gagattttacctggaagtggtagtactaactacaatgagaagttcaagggc

SEQ ID NO: 248 - DNA, 48 bp, Artificial Sequence
  mAb 14 HCDR3
gggagtgcaggtaattactacggtagtagctacgaggctatggactac

SEQ ID NO: 249 - DNA, 363 bp, Artificial Sequence
  mAb 15 VH
caggtgcagctgaaggagtcaggacctggcctggtggcgccctcacagagcctgtccatcacatgcactgtctcagggttctcattaaccagctatggtgtaagctgggttcgccagcctccaggaaagggtctggagtggctgggagtaatatggggtgacgggagcacaaattatcattcagctctcatatccagactgagcatcagcaaggataactccaagagccaagttttcttaaaactgaacagtctgcaaactgatgacacagccacgtactactgtgccaaaccggatctctactatgattctccctatgctatggactactggggtcaaggaacctcagtcaccgtctcctca

SEQ ID NO: 250 - DNA, 15 bp, Artificial Sequence
  mAb 15 HCDR1
agctatggtgtaagc

SEQ ID NO: 251 - DNA, 48 bp, Artificial Sequence
  mAb 15 HCDR2
gtaatatggggtgacgggagcacaaattatcattcagctctcatatcc

SEQ ID NO: 252 - DNA, 39 bp, Artificial Sequence
  mAb 15 HCDR3
ccggatctctactatgattctccctatgctatggactac

SEQ ID NO: 253 - DNA, 342 bp, Artificial Sequence
  mAb 16 VH
gaggtgaagcttctcgagtctggaggtggcctggtgcagcctggaggatccctgaaactctcctgtgcagcctcaggattcgattttagtagatactggatgagttgggtccggcaggctccagggaaagggctagaatggattggagaaattaatccagatagcagtacgataaactatacgccatctctaaaggataaattcatcatctccagagacaacgccaaaaatacgctgtacctgcaaatgagcaaagtgagatctgaggacacagccctttattactgtgcaagaccccttatggactactggggtcaaggaacctcagtcaccgtctcctca

SEQ ID NO: 254 - DNA, 15 bp, Artificial Sequence
  mAb 16 HCDR1
agatactggatgagt

SEQ ID NO: 255 - DNA, 51 bp, Artificial Sequence
  mAb 16 HCDR2
gaaattaatccagatagcagtacgataaactatacgccatctctaaaggat

SEQ ID NO: 256 - DNA, 15 bp, Artificial Sequence
  mAb 16 HCDR3
ccccttatggactac

SEQ ID NO: 257 - DNA, 360 bp, Artificial Sequence
  mAb 17 VH
caggtgcagctgaaggagtcaggacctggcctggtggcgccctcacagagcctgtccatcacttgcactgtctctgggttttcattaaccagctatggtgtacactgggttcgccagcctccaggaaagggtctggagtggctgggagtaatatgggctggtggaagctcaaattataattcggctctcatgtccagactgagcatcagcaaagacaactccaagagccaagttttcttaaaaatgaacagtctgcaaactgatgacacagccatgtactactgtgccagagatggctacggctacgtttactatgctatggactactggggtcaaggaacctcagtcaccgtctcctca

SEQ ID NO: 258 - DNA, 15 bp, Artificial Sequence
  mAb 17 HCDR1
agctatggtgtacac

SEQ ID NO: 259 - DNA, 48 bp, Artificial Sequence
  mAb 17 HCDR2
gtaatatgggctggtggaagctcaaattataattcggctctcatgtcc

SEQ ID NO: 260 - DNA, 36 bp, Artificial Sequence
  mAb 17 HCDR3
gatggctacggctacgtttactatgctatggactac

SEQ ID NO: 261 - DNA, 345 bp, Artificial Sequence
  mAb 18 VH
caggtccagctgcagcagtctggagctgagctggtaaggcctgggacttcagtgaagatgtcctgcaaggctgctggatacaccttcactaactattggataggttgggtaaagcagaggcctggacatggccttgagtggattggagatatttaccctggaggtggttatactaagtacaatgagaagttcaagggcaaggccacactgactgcagacacatcctccagtacagcctacatgcagctcagcagcctgacatctgaggactctgccatctattactgtgcaagagacggttactccccctactggggccaaggcacctctctcacagtctcctca

SEQ ID NO: 262 - DNA, 15 bp, Artificial Sequence
  mAb 18 HCDR1
aactattggataggt

SEQ ID NO: 263 - DNA, 51 bp, Artificial Sequence
  mAb 18 HCDR2
gatatttaccctggaggtggttatactaagtacaatgagaagttcaagggc

SEQ ID NO: 264 - DNA, 18 bp, Artificial Sequence
  mAb 18 HCDR3
gacggttactccccctac

SEQ ID NO: 265 - DNA, 366 bp, Artificial Sequence
  mAb 19 VH
caggtccagctgcagcagtctggggctgagctggtgaggcctggggtctcagtgaagatttcctgcaagggttctggctacacattcactgattatgctatgcactgggtgaagcagagtcatgcaaagagtctagagtggattggagttattagtacttactatggtgatgctaggtacaaccagaagttcaagggcaaggccacaatgactgtagacaaatcctccagcacagcctatatggaacttgccagactgacatctgaggattctgccatctattactgtgcaagacgggggattacggtagtagccgactatgctatggactactggggtcaaggaacctcagtcaccgtctcctca

SEQ ID NO: 266 - DNA, 15 bp, Artificial Sequence
  mAb 19 HCDR1
gattatgctatgcac

SEQ ID NO: 267 - DNA, 51 bp, Artificial Sequence
  mAb 19 HCDR2
gttattagtacttactatggtgatgctaggtacaaccagaagttcaagggc

SEQ ID NO: 268 - DNA, 39 bp, Artificial Sequence
  mAb 19 HCDR3
cgggggattacggtagtagccgactatgctatggactac

SEQ ID NO: 269 - DNA, 360 bp, Artificial Sequence
  mAb 20 VH
caggtccagctgcagcagtctggagctgagctggtaaggcctgggacttcagtgaaggtgtcctgcaaggcttctggatacgccttcactaattacttgatagagtgggtaaagcagaggcctggacagggccttgagtggattggagtgattaatcctggaagtggtggtactaactacaatgagaagttcaagggcaaggcaacactgactgcagacaaatcctccagcactgcctacatgcagctcagcagcctgacatctgatgactctgcggtttatttctgtgcaagatcgcactatggtaactactatgctatggactactggggtcaaggaacctcagtcaccgtctcctca

SEQ ID NO: 270 - DNA, 15 bp, Artificial Sequence
  mAb 20 HCDR1
aattacttgatagag

SEQ ID NO: 271 - DNA, 51 bp, Artificial Sequence; identical to SEQ ID NO: 223
  mAb 20 HCDR2

SEQ ID NO: 272 - DNA, 33 bp, Artificial Sequence
  mAb 20 HCDR3
tcgcactatggtaactactatgctatggactac

SEQ ID NO: 273 - DNA, 372 bp, Artificial Sequence
  mAb 24 VH
gaggtccagctgcaacagtctggacctgagctggtgaagcctggggcttcagtgaagatgtcctgcaaggcttctggatacaccttcactgactactacatgaagtgggtgaagcagagccatggaaagagccttgagtggattggagatattaatcctaacaatggtgatactttctacaaccagaagttcaagggcaaggccacattgactgtagacaaatcctccagcacagcctacatgcagctcaacagcctgacatctgaggactctgcagtctattactgtgcaagaagaggtttttattactacggtagtagagggggtattatggactactggggtcaaggaacctcagtcaccgtctcctca

SEQ ID NO: 274 - DNA, 15 bp, Artificial Sequence
  mAb 24 HCDR1
gactactacatgaag

SEQ ID NO: 275 - DNA, 51 bp, Artificial Sequence
  mAb 24 HCDR2
gatattaatcctaacaatggtgatactttctacaaccagaagttcaagggc

SEQ ID NO: 276 - DNA, 45 bp, Artificial Sequence
  mAb 24 HCDR3
agaggtttttattactacggtagtagagggggtattatggactac

SEQ ID NO: 277 - DNA, 354 bp, Artificial Sequence
  mAb 34 VH
gaagtgaagctggtggagtctgggggaggcttagtgcagcctggagggtccctgaaactctcctgtgcaacctctggattcactttcagtgactattacatgtattgggttcgccagactccagagaagaggctggagtgggtcgcatacattagtaatggtggtgatagcacctattatccagacactgtaaagggccgattcaccatctccagagacaatgccaagaacaccctgtacctgcaaatgagccgtctgaagtctgaggacacagccatgtattactgtgcaagatcctactatggtaacccttttccttactggggccaggggactctggtcactgtctctgca

SEQ ID NO: 278 - DNA, 15 bp, Artificial Sequence
  mAb 34 HCDR1
gactattacatgtat

SEQ ID NO: 279 - DNA, 51 bp, Artificial Sequence
  mAb 34 HCDR2
tacattagtaatggtggtgatagcacctattatccagacactgtaaagggc

SEQ ID NO: 280 - DNA, 27 bp, Artificial Sequence
  mAb 34 HCDR3
tcctactatggtaacccttttccttac

SEQ ID NO: 281 - DNA, 360 bp, Artificial Sequence
  mAb 35, 36 VH
caggtgcagctgaaggagtcaggacctggcctggtggcgccctcacagagcctgtccatcacttgcactgtctctgggttttcattaaccagctatggtgtacactgggttcgccagcctccaggaaagggtctggagtggctgggagtaatatgggctggtggaagcacaaattataattcggctctcatgtccagactgagcatcagcaaagacaactccaagagccaagttttcttaaaaatgaacagtctgcaaactgatgacacagccatgtactactgtgccactgatggttacaagtttttttactatgctatggactactggggtcaaggaacctcagtcaccgtctcctca

SEQ ID NO: 282 - DNA, 15 bp, Artificial Sequence
  mAb 35, 36 HCDR1
agctatggtgtacac

SEQ ID NO: 283 - DNA, 48 bp, Artificial Sequence; identical to SEQ ID NO: 243
  mAb 35, 36 HCDR2

SEQ ID NO: 284 - DNA, 36 bp, Artificial Sequence
  mAb 35, 36 HCDR3
gatggttacaagtttttttactatgctatggactac

SEQ ID NO: 285 - DNA, 354 bp, Artificial Sequence
  mAb 37 VH
gaggtccagcttcagcagtcaggacctgagctggtgaaacctggggcctcagtgaagatatcctgcaaggcttctggatacacattcactgactacaacatgcactgggtgaagcagagccatggaaagagccttgagtggattggatatatttatccttacaatggtggtactggctacaaccagaaattcaagaccaaggccacattgactgtagacaattcctccagcacagcctacatggagctccgcagcctgacatctgaggactctgcagtctattactgtgcaatccctgggtattactatgctatggactactggggtcaaggaacctcagtcaccgtctcctca

SEQ ID NO: 286 - DNA, 15 bp, Artificial Sequence
  mAb 37 HCDR1
gactacaacatgcac

SEQ ID NO: 287 - DNA, 51 bp, Artificial Sequence
  mAb 37 HCDR2
tatatttatccttacaatggtggtactggctacaaccagaaattcaagacc

SEQ ID NO: 288 - DNA, 27 bp, Artificial Sequence
  mAb 37 HCDR3
cctgggtattactatgctatggactac

SEQ ID NO: 289 - DNA, 366 bp, Artificial Sequence
  mAb 39 VH
gaagtgaagctggtggagtctgggggaggtttagtgcagcctggagggtccctgaaactctcctgtgcagcctctggattcactttcagtagctataccatgtcttgggttcgccagactccagagaagaggctggagtgggtcgcatacattagtaatggtggtggtagcacctactatccagacactgtaaagggccgattcaccatctccagagacaatgccaagaacaccctgtacctgcaaatgagcagtctgaagtctgaggacacggccatgtattactgtgcaagaggggtatcctactatggtgacctgtactactttgactactggggccaaggcaccactctcacagtctcctca

SEQ ID NO: 290 - DNA, 15 bp, Artificial Sequence
  mAb 39 HCDR1
agctataccatgtct

SEQ ID NO: 291 - DNA, 51 bp, Artificial Sequence
  mAb 39 HCDR2
tacattagtaatggtggtggtagcacctactatccagacactgtaaagggc

SEQ ID NO: 292 - DNA, 39 bp, Artificial Sequence
  mAb 39 HCDR3
ggggtatcctactatggtgacctgtactactttgactac

SEQ ID NO: 293 - DNA, 363 bp, Artificial Sequence
  mAb 40 VH
cagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacacctacactggagagccaacatatgctgatgacttcaagggacggtttgccttctctttggaaacctctgccagcactgcctatttgcagatcaacaacctcaaaaatgaggacacggctacatatttctgtgcaagatccctctatggttacgacgaggggtacttcgatgtctggggcgcagggaccacggtcaccgtctcctca

SEQ ID NO: 294 - DNA, 15 bp, Artificial Sequence
  mAb 40 HCDR1
aactatggaatgaac

SEQ ID NO: 295 - DNA, 51 bp, Artificial Sequence
  mAb 40 HCDR2
tggataaacacctacactggagagccaacatatgctgatgacttcaaggga

SEQ ID NO: 296 - DNA, 36 bp, Artificial Sequence
  mAb 40 HCDR3
tccctctatggttacgacgaggggtacttcgatgtc

SEQ ID NO: 297 - DNA, 363 bp, Artificial Sequence
  mAb 41 VH
caggtccagctgcagcagtctggagctgagctggtaaggcctgggacttcagtgaaggtgtcctgcaaggcttctggatacgccttcactaattacttgatagagtgggtaaagcagaggcctggacagggccttgagtggattggagtgattaatcctggaagtggtggtactaactacaatgagaagttcaagggcaaggcaacactgactgcagacaaatcctccagcactgcctacatgcagctcagcagcctgacatctgatgactctgcggtctatttctgtgcaagatcgggattacgacgggattactatgctatggactactggggtcaaggaacctcagtcaccgtctcctca

SEQ ID NO: 298 - DNA, 15 bp, Artificial Sequence
  mAb 41 HCDR1
aattacttgatagag

SEQ ID NO: 299 - DNA, 51 bp, Artificial Sequence; identical to SEQ ID NO: 223
  mAb 41 HCDR2

SEQ ID NO: 300 - DNA, 36 bp, Artificial Sequence
  mAb 41 HCDR3
tcgggattacgacgggattactatgctatggactac

SEQ ID NO: 301 - DNA, 351 bp, Artificial Sequence
  mAb 42 VJ
caggtgcagctgaaggagtcaggacctggcctggtggcgccctcacagagcctgtccatcacttgcactgtctctgggttttcattaaccagctatggtgtacactgggttcgccagcctccaggaaagggtctggagtggctgggagtaatatgggctggtggaagcacaaattataattcggctctcatgtccagactgagcatcagcaaagacaactccaagagccaagttttcttaaaaatgaacagtctgcaaactgatgacacagccatgtactactgtgccagagagggtaactgggacgattttgactactggggccaaggcaccactctcacagtctcctca

SEQ ID NO: 302 - DNA, 15 bp, Artificial Sequence
  mAb 42 HCDR1
agctatggtgtacac

SEQ ID NO: 303 - DNA, 48 bp, Artificial Sequence; identical to SEQ ID NO: 243
  mAb 42 HCDR2

SEQ ID NO: 304 - DNA, 27 bp, Artificial Sequence
  mAb 42 HCDR3
gagggtaactgggacgattttgactac

SEQ ID NO: 305 - DNA, 366 bp, Artificial Sequence
  mAb 43 VH
gacgtgaagttggtggagtctgggggaggcttagtgaagcctggagggtccctgaaactctcctgtgcagcctctggattcactttcagtagctataccatgtcttgggttcgccagactccggagaagaggctggagtgggtcgcaaccattagtagtggtggtagttacacctactatccagacagtgtgaagggccgattcaccatctccagagacaatgccaagaacaccctgtacctgcaaatgagcagtctgaaggctgaggacacagccatgtattactgtacaagagggggttacggtagtagcttcgagaactactttgactactggggccaaggcaccactctcacagtctcctca

SEQ ID NO: 306 - DNA, 15 bp, Artificial Sequence
  mAb 43 HCDR1
agctataccatgtct

SEQ ID NO: 307 - DNA, 51 bp, Artificial Sequence
  mAb 43 HCDR2
accattagtagtggtggtagttacacctactatccagacagtgtgaagggc

SEQ ID NO: 308 - DNA, 39 bp, Artificial Sequence
  mAb 43 HCDR3
gggggttacggtagtagcttcgagaactactttgactac

SEQ ID NO: 309 - DNA, 366 bp, Artificial Sequence
  mAb 44 VH
gaggtccagctgcagcagtctggacctgagctggagaagcctggcgcttcagtgaagatatcctgcaaggcttctggttactcattcactggctacaacatgaactgggtgaagcagagcaatggaaagagccttgagtggattggaaatattgacccttactatggtggtattaggtacaaccagaagttcaagggcaaggccacattgactgtagacaaatcctccagcacagcctacatgcagctcaagagcctgacatctgaggactctgcagtctattactgtgcaagagaggataggtacctagttcattactatgctatggactactggggtcaaggaacctcagtcaccgtctcctca

SEQ ID NO: 310 - DNA, 15 bp, Artificial Sequence
  mAb 44 HCDR1
ggctacaacatgaac

SEQ ID NO: 311 - DNA, 51 bp, Artificial Sequence
  mAb 44 HCDR2
aatattgacccttactatggtggtattaggtacaaccagaagttcaagggc

SEQ ID NO: 312 - DNA, 39 bp, Artificial Sequence
  mAb 44 HCDR3
gaggataggtacctagttcattactatgctatggactac

SEQ ID NO: 313 - DNA, 366 bp, Artificial Sequence
  mAb 45 VH
gacgtgaagctggtggagtctgggggaggcttagtgaagcctggagggtccctgaaactctcctgtgcagcctctggattcactttcagtagctataccatgtcttgggttcgccagactccggagaagaggctggagtgggtcgcaaccattagtagtggtggtagttacacctactatccagacagtgtgaagggccgattcaccatctccagagacaatgccaagaacaccctgtacctgcaaatgagcagtctgaagtctgaggacacagccatgtattactgtacaagaaactacggtagtcgacccccttactatgctatggactactggggtcaaggaacctcagtcaccgtctcctca

SEQ ID NO: 314 - DNA, 15 bp, Artificial Sequence
  mAb 45 HCDR1
agctataccatgtct

SEQ ID NO: 315 - DNA, 51 bp, Artificial Sequence; identical to SEQ ID NO: 307
  mAb 45 HCDR2

SEQ ID NO: 316 - DNA, 39 bp, Artificial Sequence
  mAb 45 HCDR3
aactacggtagtcgacccccttactatgctatggactac

SEQ ID NO: 317 - DNA, 354 bp, Artificial Sequence
  mAb 47 VH
gaggtgaagctggtggagtctggaggaggcttggtacagcctgggggttctctgagactctcctgtgcaacttctgggttcaccttcactgattactacatgagctgggtccgccagcctccaggaaaggcacttgagtggttgggttttattagaaacaaagctaatggttacacaacagagtacagtgcatctgtgaagggtcggttcaccatctccagagataattcccaaagcatcctctatcttcaaatgaacaccctgagagctgaggacagtgccacttattactgtgcaagagatcgcccgggctttgactactggggccaaggcaccactctcacagtctcctca

SEQ ID NO: 318 - DNA, 15 bp, Artificial Sequence
  mAb 47 HCDR1
gattactacatgagc

SEQ ID NO: 319 - DNA, 57 bp, Artificial Sequence
  mAb 47 HCDR2
tttattagaaacaaagctaatggttacacaacagagtacagtgcatctgtgaagggt

SEQ ID NO: 320 - DNA, 21 bp, Artificial Sequence
  mAb 47 HCDR3
gatcgcccgggctttgactac

SEQ ID NO: 321 - DNA, 387 bp, Artificial Sequence
  mAb 48 VH
caggttactctgaaagagtctggccctgggatattgcagccctcccagaccctcagtctgacttgttctttctctgggttttcactgagcacttctggtatgggtgtgagctggattcgtcagccttcaggaaagggtctggagtggctggcacacatttactgggatgatgacaagcgctataacccatccctgaagagccggctcacaatctccaaggatacctccagcaaccaggtattcctcaagatcaccagtgtggacactgcagatactgccacatactactgtgctcgaagggggttctactataggtacgacggggggcgccgctattactatgctatggactactggggtcaaggaacctcagtcaccgtctcctca

SEQ ID NO: 322 - DNA, 21 bp, Artificial Sequence
  mAb 48 HCDR1
acttctggtatgggtgtgagc

SEQ ID NO: 323 - DNA, 48 bp, Artificial Sequence
  mAb 48 HCDR2
cacatttactgggatgatgacaagcgctataacccatccctgaagagc

SEQ ID NO: 324 - DNA, 57 bp, Artificial Sequence
  mAb 48 HCDR3
agggggttctactataggtacgacggggggcgccgctattactatgctatggactac

SEQ ID NO: 325 - DNA, 333 bp, Artificial Sequence
  mAb 2 VL
gacattgtgctgacccaatctccagcttctttggctgtgtctctagggcagagggccaccatctcctgcaaggccagccaaagtgttgattatgatggtgatagttatatgaactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcagcaaagtaatgaggatccgtacacgttcggaggggggaccaagctggaaataaaa

SEQ ID NO: 326 - DNA, 45 bp, Artificial Sequence
  mAb 2 LCDR1
aaggccagccaaagtgttgattatgatggtgatagttatatgaac

SEQ ID NO: 327 - DNA, 18 bp, Artificial Sequence
  mAb 2 LCDR2
gctgcatccaatctagaa

SEQ ID NO: 328 - DNA, 27 bp, Artificial Sequence
  mAb 2 LCDR3
cagcaaagtaatgaggatccgtacacg

SEQ ID NO: 329 - DNA, 333 bp, Artificial Sequence
  mAb 3, 21 VL
gacattgtgctgacacagtctcctgcttccttagctgtatctctggggcagagggccaccatctcatgcagggccagccaaagtgtcagcacatctagctatagttatatgcactggtaccaacagaaaccaggacagccacccaaactcctcataaagtatgcatccaacctagaatctggggtccctgccaggttcagtggcagtgggtctgggacagacttcaccctcaacatccatcctgtggaggaggaggatactgcaacatattactgtcagcacagttgggagattccgtacacgttcggaggggggaccaagctggaaataaaa

SEQ ID NO: 330 - DNA, 45 bp, Artificial Sequence
  mAb 3, 21 LCDR1
agggccagccaaagtgtcagcacatctagctatagttatatgcac

SEQ ID NO: 331 - DNA, 21 bp, Artificial Sequence
  mAb 3, 21 LCDR2
tatgcatccaacctagaatct

SEQ ID NO: 332 - DNA, 27 bp, Artificial Sequence
  mAb 3, 21 LCDR3
cagcacagttgggagattccgtacacg

SEQ ID NO: 333 - DNA, 321 bp, Artificial Sequence
  mAb 5 VL
gacattgtgatgactcagtctccagccaccctgtctgtgactccaggagatagagtctctctttcctgcagggccagccagagtattagcgactacttacactggtatcaacaaaaatcacatgagtctccaaggcttctcatcaaatatgcttcccaatccatctctgggatcccctccaggttcagtggcagtggatcagggtcagatttcactctcagtatcaacagtgtggaacctgaagatgttggagtgtattactgtcaaaatggtcacagctttccgtacacgttcggaggggggaccaagctggaaataaaa

SEQ ID NO: 334 - DNA, 33 bp, Artificial Sequence
  mAb 5 LCDR1
agggccagccagagtattagcgactacttacac

SEQ ID NO: 335 - DNA, 21 bp, Artificial Sequence
  mAb 5 LCDR2
tatgcttcccaatccatctct

SEQ ID NO: 336 - DNA, 27 bp, Artificial Sequence
  mAb 5 LCDR3
caaaatggtcacagctttccgtacacg

SEQ ID NO: 337 - DNA, 339 bp, Artificial Sequence
  mAb 9 VL
gacattgtgatgtcacagtctccatcctccctagctgtgtcagttggagagaaggttactatgagctgcaagtccagtcagagccttttatatagtagcaatcaaaagaactacttggcctggtaccagcagaaaccagggcagtctcctaaactgctgatttactgggcatccactagggaatctggggtccctgatcgcttcacaggcagtggatctgggacagatttcactctcaccatcagcagtgtgaaggctgaagacctggcagtttattactgtcagcaatattatagctatcctcccacgttcggaggggggaccaagctggaaataaaa

SEQ ID NO: 338 - DNA, 48 bp, Artificial Sequence
  mAb 9 LCDR1
aagtccagtcagagccttttatatagtagcaatcaaaagaactacttg

SEQ ID NO: 339 - DNA, 21 bp, Artificial Sequence
  mAb 9 LCDR2
tgggcatccactagggaatct

SEQ ID NO: 340 - DNA, 27 bp, Artificial Sequence
  mAb 9 LCDR3
cagcaatattatagctatcctcccacg

SEQ ID NO: 341 - DNA, 333 bp, Artificial Sequence; identical to SEQ ID NO: 325
  mAb 10 VL

SEQ ID NO: 342 - DNA, 45 bp, Artificial Sequence; identical to SEQ ID NO: 326
  mAb 10 LCDR1

SEQ ID NO: 343 - DNA, 21 bp, Artificial Sequence
  mAb 10 LCDR2
gctgcatccaatctagaatct

SEQ ID NO: 344 - DNA, 27 bp, Artificial Sequence
  mAb 10 LCDR3
cagcaaagtaatgaggatccgtacacg

SEQ ID NO: 345 - DNA, 333 bp, Artificial Sequence
  mAb 12,13 VL
gacattgtgctcacccaatctccagcttctttggctgtgtctctagggcagagagccaccatctcctgcagagccagtgaaagtgttgaatattatggcacaagtttaatgcagtggtaccaacagaaaccaggacagccacccaaactcctcatctatgctgcatccaacgtagaatctggggtccctgccaggtttagtggcagtgggtctgggacacacttcagcctcaacatccatcctgtggaggaggatgatattgcaatgtatttctgtcagcaaagtaggaaggttccttcgacgttcggtggaggcaccaagctggaaatcaaa

SEQ ID NO: 346 - DNA, 45 bp, Artificial Sequence
  mAb 12, 13 LCDR1
agagccagtgaaagtgttgaatattatggcacaagtttaatgcag

SEQ ID NO: 347 - DNA, 21 bp, Artificial Sequence
  mAb 12, 13 LCDR2
gctgcatccaacgtagaatct

SEQ ID NO: 348 - DNA, 27 bp, Artificial Sequence
  mAb 12, 13 LCDR3
cagcaaagtaggaaggttccttcgacg

SEQ ID NO: 349 - DNA, 333 bp, Artificial Sequence
  mAb 14 VL
gacattgtgctgacacagtctcctgcttccttagctgtatctctggggcagagggccaccatctcatgcagggccagcaaaagtgtcagtacatctggctatagttatatgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttcctctcacgttcggtgctgggaccaagctggagctgaaa

SEQ ID NO: 350 - DNA, 45 bp, Artificial Sequence
  mAb 14 LCDR1
agggccagcaaaagtgtcagtacatctggctatagttatatgcac

SEQ ID NO: 351 - DNA, 21 bp, Artificial Sequence
  mAb 14 LCDR2
cttgcatccaacctagaatct

SEQ ID NO: 352 - DNA, 27 bp, Artificial Sequence
  mAb 14 LCDR3
cagcacagtagggagcttcctctcacg

SEQ ID NO: 353 - DNA, 366 bp, Artificial Sequence
  mAb 15 VL
caggtcctggcgttgctgctgctgtggcttacaggtgccagatgtgacatccagatgactcagtctccagcctccctatctgcatctgtgggagaaactgtcaccatcacatgtcgagcaagtgggaatattcacaattatttagcatggtatcagcagaaacagggaaaatctcctcagctcctggtctataatgcaaaaaccttagcagatggtgtgccatcaaggttcagtggcagtggatcaggaacacaatattctctcaagatcaacagcctgcagcctgaagattttgggagttattactgtcaacatttttggagtactccgtggacgttcggtggaggcaccaagctggaaatcaaa

SEQ ID NO: 354 - DNA, 33 bp, Artificial Sequence
  mAb 15 LCDR1
cgagcaagtgggaatattcacaattatttagca

SEQ ID NO: 355 - DNA, 21 bp, Artificial Sequence
  mAb 15 LCDR2
aatgcaaaaaccttagcagat

SEQ ID NO: 356 - DNA, 27 bp, Artificial Sequence
  mAb 15 LCDR3
caacatttttggagtactccgtggacg

SEQ ID NO: 357 - DNA, 336 bp, Artificial Sequence
  mAb 16 VL
gatgttgtgatgacccagactccactcactttgtcggttaccattggacaaccagcctccatctcttgcaagtcaagtcagagcctcttagatagtgatggaaagacatatttgaagtggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttccgtggacgttcggtggaggcaccaagctggaaatcaaa

SEQ ID NO: 358 - DNA, 48 bp, Artificial Sequence
  mAb 16 LCDR1
aagtcaagtcagagcctcttagatagtgatggaaagacatatttgaag

SEQ ID NO: 359 - DNA, 21 bp, Artificial Sequence
  mAb 16 LCDR2
ctggtgtctaaactggactct

SEQ ID NO: 360 - DNA, 27 bp, Artificial Sequence
  mAb 16 LCDR3
tggcaaggtacacattttccgtggacg

SEQ ID NO: 361 - DNA, 336 bp, Artificial Sequence
  mAb 17 VL
gatattgtgatgactcaggctgcaccctctgtacctgtcactcctggagagtcagtatccatctcctgcaggtctagtaagagtctcctgcatagtaatggcaacacttacttgtattggttcctgcagaggccaggccagtctcctcagctcctgatatatcggatgtccaaccttgcctcaggagtcccagacaggttcagtggcagtgggtcaggaactgctttcacactgagaatcagtagagtggaggctgaggatgtgggtgtttattactgtatgcaacatctagaatatcctttcacgttcggtgctgggaccaagctggagctgaaa

SEQ ID NO: 362 - DNA, 48 bp, Artificial Sequence
  mAb 17 LCDR1
aggtctagtaagagtctcctgcatagtaatggcaacacttacttgtat

SEQ ID NO: 363 - DNA, 21 bp, Artificial Sequence
  mAb 17 LCDR2
cggatgtccaaccttgcctca

SEQ ID NO: 364 - DNA, 27 bp, Artificial Sequence
  mAb 17 LCDR3
atgcaacatctagaatatcctttcacg

SEQ ID NO: 365 - DNA, 321 bp, Artificial Sequence
  mAb 18 VL
actattgtgatgacccagactcccaaattcctgcttgtatcagcaggagacagggttaccataacctgcaaggccagtcagagtgtgagtaatgctgtagcttggtaccaacagaagccaggccagtctcctaaactgctgatatactctgcatccaatcgctacactggagtccctgatcgcttcactggcagtggatatgggacggatttcactttcaccatcagctctgtgcaggctgaagacctggcagtttatttctgtcagcaggattatacctctccattcacgttcggctcggggacaaagttggaaataaaa

SEQ ID NO: 366 - DNA, 33 bp, Artificial Sequence
  mAb 18 LCDR1
aaggccagtcagagtgtgagtaatgctgtagct

SEQ ID NO: 367 - DNA, 21 bp, Artificial Sequence
  mAb 18 LCDR2
tctgcatccaatcgctacact

SEQ ID NO: 368 - DNA, 27 bp, Artificial Sequence
  mAb 18 LCDR3
cagcaggattatacctctccattcacg

SEQ ID NO: 369 - DNA, 318 bp, Artificial Sequence
  mAb 19 VL
caaattgttctcacccagtctccagcaatcatgtctgcttctccaggggagaaggtcaccatgtcctgcagtgccagctcaagtgtaacttacatgcactggtaccagcagaagtcaggcacctcccccaaaagatggatttatgacacatccaaactggcttctggagtccctgctcgcttcagtggcagtgggtctgggacctcttactctctcacaatcagcagcatggaggctgaagatgctgccacttattactgccagcagtggagtagtaacccaccgacgttcggtggaggcaccaagctggaaatcaaa

SEQ ID NO: 370 - DNA, 30 bp, Artificial Sequence
  mAb 19 LCDR1
agtgccagctcaagtgtaacttacatgcac

SEQ ID NO: 371 - DNA, 21 bp, Artificial Sequence
  mAb 19 LCDR2
gacacatccaaactggcttct

SEQ ID NO: 372 - DNA, 27 bp, Artificial Sequence
  mAb 19 LCDR3
cagcagtggagtagtaacccaccgacg

SEQ ID NO: 373 - DNA, 333 bp, Artificial Sequence
  mAb 20 VL
aacattgtgctgacccaatctccagcttctttggctgtgtctctagggcagagggccaccatatcctgcagagccagtgaaagtgttgatagttatggcaatagttttatgcactggtaccagcagaaaccaggacagccacccaaactcctcatctatcttgcatccaacctagaatctggggtccctgccaggttcagtggcagtgggtctaggacagacttcaccctcaccattgatcctgtggaggctgatgatgctgcaacctattactgtcagcaaaataatgaggatccgtggacgttcggtggaggcaccaagctggaaatcaaa

SEQ ID NO: 374 - DNA, 45 bp, Artificial Sequence
  mAb 20 LCDR1
agagccagtgaaagtgttgatagttatggcaatagttttatgcac

SEQ ID NO: 375 - DNA, 21 bp, Artificial Sequence
  mAb 20 LCDR2
cttgcatccaacctagaatct

SEQ ID NO: 376 - DNA, 27 bp, Artificial Sequence
  mAb 20 LCDR3
cagcaaaataatgaggatccgtggacg

SEQ ID NO: 377 - DNA, 336 bp, Artificial Sequence
  mAb 24 VL
aacattatgatgacacagtcgccatcatctctggctgtgtctgcaggagaaaaggtcactatgagctgtaagtccagtcaaagtgttttatacagttcaaatcagaagaactacttggcctggtaccagcagaaaccagggcagtctcctaaactgctgatctactgggcatccactagggaatctggtgtccctgatcgcttcacaggcagtggatctgggacagattttactcttaccatcagcagtgtacaagctgaagacctggcagtttattactgtcatcaatacctctcctcgtacacgttcggaggggggaccaagctggaaataaaa

SEQ ID NO: 378 - DNA, 50 bp, Artificial Sequence
  mAb 24 LCDR1
aagtccagtcaaagtgttttatacagttcaaatcagaagaactacttggc

SEQ ID NO: 379 - DNA, 21 bp, Artificial Sequence
  mAb 24 LCDR2
tgggcatccactagggaatct

SEQ ID NO: 380 - DNA, 24 bp, Artificial Sequence
  mAb 24 LCDR3
catcaatacctctcctcgtacacg

SEQ ID NO: 381 - DNA, 324 bp, Artificial Sequence
  mAb 34 VL
gaaaatgtgctcacccagtctccagcaatcatgtctgcatctccaggggaaaaggtcaccatgacctgcagggccagctcaagtgtaagttccagttacttgcactggtaccagcagaagtcaggtgcctcccccaaactctggatttatagcacatccaacttggcttctggagtccctgctcgcttcagtggcagtgggtctgggacctcttactctctcacaatcagcagtgtggaggctgaagatgctgccacttattactgccagcagtacagtggttacccactcacgttcggtgctgggaccaagctggagctgaaa

SEQ ID NO: 382 - DNA, 36 bp, Artificial Sequence
  mAb 34 LCDR1
agggccagctcaagtgtaagttccagttacttgcac

SEQ ID NO: 383 - DNA, 21 bp, Artificial Sequence
  mAb 34 LCDR2
agcacatccaacttggcttct

SEQ ID NO: 384 - DNA, 27 bp, Artificial Sequence
  mAb 34 LCDR3
cagcagtacagtggttacccactcacg

SEQ ID NO: 385 - DNA, 336 bp, Artificial Sequence
  mAb 35, 36 VL
gatattgtgatgactcaggctgcaccctctgtacctgtcactcctggagagtcagtatccatctcctgcaggtctagtaagagtctcctgcatagtaatggcaacacttacttgtattggttcctgcagaggccaggccagtctcctcagctcctgatatatcggatgtccaaccttgcctcaggagtcccagacaggttcagtggcagtgggtcaggaactgctttcacactgagaatcagtagagtggaggctgaggatgtgggtgtttattactgtatgcaacatctagaatatccattcacgttcggctcggggacaaagttggaaataaaa

SEQ ID NO: 386 - DNA, 48 bp, Artificial Sequence; identical to SEQ ID NO: 362
  mAb 35, 36 LCDR1

SEQ ID NO: 387 - DNA, 21 bp, Artificial Sequence
  mAb 35, 36 LCDR2
cggatgtccaaccttgcctca

SEQ ID NO: 388 - DNA, 27 bp, Artificial Sequence
  mAb 35, 36 LCDR3
atgcaacatctagaatatccattcacg

SEQ ID NO: 389 - DNA, 333 bp, Artificial Sequence
  mAb 37 VL
gacattgtgctgacacagtctcctgcttccttagctgtatctctggggcagagggccaccatctcatgcagggccagcaaaagtgtcagtacatctggctatagttatatgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgcttcaacctattactgtcatcacagtagggaacttcctcggacgttcggtggaggcaccaagctggaaatcaaa

SEQ ID NO: 390 - DNA, 45 bp, Artificial Sequence; identical to SEQ ID NO: 350
  mAb 37 LCDR1

SEQ ID NO: 391 - DNA, 21 bp, Artificial Sequence
  mAb 37 LCDR2
cttgcatccaacctagaatct

SEQ ID NO: 392 - DNA, 27 bp, Artificial Sequence
  mAb 37 LCDR3
catcacagtagggaacttcctcggacg

SEQ ID NO: 393 - DNA, 336 bp, Artificial Sequence
  mAb 39 VL
gatgttgtggtgactcaaactccactctccctgcctgtcagctttggagatcaagtttctatctcttgcaggtctagtcagagtcttgcaaacagttatgggaacacctatttgtcttggtacctgcacaagcctggccagtctccacagctcctcatctatgggatttccaacagattttctggggtgccagacaggttcagtggcagtggttcagggacagatttcacactcaagatcagcacaataaagcctgaggacttgggaatgtattactgcttacaaggtacacatcagccgtggacgttcggtggaggcaccaagctggaaatcaaa

SEQ ID NO: 394 - DNA, 48 bp, Artificial Sequence
  mAb 39 LCDR1
aggtctagtcagagtcttgcaaacagttatgggaacacctatttgtct

SEQ ID NO: 395 - DNA, 21 bp, Artificial Sequence
  mAb 39 LCDR2
gggatttccaacagattttct

SEQ ID NO: 396 - DNA, 27 bp, Artificial Sequence
  mAb 39 LCDR3
ttacaaggtacacatcagccgtggacg

SEQ ID NO: 397 - DNA, 321 bp, Artificial Sequence
  mAb 40 VL
gacatccagatgactcagtctccagcctccctatctgcatctgtgggagaaactgtcaccatcacatgtcgagcaagtgggaatattcacaattatttagcatggtatcagcagaaacagggaaaatctcctcagctcctggtctataatgcaaaaaccttagcagatggtgtgccatcaaggttcagtggcagtggatcaggaacacaatattctctcaagatcaacagcctgcagcctgaagattttgggagttattactgtcaacatttttggagtactccgtacacgttcggaggggggaccaagctggaaataaaa

SEQ ID NO: 398 - DNA, 33 bp, Artificial Sequence; identical to SEQ ID NO: 354
  mAb 40 LCDR1

SEQ ID NO: 399 - DNA, 21 bp, Artificial Sequence
  mAb 40 LCDR2
aatgcaaaaaccttagcagat

SEQ ID NO: 400 - DNA, 27 bp, Artificial Sequence
  mAb 40 LCDR3
caacatttttggagtactccgtacacg

SEQ ID NO: 401 - DNA, 333 bp, Artificial Sequence
  mAb 41 VL
gacattgtgctgacccaatctccagcttctttggctgtgtctctagggcagagggccaccatctcctgcaaggccagccaaagtgttgattatgatggtgatagttatatgaactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcagcaaagtaatgaggatccgtggacgttcggtggaggcaccaagctggaaatcaaa

SEQ ID NO: 402 - DNA, 45 bp, Artificial Sequence; identical to SEQ ID NO: 326
  mAb 41 LCDR1

SEQ ID NO: 403 - DNA, 21 bp, Artificial Sequence
  mAb 41 LCDR2
gctgcatccaatctagaatct

SEQ ID NO: 404 - DNA, 27 bp, Artificial Sequence
  mAb 41 LCDR3
cagcaaagtaatgaggatccgtggacg

SEQ ID NO: 405 - DNA, 336 bp, Artificial Sequence
  mAb 42 - L1 VL
gatgttgtgatgacccagactccactcactttgtcggttaccattggacaaccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttccgtggacgttcggtggaggcaccaagctggaaatcaaa

SEQ ID NO: 406 - DNA, 48 bp, Artificial Sequence
  mAb 42 - L1 LCDR1
aagtcaagtcagagcctcttagatagtgatggaaagacatatttgaat

SEQ ID NO: 407 - DNA, 21 bp, Artificial Sequence
  mAb 42 - L1 LCDR2
ctggtgtctaaactggactct

SEQ ID NO: 408 - DNA, 27 bp, Artificial Sequence
  mAb 42 - L1 LCDR3
tggcaaggtacacattttccgtggacg

SEQ ID NO: 409 - DNA, 321 bp, Artificial Sequence
  mAb 42 - L2 VL
gatattgtgctaactcagtctccagccaccctgtctgtgactccaggagatagagtcagtctttcctgcagggccagtcaaagtattagcaactacctacactggtatcaacaaaaatcacatgagtctccaaggcttctcatcaagtatgcttcccagtccatctctgggatcccctccaggttcagtggcagtggatcagggacagatttcactctcagtatcaacagtgtggagactgaagattttggaatgtatttctgtcaacagagtaacagctggccattcacgttcggctcggggacaaagttggaaataaaa

SEQ ID NO: 410 - DNA, 33 bp, Artificial Sequence
  mAb 42 - L2 LCDR1
agggccagtcaaagtattagcaactacctacac

SEQ ID NO: 411 - DNA, 21 bp, Artificial Sequence
  mAb 42 - L2 LCDR2
tatgcttcccagtccatctct

SEQ ID NO: 412 - DNA, 27 bp, Artificial Sequence
  mAb 42 - L2 LCDR3
caacagagtaacagctggccattcacg

SEQ ID NO: 413 - DNA, 339 bp, Artificial Sequence
  mAb 43 VL
gacattgtgatgtcacagtctccatcctccctggctgtgtcagcaggagagaaggtcactatgagctgcaaatccagtcagagtctgctcaacagtagaacccgaaagaactacttggcttggtaccagcagaaaccagggcagtctcccaaactgctgatttactgggcatccactagggaatctggggtccctgatcgcttcacaggcagtggatctgggacagatttcactctcaccatcagcagtgtgcaggctgaagacctggcagtttattcctgcaagcaatcttataatcttccgctcacgttcggtgctgggaccaagctggagctgaaa

SEQ ID NO: 414 - DNA, 51 bp, Artificial Sequence
  mAb 43 LCDR1
aaatccagtcagagtctgctcaacagtagaacccgaaagaactacttggct

SEQ ID NO: 415 - DNA, 21 bp, Artificial Sequence
  mAb 43 LCDR2
tgggcatccactagggaatct

SEQ ID NO: 416 - DNA, 27 bp, Artificial Sequence
  mAb 43 LCDR3
aagcaatcttataatcttccgctcacg

SEQ ID NO: 417 - DNA, 318 bp, Artificial Sequence
  mAb 44 VL
caaattgttctcacccagtctccagcaatcatgtctgcatctccaggggagaaggtcaccatgacctgcagtgccagctcaagtataagttacatgcactggtaccagcagaagccaggcacctcccccaaaagatggatttatgacacatccaaactggcttctggagtccctgctcgcttcagtggcagtgggtctgggacctcttattctctcacaatcagcagcatggaggctgaagatgctgccacttattactgccatcagcggagtagttacccgtacacgttcggaggggggaccaagctggaaataaaa

SEQ ID NO: 418 - DNA, 30 bp, Artificial Sequence
  mAb 44 LCDR1
agtgccagctcaagtataagttacatgcac

SEQ ID NO: 419 - DNA, 21 bp, Artificial Sequence
  mAb 44 LCDR2
gacacatccaaactggcttct

SEQ ID NO: 420 - DNA, 27 bp, Artificial Sequence
  mAb 44 LCDR3
catcagcggagtagttacccgtacacg

SEQ ID NO: 421 - DNA, 333 bp, Artificial Sequence
  mAb 45 VL
gacattgtgctgacacagtctcctgcttccttagctgtatctctggggcagagggccaccatctcatgcagggccagcaaaagtgtcagtacatctggctatagttatatgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccgtacacgttcggaggggggaccaagctggaaataaaa

SEQ ID NO: 422 - DNA, 45 bp, Artificial Sequence; identical to SEQ ID NO: 350
  mAb 45 LCDR1

SEQ ID NO: 423 - DNA, 21 bp, Artificial Sequence
  mAb 45 LCDR2
cttgcatccaacctagaatct

SEQ ID NO: 424 - DNA, 27 bp, Artificial Sequence
  mAb 45 LCDR3
cagcacagtagggagcttccgtacacg

SEQ ID NO: 425 - DNA, 336 bp, Artificial Sequence
  mAb 47 VL
aacattgtgctgacccaatctccagcttctttggctgtgtctctagggcagagggccaccatatcctgcagagccagtgaaagtgttgatagttttggcaatagttttatgcactggtaccagcagaaaccaggacagccacccaaactcctcatctatcttgcatccaacctagaatctggggtccctgccaggttcagtggcagtgggtctaggacagacttcaccctcaccattgatcctgtggaggctgatgatgctgcaacctattactgtcagcaaaataatgaggatcctccgtggacgttcggtggaggcaccaagctggaaatcaaa

SEQ ID NO: 426 - DNA, 45 bp, Artificial Sequence
  mAb 47 LCDR1
agagccagtgaaagtgttgatagttttggcaatagttttatgcac

SEQ ID NO: 427 - DNA, 21 bp, Artificial Sequence
  mAb 47 LCDR2
cttgcatccaacctagaatct

SEQ ID NO: 428 - DNA, 30 bp, Artificial Sequence
  mAb 47 LCDR3
cagcaaaataatgaggatcctccgtggacg

SEQ ID NO: 429 - DNA, 321 bp, Artificial Sequence
  mAb 48 VL
gacatcttgctgactcagtctccagccatcctgtctgtgagtccaggagaaagagtcagtttctcctgcagggccagtcagagcattggcacaagcatacactggtatcagcaaggaacaaatggttctccaaggcttctcataaagtttgcttctgagtctatctctgggatcccttccaggtttagtggcagtggatcagggacagattttactcttagcatcaacagtgtggagtctgaagatattgcagattattactgtcaacaaagtaatagctggccgctcacgttcggtgctgggaccaagctggagctgaaa

SEQ ID NO: 430 - DNA, 33 bp, Artificial Sequence
  mAb 48 LCDR1
agggccagtcagagcattggcacaagcatacac

SEQ ID NO: 431 - DNA, 21 bp, Artificial Sequence
  mAb 48 LCDR2
tttgcttctgagtctatctct

SEQ ID NO: 432 - DNA, 27 bp, Artificial Sequence
  mAb 48 LCDR3
caacaaagtaatagctggccgctcacg

SEQ ID NO: 433 - DNA, 21 bp, Artificial Sequence
  Oligo RT-mHingeG1_rev
gcaaggcttacaaccacaatc

SEQ ID NO: 434 - DNA, 21 bp, Artificial Sequence
  Oligo RT-mHingeG2a_rev
gaggacagggcttgattgtgg

SEQ ID NO: 435 - DNA, 21 bp, Artificial Sequence
  Oligo RT-mHingeG2b_rev
gaggacaggggttgattgttg

SEQ ID NO: 436 - DNA, 22 bp, Artificial Sequence
  Oligo RT-mHingeG2c_rev
ggacaggggttctgtgttatgg

SEQ ID NO: 437 - DNA, 20 bp, Artificial Sequence
  Oligo RT-mHingeG3_rev
ctgggcttgggtattctagg

SEQ ID NO: 438 - DNA, 22 bp, Artificial Sequence
  Oligo RT_mKappaCL_rev
ctcattcctgttgaagctcttg

SEQ ID NO: 439 - DNA, 20 bp, Artificial Sequence
  Oligo RT_mLambda1/4-CL_rev
gcacgggacaaactcttctc

SEQ ID NO: 440 - DNA, 22 bp, Artificial Sequence
  Oligo RT_mLambda2/3-CL_rev
ctgcaggagacagactcttctc

SEQ ID NO: 441 - DNA, 20 bp, Artificial Sequence
  Oligo RACE-5E_mG1_rev
gttagtttgggcagcagatc

SEQ ID NO: 442 - DNA, 19 bp, Artificial Sequence
  Oligo RACE-5E_mG2a.2c_rev
gaggagccagttgtayctc

SEQ ID NO: 443 - DNA, 21 bp, Artificial Sequence
  Oligo RACE-5E_mG2b_rev
gaaccagttgtatctccacac

SEQ ID NO: 444 - DNA, 19 bp, Artificial Sequence
  Oligo RACE_5E_G3_rev
gatccagatgtgtcactgc

SEQ ID NO: 445 - DNA, 21 bp, Artificial Sequence
  Oligo RACE-5E_mKappaCL_rev
gtgggaagatggatacagttg

SEQ ID NO: 446 - DNA, 20 bp, Artificial Sequence
  Oligo RACE-5E_mLambda1/4_rev
gagctcttcagaggaaggtg

SEQ ID NO: 447 - DNA, 19 bp, Artificial Sequence
  Oligo RACE-5E_mLambda2/3_rev
ctcagrggaaggtggaaac

SEQ ID NO: 448 - DNA, 20 bp, Artificial Sequence
  Oligo RACE-5E_mLambda2/3_rev3
gagctcctcagrggaaggtg

SEQ ID NO: 449 - DNA, 35 bp, Artificial Sequence
  Oligo XSCTnTag
gactcgagtcgacatcgattttttttttttttttt

SEQ ID NO: 450 - DNA, 16 bp, Artificial Sequence
  Oligo M13 Forward
gtaaaacgacggccag

SEQ ID NO: 451 - DNA, 17 bp, Artificial Sequence
  Oligo M13 Reverse
caggaaacagctatgac

(19) United States
(12) Patent Application Publication
(10) Pub. No.: US 2023/0331820 A1
(43) Pub. Date: Oct. 19, 2023

(54) HSV gE ANTIBODIES

(71) Applicant: GLAXOSMITHKLINE BIOLOGICALS SA, Rixensart (BE)

(72) Inventor: Vincent DEWAR, Rixensart (BE)

(73) Assignee: GLAXOSMITHKLINE BIOLOGICALS SA, Rixensart (BE)

(21) Appl. No.: 18/010,258

(22) PCT Filed: Jun. 25, 2021
(86) PCT No.: PCT/EP2021/067582

§ 371 Date: Dec. 14, 2022

(30) Foreign Application Priority Data
Jun. 29, 2020 (EP) 20182922.3

(51) Int. Cl. C07K 16/08; G01N 33/569; A61P 31/22

(52) U.S. Cl. CPC C07K 16/087; G01N 33/56994; A61P 31/22; C07K 2317/92; C07K 2317/76; C07K 2317/565; G01N 2469/10; G01N 2333/035

(57) ABSTRACT

The present invention relates to antigen binding protein, and in particular monoclonal antibodies, with bind to a HSV gEgI heterodimer, and to the use of such in detection and potency assays and in therapy.

### FIELD OF THE INVENTION

The present invention relates to antigen binding proteins and antibodies directed against HSV gE, and in particular against HSV gE Fc binding domain (FCBD), and to their use in in vitro detection and characterisation assays.

### BACKGROUND

Herpes Simplex Virus (HSV, including HSV1 and HSV2) are members of the subfamily Alphaherpesvirinae (a-herpesvirus) in the family Herpesviridae. They are enveloped, double-stranded DNA viruses containing at least 74 genes encoding functional proteins. HSV1 and HSV2 infect mucosal epithelial cells and establish lifelong persistent infection in sensory neurons innervating the mucosa in which the primary infection had occurred. Both HSV1 and HSV2 can reactivate periodically from latency established in neuronal cell body, leading to either herpes labialis (cold sores) or genital herpes (GH). Recurrent GH is the consequence of reactivation of HSV2 (and to some extent of HSV1) from the sacral ganglia, followed by an anterograde migration of the viral capsid along the neuron axon leading to viral particles assembly, cell to cell fusion, viral spread and infection of surrounding epithelial cells from the genital mucosa.

HSV gEgI heterodimer functions as a viral Fc gamma receptor (FcγR), meaning it has the capacity to interact with the Fc portion of human IgG.HSV1 or HSV2 gE or gEgI heterodimer, when displayed at the cell surface of HSV infected cells, bind host IgG through their Fc portion. This results in human IgGs binding to other HSV1 or HSV2 antigens (for example gD) on the virion or infected cell through their Fab domain and binding the Fc binding domain on the viral gE through their Fc domain, leading to endocytosis of the immune complex through a clathrin-mediated mechanism. This mechanism is referred to as antibody bipolar bridging and is postulated to be a major immune evasion strategy competing with innate immune cell activation. Through antibody Fc binding, the viral FcγR inhibits IgG Fc-mediated activities, including complement binding and antibody-dependent cellular cytotoxicity (ADCC) allowing the virus to circumvent the recognition by the immune system. (Ndjamen, Blaise, et al. “The herpes virus Fc receptor gEgI mediates antibody bipolar bridging to clear viral antigens from the cell surface.” PLoS pathogens 10.3 (2014): e1003961.; Dubin, G., et al. “Herpes simplex virus type 1 Fc receptor protects infected cells from antibody-dependent cellular cytotoxicity.” Journal of virology 65.12 (1991): 7046-7050.; Sprague, Elizabeth R., et al. “Crystal structure of the HSV1 Fc receptor bound to Fc reveals a mechanism for antibody bipolar bridging.” PLoS biology 4.6 (2006): e148.). HSV gE (alone or together with gI as a heterodimer) has been proposed as a potential vaccine antigen for treating patients infected with HSV. Indeed, directing an immune response against the Fc binding domain of gE could prevent or interfere with the above described immune evasion mechanism (antibody bipolar bridging), allowing natural immunity to viral proteins, in particular the immune-dominant HSV gD antigen, to become more potent.

Suitable assessments of potency, structure or immunogenicity are required in vaccine discovery, development, manufacturing and release. It is therefore desirable to provide an in vitro test to confirm that a particular antigen will be expected to have in vivo activity in human recipients. Therefore, there is a need to provide an in vitro assay for detecting HSV gE and assessing its potency and functionality. Monoclonal antibodies (mAbs) can be used in in-vitro immunoassays batch release, replacing the use of animals. Monoclonal antibodies against HSV gE also have a potential for treating patients infected with HSV, in particular mAbs which are able to compete with human IgGs for binding for the gE Fc binding domain. Therefore, a careful selection of antibodies to be used in such assays or in therapy is needed, and their functional and analytical properties must be well characterized.

### SUMMARY OF THE INVENTION

In one aspect, the invention provides an antigen binding protein, antibody or mAb which binds to a HSV gE antigen.

In one aspect, the invention provides an antigen binding protein, antibody or mAb which binds to a conformational epitope on the HSV gEgI heterodimer which is located at the interface between HSV gE and HSV gI.

In one aspect, there is provided an antigen binding protein, antibody or mAb which binds to an epitope located on HSV gI.

In one aspect, the invention provides a nucleic acid encoding the antigen binding protein, antibody or mAb of the invention.

In one aspect, the invention provides an expression vector comprising the nucleic acid of the invention.

In one aspect, the invention provides a host cell comprising the nucleic acid sequence or the expression vector of the invention.

In one aspect, the invention provides a method for the production of an antigen binding protein, antibody or mAb of the invention, comprising culturing the recombinant host cell of the invention conditions suitable for expression of the nucleic acid sequence or vector, whereby a polypeptide comprising the antigen binding protein is produced.

In one aspect, the invention provides an antigen binding protein, antibody or mAb produced by the method of the invention.

In one aspect, the invention provides the use of the antigen binding protein, antibody or mAb of the invention in the in vitro detection of a HSV gE antigen or gEgI heterodimer, confirmation of its ability or loss of ability to bind to human IgGs, and/or detection of a change in its conformation.

In one aspect, the invention provides an assay comprising exposing a sample comprising a HSV gE antigen or gEgI heterodimer to an antigen binding protein, antibody or mAb of the invention under conditions sufficient to form an immune complex and detecting the immune complex.

In one aspect, the invention provides a binding assay for in vitro analysis of a sample comprising a HSV gE antigen or gEgI heterodimer comprising the steps of:
i) contacting the sample with a detection antibody directed against a HSV gE or gEgI epitope under conditions sufficient to form an immune complex; and
ii) measuring the interaction between the HSV gE or gEgI antigen and detection antibody from step (i).

In one aspect, the invention provides a pharmaceutical composition comprising an antigen binding protein, antibody or mAb of the invention and a pharmaceutically acceptable carrier.

In one aspect, the invention provides the antigen binding protein, antibody, mAb or pharmaceutical composition of the invention for use in therapy.

In one aspect, the invention provides the antigen binding protein, antibody, mAb or pharmaceutical composition of the invention for use in the treatment of recurrent herpes infection, or, for use in a method for prevention or reduction of the frequency of recurrent herpes virus infection in a subject, preferably a human subject.

In one aspect, the invention provides the antigen binding protein, antibody, mAb or pharmaceutical composition of the invention for use in the manufacture of an immunogenic composition.

In one aspect, the invention provides the use of the antigen binding protein, antibody, mAb or pharmaceutical composition of the invention in the manufacture of a medicament for the treatment of herpes infection or herpes-related disease.

In one aspect, the invention provides a method of treating a herpes virus infection or herpes virus related disease in a subject in need thereof comprising administering an immunologically effective amount of the antigen binding protein, antibody, mAb or pharmaceutical composition of the invention to the subject.

### BRIEF DESCRIPTION OF THE DRAWINGS

FIG. 1—Repetitive Immunization Multiple site-Immunization and lymph nodes sites

FIG. 2—Hybridoma generation principle

FIG. 3—Principle of Surface Plasmon Resonance

FIG. 4—Sensogram and associated parameters

FIG. 5—Description of binding chart assay principle

FIG. 6—Description of binning assay principle

FIG. 7—Schematic of antibody molecule and of the heavy chain. The red box highlights the specific region obtained and analyzed with the sequencing method developed, namely the VH domain and a fragment of the CH1 (VL and CL for the light chain).

FIG. 8. Overview of the sequencing procedure, from RNA extraction to Sanger and Next Generation Sequencing

FIG. 9—Comparative immunoreactivity for a panel of mabs on gEgI produced on HEK293 vs CHO in ELISA and Biacore SPR. A) HEK293 ELISA B) CHO ELISA C) Biacore SPR

FIG. 10—Binding chart of Fc binding domain specific mAbs on FCBD protein sub-domain

FIG. 11—Binding chart of Fc Binding domain specific mAbs on gEgI protein

FIG. 12-2-D surface like map of epitopes for FCBD specific mAbs

FIG. 13-2-D surface like map of epitopes for gEgI heterodimer specific mAbs

FIG. 14—HSV gEgI immune-evasion mechanism

FIG. 15—Phylogenetic tree of VH sequences

FIG. 16—Phylogenetic tree of VL sequences

FIG. 17—Gyrolab assay designs 1, 2, 3, 4

FIG. 18—Gyrolab assay design 1. A) Human IgG binding on WT gEgI. Test antibodies: pAb (polyclonal), mAb 5, mAb 12, mAb 18. B) Human IgG binding on WT gEgI & mutant gEgI (P317R). Test antibody: mAb 18. C) Human IgG binding on WT gEgI & mutant gEgI (P317R). Test antibodies: mAb 18, GP KO pAb (polyclonal antibodies generated in guinea pig against mutant (P317R) gEgI), Rabbit FcBD pAb (polyclonal antibodies generated in rabbit against gE FCBD domain).

FIG. 19—Gyrolab assay design 2. A) Human IgG binding on WT gEgI. Test antibodies: pAb (polyclonal), mAb 5, mAb 12, mAb 18. B) Human IgG binding on WT gEgI & mutant gEgI (P317R). Test antibody: mAb 18.

FIG. 20—Gyrolab assay design 3. A) Human IgG binding on WT gEgI. Test antibodies: pAb (polyclonal), mAb 5, mAb 12, mAb 18. B) Human IgG binding on WT gEgI & mutant gEgI (P317R). Test antibody: mAb 18.

FIG. 21—Gyrolab assay design 4. mAb sandwich design. A) WT gEgI. B) mutant gEgI (P317R).

FIG. 22—Gyrolab assay design 4. mAb sandwich competition design.

FIG. 23—Human IgG binding on WT gEgI Pr02. 4-steps immunoassay by Gyros. The biotinylated gEgI is captured at 100 μg/ml (20 ng/column) to the streptavidin bead. Then the antibody was added at 100 μg/ml (20 ng/column). After, the human IgG is loaded at 50 μg/ml (50 ng/column) and diluted to 0.21 μg/ml (0.21 ng/column). Anti-human fluo Ab is used at the end as revelation.

FIG. 24—Direct detection by a 2-steps Gyros immunoassay. The different mAbs are labeled with fluorochrome. BMP1135 is the HSV-2 gEgI HEK293 WT construct, the BMP1203 is the HSV-2 gEgI CHO WT construct (the integrity of this aliquot can be compromised) and the BMP1265 is the HSV-2 gEgI CHO KO construct. Fluorescent mAb are loaded at 25 nM.

FIG. 25—Direct detection by a 2-steps Gyros immunoassay. The different mAbs are labeled with the fluorochrome. Pr02 is the HSV-2 gEgI CHO WT construct and the Pr05 is the HSV-2 gEgI CHO KO construct. Fluorescent mAb are load at 10 nM.

FIG. 26—crystal hits for gEgI-Fab12 (left) and gEgI-Fab18 (right).

FIG. 27—Schematic showing topological domain of gE which was used (along with gI) for crystallization experiments. Only the Fc Binding domain (gE_FcBD) was present in the x-ray structure.

FIG. 28—X-ray structure of the gE_FcBD-Fab 12 complex, with a zoomed in view of two orientations of paratope/epitope interactions. gE is shown in cartoon representation and colored gold and Fab 12 is colored dark red for the heavy chain and pink for the light chain. Fab 12 buries a total of 1167 Å² on gE. The bar graph displays the total buried surface area on gE for the indicated portion of Fab 12.

FIG. 29—HCDR3, LCDR1, and LCDR3 interactions with gE.

FIG. 30—Hydrogen bond interactions of HCDR2 with gE residues Gln350 (left) and Asp264 (right).

FIG. 31—X-ray structure of the gE_FcBD-Fab 18 complex is shown on the left, with gE_FcBD shown as yellow cartoon, and the Fab18 heavy and light chain shown as surface and colored dark blue and cyan, respectively. On the right-hand side, a bar graph representing the buried surface area (BSA) for Fab 18 heavy and light chain CDRs on gE.

FIG. 32—Open book view of the gE_FcBD-Fab 18 paratope/epitope contacts. Proteins are shown in surface representation, with the residues at the interface of either gE or Fab 18 colored dark red. gE residue Arg30 is circled with a green dashed line and protrudes into a pocket formed between the Fab 18 heavy and light chains, also circled with a green dashed line.

FIG. 33—Selected hydrogen bond interactions between gE_FcBD and Fab 18. Arg320 forms five hydrogen bonds, three with the Fab 18 heavy chain and two with the light chain. gE_FcBD is colored gold, Fab 18 heavy chain is colored blue, and the Fab 18 light chain is colored cyan. Hydrogen bonds are shown as black dashes.

### DETAILED DESCRIPTION OF THE INVENTION

In one aspect, the invention provides an antigen binding protein which binds to a HSV gE antigen.

As used herein, an “antigen binding protein” is a protein which is capable of binding to an antigen, in the present case, to a HSV gE antigen.

In a preferred embodiment, the antigen binding protein is an antibody which binds to an epitope on a HSV gE antigen.

As used herein, “antibody” refers to an immunoglobulin that specifically binds to an analyte (such as a protein or protein complex). In mammals, there are five antibody isotypes known as IgA, IgD, IgE, IgG, and IgM. The isotypes denote the different types of heavy chains the antibody contains, with each heavy chain class named alphabetically: α (alpha), γ (gamma), δ (delta), ε (epsilon), and μ (mu). Distinct heavy chains differ in size and composition; α and γ contain approximately 450 amino acids, whereas μ and ε have approximately 550 amino acids. Human IgG comprise four subclasses (IgG1, IgG2, IgG3 and IgG4). In mice the IgG class is divided into five sub-classes (IgG1, IgG2a, IgG2b, IgG2c and IgG3) and in rat there are four (IgG1, IgG2a, IgG2b, IgG2c). Sub-class nomenclature has arisen independently for each species and so there is no general relationship between the sub-classes from each species. In mammals, there are two types of immunoglobulin light chains, lambda (λ) and kappa (κ). The approximate length of a light chain is 211 to 217 amino acids. Non-limiting examples of antibodies include intact monoclonal or polyclonal immunoglobulins as well as variants and/or fragments thereof that retain the binding affinity of the intact immunoglobulin for the HSV gEgI heterodimer. Native antibodies are usually heterotetrameric glycoproteins of about 150,000 daltons, composed of two identical light (L) chains and identical heavy (H) chains. Each light chain is linked to a heavy chain by disulfide bonds. Each light and heavy chain contains a constant region and a variable domain. “V_{H}” or “VH” refers to an antibody heavy chain variable domain. “V_{L}” or “VL” refers to an antibody light chain variable domain. V_{H} and V_{L} domains contain three hypervariable domains (also referred to as complementarity-determining regions or “CDRs”) interspaced by four framework regions (see e.g., Kabat et al., Sequences of Proteins of Immunological Interest, 5^{th} Ed. Public Health Service, National Institutes of Health, Bethesda, MD, 1991). The sequences of the framework regions of different light or heavy chains are relatively conserved within a species. The framework regions function to position the CDRs in three-dimensional space. Heavy chain CDRs are typically referred to as HCDR1, HCDR2 and HCDR3 (from N- to C-terminus). Light chain CDRs are typically referred to as LCDR1, LCDR2 and LCDR3 (from N- to C-terminus). Somatic recombination of immunoglobulins, also known as V(D)J recombination, involves the generation of a unique immunoglobulin variable region. The variable region of each immunoglobulin heavy or light chain is encoded in several pieces-known as gene segments (subgenes). These segments are called variable (V), diversity (D) and joining (J) segments. V, D and J segments are found in Ig heavy chains, but only V and J segments are found in Ig light chains. In the bone marrow, each developing B cell will assemble an immunoglobulin variable region by randomly selecting and combining one V, one D and one J gene segment (or one V and one J segment in the light chain). As there are multiple copies of each type of gene segment, and different combinations of gene segments can be used to generate each immunoglobulin variable region, this process generates a huge number of antibodies, each with different paratopes, and thus different antigen specificities.

Herein, the term “antibody” is used in its broadest sense to refer to molecules with an immunoglobulin-like domain (for example IgG, IgM, IgA, IgD or IgE) and includes monoclonal (mAb), recombinant, polyclonal (pAB), chimeric, human, humanized, multispecific antibodies, including bispecific antibodies, and heteroconjugate antibodies; a single variable domain (e.g., VH, VHH, VL, domain antibody (dAb™)), antigen binding antibody fragments, Fab, F(ab′)₂, Fv, disulphide linked Fv, single chain Fv, disulphide-linked scFv, diabodies, TANDABS™, etc. and modified versions of any of the foregoing (for a summary of alternative “antibody” formats see [Holliger P, Hudson P J. Engineered antibody fragments and the rise of single domains. Nat Biotechnol. 2005; 23(9):1126-36]). Alternative antibody formats include alternative scaffolds in which the one or more CDRs of the antigen binding protein can be arranged onto a suitable non-immunoglobulin protein scaffold or skeleton, such as an affibody, a SpA scaffold, an LDL receptor class A domain, an avimer or an EGF domain.

In one embodiment, the antibody is a mammalian antibody, preferably a murine antibody. Alternatively, the antibody is a human or humanized antibody. In one embodiment, the antibody is a type G immunoglobulin (IgG), suitably a murine, human or humanized IgG. In a preferred embodiment, the IgG is a murine IgG from isotype 1, 2a or 3. In a preferred embodiment, the light chain is a Kappa light chain class. In a preferred embodiment, the IgG is a murine IgG from isotype 1, 2a or 3, and the light chain is a Kappa light chain class.

As used herein ‘specific binding’ of an antibody refers to a binding reaction to a target protein such as gE or protein complex such as the gEgI heterodimer. Under designated conditions, an antibody binds preferentially to its target protein or protein complex and does not bind in significant amounts to other proteins or molecules present in a sample being tested for the presence of the target protein or protein complex.

As used herein, the term “epitope” refers to the portion of a macromolecule (antigen) which is specifically recognised by a component of the immune system e.g. an antibody or a T-cell antigen receptor. The term epitope may refer to that portion of the antigen that makes contact with a particular binding domain of the antigen binding protein. An epitope may be linear or conformational. Particular residues comprised within an epitope can be determined through computer modelling programs or via three-dimensional structures obtained through methods known in the art, such as X-ray crystallography. An epitope may reside within the consensus sequence of the invention.

As used herein, the term “conformational epitope” (or “discontinuous epitope”) refers to an epitope which comprises amino acid residues that are separated by other sequences, i.e. not in a continuous sequence in the antigen's primary sequence. Although the residues may be from different regions of the peptide chain, they are in close proximity in the three-dimensional structure of the antigen. In the case of multimeric antigens (such as the HSV gEgI heterodimer), a conformational epitope may include residues from different peptide chains. A conformational epitope may be formed when amino acid residues (to which antibodies can bind) are formed as a result of the polypeptides three-dimensional conformation. The amino acids are located at distinct sites along the linear length of a polypeptide but are co-localised in the 3-D crystal structure. Identifying conformational epitopes can be achieved by numerous methods known in the art. For example, conformational epitopes can be identified using epitope mapping techniques such as Hydrogen Deuterium Exchange Mass Spectrometry (HDX-MS). Instances where a single monoclonal antibody binds to two or more distinct areas of the same linear chain suggest the presence of a conformational epitope. This can be further analysed by mapping said distinct areas onto the 3D crystal structure of a polypeptide molecule. Close structural localisation of the distinct epitopes confirms the presence of a conformational epitope. Conformational epitopes might be preferred for applications involving protein targets in their native state, such as therapeutic applications or flow cytometry. On the other hand, linear epitopes might be preferred for applications in which the protein target is wholly or partially denatured during the sample preparation prior to the immuno assay, such as in Western blot (WB), immunohistochemistry (IHC) or immunofluorescence-based confocal microscopy [Forsstrom et al 2015, PloS One 10(3) e0121673].

In a preferred embodiment, the gE antibody is a monoclonal antibody (mAb) which binds to an epitope on a HSV2 gE antigen.

A ‘monoclonal antibody’ (or ‘mAb’) is an antibody obtained from a population of substantially identical antibodies, i.e., the antibodies in the population bind the same epitope. It is however recognized that a population of mAbs may contain a minority of variant antibodies (e.g., antibodies containing naturally-occurring mutations such as those arising during production of mAbs). In contrast, polyclonal antibody preparations typically include different antibodies directed against different epitopes. Monoclonal antibodies of the present invention may be produced or obtained by any method as is known in the art, including hybridoma methods, recombinant DNA production, phage-display methods, and use of transgenic animals containing all or part of the human immunoglobulin loci. Monoclonal antibodies of the present invention may contain conservative amino acid substitutions, where such substitutions have substantially no effect on the antibody function.

A “conservative substitution” comprises the substitution of an amino acid with another amino acid having a physico-chemical property similar to the amino acid that is substituted (see, for example, Stryer et al, Biochemistry, 5th Edition 2002, pages 44-49). Preferably, the conservative substitution is a substitution selected from the group consisting of: (i) a substitution of a basic amino acid with another, different basic amino acid; (ii) a substitution of an acidic amino acid with another, different acidic amino acid; (iii) a substitution of an aromatic amino acid with another, different aromatic amino acid; (iv) a substitution of a non-polar, aliphatic amino acid with another, different non-polar, aliphatic amino acid; and (v) a substitution of a polar, uncharged amino acid with another, different polar, uncharged amino acid. A basic amino acid is preferably selected from the group consisting of arginine, histidine, and lysine. An acidic amino acid is preferably aspartate or glutamate. An aromatic amino acid is preferably selected from the group consisting of phenylalanine, tyrosine and tryptophane. A non-polar, aliphatic amino acid is preferably selected from the group consisting of alanine, valine, leucine, methionine and isoleucine. A polar, uncharged amino acid is preferably selected from the group consisting of serine, threonine, cysteine, proline, asparagine and glutamine. In contrast to a conservative amino acid substitution, a non-conservative amino acid substitution is the exchange of one amino acid with any amino acid that does not fall under the above-outlined conservative substitutions (i) through (v).

Suitably, the antigen binding protein, antibody or mAb of the invention is isolated. As used herein, ‘isolated’ refers to a biological component (such as a nucleic acid molecule, a peptide or protein, an antibody or antigen binding fragment thereof) that has been substantially separated, produced separately from, or purified from other biological components in the cell in which the component naturally occurs. Isolated peptides and proteins include those purified by standard purification methods, e.g., proteins produced recombinantly in a cell and purified from the cell culture, or those chemically synthesized. ‘Purification’ does not necessarily imply 100% purity, i.e., the complete exclusion of all other components. An isolated nucleic acid molecule, peptide, or protein is at least 50%, at least 60%, at least 70%, at least 80%, at least 90%, at least 95%, at least 96%, at least 97%, at least 98%, or at least 99% pure.

Herein, a “HSV gE antigen” is a HSV membrane glycoprotein gE or an immunogenic fragment thereof. Suitably, the HSV gE antigen is selected from a HSV2 gE antigen or an immunogenic fragment thereof, and a HSV1 gE antigen or an immunogenic fragment thereof. As used herein, an “immunogenic fragment” refers to a molecule containing one or more epitopes (e.g., linear, conformational or both) capable of stimulating a host's immune system to make a humoral and/or cellular antigen-specific immunological response (i.e. an immune response which specifically recognizes a naturally occurring polypeptide, e.g., a viral or bacterial protein). Suitably, the HSV gE antigen or immunogenic fragment thereof does not comprise a functional transmembrane domain. Suitably, the HSV gE antigen or immunogenic fragment thereof does not comprise a cytoplasmic domain.

Preferably, the HSV gE antigen or immunogenic fragment thereof comprises or consists essentially of a HSV gE ectodomain. In a preferred embodiment, the gE antigen a HSV2 gE antigen. In a more preferred embodiment, the HSV gE antigen consists essentially of a HSV2 gE ectodomain.

Exemplary HSV2 gE sequences include Genbank accession numbers AHG54732.1 (SEQ ID NO: 1), AKC59449.1, AKC42830.1, ABU45436.1, ABU45439.1, ABU45437.1, ABU45438.1, AMB66104.1, AMB66173.1, AMB66246.1, AKC59520.1, AKC59591.1, AKC59307.1, AMB66465.1, AKC59378.1, AEV91407.1, CAB06715.1, YP_009137220.1, ABW83306.1, ABW83324.1, ABW83308.1, ABW83310.1, ABW83312.1, ABW83314.1, ABW83316.1, ABW83318.1, ABW83320.1, ABW83322.1, ABW83398.1, ABW83380.1, ABW83396.1, ABW83382.1, ABW83384.1, ABW83394.1, ABW83386.1, ABW83388.1, ABW83390.1, ABW83392.1, ABW83400.1, ABW83342.1, ABW83340.1, ABW83346.1, ABW83348.1, ABW83326.1, ABW83350.1, ABW83352.1, ABW83336.1, ABW83334.1, ABW83354.1, ABW83338.1.

In a preferred embodiment, the HSV2 gE ectodomain comprises or consists of the amino acid sequence shown on SEQ ID NO: 5 (corresponding to amino acid residues 1-419 of SEQ ID NO: 1), or a sequence which is at least 60%, 65%, 70%, 75% 80%, 85%, 90%, 95%, 96%, 97%, 98%, 99% or 99.5% identical thereto. In a preferred embodiment, the HSV2 gE ectodomain is at least 90%, 95%, 96%, 97%, 98%, 99%, 99.5% or 100% identical to SEQ ID NO: 5. Suitably, the HSV2 gE ectodomain may comprise one or more amino acid residue substitution, deletion, or insertion relative to the amino acid sequence shown at SEQ ID NO: 5, for example 1, 2, 3, 4, 5, 6, 7, 8, 9 or 10 amino acid residue substitution, deletion, or insertions.

In another embodiment, the gE antigen is a HSV1 gE antigen. In one embodiment, the HSV gE antigen consists essentially of a HSV1 gE ectodomain. An exemplary HSV1 gE is the gE from HSV1 strain KOS321 (UniProtKB accession number: Q703E9) which comprises or consists of the amino acid sequence shown in SEQ ID NO:3.

Suitably, the HSV1 gE ectodomain comprises or consists of the amino acid sequence shown on SEQ ID NO: 7 (corresponding to amino acid residues 1-419 of SEQ ID NO: 3), or a sequence which is at least 60%, 65%, 70%, 75% 80%, 85%, 90%, 95%, 96%, 97%, 98%, 99% or 99.5% identical thereto. In a preferred embodiment, the HSV1 gE ectodomain is at least 90%, 95%, 96%, 97%, 98%, 99%, 99.5% or 100% identical to SEQ ID NO: 7. Suitably, the HSV1 gE ectodomain may comprise one or more amino acid residue substitutions, deletions, or insertions relative to the amino acid sequence shown at SEQ ID NO: 7, for example 1, 2, 3, 4, 5, 6, 7, 8, 9 or amino acid residue substitution, deletion, or insertions.

In one embodiment, the HSV gE antigen is associated with a HSV gI antigen to form a gEgI heterodimer. Herein, a “HSV gEgI heterodimer” is a noncovalent heterodimer formed from two HSV membrane glycoproteins, gE and gI, or from immunogenic fragments thereof. Suitably, the HSV gEgI heterodimer is selected from a HSV2 gEgI heterodimer comprising a HSV2 gE antigen and a HSV2 gI antigen, or immunogenic fragments thereof, and a HSV1 gEgI heterodimer comprising a HSV1 gE antigen and a HSV1 gI antigen, or immunogenic fragments thereof. As used herein, an “immunogenic fragment” refers to a molecule containing one or more epitopes (e.g., linear, conformational or both) capable of stimulating a host's immune system to make a humoral and/or cellular antigen-specific immunological response (i.e. an immune response which specifically recognizes a naturally occurring polypeptide, e.g., a viral or bacterial protein). Suitably, the HSV gE antigen or immunogenic fragment thereof does not comprise a functional transmembrane domain. Suitably, the HSV gE antigen or immunogenic fragment thereof does not comprise a cytoplasmic domain. Preferably, the HSV gE antigen or immunogenic fragment thereof comprises or consists essentially of a HSV gE ectodomain. Suitably, the HSV gI antigen or immunogenic fragment thereof does not comprise a functional transmembrane domain. Suitably, the HSV gI antigen or immunogenic fragment thereof does not comprise a cytoplasmic domain. Preferably, the HSV gI antigen or immunogenic fragment thereof comprises or consists essentially of a HSV gI ectodomain. In a preferred embodiment, the gEgI heterodimer is a HSV2 gEgI heterodimer. In a more preferred embodiment, the HSV gEgI heterodimer consists essentially of a HSV2 gE ectodomain and a HSV2 gI ectodomain.

Exemplary HSV2 gI sequences include Genbank accession numbers AHG54731.1 (SEQ ID NO: 2), AKC42829.1, AKC59519.1, AKC59590.1, AKC59306.1, AKC59377.1, ABW83313.1, ABW83397.1, ABW83385.1, ABW83327.1, ABW83341.1, ABW83339.1, ABW83325.1, ABW83351.1, ABW83337.1, ABW83355.1, ABW83343.1, ABW83329.1, ABW83357.1, ABW83365.1, ABW83367.1, ABW83371.1, ABW83377.1, AKC59448.1, ABW83319.1, ABW83379.1, ABW83381.1, ABW83383.1, ABW83389.1, ABW83347.1, ABW83349.1, ABW83335.1, ABW83333.1, ABW83353.1, ABW83359.1, ABW83363.1, ABW83331.1, ABW83369.1, ABW83375.1, AMB66172.1, YP_009137219.1, CAB06714.1, AEV91406.1, ABW83305.1, ABW83323.1, ABW83307.1, ABW83311.1, ABW83315.1, ABW83317.1, ABW83321.1, ABW83395.1, ABW83393.1, ABW83387.1, ABW83391.1, ABW83399.1, ABW83345.1, ABW83361.1, ABW83309.1, ABW83373.1, AMB66029.1, AM1B66103.1, AMB66322.1, AMB66245.1

In a preferred embodiment, the HSV2 gI ectodomain comprises or consists of the amino acid sequence shown on SEQ ID NO: 6 (corresponding to amino acid residues 1-256 of SEQ ID NO: 2), or a sequence which is at least 60%, 65%, 70%, 75% 80%, 85%, 90%, 95%, 96%, 97%, 98%, 99% or 99.5% identical thereto. In a preferred embodiment, the HSV2 gI ectodomain is at least 90%, 95%, 96%, 97%, 98%, 99%, 99.5% or 100% identical to SEQ ID NO: 6. Suitably, the HSV2 gI ectodomain may comprise one or more amino acid residue substitutions, deletions, or insertions relative to the amino acid sequence shown at SEQ ID NO: 6, for example 1, 2, 3, 4, 5, 6, 7, 8, 9 or 10 amino acid residue substitution, deletion, or insertions.

In another embodiment, the gEgI heterodimer is a HSV1 gEgI heterodimer. In one embodiment, the HSV gEgI heterodimer consists essentially of a HSV1 gE ectodomain and a HSV1 gI ectodomain.

An exemplary HSV1 gI is the gI from HSV1 strain 17 (UniProtKB accession number: P06487) which comprises or consists of the amino acid sequence shown in SEQ ID NO: 4. Suitably, the HSV1 gI ectodomain comprises or consists of the amino acid sequence shown on SEQ ID NO: 8 (corresponding to amino acid residues 1-256 of SEQ ID NO: 2), or a sequence which is at least 60%, 65%, 70%, 75% 80%, 85%, 90%, 95%, 96%, 97%, 98%, 99% or 99.5% identical thereto. In a preferred embodiment, the HSV1 gI ectodomain is at least 90%, 95%, 96%, 97%, 98%, 99%, 99.5% or 100% identical to SEQ ID NO: 8. Suitably, the HSV1 gI ectodomain may comprise one or more amino acid residue substitutions, deletions, or insertions relative to the amino acid sequence shown at SEQ ID NO: 8, for example 1, 2, 3, 4, 5, 6, 7, 8, 9 or 10 amino acid residue substitution, deletion, or insertions.

In a preferred embodiment, the dissociation constant (K_{D}) between the HSV gE binding protein and the HSV gE antigen or gEgI heterodimer is lower than 5×10⁻⁷ M, preferably lower than 1×10⁻⁷ M, more preferably lower than 5×10⁻⁸ M.

The binding affinity between the HSV gE binding protein and HSV gE antigen or gEgI heterodimer can be determined by methods well known to those skilled in the art. For example, the association rate (kₒₙ or ka), dissociation rate (k_{off} or kd), dissociation constant (K_{D}=k_{off}/kₒₙ) and association constant (K_{A}=1/K_{D}=kₒₙ/k_{off}) be determined by BiLayer Interferometry or by Surface Plasmon Resonance (SPR) as described in example 1.

The inventors have identified and characterized 26 clones expressing monoclonal antibodies which bind to an epitope on a HSV gE antigen, a gI antigen and/or an gEgI heterodimer (see example section and Tables 7-11).

In a preferred embodiment, the monoclonal antibody binds to an epitope located on HSV gE. Preferably, such a mAb binds to free HSV gE and to the HSV gEgI heterodimer. In a preferred embodiment, the mAb binds to an epitope located on the HSV gE Fc binding domain (FCBD). Examples of mAbs which bind to the HSV gE Fc binding domain include mAbs from clones 18, 12/13, 14, 43 and 47 (see example section and tables 7-11).

In a preferred embodiment, the mAb which binds to an epitope located on the HSV gE FCBD comprises any one or a combination of CDRs selected from SEQ ID NOs: 50-52, 30-32, 34-36, 94-96, 48-50, 154-156, 134-136, 138-140, 202-204 and 214-216, or variants thereof, wherein the variant has 1, 2, or 3 amino acid deletions, substitutions or insertions. In a preferred embodiment, the mAb which binds to an epitope located on the HSV gE FCBD comprises any one or a combination of CDRs encoded by SEQ ID NOs: 262-264, 242-244, 246-248, 306-308, 318-320, 366-368, 346-348, 350-352, 414-416 and 426-428, or variants thereof, wherein the variant has 1, 2, 3, 4, 5, 6, 7, 8, 9 or 10 nucleotide deletions, substitutions or insertions.

In a preferred embodiment, the mAb which binds to an epitope located on the HSV gE FCBD, comprises a heavy chain, wherein the heavy chain variable region comprises:
a) the HCDR1 shown in SEQ ID NO: 50, the HCDR2 shown in SEQ ID NO: 51 and the HCDR3 shown in SEQ ID NO: 52, or variants thereof having 1, 2, or 3 amino acid deletions, substitutions or insertions,
b) the HCDR1 shown in SEQ ID NO: 30, the HCDR2 shown in SEQ ID NO: 31 and the HCDR3 shown in SEQ ID NO: 32, or variants thereof having 1, 2, or 3 amino acid deletions, substitutions or insertions,
c) the HCDR1 shown in SEQ ID NO: 34, the HCDR2 shown in SEQ ID NO: 35 and the HCDR3 shown in SEQ ID NO:36, or variants thereof having 1, 2, or 3 amino acid deletions, substitutions or insertions,
d) the HCDR1 shown in SEQ ID NO: 94, the HCDR2 shown in SEQ ID NO: 95 and the HCDR3 shown in SEQ ID NO: 96, or variants thereof having 1, 2, or 3 amino acid deletions, substitutions or insertions, or
e) the HCDR1 shown in SEQ ID NO: 106, the HCDR2 shown in SEQ ID NO: 107 and the HCDR3 shown in SEQ ID NO: 108, or variants thereof having 1, 2, or 3 amino acid deletions, substitutions or insertions.

In a preferred embodiment, the mAb which binds to an epitope located on the HSV gE FCBD, comprises a light chain, wherein the light chain variable region comprises:
a) the LCDR1 shown in SEQ ID NO: 154, the LCDR2 shown in SEQ ID NO: 155 and the LCDR3 shown in SEQ ID NO: 156, or variants thereof having 1, 2, or 3 amino acid deletions, substitutions or insertions,
b) the LCDR1 shown in SEQ ID NO: 134, the LCDR2 shown in SEQ ID NO: 135 and the LCDR3 shown in SEQ ID NO:136, or variants thereof having 1, 2, or 3 amino acid deletions, substitutions or insertions,
c) the LCDR1 shown in SEQ ID NO: 138, the LCDR2 shown in SEQ ID NO: 139 and the LCDR3 shown in SEQ ID NO: 140, or variants thereof having 1, 2, or 3 amino acid deletions, substitutions or insertions,
d) the LCDR1 shown in SEQ ID NO: 202, the LCDR2 shown in SEQ ID NO: 203 and the LCDR3 shown in SEQ ID NO: 204, or variants thereof having 1, 2, or 3 amino acid deletions, substitutions or insertions, or
e) the LCDR1 shown in SEQ ID NO: 214, the LCDR2 shown in SEQ ID NO: 215 and the LCDR3 shown in SEQ ID NO: 216, or variants thereof having 1, 2, or 3 amino acid deletions, substitutions or insertions.

In a preferred embodiment, the mAb which binds to an epitope located on the HSV gE FCBD, comprises a heavy chain and a light chain, wherein:
a) the heavy chain variable region comprises the HCDR1 shown in SEQ ID NO: 50, the HCDR2 shown in SEQ ID NO: 51 and the HCDR3 shown in SEQ ID NO: 52, and the light chain variable region comprises the LCDR1 shown in SEQ ID NO: 154, the LCDR2 shown in SEQ ID NO: 155 and the LCDR3 shown in SEQ ID NO: 156,
b) the heavy chain variable region comprises the HCDR1 shown in SEQ ID NO: 30, the HCDR2 shown in SEQ ID NO: 31 and the HCDR3 shown in SEQ ID NO: 32, and the light chain variable region comprises the LCDR1 shown in SEQ ID NO: 134, the LCDR2 shown in SEQ ID NO: 135 and the LCDR3 shown in SEQ ID NO:136,
c) the heavy chain variable region comprises the HCDR1 shown in SEQ ID NO: 34, the HCDR2 shown in SEQ ID NO: 35 and the HCDR3 shown in SEQ ID NO:36, and the light chain variable region comprises the LCDR1 shown in SEQ ID NO: 138, the LCDR2 shown in SEQ ID NO: 139 and the LCDR3 shown in SEQ ID NO: 140,
d) the heavy chain variable region comprises the HCDR1 shown in in SEQ ID NO: 94, the HCDR2 shown in SEQ ID NO: 95 and the HCDR3 shown in SEQ ID NO: 96, and the light chain variable region comprises the LCDR1 shown in SEQ ID NO: 202, the LCDR2 shown in SEQ ID NO: 203 and the LCDR3 shown in SEQ ID NO: 204, or
e) the heavy chain variable region comprises the HCDR1 shown in SEQ ID NO: 106, the HCDR2 shown in SEQ ID NO: 107 and the HCDR3 shown in SEQ ID NO: 108, and the light chain variable region comprises the LCDR1 shown in SEQ ID NO: 214, the LCDR2 shown in SEQ ID NO: 215 and the LCDR3 shown in SEQ ID NO: 216.

In a preferred embodiment, the mAb which binds to an epitope located on the HSV gE FCBD, comprises a heavy chain, wherein the heavy chain variable region comprises:
a) the HCDR1 encoded by the nucleotide sequence SEQ ID NO: 262, the HCDR2 encoded by the nucleotide sequence SEQ ID NO: 263 and the HCDR3 encoded by the nucleotide sequence SEQ ID NO: 264, or variants thereof having 1, 2, 3, 4, 5, 6, 7, 8, 9 or 10 nucleotide deletions, substitutions or insertions,
b) the HCDR1 encoded by the nucleotide sequence SEQ ID NO: 242, the HCDR2 encoded by the nucleotide sequence SEQ ID NO: 243 and the HCDR3 encoded by the nucleotide sequence SEQ ID NO: 244, or variants thereof having 1, 2, 3, 4, 5, 6, 7, 8, 9 or 10 nucleotide deletions, substitutions or insertions,
c) the HCDR1 encoded by the nucleotide sequence SEQ ID NO: 246, the HCDR2 encoded by the nucleotide sequence SEQ ID NO: 247 and the HCDR3 encoded by the nucleotide sequence SEQ ID NO: 248, or variants thereof having 1, 2, 3, 4, 5, 6, 7, 8, 9 or 10 nucleotide deletions, substitutions or insertions,
d) the HCDR1 encoded by the nucleotide sequence SEQ ID NO: 306, the HCDR2 encoded by the nucleotide sequence SEQ ID NO: 307 and the HCDR3 encoded by the nucleotide sequence SEQ ID NO: 308, or variants thereof having 1, 2, 3, 4, 5, 6, 7, 8, 9 or 10 nucleotide deletions, substitutions or insertions, or
e) the HCDR1 encoded by the nucleotide sequence SEQ ID NO: 318, the HCDR2 encoded by the nucleotide sequence SEQ ID NO: 317 and the HCDR3 encoded by the nucleotide sequence SEQ ID NO: 320, or variants thereof having 1, 2, 3, 4, 5, 6, 7, 8, 9 or 10 nucleotide deletions, substitutions or insertions.

In a preferred embodiment, the mAb which binds to an epitope located on the HSV gE FCBD, comprises a light chain, wherein the light chain variable region comprises:
a) the LCDR1 encoded by the nucleotide sequence SEQ ID NO: 366, the LCDR2 encoded by the nucleotide sequence SEQ ID NO: 367 and the LCDR3 encoded by the nucleotide sequence SEQ ID NO: 368, or variants thereof having 1, 2, 3, 4, 5, 6, 7, 8, 9 or 10 nucleotide deletions, substitutions or insertions,
b) the LCDR1 encoded by the nucleotide sequence SEQ ID NO: 346, the LCDR2 encoded by the nucleotide sequence SEQ ID NO: 347 and the LCDR3 encoded by the nucleotide sequence SEQ ID NO:348, or variants thereof having 1, 2, 3, 4, 5, 6, 7, 8, 9 or 10 nucleotide deletions, substitutions or insertions,
c) the LCDR1 encoded by the nucleotide sequence SEQ ID NO: 350, the LCDR2 encoded by the nucleotide sequence SEQ ID NO: 351 and the LCDR3 encoded by the nucleotide sequence SEQ ID NO: 352, or variants thereof having 1, 2, 3, 4, 5, 6, 7, 8, 9 or 10 nucleotide deletions, substitutions or insertions,
d) the LCDR1 encoded by the nucleotide sequence SEQ ID NO: 414, the LCDR2 encoded by the nucleotide sequence SEQ ID NO: 415 and the LCDR3 encoded by the nucleotide sequence SEQ ID NO: 416, or variants thereof having 1, 2, 3, 4, 5, 6, 7, 8, 9 or 10 nucleotide deletions, substitutions or insertions, or
e) the LCDR1 encoded by the nucleotide sequence SEQ ID NO: 426, the LCDR2 encoded by the nucleotide sequence SEQ ID NO: 427 and the LCDR3 encoded by the nucleotide sequence SEQ ID NO: 428, or variants thereof having 1, 2, 3, 4, 5, 6, 7, 8, 9 or 10 nucleotide deletions, substitutions or insertions.

In a preferred embodiment, the mAb which to an epitope located on the HSV gE FCBD, comprises a heavy chain and a light chain, wherein:
a) the heavy chain variable region comprises the HCDR1 encoded by the nucleotide sequence SEQ ID NO: 262, the HCDR2 encoded by the nucleotide sequence SEQ ID NO: 263 and the HCDR3 encoded by the nucleotide sequence SEQ ID NO: 264, and the light chain variable region comprises the LCDR1 encoded by the nucleotide sequence SEQ ID NO: 366, the LCDR2 encoded by the nucleotide sequence SEQ ID NO: 367 and the LCDR3 encoded by the nucleotide sequence SEQ ID NO: 368,
b) the heavy chain variable region comprises the HCDR1 encoded by the nucleotide sequence SEQ ID NO: 242, the HCDR2 encoded by the nucleotide sequence SEQ ID NO: 243 and the HCDR3 encoded by the nucleotide sequence SEQ ID NO: 244, and the light chain variable region comprises the LCDR1 encoded by the nucleotide sequence SEQ ID NO: 346, the LCDR2 encoded by the nucleotide sequence SEQ ID NO: 347 and the LCDR3 encoded by the nucleotide sequence SEQ ID NO:348,
c) the heavy chain variable region comprises the HCDR1 encoded by the nucleotide sequence SEQ ID NO: 246, the HCDR2 encoded by the nucleotide sequence SEQ ID NO: 247 and the HCDR3 encoded by the nucleotide sequence SEQ ID NO: 248, and the light chain variable region comprises the LCDR1 encoded by the nucleotide sequence SEQ ID NO: 350, the LCDR2 encoded by the nucleotide sequence SEQ ID NO: 351 and the LCDR3 encoded by the nucleotide sequence SEQ ID NO: 352,
d) the heavy chain variable region comprises the HCDR1 encoded by the nucleotide sequence in SEQ ID NO: 306, the HCDR2 encoded by the nucleotide sequence SEQ ID NO: 307 and the HCDR3 encoded by the nucleotide sequence SEQ ID NO: 308, and the light chain variable region comprises the LCDR1 encoded by the nucleotide sequence SEQ ID NO: 414, the LCDR2 encoded by the nucleotide sequence SEQ ID NO: 415 and the LCDR3 encoded by the nucleotide sequence SEQ ID NO: 416, or
e) the heavy chain variable region comprises the HCDR1 encoded by the nucleotide sequence SEQ ID NO: 318, the HCDR2 encoded by the nucleotide sequence SEQ ID NO: 317 and the HCDR3 encoded by the nucleotide sequence SEQ ID NO: 320, and the light chain variable region comprises the LCDR1 encoded by the nucleotide sequence SEQ ID NO: 426, the LCDR2 encoded by the nucleotide sequence SEQ ID NO: 427 and the LCDR3 encoded by the nucleotide sequence SEQ ID NO: 428.

In one embodiment, the mAb which binds to an epitope located on the HSV gE FCBD, comprises a heavy chain, wherein the heavy chain variable region has a sequence selected from SEQ ID NO: 49, SEQ ID NO: 29, SEQ ID NO: 33, SEQ ID NO: 93 and SEQ ID NO: 105 a sequence which is at least 80%, 85%, 90%, 95%, 96%, 97%, 98% or 99% identical thereto.

In one embodiment, the mAb which binds to an epitope located on the HSV gE FCBD, comprises a light chain, wherein the light chain variable region has a sequence selected from SEQ ID NO: 153, SEQ ID NO: 133, SEQ ID NO: 137, SEQ ID NO: 201 and SEQ ID NO: 213, or a sequence which is at least 80%, 85%, 90%, 95%, 96%, 97%, 98% or 99% identical thereto.

In one embodiment, the mAb which binds to an epitope located on the HSV gE FCBD, comprises a heavy chain and a light chain, wherein the heavy chain variable region has a sequence selected from SEQ ID NO: 49, SEQ ID NO: 29, SEQ ID NO: 33, SEQ ID NO: 93 and SEQ ID NO: 105, or a sequence which is at least 80%, 85%, 90%, 95%, 96%, 97%, 98% or 99% identical thereto, and wherein the light chain variable region has a sequence selected from SEQ ID NO: 153, SEQ ID NO: 133, SEQ ID NO: 137, SEQ ID NO: 201 and SEQ ID NO: 213, or a sequence which is at least 80%, 85%, 90%, 95%, 96%, 97%, 98% or 99% identical thereto.

In one embodiment, the mAb which binds to an epitope located on the HSV gE FCBD, comprises a heavy chain, wherein the heavy chain variable region comprises or consists of a nucleotide sequence selected from SEQ ID NO: 261, SEQ ID NO: 241, SEQ ID NO: 245, SEQ ID NO: 305 and SEQ ID NO: 317 a sequence which is at least 80%, 85%, 90%, 95%, 96%, 97%, 98% or 99% identical thereto.

In one embodiment, the mAb which binds to an epitope located on the HSV gE FCBD, comprises a light chain, wherein the light chain variable region comprises or consists of a nucleotide sequence selected from SEQ ID NO: 365, SEQ ID NO: 345, SEQ ID NO: 349, SEQ ID NO: 413 and SEQ ID NO: 425, or a sequence which is at least 80%, 85%, 90%, 95%, 96%, 97%, 98% or 99% identical thereto.

In one embodiment, the mAb which binds to an epitope located on the HSV gE FCBD, comprises a heavy chain and a light chain, wherein the heavy chain variable region comprises or consists of a nucleotide sequence selected from SEQ ID NO: 261, SEQ ID NO: 241, SEQ ID NO: 245, SEQ ID NO: 305 and SEQ ID NO: 317, or a sequence which is at least 80%, 85%, 90%, 95%, 96%, 97%, 98% or 99% identical thereto, and wherein the light chain variable region comprises or consists of a nucleotide sequence selected from SEQ ID NO: 365, SEQ ID NO: 345, SEQ ID NO: 349, SEQ ID NO: 413 and SEQ ID NO: 425, or a sequence which is at least 80%, 85%, 90%, 95%, 96%, 97%, 98% or 99% identical thereto.

In one embodiment, the mAb which binds to an epitope located on the HSV gE FCBD, comprises a heavy chain and a light chain, wherein the heavy chain variable region comprises or consists of the sequence shown in SEQ ID NO: 49 or a sequence which is at least 80%, 85%, 90%, 95%, 96%, 97%, 98% or 99% identical thereto, and wherein the light chain variable region comprises or consists of the sequence shown in SEQ ID NO: 153 or a sequence which is at least 80%, 85%, 90%, 95%, 96%, 97%, 98% or 99% identical thereto. In one embodiment, the mAb which binds to an epitope located on the HSV gE FCBD, comprises a heavy chain and a light chain, wherein the heavy chain variable region comprises or consists of a nucleotide sequence selected from SEQ ID NO: 261 or a sequence which is at least 80%, 85%, 90%, 95%, 96%, 97%, 98% or 99% identical thereto, and wherein the light chain variable region comprises or consists of a nucleotide sequence selected from SEQ ID NO: 365 or a sequence which is at least 80%, 85%, 90%, 95%, 96%, 97%, 98% or 99% identical thereto. Preferably, the heavy chain has a murine IgG1 isotype. More preferably, the heavy chain has VDJ regions IGHV1-63*02, IGHD1-1*01_5-3 and IGHJ2*02. Preferably, the light chain is a Kappa light chain class. More preferably, the light chain has VJ regions IGKV6-32*01 and IGKJ4*01.

In one embodiment, the mAb which binds to an epitope located on the HSV gE FCBD, comprises a heavy chain and a light chain, wherein the heavy chain variable region comprises or consists of the sequence shown in SEQ ID NO: 29 or a sequence which is at least 80%, 85%, 90%, 95%, 96%, 97%, 98% or 99% identical thereto, and wherein the light chain variable region comprises or consists of the sequence shown in SEQ ID NO: 133 or a sequence which is at least 80%, 85%, 90%, 95%, 96%, 97%, 98% or 99% identical thereto. In one embodiment, the mAb which binds to an epitope located on the HSV gE FCBD, comprises a heavy chain and a light chain, wherein the heavy chain variable region comprises or consists of a nucleotide sequence selected from SEQ ID NO: 241 or a sequence which is at least 80%, 85%, 90%, 95%, 96%, 97%, 98% or 99% identical thereto, and wherein the light chain variable region comprises or consists of a nucleotide sequence selected from SEQ ID NO: 345 or a sequence which is at least 80%, 85%, 90%, 95%, 96%, 97%, 98% or 99% identical thereto. Preferably, the heavy chain has a murine IgG1 isotype. More preferably, the heavy chain has VDJ regions IGHV2-9-1*01, IGHD2-4*01_5-3 and IGHJ4*01. Preferably, the light chain is a Kappa light chain class. More preferably, the light chain has VJ regions IGKV3-1*01 and IGKJ1*01.

In one embodiment, the mAb which binds to an epitope located on the HSV gE FCBD, comprises a heavy chain and a light chain, wherein the heavy chain variable region comprises or consists of the sequence shown in SEQ ID NO: 33 or a sequence which is at least 80%, 85%, 90%, 95%, 96%, 97%, 98% or 99% identical thereto, and wherein the light chain variable region comprises or consists of the sequence shown in SEQ ID NO: 137 or a sequence which is at least 80%, 85%, 90%, 95%, 96%, 97%, 98% or 99% identical thereto. In one embodiment, the mAb which binds to an epitope located on the HSV gE FCBD, comprises a heavy chain and a light chain, wherein the heavy chain variable region comprises or consists of a nucleotide sequence selected from SEQ ID NO: 245 or a sequence which is at least 80%, 85%, 90%, 95%, 96%, 97%, 98% or 99% identical thereto, and wherein the light chain variable region comprises or consists of a nucleotide sequence selected from SEQ ID NO: 349 or a sequence which is at least 80%, 85%, 90%, 95%, 96%, 97%, 98% or 99% identical thereto. Preferably, the heavy chain has a murine IgG1 isotype. More preferably, the heavy chain has VDJ regions IGHV1-9*01, IGHD6-1_01_3-5 and IGHJ4*01. Preferably, the light chain is a Kappa light chain class. More preferably, the light chain has VJ regions IGKV3-12*01 and IGKJ5*01.

In one embodiment, the mAb which binds to an epitope located on the HSV gE FCBD, comprises a heavy chain and a light chain, wherein the heavy chain variable region comprises or consists of the sequence shown in SEQ ID NO: 93 or a sequence which is at least 80%, 85%, 90%, 95%, 96%, 97%, 98% or 99% identical thereto, and wherein the light chain variable region comprises or consists of the sequence shown in SEQ ID NO: 201 or a sequence which is at least 80%, 85%, 90%, 95%, 96%, 97%, 98% or 99% identical thereto. In one embodiment, the mAb which binds to an epitope located on the HSV gE FCBD, comprises a heavy chain and a light chain, wherein the heavy chain variable region comprises or consists of a nucleotide sequence selected from SEQ ID NO: 305 or a sequence which is at least 80%, 85%, 90%, 95%, 96%, 97%, 98% or 99% identical thereto, and wherein the light chain variable region comprises or consists of a nucleotide sequence selected from SEQ ID NO: 413 or a sequence which is at least 80%, 85%, 90%, 95%, 96%, 97%, 98% or 99% identical thereto. Preferably, the heavy chain has a murine IgG1 isotype. More preferably, the heavy chain has VDJ regions IGHV5-6-4*01, IGHD1-1*01_5-3 and IGHJ2*01. Preferably, the light chain is a Kappa light chain class. More preferably, the light chain has VJ regions IGKV8-21*01 and IGKJ5*01.

In one embodiment, the mAb which binds to an epitope located on the HSV gE FCBD, comprises a heavy chain and a light chain, wherein the heavy chain variable region comprises or consists of the sequence shown in SEQ ID NO: 105 or a sequence which is at least 80%, 85%, 90%, 95%, 96%, 97%, 98% or 99% identical thereto, and wherein the light chain variable region comprises or consists of the sequence shown in SEQ ID NO: 213 or a sequence which is at least 80%, 85%, 90%, 95%, 96%, 97%, 98% or 99% identical thereto. In one embodiment, the mAb which binds to an epitope located on the HSV gE FCBD, comprises a heavy chain and a light chain, wherein the heavy chain variable region comprises or consists of a nucleotide sequence selected from SEQ ID NO: 317 or a sequence which is at least 80%, 85%, 90%, 95%, 96%, 97%, 98% or 99% identical thereto, and wherein the light chain variable region comprises or consists of a nucleotide sequence selected from SEQ ID NO: 425 or a sequence which is at least 80%, 85%, 90%, 95%, 96%, 97%, 98% or 99% identical thereto. Preferably, the heavy chain has a murine IgG1 isotype. More preferably, the heavy chain has VDJ regions IGHV7-3*02, IGHD3-1*01_5-3 and IGHJ2*01. Preferably, the light chain is a Kappa light chain class. More preferably, the light chain has VJ regions IGKV3-10*01 and IGKJ1*01.

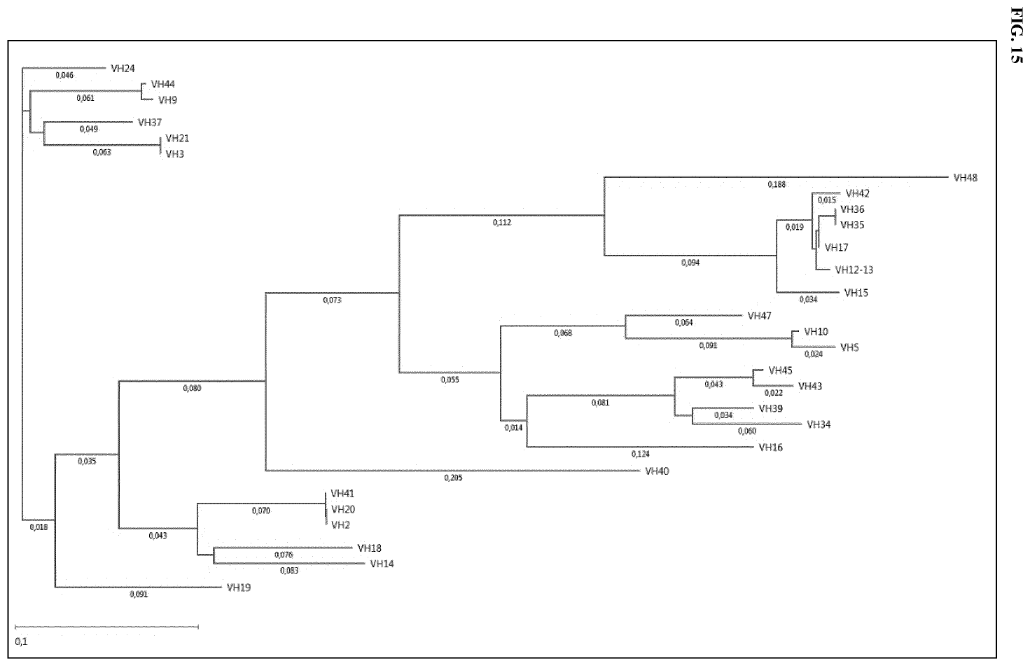

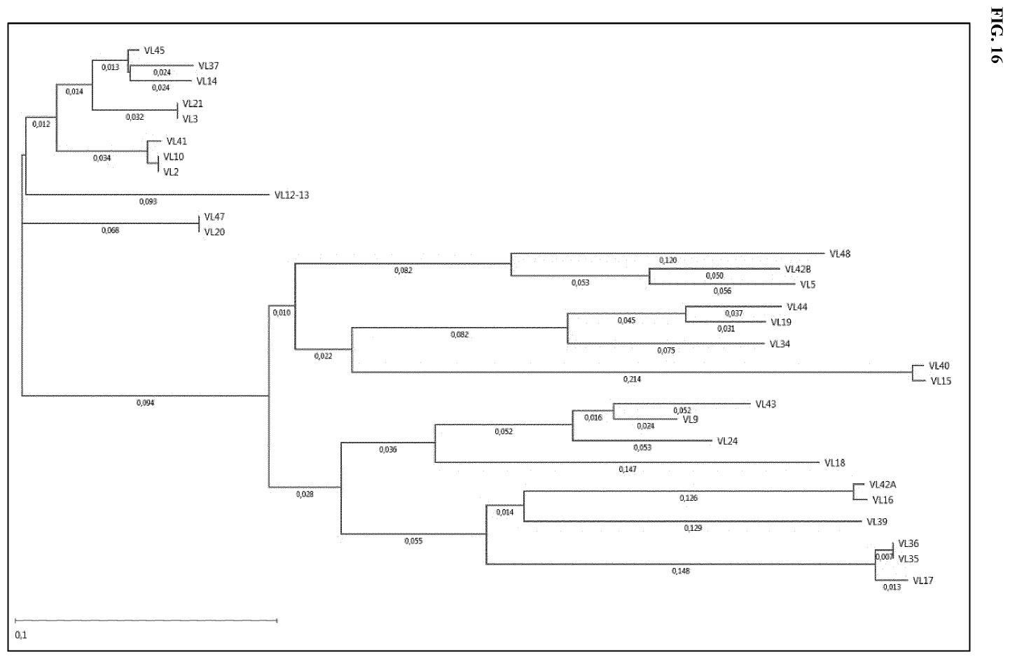

The binding of the Fc domain of human IgGs to the Fc binding domain (FCBD) of the gE protein forming part of the Fc HSV gEgI heterodimer triggers an immune-evasion mechanism. Indeed, HSV gE functions as a viral Fc gamma receptor (FcγR), meaning it has the capacity to interact with the Fc portion of human IgG. Human IgGs which can bind HSV1 or HSV2 antigens (for example gD) on the virion or infected cell through the IgG Fab domain can also bind the Fc binding domain on the viral gE through their Fc domain, leading to endocytosis of the immune complex. This mechanism is referred to as antibody bipolar bridging and is postulated to be a major immune evasion strategy competing with innate immune cell activation. Through antibody Fc binding, the viral FcγR inhibits IgG Fc-mediated activities, including complement binding and antibody-dependent cellular cytotoxicity (ADCC) allowing the virus to circumvent the recognition by the immune system (FIG. 14, Ndjamen, Blaise, et al. “The herpes virus Fc receptor gEgI mediates antibody bipolar bridging to clear viral antigens from the cell surface.” PLoS pathogens 10.3 (2014): e1003961.; Dubin, G., et al. “Herpes simplex virus type 1 Fc receptor protects infected cells from antibody-dependent cellular cytotoxicity.” Journal of virology 65.12 (1991): 7046-7050.; Sprague, Elizabeth R., et al. “Crystal structure of the HSV1 Fc receptor bound to Fc reveals a mechanism for antibody bipolar bridging.” PLoS biology 4.6 (2006): e148.).

In a preferred embodiment, the mAb which binds to an epitope located on the HSV gE FCBD is a functional antibody. Herein, a “functional antibody” is an antibody which is specific to the gE FCBD and which has the ability to block binding of the Fc domain of human IgGs and avoid the immune-evasion mechanism. Preferably, the functional antibody is a functional mAb. An example of a functional mAb is the mAb from clone 18 (see data presented in example 7).

In a preferred embodiment, the functional mAb which binds to an epitope located on the HSV gE FCBD, comprises a heavy chain, wherein the heavy chain variable region comprises the HCDR1 shown in SEQ ID NO: 50, the HCDR2 shown in SEQ ID NO: 51 and the HCDR3 shown in SEQ ID NO: 52, or variants thereof having 1, 2, or 3 amino acid deletions, substitutions or insertions.

In a preferred embodiment, the functional mAb which binds to an epitope located on the HSV gE FCBD, comprises a light chain, wherein the light chain variable region comprises the LCDR1 shown in SEQ ID NO: 154, the LCDR2 shown in SEQ ID NO: 155 and the LCDR3 shown in SEQ ID NO: 156, or variants thereof having 1, 2, or 3 amino acid deletions, substitutions or insertions.

In a preferred embodiment, the functional mAb which binds to an epitope located on the HSV gE FCBD, comprises a heavy chain and a light chain, wherein the heavy chain variable region comprises the HCDR1 shown in SEQ ID NO: 50, the HCDR2 shown in SEQ ID NO: 51 and the HCDR3 shown in SEQ ID NO: 52, and the light chain variable region comprises the LCDR1 shown in SEQ ID NO: 154, the LCDR2 shown in SEQ ID NO: 155 and the LCDR3 shown in SEQ ID NO: 156.

In a preferred embodiment, the functional mAb which binds to an epitope located on the HSV gE FCBD, comprises a heavy chain and a light chain, wherein the heavy chain variable region comprises or consists of the sequence shown in SEQ ID NO: 49 or a sequence which is at least 80%, 85%, 90%, 95%, 96%, 97%, 98% or 99% identical thereto, and wherein the light chain variable region comprises or consists of the sequence shown in SEQ ID NO: 153 or a sequence which is at least 80%, 85%, 90%, 95%, 96%, 97%, 98% or 99% identical thereto. Preferably, the heavy chain has a murine IgG1 isotype. More preferably, the heavy chain has VDJ regions IGHV1-63*02, IGHD1-1*01_5-3 and IGHJ2*02. Preferably, the light chain is a Kappa light chain class. More preferably, the light chain has VJ regions IGKV6-32*01 and IGKJ4*01.

In another embodiment, the mAb binds to a gE epitope located outside of the HSV gE FCBD. Examples of mAbs which bind to a gE epitope located outside of the HSV gE FCBD include mAbs from clones 3/21, 37, 44 and 48 (see example section and tables 7-11).

In a preferred embodiment, the mAb which binds to a gE epitope located outside of the HSV gE FCBD, comprises any one or a combination of CDRs selected from SEQ ID NOs: 14-16, 74-76, 98-100,110-112, 118-120, 178-180, 206-208 and 218-220, or variants thereof, wherein the variant has 1, 2, or 3 amino acid deletions, substitutions or insertions.

In a preferred embodiment, the mAb which binds to a gE epitope located outside of the HSV gE FCBD, comprises any one or a combination of CDRs encoded by SEQ ID NOs: 226-228, 286-288, 310-312, 322-324, 330-332, 390-392, 418-420 and 430-432, or variants thereof, wherein the variant has 1, 2, 3, 4, 5, 6, 7, 8, 9 or 10 nucleotide deletions, substitutions or insertions.

In a preferred embodiment, the mAb which binds a gE epitope located outside of the HSV gE FCBD, comprises a heavy chain, wherein the heavy chain variable region comprises:
a) the HCDR1 shown in SEQ ID NO: 14, the HCDR2 shown in SEQ ID NO: 15 and the HCDR3 shown in SEQ ID NO: 16, or variants thereof having 1, 2, or 3 amino acid deletions, substitutions or insertions,
b) the HCDR1 shown in SEQ ID NO: 74, the HCDR2 shown in SEQ ID NO: 75 and the HCDR3 shown in SEQ ID NO: 76, or variants thereof having 1, 2, or 3 amino acid deletions, substitutions or insertions, or
c) the HCDR1 shown in SEQ ID NO: 98, the HCDR2 shown in SEQ ID NO: 99 and the HCDR3 shown in SEQ ID NO: 100, or variants thereof having 1, 2, or 3 amino acid deletions, substitutions or insertions, or
d) the HCDR1 shown in SEQ ID NO: 110, the HCDR2 shown in SEQ ID NO: 111 and the HCDR3 shown in SEQ ID NO: 112, or variants thereof having 1, 2, or 3 amino acid deletions, substitutions or insertions.

In a preferred embodiment, the mAb which binds to a gE epitope located outside of the HSV gE FCBD, comprises a light chain, wherein the light chain variable region comprises:
a) the LCDR1 shown in SEQ ID NO: 118, the LCDR2 shown in SEQ ID NO: 119 and the LCDR3 shown in SEQ ID NO: 120, or variants thereof having 1, 2, or 3 amino acid deletions, substitutions or insertions,
b) the LCDR1 shown in SEQ ID NO: 178, the LCDR2 shown in SEQ ID NO: 179 and the LCDR3 shown in SEQ ID NO: 180, or variants thereof having 1, 2, or 3 amino acid deletions, substitutions or insertions,
c) the LCDR1 shown in SEQ ID NO: 206, the LCDR2 shown in SEQ ID NO: 207 and the LCDR3 shown in SEQ ID NO: 208, or variants thereof having 1, 2, or 3 amino acid deletions, substitutions or insertions, or
d) the LCDR1 shown in SEQ ID NO: 218, the LCDR2 shown in SEQ ID NO: 219 and the LCDR3 shown in SEQ ID NO: 220, or variants thereof having 1, 2, or 3 amino acid deletions, substitutions or insertions.

In a preferred embodiment, the mAb which binds to a gE epitope located outside of the HSV gE FCBD, comprises a heavy chain and a light chain, wherein:
a) the heavy chain variable region comprises the HCDR1 shown in SEQ ID NO: 14, the HCDR2 shown in SEQ ID NO: 15 and the HCDR3 shown in SEQ ID NO: 16, and the light chain variable region comprises the LCDR1 shown in SEQ ID NO: 118, the LCDR2 shown in SEQ ID NO: 119 and the LCDR3 shown in SEQ ID NO: 120,
b) the heavy chain variable region comprises the HCDR1 shown in SEQ ID NO: 74, the HCDR2 shown in SEQ ID NO: 75 and the HCDR3 shown in SEQ ID NO: 76, and the light chain variable region comprises the LCDR1 shown in SEQ ID NO: 178, the LCDR2 shown in SEQ ID NO: 179 and the LCDR3 shown in SEQ ID NO: 180,
c) the heavy chain variable region comprises the HCDR1 shown in SEQ ID NO: 98, the HCDR2 shown in SEQ ID NO: 99 and the HCDR3 shown in SEQ ID NO: 100, and the light chain variable region comprises the LCDR1 shown in SEQ ID NO: 206, the LCDR2 shown in SEQ ID NO: 207 and the LCDR3 shown in SEQ ID NO: 208, or
d) the heavy chain variable region comprises the HCDR1 shown in SEQ ID NO: 110, the HCDR2 shown in SEQ ID NO: 111 and the HCDR3 shown in SEQ ID NO: 112, and the light chain variable region comprises the LCDR1 shown in SEQ ID NO: 218, the LCDR2 shown in SEQ ID NO: 219 and the LCDR3 shown in SEQ ID NO: 220.

In a preferred embodiment, the mAb which binds a gE epitope located outside of the HSV gE FCBD, comprises a heavy chain, wherein the heavy chain variable region comprises:
a) the HCDR1 encoded by the nucleotide sequence SEQ ID NO: 226, the HCDR2 encoded by the nucleotide sequence SEQ ID NO: 227 and the HCDR3 encoded by the nucleotide sequence SEQ ID NO: 228, or variants thereof having 1, 2, 3, 4, 5, 6, 7, 8, 9 or 10 nucleotide deletions, substitutions or insertions,
b) the HCDR1 encoded by the nucleotide sequence SEQ ID NO: 286, the HCDR2 encoded by the nucleotide sequence SEQ ID NO: 287 and the HCDR3 encoded by the nucleotide sequence SEQ ID NO: 288, or variants thereof having 1, 2, 3, 4, 5, 6, 7, 8, 9 or 10 nucleotide deletions, substitutions or insertions, or
c) the HCDR1 encoded by the nucleotide sequence SEQ ID NO: 310, the HCDR2 encoded by the nucleotide sequence SEQ ID NO: 311 and the HCDR3 encoded by the nucleotide sequence SEQ ID NO: 312, or variants thereof having 1, 2, 3, 4, 5, 6, 7, 8, 9 or 10 nucleotide deletions, substitutions or insertions, or
d) the HCDR1 encoded by the nucleotide sequence SEQ ID NO: 322, the HCDR2 encoded by the nucleotide sequence SEQ ID NO: 323 and the HCDR3 encoded by the nucleotide sequence SEQ ID NO: 324, or variants thereof having 1, 2, 3, 4, 5, 6, 7, 8, 9 or 10 nucleotide deletions, substitutions or insertions.

In a preferred embodiment, the mAb which binds to a gE epitope located outside of the HSV gE FCBD, comprises a light chain, wherein the light chain variable region comprises:
a) the LCDR1 encoded by the nucleotide sequence SEQ ID NO: 330, the LCDR2 encoded by the nucleotide sequence SEQ ID NO: 331 and the LCDR3 encoded by the nucleotide sequence SEQ ID NO: 332, or variants thereof having 1, 2, 3, 4, 5, 6, 7, 8, 9 or 10 nucleotide deletions, substitutions or insertions,
b) the LCDR1 encoded by the nucleotide sequence SEQ ID NO: 390, the LCDR2 encoded by the nucleotide sequence SEQ ID NO: 391 and the LCDR3 encoded by the nucleotide sequence SEQ ID NO: 392, or variants thereof having 1, 2, 3, 4, 5, 6, 7, 8, 9 or 10 nucleotide deletions, substitutions or insertions,
c) the LCDR1 encoded by the nucleotide sequence SEQ ID NO: 418, the LCDR2 encoded by the nucleotide sequence SEQ ID NO: 419 and the LCDR3 encoded by the nucleotide sequence SEQ ID NO: 420, or variants thereof having 1, 2, 3, 4, 5, 6, 7, 8, 9 or 10 nucleotide deletions, substitutions or insertions, or
d) the LCDR1 encoded by the nucleotide sequence SEQ ID NO: 430, the LCDR2 encoded by the nucleotide sequence SEQ ID NO: 431 and the LCDR3 encoded by the nucleotide sequence SEQ ID NO: 432, or variants thereof having 1, 2, 3, 4, 5, 6, 7, 8, 9 or 10 nucleotide deletions, substitutions or insertions.

In a preferred embodiment, the mAb which to a gE epitope located outside of the HSV gE FCBD, comprises a heavy chain and a light chain, wherein:
a) the heavy chain variable region comprises the HCDR1 encoded by the nucleotide sequence SEQ ID NO: 226, the HCDR2 encoded by the nucleotide sequence SEQ ID NO: 227 and the HCDR3 encoded by the nucleotide sequence SEQ ID NO: 228, and the light chain variable region comprises the LCDR1 encoded by the nucleotide sequence SEQ ID NO: 330, the LCDR2 encoded by the nucleotide sequence SEQ ID NO: 331 and the LCDR3 encoded by the nucleotide sequence SEQ ID NO: 332,
b) the heavy chain variable region comprises the HCDR1 encoded by the nucleotide sequence SEQ ID NO: 286, the HCDR2 encoded by the nucleotide sequence SEQ ID NO: 287 and the HCDR3 encoded by the nucleotide sequence SEQ ID NO: 288, and the light chain variable region comprises the LCDR1 encoded by the nucleotide sequence SEQ ID NO: 390, the LCDR2 encoded by the nucleotide sequence SEQ ID NO: 391 and the LCDR3 encoded by the nucleotide sequence SEQ ID NO: 392,
c) the heavy chain variable region comprises the HCDR1 encoded by the nucleotide sequence SEQ ID NO: 310, the HCDR2 encoded by the nucleotide sequence SEQ ID NO: 311 and the HCDR3 encoded by the nucleotide sequence SEQ ID NO: 312, and the light chain variable region comprises the LCDR1 encoded by the nucleotide sequence SEQ ID NO: 418, the LCDR2 encoded by the nucleotide sequence SEQ ID NO: 419 and the LCDR3 encoded by the nucleotide sequence SEQ ID NO: 420, or
d) the heavy chain variable region comprises the HCDR1 encoded by the nucleotide sequence SEQ ID NO: 322, the HCDR2 encoded by the nucleotide sequence SEQ ID NO: 323 and the HCDR3 encoded by the nucleotide sequence SEQ ID NO: 324, and the light chain variable region comprises the LCDR1 encoded by the nucleotide sequence SEQ ID NO: 430, the LCDR2 encoded by the nucleotide sequence SEQ ID NO: 431 and the LCDR3 encoded by the nucleotide sequence SEQ ID NO: 432.

In one embodiment, the mAb which binds to a gE epitope located outside of the HSV gE FCBD, comprises a heavy chain, wherein the heavy chain variable region has a sequence selected from SEQ ID NO 13, SEQ ID NO 73, SEQ ID NO 97 and SEQ ID NO 109 or a sequence which is at least 80%, 85%, 90%, 95%, 96%, 97%, 98% or 99% identical thereto.

In one embodiment, the mAb which binds to a gE epitope located outside of the HSV gE FCBD, comprises a light chain, wherein the light chain variable region has a sequence selected from SEQ ID NO: 117, SEQ ID NO 177, SEQ ID NO 205 and SEQ ID NO 217 or a sequence which is at least 80%, 85%, 90%, 95%, 96%, 97%, 98% or 99% identical thereto.

In one embodiment, the mAb which binds to a gE epitope located outside of the HSV gE FCBD, comprises a heavy chain and a light chain, wherein the heavy chain variable region has a sequence selected from SEQ ID NO 13, SEQ ID NO 73, SEQ ID NO 97 and SEQ ID NO 109, or a sequence which is at least 80%, 85%, 90%, 95%, 96%, 97%, 98% or 99% identical thereto, and wherein the light chain variable region has a sequence selected from SEQ ID NO: 117, SEQ ID NO 177, SEQ ID NO 205 and SEQ ID NO 217, or a sequence which is at least 80%, 85%, 90%, 95%, 96%, 97%, 98% or 99% identical thereto.

In one embodiment, the mAb which binds to a gE epitope located outside of the HSV gE FCBD, comprises a heavy chain, wherein the heavy chain variable region comprises or consists of a nucleotide sequence selected from SEQ ID NO: 225, SEQ ID NO: 285, SEQ ID: NO 309 and SEQ ID NO: 321 or a sequence which is at least 80%, 85%, 90%, 95%, 96%, 97%, 98% or 99% identical thereto.

In one embodiment, the mAb which binds to a gE epitope located outside of the HSV gE FCBD, comprises a light chain, wherein the light chain variable region comprises or consists of a nucleotide sequence selected from SEQ ID NO: 329, SEQ ID NO: 389, SEQ ID NO: 417 and SEQ ID NO: 429 or a sequence which is at least 80%, 85%, 90%, 95%, 96%, 97%, 98% or 99% identical thereto.

In one embodiment, the mAb which binds to a gE epitope located outside of the HSV gE FCBD, comprises a heavy chain and a light chain, wherein the heavy chain variable region comprises or consists of a nucleotide sequence selected from SEQ ID NO: 225, SEQ ID NO: 285, SEQ ID: NO 309 and SEQ ID NO: 321, or a sequence which is at least 80%, 85%, 90%, 95%, 96%, 97%, 98% or 99% identical thereto, and wherein the light chain variable region comprises or consists of a nucleotide sequence selected from SEQ ID NO: 329, SEQ ID NO: 389, SEQ ID NO: 417 and SEQ ID NO: 429, or a sequence which is at least 80%, 85%, 90%, 95%, 96%, 97%, 98% or 99% identical thereto.

In one embodiment, the mAb which binds to a gE epitope located outside of the HSV gE FCBD, comprises a heavy chain and a light chain, wherein the heavy chain variable region comprises or consists of the sequence shown in SEQ ID NO: 13 or a sequence which is at least 80%, 85%, 90%, 95%, 96%, 97%, 98% or 99% identical thereto, and wherein the light chain variable region comprises or consists of the sequence shown in SEQ ID NO: 117 or a sequence which is at least 80%, 85%, 90%, 95%, 96%, 97%, 98% or 99% identical thereto. In one embodiment, the mAb which binds to a gE epitope located outside of the HSV gE FCBD, comprises a heavy chain and a light chain, wherein the heavy chain variable region comprises or consists of a nucleotide sequence selected from SEQ ID NO: 225 or a sequence which is at least 80%, 85%, 90%, 95%, 96%, 97%, 98% or 99% identical thereto, and wherein the light chain variable region comprises or consists of a nucleotide sequence selected from SEQ ID NO: 329 or a sequence which is at least 80%, 85%, 90%, 95%, 96%, 97%, 98% or 99% identical thereto. Preferably, the heavy chain has a murine IgG1 isotype. More preferably, the heavy chain has VDJ regions IGHV1-22*01, IGHD1-1*01_5-3 and IGHJ3*01. Preferably, the light chain is a Kappa light chain class. More preferably, the light chain has VJ regions IGKV3-7*01 and IGKJ2*01.

In one embodiment, the mAb which binds to a gE epitope located outside of the HSV gE FCBD, comprises a heavy chain and a light chain, wherein the heavy chain variable region comprises or consists of the sequence shown in SEQ ID NO: 73 or a sequence which is at least 80%, 85%, 90%, 95%, 96%, 97%, 98% or 99% identical thereto, and wherein the light chain variable region comprises or consists of the sequence shown in SEQ ID NO: 177 or a sequence which is at least 80%, 85%, 90%, 95%, 96%, 97%, 98% or 99% identical thereto. In one embodiment, the mAb which binds to a gE epitope located outside of the HSV gE FCBD, comprises a heavy chain and a light chain, wherein the heavy chain variable region comprises or consists of a nucleotide sequence selected from SEQ ID NO: 285 or a sequence which is at least 80%, 85%, 90%, 95%, 96%, 97%, 98% or 99% identical thereto, and wherein the light chain variable region comprises or consists of a nucleotide sequence selected from SEQ ID NO: 389 or a sequence which is at least 80%, 85%, 90%, 95%, 96%, 97%, 98% or 99% identical thereto. Preferably, the heavy chain has a murine IgG2a isotype. More preferably, the heavy chain has VDJ regions IGHV1S29*02, IGHD4-1*01_5-3 and IGHJ4*01. Preferably, the light chain is a Kappa light chain class. More preferably, the light chain has VJ regions IGKV3-12*01 and IGKJ1*01.

In one embodiment, the mAb which binds to a gE epitope located outside of the HSV gE FCBD, comprises a heavy chain and a light chain, wherein the heavy chain variable region comprises or consists of the sequence shown in SEQ ID NO: 97 or a sequence which is at least 80%, 85%, 90%, 95%, 96%, 97%, 98% or 99% identical thereto, and wherein the light chain variable region comprises or consists of the sequence shown in SEQ ID NO: 205 or a sequence which is at least 80%, 85%, 90%, 95%, 96%, 97%, 98% or 99% identical thereto. In one embodiment, the mAb which binds to a gE epitope located outside of the HSV gE FCBD, comprises a heavy chain and a light chain, wherein the heavy chain variable region comprises or consists of a nucleotide sequence selected from SEQ ID NO: 309 or a sequence which is at least 80%, 85%, 90%, 95%, 96%, 97%, 98% or 99% identical thereto, and wherein the light chain variable region comprises or consists of a nucleotide sequence selected from SEQ ID NO: 417 or a sequence which is at least 80%, 85%, 90%, 95%, 96%, 97%, 98% or 99% identical thereto. Preferably, the heavy chain has a murine IgG2a isotype. More preferably, the heavy chain has VDJ regions IGHV1S135*01, IGHD5-7_01_3-5 and IGHJ4*01. Preferably, the light chain is a Kappa light chain class. More preferably, the light chain has VJ regions IGKV4-70*01 and IGKJ2*01.

In one embodiment, the mAb which binds to a gE epitope located outside of the HSV gE FCBD, comprises a heavy chain and a light chain, wherein the heavy chain variable region comprises or consists of the sequence shown in SEQ ID NO: 109 or a sequence which is at least 80%, 85%, 90%, 95%, 96%, 97%, 98% or 99% identical thereto, and wherein the light chain variable region comprises or consists of the sequence shown in SEQ ID NO: 217 or a sequence which is at least 80%, 85%, 90%, 95%, 96%, 97%, 98% or 99% identical thereto. In one embodiment, the mAb which binds to a gE epitope located outside of the HSV gE FCBD, comprises a heavy chain and a light chain, wherein the heavy chain variable region comprises or consists of a nucleotide sequence selected from SEQ ID NO: 321 or a sequence which is at least 80%, 85%, 90%, 95%, 96%, 97%, 98% or 99% identical thereto, and wherein the light chain variable region comprises or consists of a nucleotide sequence selected from SEQ ID NO: 429 or a sequence which is at least 80%, 85%, 90%, 95%, 96%, 97%, 98% or 99% identical thereto. Preferably, the heavy chain has a murine IgG1 isotype. More preferably, the heavy chain has VDJ regions IGHV8-12*01, IGHD2-14*01_5-3 and IGHJ4*01. Preferably, the light chain is a Kappa light chain class. More preferably, the light chain has VJ regions IGKV5-48*01 and IGKJ5*01.

In another aspect, the invention provides an antigen binding protein which binds to a conformational epitope on the HSV gEgI heterodimer which is located at the interface between HSV gE and HSV gI. A conformational epitope located at the interface between HSV gE and HSV gI is an epitope which comprises amino acid residues from HSV gE and from HSV gI. Such an epitope is thus only apparent when the HSV gE and gI form a noncovalent heterodimer. An antigen binding protein which is specific to a conformational epitope located at the interface between HSV gE and HSV gI of the heterodimer does not specifically bind to free gE or to free gI. In a preferred embodiment, the antigen binding protein is an antibody which binds to a conformational epitope on the HSV gEgI heterodimer which is located at the interface between HSV gE and HSV gI. In a more preferred embodiment, the antibody is a monoclonal antibody (mAb) which binds to a conformational epitope on the HSV gEgI heterodimer which is located at the interface between HSV gE and HSV gI

Examples of mAbs which bind to the HSV gEgI heterodimer at a conformational epitope located at the interface between HSV gE and HSV gI include mAbs from clones 5, 9, 10, 15, 17, 19, 20, 24, 34, 39, 40, 42 and 45 (see example section and tables 7-11).

In a preferred embodiment, the mAb which binds to the HSV gEgI heterodimer at a conformational epitope located at the interface between HSV gE and HSV gI, comprises any one or a combination of CDRs selected from SEQ ID NOs: 18-20, 22-24, 26-28, 38-40, 46-48, 54-56, 58-60, 62-64, 66-68, 78-80, 82-84, 90-92, 102-104, 122-124, 126-128, 130-132, 142-444, 150-152, 158-160, 162-164, 166-168, 170-172, 182-184, 186-188, 194-196, 198-200 and 210-212, or variants thereof, wherein the variant has 1, 2, or 3 amino acid deletions, substitutions or insertions.

In a preferred embodiment, the mAb which binds to the HSV gEgI heterodimer at a conformational epitope located at the interface between HSV gE and HSV gI, comprises any one or a combination of CDRs encoded by SEQ ID NOs: 230-232, 234-236, 238-240, 250-252, 258-260, 266-268, 270-272, 274-276, 278-280, 290-292, 294-296, 302-304, 314-316, 334-336, 338-340, 342-344, 354-356, 362-364, 370-372, 374-376, 378-380, 382-384, 394-396, 398-400, 406-408, 410-412 and 422-424, or variants thereof, wherein the variant has 1, 2, 3, 4, 5, 6, 7, 8, 9 or nucleotide deletions, substitutions or insertions.

In a preferred embodiment, the mAb which binds to the HSV gEgI heterodimer at a conformational epitope located at the interface between HSV gE and HSV gI, comprises a heavy chain, wherein the heavy chain variable region comprises:
a) the HCDR1 shown in SEQ NO: 18, the HCDR2 shown in SEQ NO: 19 and the HCDR3 shown in SEQ NO: 20, or variants thereof having 1, 2, or 3 amino acid deletions, substitutions or insertions,
b) the HCDR1 shown in SEQ NO: 22, the HCDR2 shown in SEQ NO: 23 and the HCDR3 shown in SEQ NO: 24, or variants thereof having 1, 2, or 3 amino acid deletions, substitutions or insertions,
c) the HCDR1 shown in SEQ NO:26, the HCDR2 shown in SEQ NO: 27 and the HCDR3 shown in SEQ NO: 28, or variants thereof having 1, 2, or 3 amino acid deletions, substitutions or insertions,
d) the HCDR1 shown in SEQ NO: 38, the HCDR2 shown in SEQ NO: 39 and the HCDR3 shown in SEQ NO: 40, or variants thereof having 1, 2, or 3 amino acid deletions, substitutions or insertions,
e) the HCDR1 shown in SEQ NO: 46, the HCDR2 shown in SEQ NO: 47 and the HCDR3 shown in SEQ NO: 48, or variants thereof having 1, 2, or 3 amino acid deletions, substitutions or insertions,
f) the HCDR1 shown in SEQ NO: 54, the HCDR2 shown in SEQ NO: 55 and the HCDR3 shown in SEQ NO: 56, or variants thereof having 1, 2, or 3 amino acid deletions, substitutions or insertions,
g) the HCDR1 shown in SEQ NO: 58, the HCDR2 shown in SEQ NO: 59 and the HCDR3 shown in SEQ NO: 60, or variants thereof having 1, 2, or 3 amino acid deletions, substitutions or insertions,
h) the HCDR1 shown in SEQ NO: 62, the HCDR2 shown in SEQ NO: 63 and the HCDR3 shown in SEQ NO: 64, or variants thereof having 1, 2, or 3 amino acid deletions, substitutions or insertions,
i) the HCDR1 shown in SEQ NO: 66, the HCDR2 shown in SEQ NO: 67 and the HCDR3 shown in SEQ NO: 68, or variants thereof having 1, 2, or 3 amino acid deletions, substitutions or insertions,
j) the HCDR1 shown in SEQ NO: 78, the HCDR2 shown in SEQ NO: 79 and the HCDR3 shown in SEQ NO: 80, or variants thereof having 1, 2, or 3 amino acid deletions, substitutions or insertions,
k) the HCDR1 shown in SEQ NO: 82, the HCDR2 shown in SEQ NO: 83 and the HCDR3 shown in SEQ NO: 84, or variants thereof having 1, 2, or 3 amino acid deletions, substitutions or insertions,
1) the HCDR1 shown in SEQ NO: 90, the HCDR2 shown in SEQ NO: 91 and the HCDR3 shown in SEQ NO: 92, or variants thereof having 1, 2, or 3 amino acid deletions, substitutions or insertions, or
m) the HCDR1 shown in SEQ NO: 102, the HCDR2 shown in SEQ NO: 103 and the HCDR3 shown in SEQ NO: 104, or variants thereof having 1, 2, or 3 amino acid deletions, substitutions or insertions.

In a preferred embodiment, the mAb which binds to the HSV gEgI heterodimer at a conformational epitope located at the interface between HSV gE and HSV gI, comprises a light chain, wherein the light chain variable region comprises:
a) the LCDR1 shown in SEQ NO:122, the LCDR2 shown in SEQ NO: 123 and the LCDR3 shown in SEQ NO: 124, or variants thereof having 1, 2, or 3 amino acid deletions, substitutions or insertions,
b) the LCDR1 shown in SEQ NO: 126, the LCDR2 shown in SEQ NO: 127 and the LCDR3 shown in SEQ NO: 128, or variants thereof having 1, 2, or 3 amino acid deletions, substitutions or insertions,
c) the LCDR1 shown in SEQ NO: 130, the LCDR2 shown in SEQ NO: 131 and the LCDR3 shown in SEQ NO: 132 or variants thereof having 1, 2, or 3 amino acid deletions, substitutions or insertions,
d) the LCDR1 shown in SEQ NO: 142, the LCDR2 shown in SEQ NO: 143 and the LCDR3 shown in SEQ NO: 144, or variants thereof having 1, 2, or 3 amino acid deletions, substitutions or insertions,
e) the LCDR1 shown in SEQ NO: 150; the LCDR2 shown in SEQ NO: 151 and the LCDR3 shown in SEQ NO: 152, or variants thereof having 1, 2, or 3 amino acid deletions, substitutions or insertions,
f) the LCDR1 shown in SEQ NO: 158; the LCDR2 shown in SEQ NO: 159 and the LCDR3 shown in SEQ NO: 160, or variants thereof having 1, 2, or 3 amino acid deletions, substitutions or insertions,
g) the LCDR1 shown in SEQ NO: 162, the LCDR2 shown in SEQ NO: 163 and the LCDR3 shown in SEQ NO: 164, or variants thereof having 1, 2, or 3 amino acid deletions, substitutions or insertions,
h) the LCDR1 shown in SEQ NO: 166, the LCDR2 shown in SEQ NO: 167 and the LCDR3 shown in SEQ NO: 168, or variants thereof having 1, 2, or 3 amino acid deletions, substitutions or insertions,
i) the LCDR1 shown in SEQ NO: 170, the LCDR2 shown in SEQ NO: 171 and the LCDR3 shown in SEQ NO: 172, or variants thereof having 1, 2, or 3 amino acid deletions, substitutions or insertions,
j) the LCDR1 shown in SEQ NO: 182, the LCDR2 shown in SEQ NO: 183 and the LCDR3 shown in SEQ NO: 184, or variants thereof having 1, 2, or 3 amino acid deletions, substitutions or insertions,
k) the LCDR1 shown in SEQ NO: 186; the LCDR2 shown in SEQ NO: 187 and the LCDR3 shown in SEQ NO: 188, or variants thereof having 1, 2, or 3 amino acid deletions, substitutions or insertions,
1) the LCDR1 shown in SEQ NO: 194, the LCDR2 shown in SEQ NO: 195 and the LCDR3 shown in SEQ NO: 196, or variants thereof having 1, 2, or 3 amino acid deletions, substitutions or insertions, or
m) the LCDR1 shown in SEQ NO: 198, the LCDR2 shown in SEQ NO: 199 and the LCDR3 shown in SEQ NO: 200 or variants thereof having 1, 2, or 3 amino acid deletions, substitutions or insertions, or
n) the LCDR1 shown in SEQ NO: 210, the LCDR2 shown in SEQ NO: 211 and the LCDR3 shown in SEQ NO: 212, or variants thereof having 1, 2, or 3 amino acid deletions, substitutions or insertions.

In a preferred embodiment, the mAb which binds to the HSV gEgI heterodimer at a conformational epitope located at the interface between HSV gE and HSV gI, comprises a heavy chain and a light chain, wherein:
a) the heavy chain variable region comprises the HCDR1 shown in SEQ NO: 18, the HCDR2 shown in SEQ NO: 19 and the HCDR3 shown in SEQ NO: 20, and the light chain variable region comprises the LCDR1 shown in SEQ NO: 122, the LCDR2 shown in SEQ NO: 123 and the LCDR3 shown in SEQ NO: 124,
b) the heavy chain variable region comprises the HCDR1 shown in SEQ NO: 22, the HCDR2 shown in SEQ NO: 23 and the HCDR3 shown in SEQ NO: 24, and the light chain variable region comprises the LCDR1 shown in SEQ NO: 126, the LCDR2 shown in SEQ NO: 127 and the LCDR3 shown in SEQ NO: 128,
c) the heavy chain variable region comprises the HCDR1 shown in SEQ NO:26, the HCDR2 shown in SEQ NO: 27 and the HCDR3 shown in SEQ NO: 28, and the light chain variable region comprises the LCDR1 shown in SEQ NO: 130, the LCDR2 shown in SEQ NO: 131 and the LCDR3 shown in SEQ NO: 132,
d) the heavy chain variable region comprises the HCDR1 shown in SEQ NO: 38, the HCDR2 shown in SEQ NO: 39 and the HCDR3 shown in SEQ NO: 40, and the light chain variable region comprises the LCDR1 shown in SEQ NO: 142, the LCDR2 shown in SEQ NO: 143 and the LCDR3 shown in SEQ NO: 144,
e) the heavy chain variable region comprises the HCDR1 shown in SEQ NO: 46, the HCDR2 shown in SEQ NO: 47 and the HCDR3 shown in SEQ NO: 48, and the light chain variable region comprises the LCDR1 shown in SEQ NO: 150; the LCDR2 shown in SEQ NO: 151 and the LCDR3 shown in SEQ NO: 152,
f) the heavy chain variable region comprises the HCDR1 shown in SEQ NO: 54, the HCDR2 shown in SEQ NO: 55 and the HCDR3 shown in SEQ NO: 56, and the light chain variable region comprises the LCDR1 shown in SEQ NO: 158; the LCDR2 shown in SEQ NO: 159 and the LCDR3 shown in SEQ NO: 160,
g) the heavy chain variable region comprises the HCDR1 shown in SEQ NO: 58, the HCDR2 shown in SEQ NO: 59 and the HCDR3 shown in SEQ NO: 60, and the light chain variable region comprises the LCDR1 shown in SEQ NO: 162, the LCDR2 shown in SEQ NO: 163 and the LCDR3 shown in SEQ NO: 164,
h) the heavy chain variable region comprises the HCDR1 shown in SEQ NO: 62, the HCDR2 shown in SEQ NO: 63 and the HCDR3 shown in SEQ NO: 64, and the light chain variable region comprises the LCDR1 shown in SEQ NO: 166, the LCDR2 shown in SEQ NO: 167 and the LCDR3 shown in SEQ NO: 168,
i) the heavy chain variable region comprises the HCDR1 shown in SEQ NO: 66, the HCDR2 shown in SEQ NO: 67 and the HCDR3 shown in SEQ NO: 68, and the light chain variable region comprises the LCDR1 shown in SEQ NO: 170, the LCDR2 shown in SEQ NO: 171 and the LCDR3 shown in SEQ NO: 172,
j) the heavy chain variable region comprises the HCDR1 shown in SEQ NO: 78, the HCDR2 shown in SEQ NO: 79 and the HCDR3 shown in SEQ NO: 80, and the light chain variable region comprises the LCDR1 shown in SEQ NO: 182, the LCDR2 shown in SEQ NO: 183 and the LCDR3 shown in SEQ NO: 184,
k) the heavy chain variable region comprises the HCDR1 shown in SEQ NO: 82, the HCDR2 shown in SEQ NO: 83 and the HCDR3 shown in SEQ NO: 84, and the light chain variable region comprises the LCDR1 shown in SEQ NO: 186; the LCDR2 shown in SEQ NO: 187 and the LCDR3 shown in SEQ NO: 188,
1) the heavy chain variable region comprises the HCDR1 shown in SEQ NO: 90, the HCDR2 shown in SEQ NO: 91 and the HCDR3 shown in SEQ NO: 92, and the light chain variable region comprises the LCDR1 shown in SEQ NO: 194, the LCDR2 shown in SEQ NO: 195 and the LCDR3 shown in SEQ NO: 196,
m) the heavy chain variable region comprises the HCDR1 shown in SEQ NO: 90, the HCDR2 shown in SEQ NO: 91 and the HCDR3 shown in SEQ NO: 92, and the light chain variable region comprises the LCDR1 shown in SEQ NO: 198, the LCDR2 shown in SEQ NO: 199 and the LCDR3 shown in SEQ NO: 200, or
n) the heavy chain variable region comprises the HCDR1 shown in SEQ NO: 102, the HCDR2 shown in SEQ NO: 103 and the HCDR3 shown in SEQ NO: 104, and the light chain variable region comprises the LCDR1 shown in SEQ NO: 210, the LCDR2 shown in SEQ NO: 211 and the LCDR3 shown in SEQ NO: 212.

In a preferred embodiment, the mAb which binds to the HSV gEgI heterodimer at a conformational epitope located at the interface between HSV gE and HSV gI, comprises a heavy chain, wherein the heavy chain variable region comprises:
a) the HCDR1 encoded by the nucleotide sequence SEQ NO: 230, the HCDR2 encoded by the nucleotide sequence SEQ NO: 231 and the HCDR3 encoded by the nucleotide sequence SEQ NO: 232, or variants thereof having 1, 2, 3, 4, 5, 6, 7, 8, 9 or 10 nucleotide deletions, substitutions or insertions,
b) the HCDR1 encoded by the nucleotide sequence SEQ NO: 234, the HCDR2 encoded by the nucleotide sequence SEQ NO: 235 and the HCDR3 encoded by the nucleotide sequence SEQ NO: 236, or variants thereof having 1, 2, 3, 4, 5, 6, 7, 8, 9 or 10 nucleotide deletions, substitutions or insertions,
c) the HCDR1 encoded by the nucleotide sequence SEQ NO:238, the HCDR2 encoded by the nucleotide sequence SEQ NO: 239 and the HCDR3 encoded by the nucleotide sequence SEQ NO: 240, or variants thereof having 1, 2, 3, 4, 5, 6, 7, 8, 9 or 10 nucleotide deletions, substitutions or insertions,
d) the HCDR1 encoded by the nucleotide sequence SEQ NO: 250, the HCDR2 encoded by the nucleotide sequence SEQ NO: 251 and the HCDR3 encoded by the nucleotide sequence SEQ NO: 252, or variants thereof having 1, 2, 3, 4, 5, 6, 7, 8, 9 or 10 nucleotide deletions, substitutions or insertions,
e) the HCDR1 encoded by the nucleotide sequence SEQ NO: 258, the HCDR2 encoded by the nucleotide sequence SEQ NO: 259 and the HCDR3 encoded by the nucleotide sequence SEQ NO: 260, or variants thereof having 1, 2, 3, 4, 5, 6, 7, 8, 9 or 10 nucleotide deletions, substitutions or insertions,
f) the HCDR1 encoded by the nucleotide sequence SEQ NO: 266, the HCDR2 encoded by the nucleotide sequence SEQ NO: 267 and the HCDR3 encoded by the nucleotide sequence SEQ NO: 268, or variants thereof having 1, 2, 3, 4, 5, 6, 7, 8, 9 or 10 nucleotide deletions, substitutions or insertions,
g) the HCDR1 encoded by the nucleotide sequence SEQ NO: 270, the HCDR2 encoded by the nucleotide sequence SEQ NO: 271 and the HCDR3 encoded by the nucleotide sequence SEQ NO: 272, or variants thereof having 1, 2, 3, 4, 5, 6, 7, 8, 9 or 10 nucleotide deletions, substitutions or insertions,
h) the HCDR1 encoded by the nucleotide sequence SEQ NO: 274, the HCDR2 encoded by the nucleotide sequence SEQ NO: 275 and the HCDR3 encoded by the nucleotide sequence SEQ NO: 276, or variants thereof having 1, 2, 3, 4, 5, 6, 7, 8, 9 or 10 nucleotide deletions, substitutions or insertions,
i) the HCDR1 encoded by the nucleotide sequence SEQ NO: 278, the HCDR2 encoded by the nucleotide sequence SEQ NO: 279 and the HCDR3 encoded by the nucleotide sequence SEQ NO: 280, or variants thereof having 1, 2, 3, 4, 5, 6, 7, 8, 9 or 10 nucleotide deletions, substitutions or insertions,
j) the HCDR1 encoded by the nucleotide sequence SEQ NO: 290, the HCDR2 encoded by the nucleotide sequence SEQ NO: 291 and the HCDR3 encoded by the nucleotide sequence SEQ NO: 292, or variants thereof having 1, 2, 3, 4, 5, 6, 7, 8, 9 or 10 nucleotide deletions, substitutions or insertions,
k) the HCDR1 encoded by the nucleotide sequence SEQ NO: 294, the HCDR2 encoded by the nucleotide sequence SEQ NO: 295 and the HCDR3 encoded by the nucleotide sequence SEQ NO: 296, or variants thereof having 1, 2, 3, 4, 5, 6, 7, 8, 9 or 10 nucleotide deletions, substitutions or insertions,
1) the HCDR1 encoded by the nucleotide sequence SEQ NO: 302, the HCDR2 encoded by the nucleotide sequence SEQ NO: 303 and the HCDR3 encoded by the nucleotide sequence SEQ NO: 304, or variants thereof having 1, 2, 3, 4, 5, 6, 7, 8, 9 or 10 nucleotide deletions, substitutions or insertions, or
m) the HCDR1 encoded by the nucleotide sequence SEQ NO: 314, the HCDR2 encoded by the nucleotide sequence SEQ NO: 315 and the HCDR3 encoded by the nucleotide sequence SEQ NO: 316, or variants thereof having 1, 2, 3, 4, 5, 6, 7, 8, 9 or 10 nucleotide deletions, substitutions or insertions.

In a preferred embodiment, the mAb which binds to the HSV gEgI heterodimer at a conformational epitope located at the interface between HSV gE and HSV gI, comprises a light chain, wherein the light chain variable region comprises:
a) the LCDR1 encoded by the nucleotide sequence SEQ NO: 334, the LCDR2 encoded by the nucleotide sequence SEQ NO: 335 and the LCDR3 encoded by the nucleotide sequence SEQ NO: 336, or variants thereof having 1, 2, 3, 4, 5, 6, 7, 8, 9 or 10 nucleotide deletions, substitutions or insertions,
b) the LCDR1 encoded by the nucleotide sequence SEQ NO: 338, the LCDR2 encoded by the nucleotide sequence SEQ NO: 339 and the LCDR3 encoded by the nucleotide sequence SEQ NO: 340, or variants thereof having 1, 2, 3, 4, 5, 6, 7, 8, 9 or 10 nucleotide deletions, substitutions or insertions,
c) the LCDR1 encoded by the nucleotide sequence SEQ NO: 342, the LCDR2 encoded by the nucleotide sequence SEQ NO: 343 and the LCDR3 encoded by the nucleotide sequence SEQ NO: 344 or variants thereof having 1, 2, 3, 4, 5, 6, 7, 8, 9 or 10 nucleotide deletions, substitutions or insertions,
d) the LCDR1 encoded by the nucleotide sequence SEQ NO: 354, the LCDR2 encoded by the nucleotide sequence SEQ NO: 355 and the LCDR3 encoded by the nucleotide sequence SEQ NO: 356, or variants thereof having 1, 2, 3, 4, 5, 6, 7, 8, 9 or 10 nucleotide deletions, substitutions or insertions,
e) the LCDR1 encoded by the nucleotide sequence SEQ NO: 362; the LCDR2 encoded by the nucleotide sequence SEQ NO: 363 and the LCDR3 encoded by the nucleotide sequence SEQ NO: 364, or variants thereof having 1, 2, 3, 4, 5, 6, 7, 8, 9 or 10 nucleotide deletions, substitutions or insertions,
f) the LCDR1 encoded by the nucleotide sequence SEQ NO: 370; the LCDR2 encoded by the nucleotide sequence SEQ NO: 371 and the LCDR3 encoded by the nucleotide sequence SEQ NO: 372, or variants thereof having 1, 2, 3, 4, 5, 6, 7, 8, 9 or 10 nucleotide deletions, substitutions or insertions,
g) the LCDR1 encoded by the nucleotide sequence SEQ NO: 374, the LCDR2 encoded by the nucleotide sequence SEQ NO: 375 and the LCDR3 encoded by the nucleotide sequence SEQ NO: 376, or variants thereof having 1, 2, 3, 4, 5, 6, 7, 8, 9 or 10 nucleotide deletions, substitutions or insertions,
h) the LCDR1 encoded by the nucleotide sequence SEQ NO: 378, the LCDR2 encoded by the nucleotide sequence SEQ NO: 379 and the LCDR3 encoded by the nucleotide sequence SEQ NO: 380, or variants thereof having 1, 2, 3, 4, 5, 6, 7, 8, 9 or 10 nucleotide deletions, substitutions or insertions,
i) the LCDR1 encoded by the nucleotide sequence SEQ NO: 382, the LCDR2 encoded by the nucleotide sequence SEQ NO: 383 and the LCDR3 encoded by the nucleotide sequence SEQ NO: 384, or variants thereof having 1, 2, 3, 4, 5, 6, 7, 8, 9 or 10 nucleotide deletions, substitutions or insertions,
j) the LCDR1 encoded by the nucleotide sequence SEQ NO: 394, the LCDR2 encoded by the nucleotide sequence SEQ NO: 395 and the LCDR3 encoded by the nucleotide sequence SEQ NO: 396, or variants thereof having 1, 2, 3, 4, 5, 6, 7, 8, 9 or 10 nucleotide deletions, substitutions or insertions,
k) the LCDR1 encoded by the nucleotide sequence SEQ NO: 398; the LCDR2 encoded by the nucleotide sequence SEQ NO: 399 and the LCDR3 encoded by the nucleotide sequence SEQ NO: 400, or variants thereof having 1, 2, 3, 4, 5, 6, 7, 8, 9 or 10 nucleotide deletions, substitutions or insertions,
1) the LCDR1 encoded by the nucleotide sequence SEQ NO: 406, the LCDR2 encoded by the nucleotide sequence SEQ NO: 407 and the LCDR3 encoded by the nucleotide sequence SEQ NO: 408, or variants thereof having 1, 2, 3, 4, 5, 6, 7, 8, 9 or 10 nucleotide deletions, substitutions or insertions,
m) the LCDR1 encoded by the nucleotide sequence SEQ NO: 410, the LCDR2 encoded by the nucleotide sequence SEQ NO: 411 and the LCDR3 encoded by the nucleotide sequence SEQ NO: 412 or variants thereof having 1, 2, 3, 4, 5, 6, 7, 8, 9 or 10 nucleotide deletions, substitutions or insertions, or
n) the LCDR1 encoded by the nucleotide sequence SEQ NO: 422, the LCDR2 encoded by the nucleotide sequence SEQ NO: 423 and the LCDR3 encoded by the nucleotide sequence SEQ NO: 424, or variants thereof having 1, 2, 3, 4, 5, 6, 7, 8, 9 or 10 nucleotide deletions, substitutions or insertions.

In a preferred embodiment, the mAb which binds to the HSV gEgI heterodimer at a conformational epitope located at the interface between HSV gE and HSV gI, comprises a heavy chain and a light chain, wherein:
a) the heavy chain variable region comprises the HCDR1 encoded by the nucleotide sequence SEQ NO: 230, the HCDR2 encoded by the nucleotide sequence SEQ NO: 231 and the HCDR3 encoded by the nucleotide sequence SEQ NO: 232, and the light chain variable region comprises the LCDR1 encoded by the nucleotide sequence SEQ NO: 334, the LCDR2 encoded by the nucleotide sequence SEQ NO: 335 and the LCDR3 encoded by the nucleotide sequence SEQ NO: 336,
b) the heavy chain variable region comprises the HCDR1 encoded by the nucleotide sequence SEQ NO: 234, the HCDR2 encoded by the nucleotide sequence SEQ NO: 235 and the HCDR3 encoded by the nucleotide sequence SEQ NO: 236, and the light chain variable region comprises the LCDR1 encoded by the nucleotide sequence SEQ NO: 338, the LCDR2 encoded by the nucleotide sequence SEQ NO: 339 and the LCDR3 encoded by the nucleotide sequence SEQ NO: 340,
c) the heavy chain variable region comprises the HCDR1 encoded by the nucleotide sequence SEQ NO:238, the HCDR2 encoded by the nucleotide sequence SEQ NO: 239 and the HCDR3 encoded by the nucleotide sequence SEQ NO: 240, and the light chain variable region comprises the LCDR1 encoded by the nucleotide sequence SEQ NO: 342, the LCDR2 encoded by the nucleotide sequence SEQ NO: 343 and the LCDR3 encoded by the nucleotide sequence SEQ NO: 344,
d) the heavy chain variable region comprises the HCDR1 encoded by the nucleotide sequence SEQ NO: 250, the HCDR2 encoded by the nucleotide sequence SEQ NO: 251 and the HCDR3 encoded by the nucleotide sequence SEQ NO: 252, and the light chain variable region comprises the LCDR1 encoded by the nucleotide sequence SEQ NO: 354, the LCDR2 encoded by the nucleotide sequence SEQ NO: 355 and the LCDR3 encoded by the nucleotide sequence SEQ NO: 356,
e) the heavy chain variable region comprises the HCDR1 encoded by the nucleotide sequence SEQ NO: 258, the HCDR2 encoded by the nucleotide sequence SEQ NO: 259 and the HCDR3 encoded by the nucleotide sequence SEQ NO: 260, and the light chain variable region comprises the LCDR1 encoded by the nucleotide sequence SEQ NO: 362, the LCDR2 encoded by the nucleotide sequence SEQ NO: 363 and the LCDR3 encoded by the nucleotide sequence SEQ NO: 364,
f) the heavy chain variable region comprises the HCDR1 encoded by the nucleotide sequence SEQ NO: 266, the HCDR2 encoded by the nucleotide sequence SEQ NO: 267 and the HCDR3 encoded by the nucleotide sequence SEQ NO: 268, and the light chain variable region comprises the LCDR1 encoded by the nucleotide sequence SEQ NO: 370; the LCDR2 encoded by the nucleotide sequence SEQ NO: 371 and the LCDR3 encoded by the nucleotide sequence SEQ NO: 372,
g) the heavy chain variable region comprises the HCDR1 encoded by the nucleotide sequence SEQ NO: 270, the HCDR2 encoded by the nucleotide sequence SEQ NO: 271 and the HCDR3 encoded by the nucleotide sequence SEQ NO: 272, and the light chain variable region comprises the LCDR1 encoded by the nucleotide sequence SEQ NO: 374, the LCDR2 encoded by the nucleotide sequence SEQ NO: 375 and the LCDR3 encoded by the nucleotide sequence SEQ NO: 376,
h) the heavy chain variable region comprises the HCDR1 encoded by the nucleotide sequence SEQ NO: 274, the HCDR2 encoded by the nucleotide sequence SEQ NO: 275 and the HCDR3 encoded by the nucleotide sequence SEQ NO: 276, and the light chain variable region comprises the LCDR1 encoded by the nucleotide sequence SEQ NO: 378, the LCDR2 encoded by the nucleotide sequence SEQ NO: 379 and the LCDR3 encoded by the nucleotide sequence SEQ NO: 380,
i) the heavy chain variable region comprises the HCDR1 encoded by the nucleotide sequence SEQ NO: 278, the HCDR2 encoded by the nucleotide sequence SEQ NO: 279 and the HCDR3 encoded by the nucleotide sequence SEQ NO: 280, and the light chain variable region comprises the LCDR1 encoded by the nucleotide sequence SEQ NO: 383, the LCDR2 encoded by the nucleotide sequence SEQ NO: 383 and the LCDR3 encoded by the nucleotide sequence SEQ NO: 384,
j) the heavy chain variable region comprises the HCDR1 encoded by the nucleotide sequence SEQ NO: 290, the HCDR2 encoded by the nucleotide sequence SEQ NO: 291 and the HCDR3 encoded by the nucleotide sequence SEQ NO: 292, and the light chain variable region comprises the LCDR1 encoded by the nucleotide sequence SEQ NO: 394, the LCDR2 encoded by the nucleotide sequence SEQ NO: 395 and the LCDR3 encoded by the nucleotide sequence SEQ NO: 396,
k) the heavy chain variable region comprises the HCDR1 encoded by the nucleotide sequence SEQ NO: 294, the HCDR2 encoded by the nucleotide sequence SEQ NO: 295 and the HCDR3 encoded by the nucleotide sequence SEQ NO: 296, and the light chain variable region comprises the LCDR1 encoded by the nucleotide sequence SEQ NO: 398; the LCDR2 encoded by the nucleotide sequence SEQ NO: 399 and the LCDR3 encoded by the nucleotide sequence SEQ NO: 400,
l) the heavy chain variable region comprises the HCDR1 encoded by the nucleotide sequence SEQ NO: 302, the HCDR2 encoded by the nucleotide sequence SEQ NO: 303 and the HCDR3 encoded by the nucleotide sequence SEQ NO: 304, and the light chain variable region comprises the LCDR1 encoded by the nucleotide sequence SEQ NO: 406, the LCDR2 encoded by the nucleotide sequence SEQ NO: 407 and the LCDR3 encoded by the nucleotide sequence SEQ NO: 408,
m) the heavy chain variable region comprises the HCDR1 encoded by the nucleotide sequence SEQ NO: 302, the HCDR2 encoded by the nucleotide sequence SEQ NO: 303 and the HCDR3 encoded by the nucleotide sequence SEQ NO: 304, and the light chain variable region comprises the LCDR1 encoded by the nucleotide sequence SEQ NO: 410, the LCDR2 encoded by the nucleotide sequence SEQ NO: 411 and the LCDR3 encoded by the nucleotide sequence SEQ NO: 412, or
n) the heavy chain variable region comprises the HCDR1 encoded by the nucleotide sequence SEQ NO: 314, the HCDR2 encoded by the nucleotide sequence SEQ NO: 315 and the HCDR3 encoded by the nucleotide sequence SEQ NO: 316, and the light chain variable region comprises the LCDR1 encoded by the nucleotide sequence SEQ NO: 422, the LCDR2 encoded by the nucleotide sequence SEQ NO: 423 and the LCDR3 encoded by the nucleotide sequence SEQ NO: 424.

In one embodiment, the mAb which binds to the HSV gEgI heterodimer at a conformational epitope located at the interface between HSV gE and HSV gI, comprises a heavy chain, wherein the heavy chain variable region has a sequence selected from SEQ ID NO: 17, SEQ ID NO: 21, SEQ ID NO: 25, SEQ ID NO: 37, SEQ ID NO: 45, SEQ ID NO: 53, SEQ ID NO: 57, SEQ ID NO: 61, SEQ ID NO: 65, SEQ ID NO: 77, SEQ ID NO: 81, SEQ ID NO: 89 and SEQ ID NO: 101, or a sequence which is at least 80%, 85%, 90%, 95%, 96%, 97%, 98% or 99% identical thereto.

In one embodiment, the mAb which binds to the HSV gEgI heterodimer at a conformational epitope located at the interface between HSV gE and HSV gI, comprises a light chain, wherein the light chain variable region has a sequence selected from SEQ ID NO: 121, SEQ ID NO: 125, SEQ ID NO: 129, SEQ ID NO: 141, SEQ ID NO: 149, SEQ ID NO: 157, SEQ ID NO: 161, SEQ ID NO: 165, SEQ ID NO: 169, SEQ ID NO: 181, SEQ ID NO: 185, SEQ ID NO: 193, SEQ ID NO: 197 and SEQ ID NO: 209, or a sequence which is at least 80%, 85%, 90%, 95%, 96%, 97%, 98% or 99% identical thereto.

In one embodiment, the mAb which binds to the HSV gEgI heterodimer at a conformational epitope located at the interface between HSV gE and HSV gI, comprises a heavy chain and a light chain, wherein the heavy chain variable region has a sequence selected from SEQ ID NO: 17, SEQ ID NO: 21, SEQ ID NO: 25, SEQ ID NO: 37, SEQ ID NO: 45, SEQ ID NO: 53, SEQ ID NO: 57, SEQ ID NO: 61, SEQ ID NO: 65, SEQ ID NO: 77, SEQ ID NO: 81, SEQ ID NO: 89 and SEQ ID NO: 101, or a sequence which is at least 80%, 85%, 90%, 95%, 96%, 97%, 98% or 99% identical thereto, and wherein the light chain variable region has a sequence selected from SEQ ID NO: 121, SEQ ID NO: 125, SEQ ID NO: 129, SEQ ID NO: 141, SEQ ID NO: 149, SEQ ID NO: 157, SEQ ID NO: 161, SEQ ID NO: 165, SEQ ID NO: 169, SEQ ID NO: 181, SEQ ID NO: 185, SEQ ID NO: 193, SEQ ID NO: 197 and SEQ ID NO: 209, or a sequence which is at least 80%, 85%, 90%, 95%, 96%, 97%, 98% or 99% identical thereto.

In one embodiment, the mAb which binds to the HSV gEgI heterodimer at a conformational epitope located at the interface between HSV gE and HSV gI, comprises a heavy chain, wherein the heavy chain variable region comprises or consists of a nucleotide sequence selected from SEQ ID NO: 229, SEQ ID NO: 233, SEQ ID NO: 237, SEQ ID NO: 249, SEQ ID NO: 257, SEQ ID NO: 265, SEQ ID NO: 269, SEQ ID NO: 273, SEQ ID NO: 277, SEQ ID NO: 389, SEQ ID NO: 293, SEQ ID NO: 301 and SEQ ID NO: 313, or a sequence which is at least 80%, 85%, 90%, 95%, 96%, 97%, 98% or 99% identical thereto.

In one embodiment, the mAb which binds to the HSV gEgI heterodimer at a conformational epitope located at the interface between HSV gE and HSV gI, comprises a light chain, wherein the light chain variable region comprises or consists of a nucleotide sequence selected from SEQ ID NO: 333, SEQ ID NO: 337, SEQ ID NO: 341, SEQ ID NO: 353, SEQ ID NO: 361, SEQ ID NO: 369, SEQ ID NO: 373, SEQ ID NO: 373, SEQ ID NO: 381, SEQ ID NO: 393, SEQ ID NO: 397, SEQ ID NO: 405, SEQ ID NO: 409 and SEQ ID NO: 421, or a sequence which is at least 80%, 85%, 90%, 95%, 96%, 97%, 98% or 99% identical thereto.

In one embodiment, the mAb which binds to the HSV gEgI heterodimer at a conformational epitope located at the interface between HSV gE and HSV gI, comprises a heavy chain and a light chain, wherein the heavy chain variable region comprises or consists of a nucleotide sequence selected from SEQ ID NO: 229, SEQ ID NO: 233, SEQ ID NO: 237, SEQ ID NO: 249, SEQ ID NO: 257, SEQ ID NO: 265, SEQ ID NO: 269, SEQ ID NO: 273, SEQ ID NO: 277, SEQ ID NO: 389, SEQ ID NO: 293, SEQ ID NO: 301 and SEQ ID NO: 313, or a sequence which is at least 80%, 85%, 90%, 95%, 96%, 97%, 98% or 99% identical thereto, and wherein the light chain variable region comprises or consists of a nucleotide sequence selected from SEQ ID NO: 333, SEQ ID NO: 337, SEQ ID NO: 341, SEQ ID NO: 353, SEQ ID NO: 361, SEQ ID NO: 369, SEQ ID NO: 373, SEQ ID NO: 373, SEQ ID NO: 381, SEQ ID NO: 393, SEQ ID NO: 397, SEQ ID NO: 405, SEQ ID NO: 409 and SEQ ID NO: 421, or a sequence which is at least 80%, 85%, 90%, 95%, 96%, 97%, 98% or 99% identical thereto.

In one embodiment, the mAb which binds to the HSV gEgI heterodimer at a conformational epitope located at the interface between HSV gE and HSV gI, comprises a heavy chain and a light chain, wherein the heavy chain variable region comprises or consists of the sequence shown in SEQ ID NO: 17 or a sequence which is at least 80%, 85%, 90%, 95%, 96%, 97%, 98% or 99% identical thereto, and wherein the light chain variable region comprises or consists of the sequence shown in SEQ ID NO: 121 or a sequence which is at least 80%, 85%, 90%, 95%, 96%, 97%, 98% or 99% identical thereto. In one embodiment, the mAb which binds to the HSV gEgI heterodimer at a conformational epitope located at the interface between HSV gE and HSV gI, comprises a heavy chain and a light chain, wherein the heavy chain variable region comprises or consists of a nucleotide sequence selected from SEQ ID NO: 229 or a sequence which is at least 80%, 85%, 90%, 95%, 96%, 97%, 98% or 99% identical thereto, and wherein the light chain variable region comprises or consists of a nucleotide sequence selected from SEQ ID NO: 333 or a sequence which is at least 80%, 85%, 90%, 95%, 96%, 97%, 98% or 99% identical thereto. Preferably, the heavy chain has a murine IgG1 isotype. More preferably, the heavy chain has VDJ regions IGHV7-1*02, IGHD1-1*01_5-3 and IGHJ3*01. Preferably, the light chain is a Kappa light chain class. More preferably, the light chain has VJ regions IGKV5-39*01 and IGKJ2*01.

In one embodiment, the mAb which binds to the HSV gEgI heterodimer at a conformational epitope located at the interface between HSV gE and HSV gI, comprises a heavy chain and a light chain, wherein the heavy chain variable region comprises or consists of the sequence shown in SEQ ID NO: 21 or a sequence which is at least 80%, 85%, 90%, 95%, 96%, 97%, 98% or 99% identical thereto, and wherein the light chain variable region comprises or consists of the sequence shown in SEQ ID NO: 125 or a sequence which is at least 80%, 85%, 90%, 95%, 96%, 97%, 98% or 99% identical thereto. In one embodiment, the mAb which binds to the HSV gEgI heterodimer at a conformational epitope located at the interface between HSV gE and HSV gI, comprises a heavy chain and a light chain, wherein the heavy chain variable region comprises or consists of a nucleotide sequence selected from SEQ ID NO: 233 or a sequence which is at least 80%, 85%, 90%, 95%, 96%, 97%, 98% or 99% identical thereto, and wherein the light chain variable region comprises or consists of a nucleotide sequence selected from SEQ ID NO: 337 or a sequence which is at least 80%, 85%, 90%, 95%, 96%, 97%, 98% or 99% identical thereto. Preferably, the heavy chain has a murine IgG1 isotype. More preferably, the heavy chain has VDJ regions IGHV1S135*01, IGHD1-1*01_5-3 and IGHJ4*01. Preferably, the light chain is a Kappa light chain class. More preferably, the light chain has VJ regions IGKV8-30*01 and IGKJ2*01.

In one embodiment, the mAb which binds to the HSV gEgI heterodimer at a conformational epitope located at the interface between HSV gE and HSV gI, comprises a heavy chain and a light chain, wherein the heavy chain variable region comprises or consists of the sequence shown in SEQ ID NO: 25 or a sequence which is at least 80%, 85%, 90%, 95%, 96%, 97%, 98% or 99% identical thereto, and wherein the light chain variable region comprises or consists of the sequence shown in SEQ ID NO: 129 or a sequence which is at least 80%, 85%, 90%, 95%, 96%, 97%, 98% or 99% identical thereto. In one embodiment, the mAb which binds to the HSV gEgI heterodimer at a conformational epitope located at the interface between HSV gE and HSV gI, comprises a heavy chain and a light chain, wherein the heavy chain variable region comprises or consists of a nucleotide sequence selected from SEQ ID NO: 237 or a sequence which is at least 80%, 85%, 90%, 95%, 96%, 97%, 98% or 99% identical thereto, and wherein the light chain variable region comprises or consists of a nucleotide sequence selected from SEQ ID NO: 341 or a sequence which is at least 80%, 85%, 90%, 95%, 96%, 97%, 98% or 99% identical thereto. Preferably, the heavy chain has a murine IgG1 isotype. More preferably, the heavy chain has VDJ regions IGHV7-1*02, IGHD3-2_01_3-5, IGHJ4*01. Preferably, the light chain is a Kappa light chain class. More preferably, the light chain has VJ regions IGKV3-4*01 and IGKJ2*01.

In one embodiment, the mAb which binds to the HSV gEgI heterodimer at a conformational epitope located at the interface between HSV gE and HSV gI, comprises a heavy chain and a light chain, wherein the heavy chain variable region comprises or consists of the sequence shown in SEQ ID NO: 37 or a sequence which is at least 80%, 85%, 90%, 95%, 96%, 97%, 98% or 99% identical thereto, and wherein the light chain variable region comprises or consists of the sequence shown in SEQ ID NO: 141 or a sequence which is at least 80%, 85%, 90%, 95%, 96%, 97%, 98% or 99% identical thereto. In one embodiment, the mAb which binds to the HSV gEgI heterodimer at a conformational epitope located at the interface between HSV gE and HSV gI, comprises a heavy chain and a light chain, wherein the heavy chain variable region comprises or consists of a nucleotide sequence selected from SEQ ID NO: 249 or a sequence which is at least 80%, 85%, 90%, 95%, 96%, 97%, 98% or 99% identical thereto, and wherein the light chain variable region comprises or consists of a nucleotide sequence selected from SEQ ID NO: 353 or a sequence which is at least 80%, 85%, 90%, 95%, 96%, 97%, 98% or 99% identical thereto. Preferably, the heavy chain has a murine IgG3 isotype. More preferably, the heavy chain has VDJ regions IGHV2-3*01, IGHD2-4*01_5-3 and IGHJ4*01. Preferably, the light chain is a Kappa light chain class. More preferably, the light chain has VJ regions IGKV12-41*01 and IGKJ1*01.

In one embodiment, the mAb which binds to the HSV gEgI heterodimer at a conformational epitope located at the interface between HSV gE and HSV gI, comprises a heavy chain and a light chain, wherein the heavy chain variable region comprises or consists of the sequence shown in SEQ ID NO: 45 or a sequence which is at least 80%, 85%, 90%, 95%, 96%, 97%, 98% or 99% identical thereto, and wherein the light chain variable region comprises or consists of the sequence shown in SEQ ID NO: 149 or a sequence which is at least 80%, 85%, 90%, 95%, 96%, 97%, 98% or 99% identical thereto. In one embodiment, the mAb which binds to the HSV gEgI heterodimer at a conformational epitope located at the interface between HSV gE and HSV gI, comprises a heavy chain and a light chain, wherein the heavy chain variable region comprises or consists of a nucleotide sequence selected from SEQ ID NO: 257 or a sequence which is at least 80%, 85%, 90%, 95%, 96%, 97%, 98% or 99% identical thereto, and wherein the light chain variable region comprises or consists of a nucleotide sequence selected from SEQ ID NO: 361 or a sequence which is at least 80%, 85%, 90%, 95%, 96%, 97%, 98% or 99% identical thereto. Preferably, the heavy chain has a murine IgG3 isotype. More preferably, the heavy chain has VDJ regions IGHV2-9-1*01, IGHD1-2*01_5-3 and IGHJ4*01. Preferably, the light chain is a Kappa light chain class. More preferably, the light chain has VJ regions IGKV2-137*01 and IGKJ5*01.

In one embodiment, the mAb which binds to the HSV gEgI heterodimer at a conformational epitope located at the interface between HSV gE and HSV gI, comprises a heavy chain and a light chain, wherein the heavy chain variable region comprises or consists of the sequence shown in SEQ ID NO: 53 or a sequence which is at least 80%, 85%, 90%, 95%, 96%, 97%, 98% or 99% identical thereto, and wherein the light chain variable region comprises or consists of the sequence shown in SEQ ID NO: 157 or a sequence which is at least 80%, 85%, 90%, 95%, 96%, 97%, 98% or 99% identical thereto. In one embodiment, the mAb which binds to the HSV gEgI heterodimer at a conformational epitope located at the interface between HSV gE and HSV gI, comprises a heavy chain and a light chain, wherein the heavy chain variable region comprises or consists of a nucleotide sequence selected from SEQ ID NO: 265 or a sequence which is at least 80%, 85%, 90%, 95%, 96%, 97%, 98% or 99% identical thereto, and wherein the light chain variable region comprises or consists of a nucleotide sequence selected from SEQ ID NO: 369 or a sequence which is at least 80%, 85%, 90%, 95%, 96%, 97%, 98% or 99% identical thereto. Preferably, the heavy chain has a murine IgG1 isotype. More preferably, the heavy chain has VDJ regions IGHV1S137*01, IGHD1-1*01_5-3 and IGHJ4*01. Preferably, the light chain is a Kappa light chain class. More preferably, the light chain has VJ regions IGKV4-59*01 and IGKJ1*01.

In one embodiment, the mAb which binds to the HSV gEgI heterodimer at a conformational epitope located at the interface between HSV gE and HSV gI, comprises a heavy chain and a light chain, wherein the heavy chain variable region comprises or consists of the sequence shown in SEQ ID NO: 57 or a sequence which is at least 80%, 85%, 90%, 95%, 96%, 97%, 98% or 99% identical thereto, and wherein the light chain variable region comprises or consists of the sequence shown in SEQ ID NO: 161 or a sequence which is at least 80%, 85%, 90%, 95%, 96%, 97%, 98% or 99% identical thereto. In one embodiment, the mAb which binds to the HSV gEgI heterodimer at a conformational epitope located at the interface between HSV gE and HSV gI, comprises a heavy chain and a light chain, wherein the heavy chain variable region comprises or consists of a nucleotide sequence selected from SEQ ID NO: 269 or a sequence which is at least 80%, 85%, 90%, 95%, 96%, 97%, 98% or 99% identical thereto, and wherein the light chain variable region comprises or consists of a nucleotide sequence selected from SEQ ID NO: 373 or a sequence which is at least 80%, 85%, 90%, 95%, 96%, 97%, 98% or 99% identical thereto. Preferably, the heavy chain has a murine IgG1 isotype. More preferably, the heavy chain has VDJ regions IGHV1-54*01, IGHD2-11*02_5-3 and IGHJ4*01. Preferably, the light chain is a Kappa light chain class. More preferably, the light chain has VJ regions IGKV3-10*01 and IGKJ1*01.

In one embodiment, the mAb which binds to the HSV gEgI heterodimer at a conformational epitope located at the interface between HSV gE and HSV gI, comprises a heavy chain and a light chain, wherein the heavy chain variable region comprises or consists of the sequence shown in SEQ ID NO: 61 or a sequence which is at least 80%, 85%, 90%, 95%, 96%, 97%, 98% or 99% identical thereto, and wherein the light chain variable region comprises or consists of the sequence shown in SEQ ID NO: 165 or a sequence which is at least 80%, 85%, 90%, 95%, 96%, 97%, 98% or 99% identical thereto. In one embodiment, the mAb which binds to the HSV gEgI heterodimer at a conformational epitope located at the interface between HSV gE and HSV gI, comprises a heavy chain and a light chain, wherein the heavy chain variable region comprises or consists of a nucleotide sequence selected from SEQ ID NO: 273 or a sequence which is at least 80%, 85%, 90%, 95%, 96%, 97%, 98% or 99% identical thereto, and wherein the light chain variable region comprises or consists of a nucleotide sequence selected from SEQ ID NO: 377 or a sequence which is at least 80%, 85%, 90%, 95%, 96%, 97%, 98% or 99% identical thereto. Preferably, the heavy chain has a murine IgG1 isotype. More preferably, the heavy chain has VDJ regions IGHV1-26*01, IGHD5-6_01_3-5 and IGHJ4*01. Preferably, the light chain is a Kappa light chain class. More preferably, the light chain has VJ regions IGKV8-27*01 and IGKJ2*01.

In one embodiment, the mAb which binds to the HSV gEgI heterodimer at a conformational epitope located at the interface between HSV gE and HSV gI, comprises a heavy chain and a light chain, wherein the heavy chain variable region comprises or consists of the sequence shown in SEQ ID NO: 65 or a sequence which is at least 80%, 85%, 90%, 95%, 96%, 97%, 98% or 99% identical thereto, and wherein the light chain variable region comprises or consists of the sequence shown in SEQ ID NO: 169 or a sequence which is at least 80%, 85%, 90%, 95%, 96%, 97%, 98% or 99% identical thereto. In one embodiment, the mAb which binds to the HSV gEgI heterodimer at a conformational epitope located at the interface between HSV gE and HSV gI, comprises a heavy chain and a light chain, wherein the heavy chain variable region comprises or consists of a nucleotide sequence selected from SEQ ID NO: 277 or a sequence which is at least 80%, 85%, 90%, 95%, 96%, 97%, 98% or 99% identical thereto, and wherein the light chain variable region comprises or consists of a nucleotide sequence selected from SEQ ID NO: 381 or a sequence which is at least 80%, 85%, 90%, 95%, 96%, 97%, 98% or 99% identical thereto. Preferably, the heavy chain has a murine IgG1 isotype. More preferably, the heavy chain has VDJ regions IGHV5-12*02, IGHD2-11*02_5-3 and IGHJ3*01. Preferably, the light chain is a Kappa light chain class. More preferably, the light chain has VJ regions IGKV4-57-1*01 and IGKJ5*01.

In one embodiment, the mAb which binds to the HSV gEgI heterodimer at a conformational epitope located at the interface between HSV gE and HSV gI, comprises a heavy chain and a light chain, wherein the heavy chain variable region comprises or consists of the sequence shown in SEQ ID NO: 77 or a sequence which is at least 80%, 85%, 90%, 95%, 96%, 97%, 98% or 99% identical thereto, and wherein the light chain variable region comprises or consists of the sequence shown in SEQ ID NO: 181 or a sequence which is at least 80%, 85%, 90%, 95%, 96%, 97%, 98% or 99% identical thereto. In one embodiment, the mAb which binds to the HSV gEgI heterodimer at a conformational epitope located at the interface between HSV gE and HSV gI, comprises a heavy chain and a light chain, wherein the heavy chain variable region comprises or consists of a nucleotide sequence selected from SEQ ID NO: 289 or a sequence which is at least 80%, 85%, 90%, 95%, 96%, 97%, 98% or 99% identical thereto, and wherein the light chain variable region comprises or consists of a nucleotide sequence selected from SEQ ID NO: 393 or a sequence which is at least 80%, 85%, 90%, 95%, 96%, 97%, 98% or 99% identical thereto. Preferably, the heavy chain has a murine IgG1 isotype. More preferably, the heavy chain has VDJ regions IGHV5-12-2*01, IGHD2-13*01_5-3 and IGHJ2*01. Preferably, the light chain is a Kappa light chain class. More preferably, the light chain has VJ regions IGKV1-88*01 and IGKJ1*01.

In one embodiment, the mAb which binds to the HSV gEgI heterodimer at a conformational epitope located at the interface between HSV gE and HSV gI, comprises a heavy chain and a light chain, wherein the heavy chain variable region comprises or consists of the sequence shown in SEQ ID NO: 81 or a sequence which is at least 80%, 85%, 90%, 95%, 96%, 97%, 98% or 99% identical thereto, and wherein the light chain variable region comprises or consists of the sequence shown in SEQ ID NO: 185 or a sequence which is at least 80%, 85%, 90%, 95%, 96%, 97%, 98% or 99% identical thereto. In one embodiment, the mAb which binds to the HSV gEgI heterodimer at a conformational epitope located at the interface between HSV gE and HSV gI, comprises a heavy chain and a light chain, wherein the heavy chain variable region comprises or consists of a nucleotide sequence selected from SEQ ID NO: 293 or a sequence which is at least 80%, 85%, 90%, 95%, 96%, 97%, 98% or 99% identical thereto, and wherein the light chain variable region comprises or consists of a nucleotide sequence selected from SEQ ID NO: 397 or a sequence which is at least 80%, 85%, 90%, 95%, 96%, 97%, 98% or 99% identical thereto. Preferably, the heavy chain has a murine IgG1 isotype. More preferably, the heavy chain has VDJ regions IGHV9-3-1*01, IGHD2-9*01_5-3 and IGHJ1*01. Preferably, the light chain is a Kappa light chain class. More preferably, the light chain has VJ regions IGKV12-41*01 and IGKJ2*01.

In one embodiment, the mAb which binds to the HSV gEgI heterodimer at a conformational epitope located at the interface between HSV gE and HSV gI, comprises a heavy chain and a light chain, wherein the heavy chain variable region comprises or consists of the sequence shown in SEQ ID NO: 89 or a sequence which is at least 80%, 85%, 90%, 95%, 96%, 97%, 98% or 99% identical thereto, and wherein the light chain variable region comprises or consists of the sequence shown in SEQ ID NO: 193 or a sequence which is at least 80%, 85%, 90%, 95%, 96%, 97%, 98% or 99% identical thereto. In one embodiment, the mAb which binds to the HSV gEgI heterodimer at a conformational epitope located at the interface between HSV gE and HSV gI, comprises a heavy chain and a light chain, wherein the heavy chain variable region comprises or consists of a nucleotide sequence selected from SEQ ID NO: 301 or a sequence which is at least 80%, 85%, 90%, 95%, 96%, 97%, 98% or 99% identical thereto, and wherein the light chain variable region comprises or consists of a nucleotide sequence selected from SEQ ID NO: 405 or a sequence which is at least 80%, 85%, 90%, 95%, 96%, 97%, 98% or 99% identical thereto. Preferably, the heavy chain has a murine IgG1 isotype. More preferably, the heavy chain has VDJ regions IGHV2-9-1*01, IGHD4-1*01_5-3 and IGHJ2*01. Preferably, the light chain is a Kappa light chain class. More preferably, the light chain has VJ regions Light chain 1 (L1): IGKV1-135*01 and IGKJ1*01.

In one embodiment, the mAb which binds to the HSV gEgI heterodimer at a conformational epitope located at the interface between HSV gE and HSV gI, comprises a heavy chain and a light chain, wherein the heavy chain variable region comprises or consists of the sequence shown in SEQ ID NO: 89 or a sequence which is at least 80%, 85%, 90%, 95%, 96%, 97%, 98% or 99% identical thereto, and wherein the light chain variable region comprises or consists of the sequence shown in SEQ ID NO: 197 or a sequence which is at least 80%, 85%, 90%, 95%, 96%, 97%, 98% or 99% identical thereto. In one embodiment, the mAb which binds to the HSV gEgI heterodimer at a conformational epitope located at the interface between HSV gE and HSV gI, comprises a heavy chain and a light chain, wherein the heavy chain variable region comprises or consists of a nucleotide sequence selected from SEQ ID NO: 301 or a sequence which is at least 80%, 85%, 90%, 95%, 96%, 97%, 98% or 99% identical thereto, and wherein the light chain variable region comprises or consists of a nucleotide sequence selected from SEQ ID NO: 409 or a sequence which is at least 80%, 85%, 90%, 95%, 96%, 97%, 98% or 99% identical thereto. Preferably, the heavy chain has a murine IgG1 isotype. More preferably, the heavy chain has VDJ regions IGHV2-9-1*01, IGHD4-1*01_5-3 and IGHJ2*01. Preferably, the light chain is a Kappa light chain class. More preferably, the light chain has VJ regions Light chain 1 (L1): IGKV5-45*01 and IGKJ4*01.

In one embodiment, the mAb which binds to the HSV gEgI heterodimer at a conformational epitope located at the interface between HSV gE and HSV gI, comprises a heavy chain and a light chain, wherein the heavy chain variable region comprises or consists of the sequence shown in SEQ ID NO: 101 or a sequence which is at least 80%, 85%, 90%, 95%, 96%, 97%, 98% or 99% identical thereto, and wherein the light chain variable region comprises or consists of the sequence shown in SEQ ID NO: 209 or a sequence which is at least 80%, 85%, 90%, 95%, 96%, 97%, 98% or 99% identical thereto. In one embodiment, the mAb which binds to the HSV gEgI heterodimer at a conformational epitope located at the interface between HSV gE and HSV gI, comprises a heavy chain and a light chain, wherein the heavy chain variable region comprises or consists of a nucleotide sequence selected from SEQ ID NO: 313 or a sequence which is at least 80%, 85%, 90%, 95%, 96%, 97%, 98% or 99% identical thereto, and wherein the light chain variable region comprises or consists of a nucleotide sequence selected from SEQ ID NO: 421 or a sequence which is at least 80%, 85%, 90%, 95%, 96%, 97%, 98% or 99% identical thereto. Preferably, the heavy chain has a murine IgG1 isotype. More preferably, the heavy chain has VDJ regions IGHV5-6-4*01, IGHD1-1*01_5-3 and IGHJ4*01. Preferably, the light chain is a Kappa light chain class. More preferably, the light chain has VJ regions IGKV3-12*01 and IGKJ2*01.

In another aspect, there is provided an antigen binding protein which binds to an epitope located on HSV gI. In a preferred embodiment, the antigen binding protein is an antibody which binds to an epitope located on HSV gI. In a more preferred embodiment, the antibody is a monoclonal antibody (mAb) which binds to an epitope located on HSV gI Preferably, such a mAb binds to free HSV gI and to the HSV gEgI heterodimer. Examples of mAbs which bind to a HSV gI epitope include mAbs from clones 2, 41, 44 and 35/36 (see example section and tables 7-11).

In a preferred embodiment, the mAb which binds to an epitope located on HSV gI, comprises any one or a combination of CDRs selected from SEQ ID NOs: 10-12, 86-88, 98-100, 70-72, 114-116, 190-192, 206-208 and 174-176 or variants thereof, wherein the variant has 1, 2, or 3 amino acid deletions, substitutions or insertions.

In a preferred embodiment, the mAb which binds to an epitope located on HSV gI, comprises any one or a combination of CDRs encoded by SEQ ID NOs: 222-224, 298-300, 310-312, 282-284, 326-328, 402-404, 418-420 and 386-388 or variants thereof, wherein the variant has 1, 2, 3, 4, 5, 6, 7, 8, 9 or 10 nucleotide deletions, substitutions or insertions.

In a preferred embodiment, the mAb which binds to an epitope located on HSV gI, comprises a heavy chain, wherein the heavy chain variable region comprises:
a) the HCDR1 shown in SEQ ID NO: 10, the HCDR2 shown in SEQ ID NO: 11 and the HCDR3 shown in SEQ ID NO: 12, or variants thereof having 1, 2, or 3 amino acid deletions, substitutions or insertions,
b) the HCDR1 shown in SEQ ID NO: 86, the HCDR2 shown in SEQ ID NO: 87 and the HCDR3 shown in SEQ ID NO: 88, or variants thereof having 1, 2, or 3 amino acid deletions, substitutions or insertions,
c) the HCDR1 shown in SEQ ID NO: 98, the HCDR2 shown in SEQ ID NO: 99 and the HCDR3 shown in SEQ ID NO: 100, or variants thereof having 1, 2, or 3 amino acid deletions, substitutions or insertions, or
d) the HCDR1 shown in SEQ ID NO: 70, the HCDR2 shown in SEQ ID NO: 71 and the HCDR3 shown in SEQ ID NO: 72, or variants thereof having 1, 2, or 3 amino acid deletions, substitutions or insertions.

In a preferred embodiment, the mAb which binds to an epitope located on HSV gI, comprises a light chain, wherein the light chain variable region comprises:
a) the LCDR1 shown in SEQ ID NO: 114, the LCDR2 shown in SEQ ID NO: 115 and the LCDR3 shown in SEQ ID NO: 116, or variants thereof having 1, 2, or 3 amino acid deletions, substitutions or insertions,
b) the LCDR1 shown in SEQ ID NO: 190, the LCDR2 shown in SEQ ID NO: 191 and the LCDR3 shown in SEQ ID NO: 192, or variants thereof having 1, 2, or 3 amino acid deletions, substitutions or insertions,
c) the LCDR1 shown in SEQ ID NO: 206, the LCDR2 shown in SEQ ID NO: 207 and the LCDR3 shown in SEQ ID NO: 208, or variants thereof having 1, 2, or 3 amino acid deletions, substitutions or insertions, or
d) the LCDR1 shown in SEQ ID NO: 174, the LCDR2 shown in SEQ ID NO: 175 and the LCDR3 shown in SEQ ID NO: 176, or variants thereof having 1, 2, or 3 amino acid deletions, substitutions or insertions.

In a preferred embodiment, the mAb which binds to an epitope located on HSV gI, comprises a heavy chain and a light chain, wherein:
a) the heavy chain variable region comprises the HCDR1 shown in SEQ ID NO: 10, the HCDR2 shown in SEQ ID NO: 11 and the HCDR3 shown in SEQ ID NO: 12, and the light chain variable region comprises the LCDR1 shown in SEQ ID NO: 114, the LCDR2 shown in SEQ ID NO: 115 and the LCDR3 shown in SEQ ID NO: 116, the heavy chain variable region comprises the HCDR1 shown in SEQ ID NO: 86, the HCDR2 shown in SEQ ID NO: 87 and the HCDR3 shown in SEQ ID NO: 88, and the light chain variable region comprises the LCDR1 shown in SEQ ID NO: 190, the LCDR2 shown in SEQ ID NO: 191 and the LCDR3 shown in SEQ ID NO: 192,
e) the heavy chain variable region comprises the HCDR1 shown in SEQ ID NO: 98, the HCDR2 shown in SEQ ID NO: 99 and the HCDR3 shown in SEQ ID NO: 100, and the light chain variable region comprises the LCDR1 shown in SEQ ID NO: 206, the LCDR2 shown in SEQ ID NO: 207 and the LCDR3 shown in SEQ ID NO: 208, or
f) the heavy chain variable region comprises the HCDR1 shown in SEQ ID NO: 70, the HCDR2 shown in SEQ ID NO: 71 and the HCDR3 shown in SEQ ID NO: 72, and the light chain variable region comprises the LCDR1 shown in SEQ ID NO: 174, the LCDR2 shown in SEQ ID NO: 175 and the LCDR3 shown in SEQ ID NO: 176.

In a preferred embodiment, the mAb which binds to an epitope located on HSV gI, comprises a heavy chain, wherein the heavy chain variable region comprises:
a) the HCDR1 encoded by the nucleotide sequence SEQ ID NO: 222, the HCDR2 encoded by the nucleotide sequence SEQ ID NO: 223 and the HCDR3 encoded by the nucleotide sequence SEQ ID NO: 224, or variants thereof having 1, 2, 3, 4, 5, 6, 7, 8, 9 or 10 nucleotide deletions, substitutions or insertions,
b) the HCDR1 encoded by the nucleotide sequence SEQ ID NO: 298, the HCDR2 encoded by the nucleotide sequence SEQ ID NO: 299 and the HCDR3 encoded by the nucleotide sequence SEQ ID NO: 300, or variants thereof having 1, 2, 3, 4, 5, 6, 7, 8, 9 or 10 nucleotide deletions, substitutions or insertions,
c) the HCDR1 encoded by the nucleotide sequence SEQ ID NO: 310, the HCDR2 encoded by the nucleotide sequence SEQ ID NO: 311 and the HCDR3 encoded by the nucleotide sequence SEQ ID NO: 312, or variants thereof having 1, 2, 3, 4, 5, 6, 7, 8, 9 or 10 nucleotide deletions, substitutions or insertions, or
d) the HCDR1 encoded by the nucleotide sequence SEQ ID NO: 282, the HCDR2 encoded by the nucleotide sequence SEQ ID NO: 283 and the HCDR3 encoded by the nucleotide sequence SEQ ID NO: 284, or variants thereof having 1, 2, 3, 4, 5, 6, 7, 8, 9 or 10 nucleotide deletions, substitutions or insertions.

In a preferred embodiment, the mAb which binds to an epitope located on HSV gI, comprises a light chain, wherein the light chain variable region comprises:
a) the LCDR1 encoded by the nucleotide sequence SEQ ID NO: 326, the LCDR2 encoded by the nucleotide sequence SEQ ID NO: 327 and the LCDR3 encoded by the nucleotide sequence SEQ ID NO: 328, or variants thereof having 1, 2, 3, 4, 5, 6, 7, 8, 9 or 10 nucleotide deletions, substitutions or insertions,
b) the LCDR1 encoded by the nucleotide sequence SEQ ID NO: 402, the LCDR2 encoded by the nucleotide sequence SEQ ID NO: 403 and the LCDR3 encoded by the nucleotide sequence SEQ ID NO: 404, or variants thereof having 1, 2, 3, 4, 5, 6, 7, 8, 9 or 10 nucleotide deletions, substitutions or insertions,
c) the LCDR1 encoded by the nucleotide sequence SEQ ID NO: 418, the LCDR2 encoded by the nucleotide sequence SEQ ID NO: 419 and the LCDR3 encoded by the nucleotide sequence SEQ ID NO: 420, or variants thereof having 1, 2, 3, 4, 5, 6, 7, 8, 9 or 10 nucleotide deletions, substitutions or insertions, or
d) the LCDR1 encoded by the nucleotide sequence SEQ ID NO: 386, the LCDR2 encoded by the nucleotide sequence SEQ ID NO: 387 and the LCDR3 encoded by the nucleotide sequence SEQ ID NO: 388, or variants thereof having 1, 2, 3, 4, 5, 6, 7, 8, 9 or 10 nucleotide deletions, substitutions or insertions.

In a preferred embodiment, the mAb which to an epitope located on HSV gI, comprises a heavy chain and a light chain, wherein:
a) the heavy chain variable region comprises the HCDR1 encoded by the nucleotide sequence SEQ ID NO: 222, the HCDR2 encoded by the nucleotide sequence SEQ ID NO: 223 and the HCDR3 encoded by the nucleotide sequence SEQ ID NO: 224, and the light chain variable region comprises the LCDR1 encoded by the nucleotide sequence SEQ ID NO: 326, the LCDR2 encoded by the nucleotide sequence SEQ ID NO: 327 and the LCDR3 encoded by the nucleotide sequence SEQ ID NO: 328,
b) the heavy chain variable region comprises the HCDR1 encoded by the nucleotide sequence SEQ ID NO: 298, the HCDR2 encoded by the nucleotide sequence SEQ ID NO: 299 and the HCDR3 encoded by the nucleotide sequence SEQ ID NO: 300, and the light chain variable region comprises the LCDR1 encoded by the nucleotide sequence SEQ ID NO: 402, the LCDR2 encoded by the nucleotide sequence SEQ ID NO: 403 and the LCDR3 encoded by the nucleotide sequence SEQ ID NO: 404,
c) the heavy chain variable region comprises the HCDR1 encoded by the nucleotide sequence SEQ ID NO: 310, the HCDR2 encoded by the nucleotide sequence SEQ ID NO: 311 and the HCDR3 encoded by the nucleotide sequence SEQ ID NO: 312, and the light chain variable region comprises the LCDR1 encoded by the nucleotide sequence SEQ ID NO: 418, the LCDR2 encoded by the nucleotide sequence SEQ ID NO: 419 and the LCDR3 encoded by the nucleotide sequence SEQ ID NO: 420, or
d) the heavy chain variable region comprises the HCDR1 encoded by the nucleotide sequence SEQ ID NO: 282, the HCDR2 encoded by the nucleotide sequence SEQ ID NO: 283 and the HCDR3 encoded by the nucleotide sequence SEQ ID NO: 284, and the light chain variable region comprises the LCDR1 encoded by the nucleotide sequence SEQ ID NO: 386, the LCDR2 encoded by the nucleotide sequence SEQ ID NO: 387 and the LCDR3 encoded by the nucleotide sequence SEQ ID NO: 388.

In one embodiment, the mAb which binds to an epitope located on HSV gI, comprises a heavy chain, wherein the heavy chain variable region has a sequence selected from SEQ ID NO: 9, SEQ ID NO: 85, SEQ ID NO: 97 and SEQ ID NO: 69, or a sequence which is at least 80%, 85%, 90%, 95%, 96%, 97%, 98% or 99% identical thereto.

In one embodiment, the mAb which binds to an epitope located on HSV gI, comprises a light chain, wherein the light chain variable region has a sequence selected from SEQ ID NO: 113, SEQ ID NO: 189, SEQ ID NO: 205 and SEQ ID NO: 173, or a sequence which is at least 80%, 85%, 90%, 95%, 96%, 97%, 98% or 99% identical thereto.

In one embodiment, the mAb which binds to an epitope located on HSV gI, comprises a heavy chain and a light chain, wherein the heavy chain variable region has a sequence selected from SEQ ID NO: 9, SEQ ID NO: 85, SEQ ID NO: 97 and SEQ ID NO: 69, or a sequence which is at least 80%, 85%, 90%, 95%, 96%, 97%, 98% or 99% identical thereto, and wherein the light chain variable region has a sequence selected from SEQ ID NO: 113, SEQ ID NO: 189, SEQ ID NO: 205 and SEQ ID NO: 173, or a sequence which is at least 80%, 85%, 90%, 95%, 96%, 97%, 98% or 99% identical thereto.

In one embodiment, the mAb which binds to an epitope located on HSV gI, comprises a heavy chain, wherein the heavy chain variable region comprises or consists of a nucleotide sequence selected from SEQ ID NO: 221, SEQ ID NO: 297, SEQ ID NO: 309 and SEQ ID NO: 281, or a sequence which is at least 80%, 85%, 90%, 95%, 96%, 97%, 98% or 99% identical thereto.

In one embodiment, the mAb which binds to an epitope located on HSV gI, comprises a light chain, wherein the light chain variable region comprises or consists of a nucleotide sequence selected from SEQ ID NO: 325, SEQ ID NO: 401, SEQ ID NO: 417 and SEQ ID NO: 385, or a sequence which is at least 80%, 85%, 90%, 95%, 96%, 97%, 98% or 99% identical thereto.

In one embodiment, the mAb which binds to an epitope located on HSV gI, comprises a heavy chain and a light chain, wherein the heavy chain variable region comprises or consists of a nucleotide sequence selected from SEQ ID NO: 221, SEQ ID NO: 297, SEQ ID NO: 309 and SEQ ID NO: 281, or a sequence which is at least 80%, 85%, 90%, 95%, 96%, 97%, 98% or 99% identical thereto, and wherein the light chain variable region comprises or consists of a nucleotide sequence selected from SEQ ID NO: 325, SEQ ID NO: 401, SEQ ID NO: 417 and SEQ ID NO: 385, or a sequence which is at least 80%, 85%, 90%, 95%, 96%, 97%, 98% or 99% identical thereto.

In one embodiment, the mAb which binds to an epitope located on HSV gI, comprises a heavy chain and a light chain, wherein the heavy chain variable region comprises or consists of the sequence shown in SEQ ID NO: 9 or a sequence which is at least 80%, 85%, 90%, 95%, 96%, 97%, 98% or 99% identical thereto, and wherein the light chain variable region comprises or consists of the sequence shown in SEQ ID NO: 113 or a sequence which is at least 80%, 85%, 90%, 95%, 96%, 97%, 98% or 99% identical thereto. In one embodiment, the mAb which binds to an epitope located on HSV gI, comprises a heavy chain and a light chain, wherein the heavy chain variable region comprises or consists of a nucleotide sequence selected from SEQ ID NO: 221 or a sequence which is at least 80%, 85%, 90%, 95%, 96%, 97%, 98% or 99% identical thereto, and wherein the light chain variable region comprises or consists of a nucleotide sequence selected from SEQ ID NO: 325 or a sequence which is at least 80%, 85%, 90%, 95%, 96%, 97%, 98% or 99% identical thereto. Preferably, the heavy chain has a murine IgG1 isotype. More preferably, the heavy chain has VDJ regions IGHV1-54*01, IGHD4-1*01_5-3 and IGHJ4*0. Preferably, the light chain is a Kappa light chain class. More preferably, the light chain has VJ regions IGKV3-4*01 and IGKJ2*01.

In one embodiment, the mAb which binds to an epitope located on HSV gI, comprises a heavy chain and a light chain, wherein the heavy chain variable region comprises or consists of the sequence shown in SEQ ID NO: 85 or a sequence which is at least 80%, 85%, 90%, 95%, 96%, 97%, 98% or 99% identical thereto, and wherein the light chain variable region comprises or consists of the sequence shown in SEQ ID NO: 189 or a sequence which is at least 80%, 85%, 90%, 95%, 96%, 97%, 98% or 99% identical thereto. In one embodiment, the mAb which binds to an epitope located on HSV gI, comprises a heavy chain and a light chain, wherein the heavy chain variable region comprises or consists of a nucleotide sequence selected from SEQ ID NO: 297 or a sequence which is at least 80%, 85%, 90%, 95%, 96%, 97%, 98% or 99% identical thereto, and wherein the light chain variable region comprises or consists of a nucleotide sequence selected from SEQ ID NO: 401 or a sequence which is at least 80%, 85%, 90%, 95%, 96%, 97%, 98% or 99% identical thereto. Preferably, the heavy chain has a murine IgG1 isotype. More preferably, the heavy chain has VDJ regions IGHV1-54*01, IGHD2-9*02_5-3 and IGHJ4*01. Preferably, the light chain is a Kappa light chain class. More preferably, the light chain has VJ regions IGKV3-4*01 and IGKJ1*01.

In one embodiment, the mAb which binds to an epitope located on HSV gI, comprises a heavy chain and a light chain, wherein the heavy chain variable region comprises or consists of the sequence shown in SEQ ID NO: 97 or a sequence which is at least 80%, 85%, 90%, 95%, 96%, 97%, 98% or 99% identical thereto, and wherein the light chain variable region comprises or consists of the sequence shown in SEQ ID NO: 205 or a sequence which is at least 80%, 85%, 90%, 95%, 96%, 97%, 98% or 99% identical thereto. In one embodiment, the mAb which binds to an epitope located on HSV gI, comprises a heavy chain and a light chain, wherein the heavy chain variable region comprises or consists of a nucleotide sequence selected from SEQ ID NO: 309 or a sequence which is at least 80%, 85%, 90%, 95%, 96%, 97%, 98% or 99% identical thereto, and wherein the light chain variable region comprises or consists of a nucleotide sequence selected from SEQ ID NO: 417 or a sequence which is at least 80%, 85%, 90%, 95%, 96%, 97%, 98% or 99% identical thereto. Preferably, the heavy chain has a murine IgG2a isotype. More preferably, the heavy chain has VDJ regions IGHV1S135*01, IGHD5-7_01_3-5 and IGHJ4*01. Preferably, the light chain is a Kappa light chain class. More preferably, the light chain has VJ regions IGKV4-70*01 and IGKJ2*01.

In one embodiment, the mAb which binds to an epitope located on HSV gI, comprises a heavy chain and a light chain, wherein the heavy chain variable region comprises or consists of the sequence shown in SEQ ID NO: 69 or a sequence which is at least 80%, 85%, 90%, 95%, 96%, 97%, 98% or 99% identical thereto, and wherein the light chain variable region comprises or consists of the sequence shown in SEQ ID NO: 173 or a sequence which is at least 80%, 85%, 90%, 95%, 96%, 97%, 98% or 99% identical thereto. In one embodiment, the mAb which binds to an epitope located on HSV gI, comprises a heavy chain and a light chain, wherein the heavy chain variable region comprises or consists of a nucleotide sequence selected from SEQ ID NO: 281 or a sequence which is at least 80%, 85%, 90%, 95%, 96%, 97%, 98% or 99% identical thereto, and wherein the light chain variable region comprises or consists of a nucleotide sequence selected from SEQ ID NO: 385 or a sequence which is at least 80%, 85%, 90%, 95%, 96%, 97%, 98% or 99% identical thereto. Preferably, the heavy chain has a murine IgG1 isotype. More preferably, the heavy chain has VDJ regions IGHV2-9-1*01, IGHD2-3*01_5-3 and IGHJ4*01. Preferably, the light chain is a Kappa light chain class. More preferably, the light chain has VJ regions IGKV2-137*01 and IGKJ4*01.

In one aspect, the invention provides a nucleic acid encoding the antigen binding protein of the invention. In one embodiment, the antigen binding protein is an antibody. In a preferred embodiment, the antigen binding protein is a monoclonal antibody.

The term “nucleic acid” (or “polynucleotide”) in general means a polymeric form of nucleotides of any length, which contain deoxyribonucleotides, ribonucleotides, and/or their analogs. It includes DNA, RNA, DNA/RNA hybrids. It also includes DNA or RNA analogs, such as those containing modified backbones (e.g. peptide nucleic acids (PNAs) or phosphorothioates) or modified bases. The nucleic acids used herein are preferably provided in purified or substantially purified form i.e. substantially free from other nucleic acids (e.g. free from naturally-occurring nucleic acids), generally being at least about 50% pure (by weight), and usually at least about 90% pure. The nucleic acid molecules of the invention may be produced by any suitable means, including recombinant production, chemical synthesis, or other synthetic means. Suitable production techniques are well known to those of skill in the art. Typically, the nucleic acids of the invention will be in recombinant form, i. e. a form which does not occur in nature. For example, the nucleic acid may comprise one or more heterologous nucleic acid sequences (e.g. a sequence encoding another antigen and/or a control sequence such as a promoter or an internal ribosome entry site) in addition to the nucleic acid sequences encoding the antigen binding protein. The sequence or chemical structure of the nucleic acid may be modified compared to naturally-occurring sequences which encode the antigen binding protein, e.g. to increase the efficacy of expression or replication of the nucleic acid, or to provide additional stability or resistance to degradation. The nucleic acid molecule encoding the antigen binding protein may be codon optimized. By “codon optimized” is intended modification with respect to codon usage that may increase translation efficacy and/or half-life of the nucleic acid. Herein, “encoding” or “encodes” refers to the inherent property of specific sequences of nucleotides in a polynucleotide, to act as a template for synthesis of other polymers and macromolecules in biological processes, e.g., synthesis of peptides or proteins. Both the coding strand of a double-stranded nucleotide molecule (the sequence of which is usually provided in sequence listings), and the non-coding strand (used as the template for transcription of a gene or cDNA), can be referred to as encoding the peptide or protein. Unless otherwise specified, as used herein a “nucleotide sequence encoding an amino acid sequence” includes all nucleotide sequences that are degenerate versions of each other and that encode the same amino acid sequence.

In one aspect, the invention provides an expression vector comprising the nucleic acid of the invention.

As used herein, ‘expression’ refers to transcription or translation of a nucleic acid sequence. An ‘expression vector’ is a vector comprising a recombinant polynucleotide sequence to be expressed and comprising expression control sequences operatively linked to the recombinant polynucleotide sequence to be expressed. An expression vector for use according to the invention may be any suitable nucleic acid molecule including naked DNA or RNA, a plasmid, a virus, a cosmid, phage vector such as lambda vector, an artificial chromosome such as a BAC (bacterial artificial chromosome), or an episome. Alternatively, a vector may be a transcription and/or expression unit for cell-free in vitro transcription or expression, such as a T7-compatible system. Suitably, the vector has been substantially altered (e.g., having a gene or functional region deleted and/or inactivated) relative to a wild type sequence, and replicates and expresses the inserted polynucleotide sequence, when introduced into a host cell. “Recombinant” means that the polynucleotide is the product of at least one of cloning, restriction or ligation steps, or other procedures that result in a polynucleotide that is distinct from a polynucleotide found in nature.

In one aspect, the invention provides a host cell comprising the nucleic acid sequence or the expression vector of the invention.

In one aspect, the invention provides a method for the production of an antigen binding protein of the invention, comprising culturing the recombinant host cell of the invention conditions suitable for expression of the nucleic acid sequence or vector, whereby a polypeptide comprising the antigen binding protein is produced. In one embodiment, the antigen binding protein is an antibody. In a preferred embodiment, the antigen binding protein is a monoclonal antibody.

In one aspect, the invention provides an antigen binding protein produced by the method of the invention. In one embodiment, the antigen binding protein is an antibody. In a preferred embodiment, the antigen binding protein is a monoclonal antibody.

Assays

In one aspect, the invention provides the use of the antigen binding protein of the invention in the in vitro detection of a HSV gE antigen or gEgI heterodimer, confirmation of its ability or loss of ability to bind to human IgGs, and/or detection of a change in its conformation. In one embodiment, the antigen binding protein is an antibody. In a preferred embodiment, the antigen binding protein is a monoclonal antibody.

In one aspect, the invention provides an assay comprising exposing a sample comprising a HSV gE antigen or gEgI heterodimer to an antigen binding protein of the invention under conditions sufficient to form an immune complex and detecting the immune complex. In one embodiment, the antigen binding protein is an antibody. In a preferred embodiment, the antigen binding protein is a monoclonal antibody. Preferably, the assay of the invention is an in vitro assay.

As used herein, an ‘immune complex’ refers to a complex created by the binding of an antibody (or antigen binding protein) to an antigen. As used herein, ‘conditions sufficient to form an immune complex’ are conditions that allow an antibody (or antigen binding protein) to bind to an epitope on the antigen at a detectable degree. Such conditions depend upon the format of the binding reaction and may be determined using methods as are known in the art. See, e.g., Harlow and Lane, Antibodies: A Laboratory Manual, 2^{nd} ed. Cold Spring Harbor Laboratory Press, New York (2013). Immune complexes can be detected through conventional methods as are known in the art, e.g., immunohistochemistry, immunoprecipitations, flow cytometry, immunofluorescence microscopy, ELISA, immunoblotting, and chromatography. Methods are also known in the art for quantifying the immunological binding properties of an antibody.

In one aspect, the invention provides a binding assay for in vitro analysis of a sample comprising a HSV gE antigen or gEgI heterodimer comprising the steps of:
i) contacting the sample with a detection antibody directed against a HSV gE or gEgI epitope under conditions sufficient to form an immune complex; and
ii) measuring the interaction between the HSV gE antigen or gEgI heterodimer and detection antibody from step (i).

By comparison with values obtained with standard samples of known potency, results from step (ii) can be used to determine the potency of a test antigen. As used herein, “potency” relates to a measure of biological activity using a quantitative biological assay (also called a potency assay or bioassay), based on an attribute of a product which is linked to relevant biological properties. For example, a vaccine potency assay may be a measure which estimates/predicts whether the biologic will elicit the desired effect in patients and such an assay may be used in releasing a vaccine lot to the market. Suitably, “potency” is determined by reference to a reference standard.

In one embodiment the assay of the invention further comprises comparing the amount of antibody bound to the test HSV gE antigen or gEgI heterodimer to the amount of antigen binding protein bound to a reference HSV gE or gEgI heterodimer sample. In one embodiment the assay of the invention is carried out in duplicate, triplicate or more. In one embodiment an acceptable potency will be demonstrated when the test antigen is within the specification limits of the assay, as compared to the reference sample, wherein the specification limit is set as approximately 75%-125% of the reference sample. In one embodiment an acceptable potency is achieved when no statistically significant difference is observed between the data of the test antigen compared to the data of the reference sample. In one embodiment the sample containing the test antigen is diluted prior to or during the assay of the invention. In one embodiment the sample containing test antigen will be diluted optionally 2-fold, optionally 10-fold, optionally 50-fold, optionally 100-fold, optionally 1000-fold, optionally 10,000-fold or greater.

In a preferred embodiment, the dissociation constant (K_{D}) between the detection antibody and HSV gE antigen or gEgI heterodimer is lower than 5×10⁻⁷ M, preferably lower than 1×10⁻⁷ M, more preferably lower than 5×10⁻⁸ M.

In a preferred embodiment, the detection antibody is directed against an epitope on HSV gE, preferably on the HSV gE binding domain. Alternatively, the detection antibody is directed against a conformational epitope at the interface between HSV gE and HSV gI.

In a preferred embodiment, the detection antibody is an antibody of the invention.

The assays of the invention may be used to detect a HSV gE antigen, to confirm its three-dimensional conformation, to confirm its ability or loss of ability to bind to human IgGs, and/or to detect a change in its conformation. When the gE antigen is part of a gEgI heterodimer, the assays of the invention may also be used to confirm its heterodimer form.

In a preferred embodiment of the assays of the invention, the sample comprises a HSV2 gE antigen, or an immunogenic fragment thereof. Suitably, the HSV2 gE antigen or immunogenic fragment thereof does not comprise a functional transmembrane domain. Suitably, the HSV2 gE antigen or immunogenic fragment thereof does not comprise a cytoplasmic domain. Preferably, the HSV2 gE antigen or immunogenic fragment thereof comprises or consists essentially of a HSV2 gE ectodomain. In a preferred embodiment, the HSV2 gE ectodomain is at least 90%, 95%, 96%, 97%, 98%, 99%, 99.5% or 100% identical to SEQ ID NO: 5. Suitably, the HSV2 gE ectodomain may comprise one or more amino acid residue substitutions, deletions, or insertions relative to the amino acid sequence shown at SEQ ID NO: 5, for example 1, 2, 3, 4, 5, 6, 7, 8, 9 or amino acid residue substitution, deletion, or insertions. Suitably when the gE antigen is part of a HSV2 gEgI heterodimer, the HSV2 gI antigen or immunogenic fragment thereof does not comprise a functional transmembrane domain. Suitably, the HSV2 gI antigen or immunogenic fragment thereof does not comprise a cytoplasmic domain. Preferably, the HSV2 gI antigen or immunogenic fragment thereof comprises or consists essentially of a HSV2 gI ectodomain. In a preferred embodiment, the HSV2 gI ectodomain is at least 90%, 95%, 96%, 97%, 98%, 99%, 99.5% or 100% identical to SEQ ID NO: 6. Suitably, the HSV2 gI ectodomain may comprise one or more amino acid residue substitutions, deletions, or insertions relative to the amino acid sequence shown at SEQ ID NO: 6, for example 1, 2, 3, 4, 5, 6, 7, 8, 9 or 10 amino acid residue substitution, deletion, or insertions.

In one embodiment, the Fc binding domain (FCBD) of the gE antigen comprises one or more mutations compared to the native gE protein which diminish or abolish its ability to bind to the Fc domain of human IgGs. Examples of such mutations include point mutations P317R, R320D or P319D of SEQ NO: 5, or the insertion of amino acids “ARAA” between residues S338 and T339 of SEQ NO: 5. Other examples of suitable mutations to the gE FCBD are disclosed in European application n° 19191842.4, which is incorporated by reference. Without wishing to be bound by theory, it is hypothesized that in a subject administered with a vaccine composition comprising the HSV gE antigen or gEgI heterodimer, such mutations will prevent or reduce binding of human IgGs to the HSV gE antigen and thereby avoid the risk of blocking the action of the HSV gE antigen.

In one embodiment, the HSV2 gE antigen comprises a gE ectodomain, and the gE ectodomain comprises a point mutation at position P317 of SEQ ID NO: 5, preferably a P317R mutation, or a corresponding point mutation in another gE sequence. In one embodiment, the HSV2 gE antigen comprises a gE ectodomain, and the gE ectodomain comprises a point mutation at position R320 of SEQ ID NO: 5, preferably a R320D mutation, or a corresponding point mutation in another gE sequence. In one embodiment, the HSV2 gE antigen comprises a gE ectodomain, and the gE ectodomain comprises a point mutation at position P319 of SEQ ID NO: 5, preferably a P319D mutation, or a corresponding point mutation in another gE sequence. In one embodiment, the HSV2 gE antigen comprises a gE ectodomain, and the gE ectodomain comprise an insertion mutation, preferably the insertion of amino acids “ARAA”, between residues S338 and T339 of SEQ ID NO: 5, or a corresponding insertion mutation in another gE sequence.

In a preferred embodiment of the assays of the invention, the gE FCBD comprises one or more mutations compared to the native gE protein which diminish or abolish its ability to bind to the Fc domain of human IgGs, the assay, and the detection antibody or mAb is a functional antibody or mAb. Herein, a “functional antibody” is an antibody which is specific to the gE FCBD and which has the ability to block binding of the Fc domain of human IgGs. An example of a functional mAb is the mAb from clone 18 (see data presented in example 7).

In a preferred embodiment, the functional mAb which binds to an epitope located on the HSV gE FCBD, comprises a heavy chain, wherein the heavy chain variable region comprises the HCDR1 shown in SEQ ID NO: 50, the HCDR2 shown in SEQ ID NO: 51 and the HCDR3 shown in SEQ ID NO: 52, or variants thereof having 1, 2, or 3 amino acid deletions, substitutions or insertions.

In a preferred embodiment, the functional mAb which binds to an epitope located on the HSV gE FCBD, comprises a light chain, wherein the light chain variable region comprises the LCDR1 shown in SEQ ID NO: 154, the LCDR2 shown in SEQ ID NO: 155 and the LCDR3 shown in SEQ ID NO: 156, or variants thereof having 1, 2, or 3 amino acid deletions, substitutions or insertions.

In a preferred embodiment, the functional mAb which binds to an epitope located on the HSV gE FCBD, comprises a heavy chain and a light chain, wherein the heavy chain variable region comprises the HCDR1 shown in SEQ ID NO: 50, the HCDR2 shown in SEQ ID NO: 51 and the HCDR3 shown in SEQ ID NO: 52, and the light chain variable region comprises the LCDR1 shown in SEQ ID NO: 154, the LCDR2 shown in SEQ ID NO: 155 and the LCDR3 shown in SEQ ID NO: 156.

In a preferred embodiment, the functional mAb which binds to an epitope located on the HSV gE FCBD, comprises a heavy chain and a light chain, wherein the heavy chain variable region comprises or consists of the sequence shown in SEQ ID NO: 49 or a sequence which is at least 80%, 85%, 90%, 95%, 96%, 97%, 98% or 99% identical thereto, and wherein the light chain variable region comprises or consists of the sequence shown in SEQ ID NO: 153 or a sequence which is at least 80%, 85%, 90%, 95%, 96%, 97%, 98% or 99% identical thereto. Preferably, the heavy chain has a murine IgG1 isotype. More preferably, the heavy chain has VDJ regions IGHV1-63*02, IGHD1-1*01_5-3 and IGHJ2*02. Preferably, the light chain is a Kappa light chain class. More preferably, the light chain has VJ regions IGKV6-32*01 and IGKJ4*01.

By using a monoclonal antibody which binds to a conformational epitope, the result in step (ii) indicates the concentration of the corresponding conformational epitope in the sample and can distinguish between immunogens which retain the relevant conformational epitope (and function) and those which have lost the conformational epitope (e.g. due to denaturation, aggregation or breakdown during storage or by mishandling). In one embodiment the assay of the invention is used to determine or measure the presence of a test antigen in its functional tridimensional conformation.

By using a monoclonal antibody which binds to the gEgI heretodimer at a conformational epitope located at the interface between HSV gE and gI, the result in step (ii) will indicate the concentration of gEgI in the sample which is in the heterodimer conformation and can distinguish between gEgI heterodimer and free gE and free gI. In a preferred embodiment, the detection antibody is an antibody which binds to the gEgI heretodimer at a conformational epitope located at the interface between HSV gE and gI as described herein, preferably a monoclonal antibody. Examples of mAbs which bind to the HSV gEgI heterodimer at a conformational epitope located at the interface between HSV gE and HSV gI include mAbs from clones 5, 9, 10, 15, 17, 19, 20, 24, 34, 39, 40, 42 and 45 (see example section and tables 7-11).

Suitably, the assay is an in vitro relative potency (IVRP) assay in which capture and detection antibodies against two different epitopes of the HSV gE antigen or gEgI heterodimer are used, for example in a sandwich ELISA. Monoclonal or polyclonal antibodies can be used as the capture and detection antibodies although monoclonal antibodies may allow quantification of smaller differences. Dilution curves of antigen test samples are compared against a reference curve by parallel-line analysis. The relative potency can then be determined by multiplying the reference standard potency by the ratio of the sample ED50 versus the reference ED50. Generally, a low sample ED50 indicates lower vaccine potency.

In a preferred embodiment, the assay is an IVRP comprising the steps of:
i) permitting the HSV gE antigen or gEgI heterodimer within the sample to interact with a capture antibody directed against a first epitope on the HSV gE antigen or gEgI heterodimer and with a detection antibody directed against a second epitope on the HSV gE antigen or gEgI heterodimer; and
ii) measuring the interaction between the HSV gE antigen or gEgI heterodimer and detection antibody from step (i).

Suitably, the first and second epitopes do not overlap. In one embodiment where the gE antigen is part of a gEgI heterodimer, the first epitope is a conformational epitope located at the interface between HSV gE and HSV gI, and the second epitope is an epitope on HSV gE, preferably on the HSV gE Fc binding domain. In another embodiment, the first epitope is an epitope on HSV gE, preferably on the HSV gE Fc binding domain, and the second epitope is a conformational epitope located at the interface between HSV gE and HSV gI. In a preferred embodiment, the capture and detection antibodies are monoclonal antibodies. In a preferred embodiment, the capture and detection antibodies are mAbs according to the invention.

In a preferred embodiment, the dissociation constant (K_{D}) between the capture antibody and HSV gE antigen or gEgI heterodimer is lower than 5×10⁻⁷ M, preferably lower than 1×10⁻⁷ M, more preferably lower than 5×10⁻⁸ M. In a preferred embodiment, the dissociation constant (K_{D}) between the detection antibody and HSV gE antigen or gEgI heterodimer is lower than 5×10⁻⁷ M, preferably lower than 1×10⁻⁷ M, more preferably lower than 5×10⁻⁸ M.

In one embodiment of the IVRP assay, the capture antibody is a mAb which binds to an epitope located on the HSV gE FCBD. Examples of mAbs which bind to the HSV gE FCBD domain include mAbs from clones 18, 12/13, 14, 43 and 47 (see example section and tables 7-11). A preferred example is the mAb from clone 12 (see data presented in example 7). In a preferred embodiment, the mAb which binds to an epitope located on the HSV gE FCBD, comprises a heavy chain, wherein the heavy chain variable region comprises the HCDR1 shown in SEQ ID NO: 30, the HCDR2 shown in SEQ ID NO: 31 and the HCDR3 shown in SEQ ID NO: 32, or variants thereof having 1, 2, or 3 amino acid deletions, substitutions or insertions. In a preferred embodiment, the mAb which binds to an epitope located on the HSV gE FCBD, comprises a light chain, wherein the light chain variable region comprises the LCDR1 shown in SEQ ID NO: 134, the LCDR2 shown in SEQ ID NO: 135 and the LCDR3 shown in SEQ ID NO:136, or variants thereof having 1, 2, or 3 amino acid deletions, substitutions or insertions. In a preferred embodiment, the mAb which binds to an epitope located on the HSV gE FCBD, comprises a heavy chain and a light chain, wherein the heavy chain variable region comprises the HCDR1 shown in SEQ ID NO: 30, the HCDR2 shown in SEQ ID NO: 31 and the HCDR3 shown in SEQ ID NO: 32, and the light chain variable region comprises the LCDR1 shown in SEQ ID NO: 134, the LCDR2 shown in SEQ ID NO: 135 and the LCDR3 shown in SEQ ID NO:136. In one embodiment, the mAb which binds to an epitope located on the HSV gE FCBD, comprises a heavy chain and a light chain, wherein the heavy chain variable region comprises or consists of the sequence shown in SEQ ID NO: 29 or a sequence which is at least 80%, 85%, 90%, 95%, 96%, 97%, 98% or 99% identical thereto, and wherein the light chain variable region comprises or consists of the sequence shown in SEQ ID NO: 133 or a sequence which is at least 80%, 85%, 90%, 95%, 96%, 97%, 98% or 99% identical thereto. Preferably, the heavy chain has a murine IgG1 isotype. More preferably, the heavy chain has VDJ regions IGHV2-9-1*01, IGHD2-4*01_5-3 and IGHJ4*01. Preferably, the light chain is a Kappa light chain class. More preferably, the light chain has VJ regions IGKV3-1*01 and IGKJ1*01.

In one embodiment of the IVRP assay, the detection antibody is directed against a conformational epitope located at the interface between HSV gE and HSV gI. Examples of antibodies directed against a conformational epitope located at the interface between HSV gE and HSV gI include mAbs from clones 5, 9, 10, 15, 17, 19, 20, 24, 34, 39, 40, 42 and 45 (see example section and tables 7-11).

In a preferred embodiment, the mAb which binds to the HSV gEgI heterodimer at a conformational epitope located at the interface between HSV gE and HSV gI, comprises a heavy chain, wherein the heavy chain variable region comprises the HCDR1 shown in SEQ NO: 18, the HCDR2 shown in SEQ NO: 19 and the HCDR3 shown in SEQ NO: 20, or variants thereof having 1, 2, or 3 amino acid deletions, substitutions or insertions. In a preferred embodiment, the mAb which binds to the HSV gEgI heterodimer at a conformational epitope located at the interface between HSV gE and HSV gI, comprises a light chain, wherein the light chain variable region comprises the LCDR1 shown in SEQ NO:122, the LCDR2 shown in SEQ NO: 123 and the LCDR3 shown in SEQ NO: 124, or variants thereof having 1, 2, or 3 amino acid deletions, substitutions or insertions. In a preferred embodiment, the mAb which binds to the HSV gEgI heterodimer at a conformational epitope located at the interface between HSV gE and HSV gI, comprises a heavy chain and a light chain, wherein the heavy chain variable region comprises the HCDR1 shown in SEQ NO: 18, the HCDR2 shown in SEQ NO: 19 and the HCDR3 shown in SEQ NO: 20, and the light chain variable region comprises the LCDR1 shown in SEQ NO: 122, the LCDR2 shown in SEQ NO: 123 and the LCDR3 shown in SEQ NO: 124. In one embodiment, the mAb which binds to the HSV gEgI heterodimer at a conformational epitope located at the interface between HSV gE and HSV gI, comprises a heavy chain and a light chain, wherein the heavy chain variable region comprises or consists of the sequence shown in SEQ ID NO: 17 or a sequence which is at least 80%, 85%, 90%, 95%, 96%, 97%, 98% or 99% identical thereto, and wherein the light chain variable region comprises or consists of the sequence shown in SEQ ID NO: 121 or a sequence which is at least 80%, 85%, 90%, 95%, 96%, 97%, 98% or 99% identical thereto. Preferably, the heavy chain has a murine IgG1 isotype. More preferably, the heavy chain has VDJ regions IGHV7-1*02, IGHD1-1*01_5-3 and IGHJ3*01. Preferably, the light chain is a Kappa light chain class. More preferably, the light chain has VJ regions IGKV5-39*01 and IGKJ2*01.

In one embodiment of the IVRP assay, the detection antibody is a functional antibody or mAb. An example of a functional mAb is the mAb from clone 18 (see data presented in example 7). In a preferred embodiment, the functional mAb which binds to an epitope located on the HSV gE FCBD, comprises a heavy chain, wherein the heavy chain variable region comprises the HCDR1 shown in SEQ ID NO: 50, the HCDR2 shown in SEQ ID NO: 51 and the HCDR3 shown in SEQ ID NO: 52, or variants thereof having 1, 2, or 3 amino acid deletions, substitutions or insertions. In a preferred embodiment, the functional mAb which binds to an epitope located on the HSV gE FCBD, comprises a light chain, wherein the light chain variable region comprises the LCDR1 shown in SEQ ID NO: 154, the LCDR2 shown in SEQ ID NO: 155 and the LCDR3 shown in SEQ ID NO: 156, or variants thereof having 1, 2, or 3 amino acid deletions, substitutions or insertions. In a preferred embodiment, the functional mAb which binds to an epitope located on the HSV gE FCBD, comprises a heavy chain and a light chain, wherein the heavy chain variable region comprises the HCDR1 shown in SEQ ID NO: 50, the HCDR2 shown in SEQ ID NO: 51 and the HCDR3 shown in SEQ ID NO: 52, and the light chain variable region comprises the LCDR1 shown in SEQ ID NO: 154, the LCDR2 shown in SEQ ID NO: 155 and the LCDR3 shown in SEQ ID NO: 156. In a preferred embodiment, the functional mAb which binds to an epitope located on the HSV gE FCBD, comprises a heavy chain and a light chain, wherein the heavy chain variable region comprises or consists of the sequence shown in SEQ ID NO: 49 or a sequence which is at least 80%, 85%, 90%, 95%, 96%, 97%, 98% or 99% identical thereto, and wherein the light chain variable region comprises or consists of the sequence shown in SEQ ID NO: 153 or a sequence which is at least 80%, 85%, 90%, 95%, 96%, 97%, 98% or 99% identical thereto. Preferably, the heavy chain has a murine IgG1 isotype. More preferably, the heavy chain has VDJ regions IGHV1-63*02, IGHD1-1*01_5-3 and IGHJ2*02. Preferably, the light chain is a Kappa light chain class. More preferably, the light chain has VJ regions IGKV6-32*01 and IGKJ4*01.

Suitably, the assay of the invention is an enzyme linked immunosorbent assay (ELISA). The invention can use any ELISA format, including those conventionally known as direct ELISA, indirect ELISA, sandwich ELISA, and competitive ELISA. Suitably, the assay of the invention uses the gyrolab technology as described in examples 1 and 7.

Suitably, the detection antibody is labelled. Labelling of antibodies can take various forms. The antibody can be labelled with an enzyme, which is then used to catalyze a reaction whose product is readily detectable. The linked enzyme can cause a detectable change in an enzyme substrate which is added to the labelled antibody after it becomes immobilized e.g. to modify a substrate in a manner which causes a colour change. For example, the enzyme may be a peroxidase (e.g. horseradish peroxidase, HRP), or a phosphatase (e.g. alkaline phosphatase, AP). Other enzymes can also be used e.g. laccase, β-galactosidase, etc.

The choice of substrate will depend on the choice of linked enzyme. Preferred substrates undergo a colorimetric change, a chemiluminescent change, or a chemifluorescent change when contacted with the linked enzyme. Colorimetric substrates (and their enzymatic partners) include but are not limited to: PNPP or p-Nitrophenyl Phosphate (AP); ABTS or 2,2′-Azinobis [3-ethylbenzothiazoline-6-sulfonic acid] (HRP); OPD or o-phenylenediamine dihydrochloride (HRP); and TMB or 3,3′,5,5′-tetramethylbenzidine (HRP). Chemiluminescent substrates include luminol or 5-amino-2,3-dihydro-1,4-phthalazinedione (HRP), particularly in the presence of modified phenols such as p-iodophenol. Chemifluorescent substrates include p-hydroxyhydrocinnamic acid. Various proprietary substrates are also available, and these can be used with the invention if desired e.g. QuantaBlu, QuantaRed, SuperSignal, Turbo TMB, etc.

Where an ELISA reagent is immobilized on a solid surface, this surface can take various forms. Usually the reagent is immobilized on a plastic surface, such as a surface made from polystyrene, polypropylene, polycarbonate, or cyclo-olefin. The plastic will usually be transparent and colourless, particularly when using chromogenic enzyme substrates. White or black plastics may be preferred used when using luminescent or fluorescent substrates, as known in the art. The plastic will generally be used in the form of a microwell plate (microtiter plate) as known in the art for ELISA (a flat plate having multiple individual and reaction wells). Such plates include those with 6, 24, 96, 384 or 1536 sample wells, usually arranged in a 2:3 rectangular matrix. Microwell plates facilitate the preparation of dilution series and also the transfer of materials from one plate to another while maintaining spatial relationships e.g. in the step of transferring a mixture of antibody and vaccine into a different microwell plate for measuring the interaction between the antibody and vaccine.

During an ELISA it may be desirable to add a blocking reagent and/or detergent e.g. to reduce non-specific binding interactions which might distort the assay's results. Blocking procedures are familiar to people working in the ELISA field. In an embodiment the assay of the invention uses a 1% bovine serum albumin blocking solution to reduce non-specific binding.

In addition to the ELISA formats discussed above, the invention can also be extended to use alternatives to ELISA, such as flow injection immunoaffinity analysis (FIIAA), AlphaLISA or AlphaScreen [6], dissociation-enhanced lanthanide fluorescent immunoassay (DELFIA), ELAST, the BIO-PLEX Suspension Array System, MSD, etc. Any suitable antibody-antigen complex binding assays can be used.

As an alternative to using a conjugated enzyme as the label, other labelling is possible. For instance, other indirect labels {i.e. alternative to enzymes) can be used, but it is also possible to label the antibody by conjugation to a direct label such as a coloured particle, an electrochemically active reagent, a redox reagent, a radioactive isotope, a fluorescent label or a luminescent label. As a further alternative, the antibody can be conjugated to a high affinity tag such as biotin, avidin or streptavidin. An enzyme conjugated to a ligand for the tag, such as avidin, streptavidin or biotin can then be used to detect immobilized antibody. Any of these variations can be used within the scope and spirit of the overall invention.

In one aspect, the invention provides a pharmaceutical composition comprising an antigen binding protein, antibody or mAb of the invention and a pharmaceutically acceptable carrier.

A “pharmaceutically acceptable carrier” includes any carrier that does not itself induce a therapeutic effect in the individual receiving the composition. Suitable carriers are typically large, slowly metabolized macromolecules such as proteins, polysaccharides, polylactic acids, polyglycolic acids, polymeric amino acids, amino acid copolymers, sucrose, trehalose, lactose, and lipid aggregates (such as oil droplets or liposomes). Such carriers are well known to those of ordinary skill in the art. The compositions may also contain a pharmaceutically acceptable diluent, such as water, saline, glycerol, etc. Additionally, auxiliary substances, such as wetting or emulsifying agents, pH buffering substances, and the like, may be present. The appropriate carrier may depend in large part upon the route of administration.

In one aspect, the invention provides the antigen binding protein, antibody, mAb or pharmaceutical composition of the invention for use in therapy.

In one aspect, the invention provides the antigen binding protein, antibody, mAb or pharmaceutical composition of the invention for use in the treatment of recurrent herpes infection, or for use in a method for prevention or reduction of the frequency of recurrent herpes virus infection in a subject, preferably a human subject.

In one aspect, the invention provides the antigen binding protein, antibody, mAb or pharmaceutical composition of the invention for use in the manufacture of an immunogenic composition.

In one aspect, the invention provides the use of the antigen binding protein, antibody, mAb or pharmaceutical composition of the invention in the manufacture of a medicament for the treatment of herpes infection or herpes-related disease.

In one aspect, the invention provides a method of treating a herpes virus infection or herpes virus related disease in a subject in need thereof comprising administering an immunologically effective amount of the antigen binding protein, antibody, mAb or pharmaceutical composition of the invention to the subject.

As used herein, the terms “treat” and “treatment” as well as words stemming therefrom, are not meant to imply a “cure” of the condition being treated in all individuals, or 100% effective treatment in any given population. Rather, there are varying degrees of treatment which one of ordinary skill in the art recognizes as having beneficial therapeutic effect(s). In this respect, the inventive methods and uses can provide any level of treatment of herpes virus infection and in particular HSV2 or HSV1 related disease in a subject in need of such treatment, and may comprise reduction in the severity, duration, or number of recurrences over time, of one or more conditions or symptoms of herpes virus infection, and in particular HSV2 or HSV1 related disease.

In a preferred embodiment of the pharmaceutical composition, use in therapy or treatment or method of treatment described above, the antibody is an antibody which binds to an epitope located on the HSV gE FCBD, preferably a functional mAb. Without wishing to be bound by theory, it is hypothesized that such functional antibodies or mAbs have the ability to prevent or reduce binding of the human IgGs to HSV gEgI in a subject infected by HSV, and thereby limit or abolish the immune evasion mechanism and allow natural immunity to play its role. An example of a functional mAb is the mAb from clone 18 (see data presented in example 7).

In a preferred embodiment, the functional mAb which binds to an epitope located on the HSV gE FCBD, comprises a heavy chain, wherein the heavy chain variable region comprises the HCDR1 shown in SEQ ID NO: 50, the HCDR2 shown in SEQ ID NO: 51 and the HCDR3 shown in SEQ ID NO: 52, or variants thereof having 1, 2, or 3 amino acid deletions, substitutions or insertions.

In a preferred embodiment, the functional mAb which binds to an epitope located on the HSV gE FCBD, comprises a light chain, wherein the light chain variable region comprises the LCDR1 shown in SEQ ID NO: 154, the LCDR2 shown in SEQ ID NO: 155 and the LCDR3 shown in SEQ ID NO: 156, or variants thereof having 1, 2, or 3 amino acid deletions, substitutions or insertions.

In a preferred embodiment, the functional mAb which binds to an epitope located on the HSV gE FCBD, comprises a heavy chain and a light chain, wherein the heavy chain variable region comprises the HCDR1 shown in SEQ ID NO: 50, the HCDR2 shown in SEQ ID NO: 51 and the HCDR3 shown in SEQ ID NO: 52, and the light chain variable region comprises the LCDR1 shown in SEQ ID NO: 154, the LCDR2 shown in SEQ ID NO: 155 and the LCDR3 shown in SEQ ID NO: 156.

In a preferred embodiment, the functional mAb which binds to an epitope located on the HSV gE FCBD, comprises a heavy chain and a light chain, wherein the heavy chain variable region comprises or consists of the sequence shown in SEQ ID NO: 49 or a sequence which is at least 80%, 85%, 90%, 95%, 96%, 97%, 98% or 99% identical thereto, and wherein the light chain variable region comprises or consists of the sequence shown in SEQ ID NO: 153 or a sequence which is at least 80%, 85%, 90%, 95%, 96%, 97%, 98% or 99% identical thereto. Preferably, the heavy chain has a murine IgG1 isotype. More preferably, the heavy chain has VDJ regions IGHV1-63*02, IGHD1-1*01_5-3 and IGHJ2*02. Preferably, the light chain is a Kappa light chain class. More preferably, the light chain has VJ regions IGKV6-32*01 and IGKJ4*01.

Sequence Comparison

For the purposes of comparing two closely-related polynucleotide or polypeptide sequences, the “sequence identity” or “% identity” between a first sequence and a second sequence may be calculated using an alignment program, such as BLAST® (available at blast.ncbi.nlm.nih.gov, last accessed 12 Sep. 2016) using standard settings. Alternative methods include using a gapped method in which gaps in the alignment, for example deletions in one sequence relative to the other sequence, are considered. Identity between polypeptides may be calculated by various algorithms. For example, the Needle program, from the EMBOSS package (Free software; EMBOSS: The European Molecular Biology Open Software Suite (2000). Trends in Genetics 16(6): 276-277) and the Gap program from the GCG® package (Accelrys Inc.) may be used. This Gap program is an implementation of the Needleman-Wunsch algorithm described in: [Needleman, S. B. and Wunsch, C. D. (1970) J. Mol. Biol. 48, 443-453]. The BLOSUM62 scoring matrix can be used, and the gap open and extension penalties were respectively 8 and 2. Identity between two sequences is calculated across the entire length of both sequences and is expressed as a percentage of the reference sequence.

A “difference” between two sequences refers to an insertion, deletion or substitution, e.g., of a single amino acid residue in a position of one sequence, compared to the other sequence.

For the purposes of comparing a first, reference polypeptide sequence to a second, comparison polypeptide sequence, the number of insertions, substitutions and/or deletions made to the first sequence to produce the second sequence may be ascertained. An insertion is the addition of one amino acid residue into the sequence of the first polypeptide (including addition at either terminus of the first polypeptide). A substitution is the substitution of one amino acid residue in the sequence of the first polypeptide with one different amino acid residue. A deletion is the deletion of one amino acid residue from the sequence of the first polypeptide (including deletion at either terminus of the first polypeptide).

As used herein, a “Variant” is a peptide sequence that differs in sequence from a reference peptide sequence but retains essential properties of the reference molecule. Changes in the sequence of peptide variants are limited or conservative, so that the sequences of the reference peptide and the variant are closely similar overall and, in many regions, identical. A variant and reference peptide can differ in amino acid sequence by one or more substitutions, additions or deletions in any combination. A variant of a peptide can be naturally occurring such as an allelic variant, or can be a variant that is not known to occur naturally. Non-naturally occurring variants of nucleic acids and peptides may be made by mutagenesis techniques or by direct synthesis.

Terms

Unless otherwise explained in the context of this disclosure, all technical and scientific terms used herein have the same meaning as commonly understood by one of ordinary skill in the art to which this disclosure belongs. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The singular terms “a,” “an,” and “the” include plural referents unless context clearly indicates otherwise. Similarly, the word “or” is intended to include “and” unless the context clearly indicates otherwise. The term “plurality” refers to two or more. It is further to be understood that all base sizes or amino acid sizes, and all molecular weight or molecular mass values, given for nucleic acids or polypeptides are approximate, and are provided for description. Additionally, numerical limitations given with respect to concentrations or levels of a substance, such as an antigen, are intended to be approximate. Thus, where a concentration is indicated to be at least (for example) 200 pg, it is intended that the concentration be understood to be at least approximately (or “about” or “˜”) 200 pg.

The term “comprises” means “includes.” Thus, unless the context requires otherwise, the word “comprises,” and variations such as “comprise” and “comprising” will be understood to imply the inclusion of a stated compound or composition (e.g., nucleic acid, polypeptide, antigen) or step, or group of compounds or steps, but not to the exclusion of any other compounds, composition, steps, or groups thereof. The abbreviation, “e.g.” is derived from the Latin exempli gratia and is used herein to indicate a non-limiting example. Thus, the abbreviation “e.g.” is synonymous with the term “for example.”

Amino acid sequences provided herein are designated by either single-letter or three-letter nomenclature, as is known in the art (see, e.g., Eur. J. Biochem. 138:9-37(1984)).

Although methods and materials similar or equivalent to those described herein can be used in the practice or testing of this disclosure, suitable methods and materials are described below.

All references or patent applications cited within this patent specification are incorporated by reference herein.

The present invention will now be further described by means of the following non-limiting examples.

### EXAMPLES

### Example 1—Materials and Methods

Antigens

The following test antigens were produced in HEK293 cells or in CHO cells:
gEgI WT: HSV2 gEgI heterodimer comprising gE and gI wildtype ectodomains (SEQ ID NO: 5 and SEQ ID NO: 6 respectively),
gEgI mutant (P317R): HSV2 gEgI mutant heterodimer comprising gE and gI ectodomains with mutation P317R in gE,
gEgI mutant (R320D): HSV2 gEgI mutant heterodimer comprising gE and gI ectodomains with mutation R320D in gE,
gEgI mutant (P319D): HSV2 gEgI mutant heterodimer comprising gE and gI ectodomains with mutation P319D in gE,
gEgI mutant (338): HSV2 gEgI mutant heterodimer comprising gE and gI ectodomains with insertion of amino acids “ARAA” between residues S338 and T339 of gE,
gE WT: HSV2gE wildtype ectodomain,
gI WT: HSV2gI wildtype ectodomain,
gE FCBD: HSV2gE wildtype Fc binding domain.

Generation of gEgI Antigen Specific Monoclonal Antibodies (mAbs)

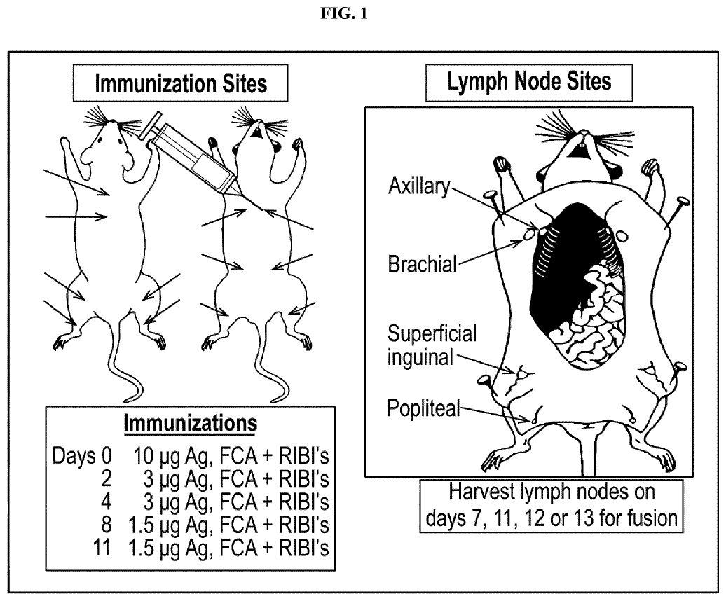

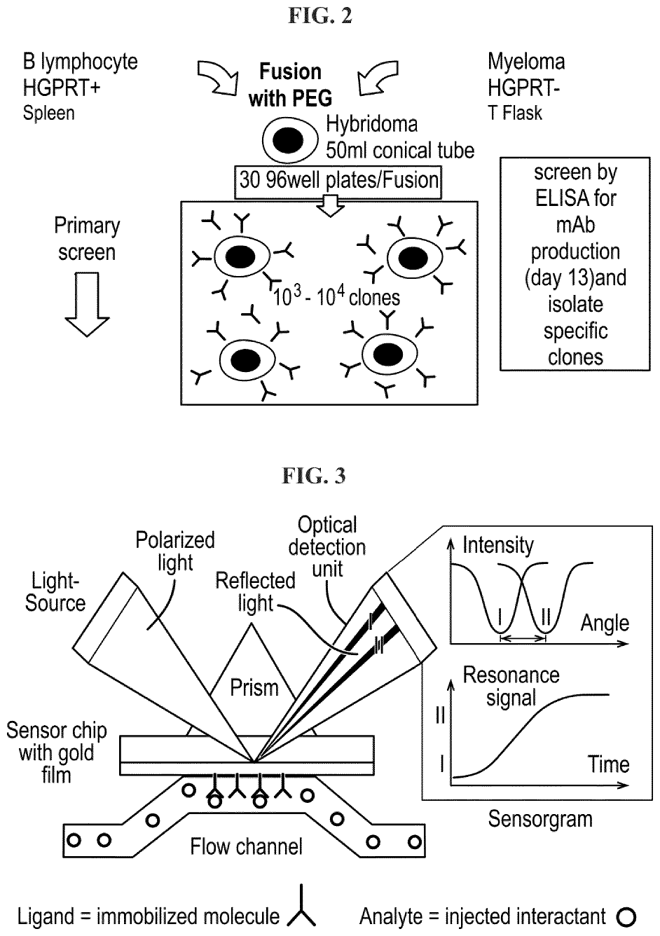

5 Female Balb C mice aged from 6-8 weeks were immunized using Repetitive Immunization Multiple Sites (RIMMS) protocol (FIG. 1). 100 μl of gEgI antigen produced in HEK 293 cells were injected at the following doses: 20-5-5-2.5 μg subcutaneously in multiple points on days 0, 4, 8, 11 in GSK AS02 adjuvant system. On day 13, lymph nodes of the 5 mice were collected and processed following the appropriate hybridoma generation protocol (FIG. 2).
Hybridoma supernatants were tested on day 25 by ELISA on gEgI and on gEgI mutants. Hybridoma supernatants were also tested on gE and gI proteins alone and on the gE Fₑ binding domain (FCBD) sub-domain. Western blot analysis in reduced and non-reduced conditions was performed to evaluate the putative type of epitope (conformational or linear) recognized by each mAb.

Specificity Screening by ELISA

To determine specificity by ELISA, target antigen (gEgI), antigen mutants or antigen sub-domains were directly coated on NUNC Maxisorp 96 wells microplates. Typically, 100 μl of antigen at a concentration between 1 to 5 μg/ml were coated overnight in PBS at 4° C. The coating solution was then removed by washing 3 times in NaCl/tween 0.05% solution, and 200 μl of post coating solution (PBS+BSA1%) was added to the microplate and incubated at room temperature (RT) for 30 minutes. After washing 3 times, 100 μl of cell culture supernatant (undiluted) were added to the wells and incubated 30 minutes at RT. After appropriate washing steps, visualisation was performed using Rabbit anti-mouse-Biotin antibody as secondary system, Streptavidin peroxydase and Ortho Phenylene Di-Amine as chromogen in the presence of hydrogen peroxyde in citrate buffer. Plates were read at 490 nm.

Western Blot Analysis

Western blot was conducted using 4-12% Nu-Page Bis-Tris 1.0 mm×10 gels. Sample buffer with or without b-mercapto ethanol was used. 20 μl of sample containing 2 μg of protein was added in each well. Migration was performed for 45 min at 200 Volts. At the end of migration, transfer on Nitrocellulose membrane was performed using I blot® system from invitrogen. Revelation was done after membrane postcoating with PBS+Milk Difco 1%. mAbs supernatant were added at 1/1 dilution for 1 h. Revelation was performed with Rabbit anti-mouse antibody conjugated to Alclaine Phosphatase and NBT+BCIP as revelators.

Surface Plasmon Resonance (SPR) Analysis

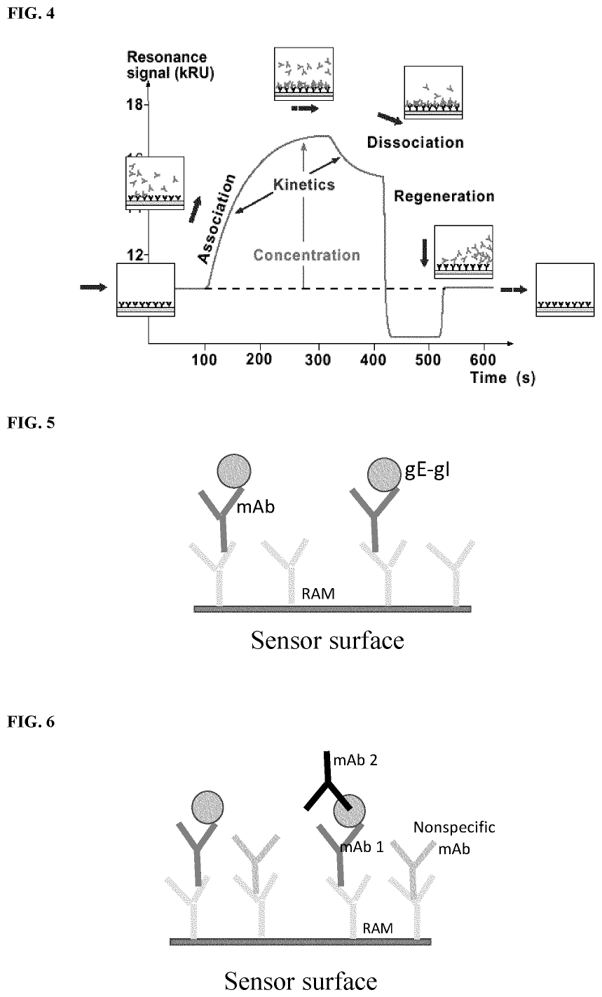

BIAcore technology can be used to study protein-protein, protein-oligonucleotide, protein-carbohydrate, and oligonucleotide-oligonucleotide interactions. BIAcore biosensor technology is a versatile, highly sensitive, label-free, and easy-to-use approach to study binding interactions quantitatively, under strictly controlled conditions. The technology relies on the phenomenon of “surface plasmon resonance” (SPR) (FIG. 3), small changes in the reflection intensity of monochromatic light occur when a protein or other molecule binds at the sensor chip surface. SPR (Surface plasmon resonance) detection monitors changes in refractive index close to the surface, and differences in refractive index between running buffer and injected sample will be recorded as a rapid shift in response at the beginning and end of the injection. This monitoring corresponds to a sensorgram that is a plot of response against time, showing the progress of the interaction (FIG. 4). This curve is displayed directly on the computer screen during the course of analysis. The readout allows to follow the entire binding event. The analysis is based on binding responses obtained at one or several specific time points (report point analysis). Individual contribution to binding corresponding to the mass of antigen that could be associated to a fixed amount of mAb (normalization). That parameter is dependent of epitope concentration, accessibility and kinetic constants of the mAbs.
Biacore Binding chart assay (FIG. 5)—4000-6000 RU of rabbit anti-mouse (RAM) antibodies (GE healthcare) were covalently coupled on flowcells of a CM3 sensorchip by the EHS/EDC chemistry. Relevant mAbs were pumped over sensor surface for specific time at a 101l/min flow rate at 25° C. over flowcells resulting in the binding of 100-250 RU for each antibody. Antigen at a concentration around 10 μg/ml for gEgI and 6 μg/ml for FCBD was then pumped over sensor surface for 180 s at a 30-601l/min flow rate at 25° C. RU associated with the antigen binding for the specific mAbs are then monitored and normalized for 200 RU of mAbs. All experiments were performed with PBS+P20 0.005% as running buffer and with PBS+P20 0.0005% as sample buffer, surface regeneration was achieved by 3 washes of 30 s with glycine pH 1.5 at 30 μl/min flow rate. Report points were monitored 15 s after end of injection. Results were expressed as binding activity of mAb to antigen. This corresponds to individual contribution to binding corresponding to the mass of antigen that could be associated to a fixed amount of mAb normalized to 200 RU.
Biacore Binning assay (FIG. 6)—mAbs were tested in a matrix mode (one to each other) to evaluate the capacity of the second antibody to bind when antigen is captured by the first antibody. 4000-6000 RU of rabbit anti-mouse (RAM) antibodies (GE healthcare) were covalently coupled on flowcells of a CM3 sensorchip by the EHS/EDC chemistry. A first mAb was pumped over the sensor surface for specific time at a 10 μl/min flow rate at 25° C. over flowcells resulting in the binding of 250-400 RU for each mAb. To avoid false positives, free sites of the RAM were quenched using a pool of non relevant mAbs pumped over the sensor surface until no binding on RAM was observed. Antigen at a concentration around 25 μg/ml for gEgI or FCBD was then pumped over the sensor surface for 120 s at a 101l/min flow rate at 25° C. The second mAb was pumped over the sensor surface and binding was monitored. All experiments were performed with PBS+P20 0.005% as running buffer and with PBS+P20 0.0005% as sample buffer, surface regeneration was achieved by 3 washes of 30 s with glycine pH 1.5 at 30 μl/min flow rate. Binding results were reported in a matrix and a surface like map of epitopes was derived to facilitate interpretation.
Determination of affinity constants—Affinity constants of gEgI mAbs were determined by SPR using the single cycle kinetic mode. The method consists in injecting sequentially increasing concentrations (up to 5) of the analyte, with only one regeneration step performed at the end of the complete binding cycle: ±4000 RU of rabbit anti-mouse antibodies (GE healthcare) were covalently coupled on FC1/FC2 and FC3/FC4 flowcells of a CM3 sensorchip by the EHS/EDC chemistry. Relevant mAbs (clones 12, 18) were pumped over sensor surface for a specific time at a 10p/min flow rate at 25° C. over appropriate flowcells resulting in the binding of ±100 RU for each antibody. All experiments were performed with PBS+P20 0.005% as running buffer and sample buffer, surface regeneration was achieved by 3 washes of 30 s with glycine pH 1.7 at 30 μl/min flow rate at the end of run. The gEgI antigen at 5 different concentrations was then pumped over the sensor surface (180 s for clone 12 mAb and 120 sec for clone 18 mAb) at a 30 μl/min flow rate at 25° C. RU associated with the antigen binding for the specific mAbs were then monitored and kinetics parameters were calculated by the Biacore Kinetics summary software. This software allows the visualisation of kinetics properties of the interaction using a selected mathematical binding model. The results were fitted with the chosen model corresponding to the binding model 1:1 (ligand:analyte).

Sanger and Next Generation Sequencing (NGS) of mAbs VH and VL

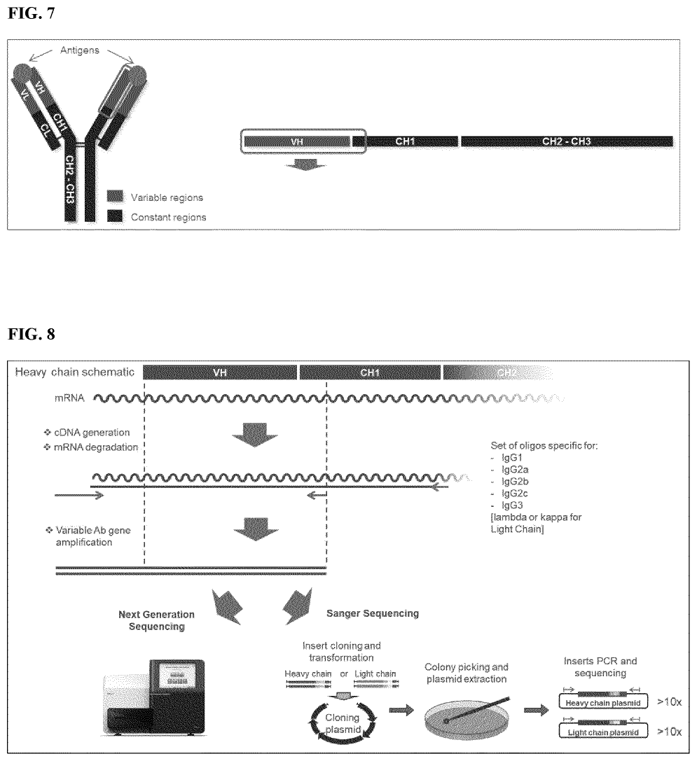

The whole procedure aims to sequence exclusively the variable regions of the light and heavy antibody chains (VL and VH, FIG. 7). Those regions form the binding pocket, which binds the specific antigens and contains the major diversity of the immunoglobulin. The sequencing strategy was designed to obtain also the sequence of a small region of the constant domain (˜50-60 bp) for the identification of the antibody class/subtype. The full sequence can be retrieved from available antibody databases (e.g. IMGT).
Different molecular biology methods were combined, each applied to process the template in specific steps (FIG. 8), which can be summarized as follows:
1. Thawing and growth of hybridoma cell clone
2. RNA extraction
3. cDNA generation by retro-transcription
4. 3′ polyA tailing
5. 5′ Rapid Amplification of cDNA Ends (RACE) PCR
6. Cloning into commercial plasmid (TOPO PCR cloning)
7. Colony picking, bacterial growth and plasmid extraction
8. Sanger sequencing
9. Next Generation Sequencing
   Internal protocols and SOPs were used for steps 1 and 8, while manufacturer's instructions were followed for the other steps.
   Thawing and growth of hybridoma cell clone—Cells were thawed and growth for 10 days following internal laboratory protocol.
   RNA extraction—RNA extracted with Qiagen kit RNeasy Mini (Qiagen)
   cDNA generation by retro-transcription—Retro transcription performed using SuperScript IV first-strand synthesis system (Invitrogen) and a set of oligos specific for either the light chain or heavy chain amplification:

HEAVY CHAIN oligos (for all isotypes):
RT-mHingeG1_rev
(SEQ ID NO: 433)
5′-gcaaggcttacaaccacaatc-3′
RT-mHingeG2a_rev
(SEQ ID NO: 434)
5′-gaggacagggcttgattgtgg-3′
RT-mHingeG2b_rev
(SEQ ID NO: 435)
5′-gaggacaggggttgattgttg-3′
RT-mHingeG2c_rev
(SEQ ID NO: 436)
5′-ggacaggggttctgtgttatgg-3′
RT-mHingeG3_rev
(SEQ ID NO: 437)
5′-ctgggcttgggtattctagg-3′
LIGHT CHAIN oligos (for both kappa and lambda
classes):
RT_mKappaCL_rev
(SEQ ID NO: 438)
5′-ctcattcctgttgaagctcttg-3′
RT_mLambda1/4-CL_rev
(SEQ ID NO: 439)
5′-gcacgggacaaactcttctc-3′
RT_mLambda2/3-CL_rev
(SEQ ID NO: 440)
5′-ctgcaggagacagactcttctc-3′

3′ polyA tailing—Used Terminal Deoxynucleotidyl Transferase (ThermoScientific) and dATP (Invitrogen)
5′ Rapid Amplification of cDNA Ends (RACE) PCR—Performed using Platinum SuperFi polymerase (Invitrogen), and a set of oligos specific for either the light chain or heavy chain amplification:

HEAVY CHAIN oligos (for all isotypes):
RACE-5E_mG1_rev
(SEQ ID NO: 441)
5′-gttagtttgggcagcagatc-3′
RACE-5E_mG2a.2c_rev
(SEQ ID NO: 442)
5′-gaggagccagttgtayctc-3′
RACE-5E mG2b_rev
(SEQ ID NO: 443)
5′-gaaccagttgtatctccacac-3′
RACE_5E_G3_rev
(SEQ ID NO: 444)
5′-gatccagatgtgtcactgc-3′
LIGHT CHAIN oligos (for all classes):
RACE-5E_mKappaCL_rev
(SEQ ID NO: 445)
5′-gtgggaagatggatacagttg-3′
RACE-5E mLambda1/4_rev
(SEQ ID NO: 446)
5′-gagctcttcagaggaaggtg-3′
RACE-5E_mLambda2/3_rev
(SEQ ID NO: 447)
5′-ctcagrggaaggtggaaac-3′
RACE-5E_mLambda2/3_rev3
(SEQ ID NO: 448)
5′-gagctcctcagrggaaggtg-3'

A common forward oligo was used:

XSCTnTag
(SEQ ID NO: 449)
5′-gactcgagtcgacatcgattttttttttttttttt-3′

Cloning into commercial plasmid (TOPO PCR cloning)—Cloning in plasmid and transformation performed using ZeroBlunt TOPO PCR kit (Invitrogen).
Colony picking, bacterial growth and plasmid extraction—Plasmid extraction performed with kit Qiaprep Miniprep (Qiagen).
Sanger sequencing—Templates for sequencing were prepared following internal procedure and using 100 ng of plasmid and the following oligos:

M13 Forward
(SEQ ID NO: 450)
5′-GTAAAACGACGGCCAG-3′
M13 Reverse
(SEQ ID NO: 451
5′-CAGGAAACAGCTATGAC-3′

QIAquick Gel Extraction Kit and MinElute PCR Purification Kit (Qiagen) were used for the DNA purification steps.
Next Generation Sequencing—DNA material generated by RACE PCR was used as template for Next Generation Sequencing using NextSeq and TruSeq library preparation methods, and subsequently run on MiSeq sequencing device.

Gyrolab Assay

The gyrolab workstation (Gyros) is a full automatized flexible platform allowing the miniaturization of immunoassays at a nanoliter scale in a microfluidics system. The gyrolab instrument also incorporates automated liquid handling and a fluorescence detection system. The gyrolab platform uses compact discs (CD) designed with microfluidic channeling structures that enable parallel nanoliter-scale immunoassays. The Bioaffy CD1000 (equal to the volume capacity, in nl, of the chamber) contains twelve segments each comprised of eight microstructures. Each microstructure contains a sample delivery channel, a column packed with streptavidin-coated beads as well as a common reagent channel. Gyrolab technology utilizes capillary action as well as centrifugal force for delivery of reagents and samples. Within each microstructure are multiple hydrophobic barriers, allowing for containment of reagents and buffers and facilitating parallel processing of samples on the CD. These hydrophobic barriers are overcome by an initial short, high velocity spin, followed by a decrease and subsequent incremental increase in speed, which allows reagents and samples to pass through the column.
All reagents, standard and samples were diluted to a defined working concentration and transferred to a microplate following the Gyros transfer list. Once the microplate is loaded into the gyrolab workstation, the process of the assay is fully automated.

### Instrument Method Used was:

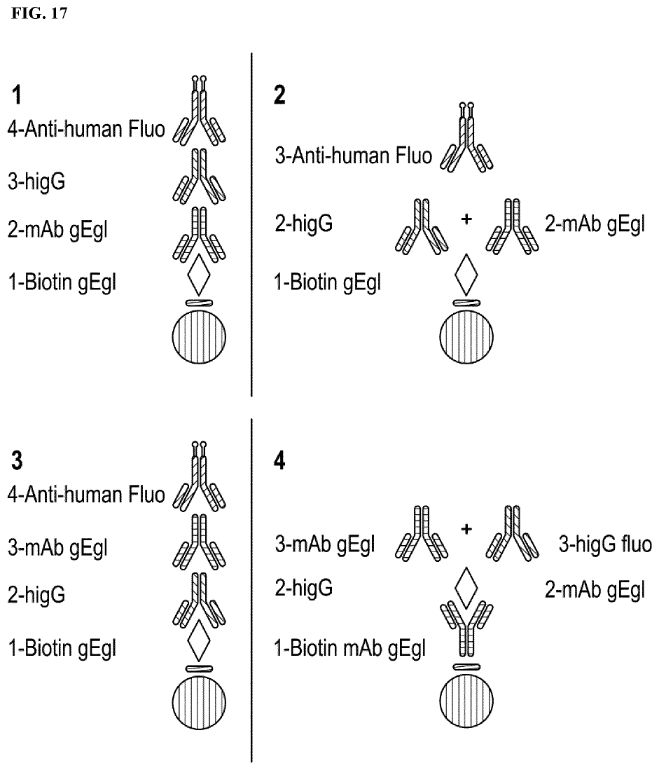

Design 1: 1000-4W-003-SSA/2CAD (2 captures-analyte-detection) is a 4 steps immunoassay
Design 2 1000-3W-007-X/C-A-D (capture-analyte-detection) is a 3 steps immunoassay:
Design 3: 1000-4W-004-A/C-A-2D (capture-analyte-2 detections) is a 4 steps immunoassay
   Design 4: 1000-3W-005/C-A-D (capture-analyte-detections) is a 3 steps immunoassay: The following designs were used (FIG. 17):
Design 1: 4-steps immunoassay by Gyros. The biotinylated HSV-2 gEgI construct is captured in the μcolumn at 100 μg/ml (20 ng/column) to the streptavidin bead. Then, the antibody was added at 100 μg/ml (20 ng/column). After, the human IgG is loaded at 50 μg/ml (50 ng/column) and serial diluted to 0.21 μg/ml (0.21 ng/column). At the end, the fluorescent secondary antibody is used as revelation to be read by the laser. Between each step, a wash of the column is performed by the automate.
Design 2: 3-steps immunoassay by Gyros. The biotinylated HSV-2 gEgI construct is captured in the μcolumn at 100 μg/ml (20 ng/column) to the streptavidin bead. Then, the antibody was added at 50 μg/ml (25 ng/column kept constant) simultaneously with the human IgG loaded at 25 μg/ml (25 ng/column) and serial diluted to 1 μg/ml (1ng/column). At the end, the fluorescent secondary antibody is used as revelation to be read by the laser. Between each step, a wash of the column is performed by the automate.
Design 3: 4-steps immunoassay by Gyros. The biotinylated HSV-2 gEgI construct is captured in the μcolumn at 100 μg/ml (20 ng/column) to the streptavidin bead. Then, the human IgG is loaded at 50 μg/ml (50 ng/column) and serial diluted to 0.21 μg/ml (0.21 ng/column). After, the antibody was added at 100 μg/ml (20 ng/column). At the end, the fluorescent secondary antibody is used as revelation to be read by the laser. Between each step, a wash of the column is performed by the automate.
Design 4: 3-steps immunoassay by Gyros. The biotinylated antibody specific of the HSV-2 gEgI construct is captured in the μcolumn at 100 μg/ml (20 ng/column) to the streptavidin bead. Then, the HSV-2 gEgI is loaded at 10 μg/ml (10 ng/column) or 1 μg/ml (1 ng/column) and serial diluted to a defined concentration. After, the antibody was added at 75 nM (15 pm/column) alone or in competition with the human IgG at equal concentration. At the end, the fluorescent antibody is used as revelation to be read by the laser. Only one antibody is labeled with fluorochrome in each evaluation. Between each step, a wash of the column is performed by the automate.

### Example 2—Screening of Hybridoma Supernatant (ELISA and WB)

New hybridomas expressing antibodies were generated and then recloned and diluted to obtain stable monoclonal cell cultures secreting the desired monoclonal antibody (mAb) in the culture supernatant. The supernatants were tested in enzyme-linked immunosorbent assays (ELISA) for antigen specificity. 29 mAbs were obtained (table 1).

TABLE 1
Specificity of selected mAbs

| mAb clone | gEgI | gE | gI | FCBD | R320D | 338 | P319D | P317R | Specificity |
|---|---|---|---|---|---|---|---|---|---|
| 2 | +++ | − | ++ | − | +++ | ++++ | +++ | +++ | gI |
| 41 | +++ | − | + | − | +++ | ++++ | +++ | +++ | gI |
| 3 | ++++ | ++++ | − | − | ++++ | ++++ | ++++ | ++++ | gE |
| 21 | ++++ | ++++ | − | − | ++++ | ++++ | ++++ | ++++ | gE |
| 37 | ++++ | ++++ | − | − | ++++ | ++++ | ++++ | ++++ | gE |
| 48 | ++++ | ++++ | − | − | ++++ | ++++ | ++++ | ++++ | gE |
| 5 | ++++ | − | − | − | +++ | +++ | +++ | +++ | gEgI |
| 9 | +++ | − | − | − | +++ | +++ | +++ | +++ | gEgI |
| 10 | ++++ | − | − | − | ++++ | ++++ | ++++ | ++++ | gEgI |
| 15 | ++++ | − | − | − | ++++ | ++++ | ++++ | ++++ | gEgI |
| 17 | ++++ | − | − | − | ++++ | ++++ | ++++ | ++++ | gEgI |
| 19 | ++++ | − | − | − | ++++ | ++++ | ++++ | ++++ | gEgI |
| 20 | +++ | − | − | − | +++ | ++++ | ++++ | ++++ | gEgI |
| 24 | ++++ | − | − | − | ++++ | ++++ | ++++ | ++++ | gEgI |
| 34 | +++ | − | − | − | +++ | +++ | +++ | +++ | gEgI |
| 39 | + | − | − | − | + | ++ | ++ | + | gEgI |
| 40 | ++++ | − | − | − | ++++ | ++++ | ++++ | ++++ | gEgI |
| 42 | +++ | − | − | − | +++ | +++ | +++ | +++ | gEgI |
| 45 | ++++ | − | − | − | ++++ | ++++ | ++++ | ++++ | gEgI |
| 12/13 | ++++ | ++++ | − | +++ | ++++ | +++ | ++++ | ++++ | gE/FCBD |
| 14 | ++++ | ++++ | − | ++++ | ++++ | ++++ | ++++ | ++++ | gE/FCBD |
| 18 | ++++ | ++++ | − | ++ | − | ++ | ++++ | + | gE/FCBD |
| 43 | ++++ | ++++ | − | +++ | ++++ | ++++ | ++++ | ++++ | gE/FCBD |
| 47 | ++++ | ++++ | − | + | − | − | − | − | gE/FCBD |
| 35 | ++++ | + | ++++ | + | ++++ | ++++ | ++++ | ++++ | gI (gE/FCBD) |
| 36 | ++++ | + | ++++ | + | ++++ | ++++ | ++++ | ++++ | gI (gE/FCBD) |
| 44 | ++++ | + | + | − | +++ | +++ | +++ | +++ | (gE and gI) |

A majority of the clones isolated are directed against epitopes that are putatively conformational as they were positive in non reduced conditions in WB and negative in reduced conditions. Five mAbs (clones 5, 19, 34, 39 and 40) from the heterodimer specific sub family were negative in both reduced and non reduced conditions suggesting a discontinous conformational epitope (strictly conformational). Four mAbs (mAb clones 3, 21, 48 and 47) were positive in reduced and non reduced conditions, suggesting that they are directed against a linear continuous epitopes (table 2).

TABLE 2
Western Blot profile of selected mAbs

| mAb | | | | | WB Non | WB | |
|---|---|---|---|---|---|---|---|
| clone | gEgI | gE | gI | FCBD | reduced | reduced | Epitope |
| 2 | +++ | − | ++ | − | + | − | Conformational |
| 41 | +++ | − | + | − | + | − | Conformational |
| 3 | ++++ | ++++ | − | − | + | + | Linear (TBC) |
| 21 | ++++ | ++++ | − | − | + | + | Linear (TBC) |
| 37 | ++++ | ++++ | − | − | | | ND |
| 48 | ++++ | ++++ | − | − | + | + | Linear (TBC) |
| 5 | ++++ | − | − | − | − | − | Strictly conformational |
| 9 | +++ | − | − | − | + | − | Conformational |
| 10 | ++++ | − | − | − | + | − | Conformational |
| 15 | ++++ | − | − | − | + | − | Conformational |
| 17 | ++++ | − | − | − | + | − | Conformational |
| 19 | ++++ | − | − | − | − | − | Strictly conformational |
| 20 | +++ | − | − | − | + | − | Conformational |
| 24 | ++++ | − | − | − | + | − | Conformational |
| 34 | +++ | − | − | − | − | − | Strictly conformational |
| 39 | + | − | − | − | − | − | Strictly conformational |
| 40 | ++++ | − | − | − | − | − | Strictly conformational |
| 42 | +++ | − | − | − | + | − | Conformational |
| 45 | ++++ | − | − | − | + | − | Conformational |
| 12/13 | ++++ | ++++ | − | +++ | + | − | Conformational |
| 14 | ++++ | ++++ | − | ++++ | + | − | Conformational |
| 18 | ++++ | ++++ | − | ++ | + | − | Conformational |
| 43 | ++++ | ++++ | − | +++ | + | − | Conformational |
| 47 | ++++ | ++++ | − | + | + | + | Linear (TBC) |
| 35 | ++++ | + | ++++ | + | + | − | Conformational |
| 36 | ++++ | + | ++++ | + | + | − | Conformational |
| 44 | ++++ | + | + | − | + | − | Conformational |

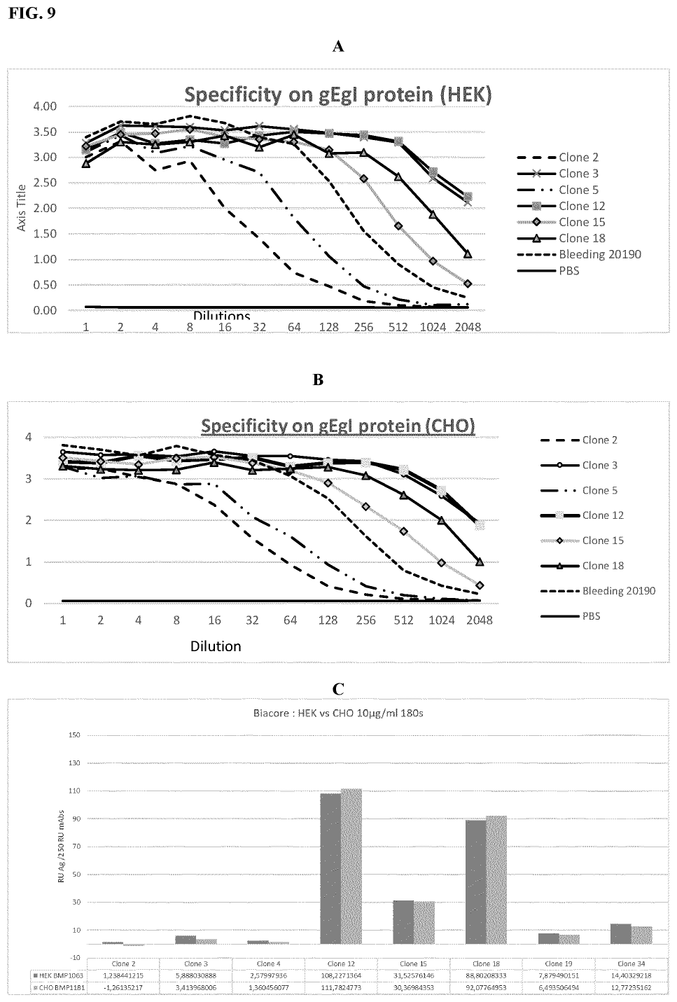

In a direct ELISA and in Biacore SPR, no significative difference was observed between gEgI produced HEK293 cells and gEgI produced in CHO cells for each mAbs tested (FIG. 9).

### Example 3—Binding Chart and Binning Assay-Surface Like Map of Epitopes

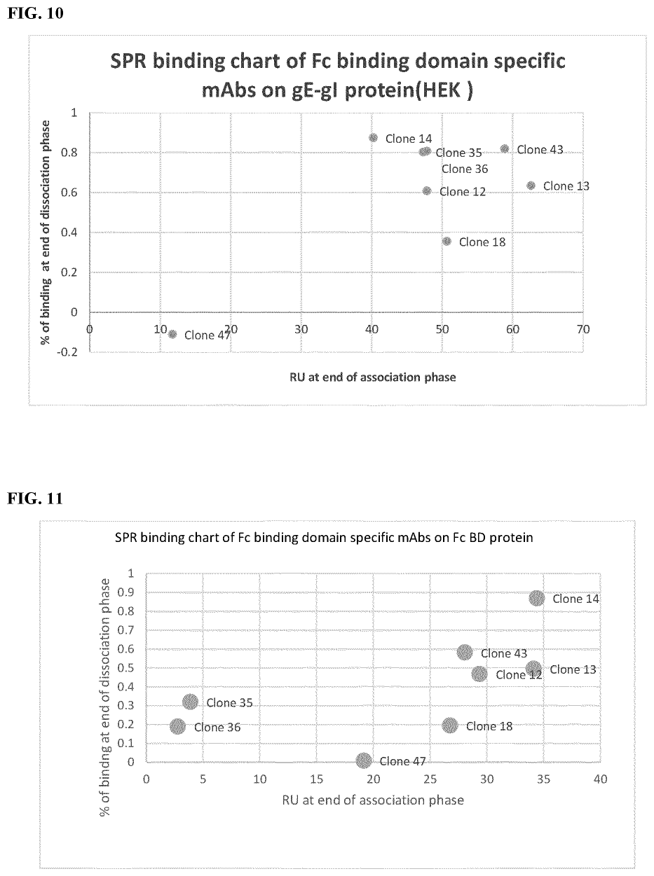

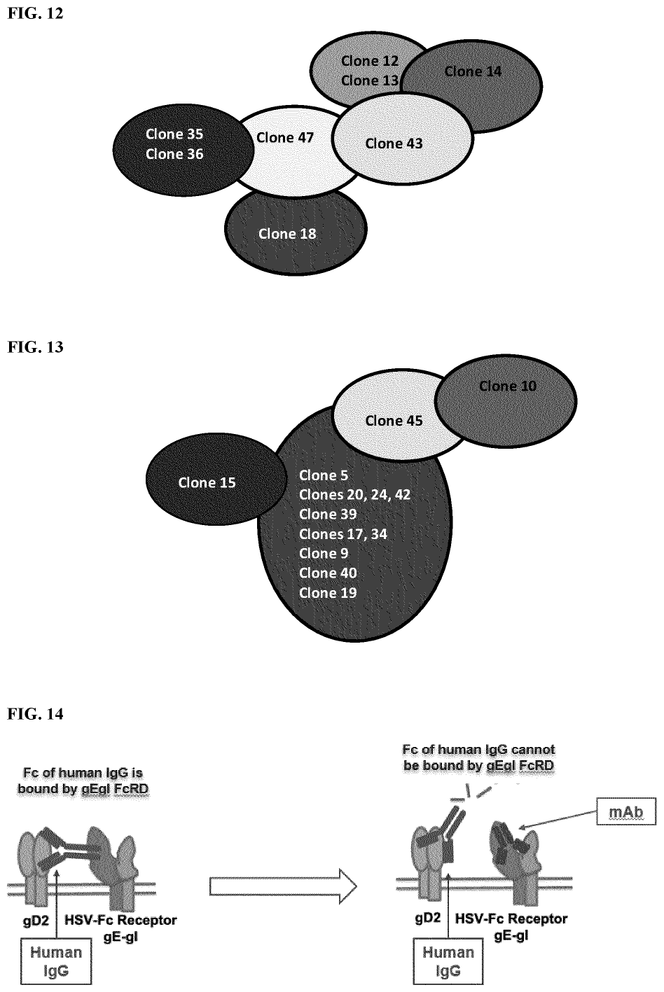

Epitope binning and binding properties experiments by surface plasmon resonance (SPR) were conducted on FCBD specific mAbs to build a two-dimension surface like map of epitopes and evaluate potential affinity (Table 3; FIG. 10, FIG. 11, FIG. 12).
mAb clone 14 was the best binder on Fc binding domain (FCBD);
mAb clones 35 and 36 were low binders on FCBD while good binders on gEgI suggesting potential epitope masking on sub domain protein;
mAb clones 43,12,13,14 and 18 were good binders and presented the same behavior on FCBD subdomain and full protein;
mAb clone 47 presented a high dissociation rate on both full protein and subdomain suggesting low affinity constant

TABLE 3
Binning chart matrix results expressed
in RU for FCBD specific mAbs

| | | Revelation mAb clone | | | | | |
|---|---|---|---|---|---|---|---|
| | | 12/13 | 14 | 18 | 35/36 | 43 | 47 |
| Capture | 12/13 | 0 | 28 | 134 | 88.9 | −2.8 | 29.1 |
| mAb | 14 | −0.9 | 0 | 110.3 | 66.1 | −3.6 | 42.2 |
| clone | 18 | 62 | 24 | 0 | 44.8 | 41.5 | −13.7 |
| | 35 | 88.9 | 47.8 | 114.5 | 0 | 81.3 | 30.9 |
| | 43 | 16.3 | 35.5 | 128.5 | 80.3 | 0 | 47.3 |
| | 47 | 8.1 | 21.6 | 41.2 | −0.3 | −2.7 | 0 |

The FCBD specific mAb clones can be associated in 3 groups:
Group 1: mAb clones 12, 13, 14 and 43 are directed against epitopes close to each other with mAb clones 12 and 13 sharing the same epitope (confirmed by hybridoma sequencing);
Group 2: mAb clones 35, 36 and 18 are located in a different epitope area on FC binding subdomain than group 1 mAb clones. mAb clones 35 and 36 share the same epitope located in a different area than mAb clone 18 epitope;
Group 3: mAb clone 47 at interface between group 1 and 2.

Epitope binning and binding properties experiments by surface plasmon resonance (SPR) were conducted on heterodimer specific mAbs. Heterodimer specific mAbs can be associated in 4 groups corresponding to 4 different regions on the gEgI antigen (Table 4, FIG. 13). Most of them, 10 out of 13 target the same region. mAb clone 10 exhibited an unexpected behaviour as that antibody was able to detect the antigen while already captured by the same antibody.

TABLE 4
Binning chart matrix results expressed in RU for gEgI heterodimer specific mAbs

| | | Revelation mAb clone | | | | | | | | | | | | |
|---|---|---|---|---|---|---|---|---|---|---|---|---|---|---|
| | | 5 | 20 | 39 | 17 | 42 | 9 | 24 | 34 | 45 | 40 | 19 | 15 | 10 |
| Capture | 5 | −3 | 25.3 | 0.6 | 0.5 | 10.1 | 9.4 | 34.4 | −2.1 | 12.9 | 9 | 17.2 | 13.5 | 10.6 |
| mAb | 20 | −1 | 9.7 | 3.1 | 2.2 | 12.5 | 4.8 | 17.7 | 0.4 | 11.4 | 10 | 9 | 13 | 10.8 |
| clone | 39 | −1.6 | 23.8 | 1.3 | 0.5 | 11.3 | 9 | 30.5 | −1.5 | 13.1 | 9 | 16.2 | 11.7 | 10.3 |
| | 17 | −2.9 | 24 | −0.6 | −8.7 | 8.7 | 7.8 | 32.1 | −3.3 | 12.4 | 8.8 | 18.1 | 3.4 | 8.2 |
| | 42 | −1.9 | 24.5 | 0.1 | −0.8 | 8.7 | 7 | 30.7 | −2.4 | 11.9 | 7.4 | 16.3 | 9.8 | 9.4 |
| | 9 | −4.4 | 13 | −1.5 | −0.5 | 8.6 | −0.4 | 17 | −3.9 | 11 | 8.8 | 6.4 | 12.7 | 8.3 |
| | 24 | −3.4 | 7.3 | −0.9 | −2 | 7.8 | −0.1 | 6.4 | −3.6 | 10.1 | 7.6 | 3.2 | 9.9 | 8.2 |
| | 34 | −9.1 | 13.4 | −6.4 | −9.4 | 4.1 | 0.9 | 20.7 | −11.1 | 6.1 | 5.2 | 9.3 | 5.9 | 2 |
| | 45 | −7.7 | 16.4 | −5.4 | −2.6 | 5.5 | 1.8 | 26.8 | −6.3 | −6.5 | 7.3 | 9.2 | 8.7 | −2 |
| | 40 | −3 | 21.3 | −1.2 | −2.4 | 9.2 | 6 | 28.3 | −4.1 | 10.9 | 2.6 | 13.9 | 6.9 | 8.3 |
| | 19 | −11.1 | 6.5 | −7.2 | −6.3 | 1.9 | −5.1 | 13.3 | −8.4 | 4.2 | 6.4 | −3.4 | 7.6 | 2.3 |
| | 15 | 2.3 | 18.5 | −4.8 | −6.7 | 4.9 | 5.5 | 42.6 | −2.7 | 9.9 | −0.4 | 23 | −4.9 | 3.3 |
| | 10 | 2.2 | 32.8 | 2.3 | 4.6 | 11.6 | 11 | 39.1 | 1.3 | 5.2 | 9.8 | 24.4 | 13.4 | 4 |

### Example 4—Determination of Affinity Constant (KD) of Anti-gEgI Monoclonal Antibodies (Clones 12 and 18) by SPR Technology

### Set-Up of Assay

### Samples Analyzed (Analyte):

Wild-type gEgI heterodimer
Mutated gEgI batch 1 (P317R mutation in gE FCBD)
Mutated gEgI batch 2 (P317R mutation in gE FCBD)
   Clone 12 or 18 mAb was injected with a flow rate of 10 μL/min for 25 sec to obtain ±100 RU of mAb bound. The WT gEgI analyte was used at 3 concentrations (2.5 nM, 5 nM, and 15 nM) and injected with a flow rate of 30 μL/min for 240 sec. The results showed there was a progressive fixation of the analyte when the clone 12 or 18 mAb was used as ligand, indicating that the selected conditions (lower ligand concentrations) were suitable for the determination of the KD (data not shown). Five concentrations were selected to assess the KD for each analyte (Table 5):

TABLE 5
Ligand concentrations used for KD measurement
of clone 12 and 18 mAbs.

| | | Conc. 1 | Conc. 2 | Conc. 3 | Conc. 4 | Conc. 5 |
|---|---|---|---|---|---|---|
| | | (nM) | (nM) | (nM) | (nM) | (nM) |
| Clone 12 | WT gEgI | 2.5 | 5 | 15 | 30 | 50 |
| mAb | gEgI mutant | 2.5 | 5 | 15 | 30 | 50 |
| | batch 1 | | | | | |
| | gEgI mutant | 2.5 | 5 | 15 | 30 | 50 |
| | batch 2 | | | | | |
| Clone 18 | WT gEgI | 0.75 | 1.5 | 2.5 | 5 | 15 |
| mAb | gEgI mutant | 0.85 | 1.25 | 2.5 | 5 | 17 |
| | batch 1 | | | | | |
| | gEgI mutant | 0.75 | 1.5 | 2.5 | 5 | 15 |
| | batch 2 | | | | | |

Clone 12 mAb (2 μg/mL) was injected with a flow rate of 10 μL/min for 25 sec to obtain ±100 RU of mAb bound. WT or mutated gEgI analyte was injected with a flow rate of 30 μL/min for 180 sec at five increasing concentrations (Table 5). Clone 18 mAb (2 μg/mL) was injected with a flow rate of 10 μL/min for 25 sec to obtain ±100 RU of mAb bound. WT or mutant gEgI analyte was injected with a flow rate of 30 μL/min for 120 sec at five increasing concentrations (Table 5). Results are presented in table 6.

TABLE 6
KD, ka, kd and Rmax measured for clones 12 and 18 mAb with gEgI

| | | KD (M) | ka (1/Ms) | kd (1/s) | Rmax (RU) |
|---|---|---|---|---|---|
| Clone 12 | WT gEgI | 6.89 × 10⁻⁹ | 2.85 × 10⁵ | 1.97 × 10⁻³ | 55 |
| mAb | gEgI mutant batch 1 | 7.68 × 10⁻⁹ | 4.22 × 10⁵ | 3.25 × 10⁻³ | 60 |
| | gEgI mutant batch 2 | 11.53 × 10⁻⁹ | 2.80 × 10⁵ | 3.24 × 10⁻³ | 68 |
| Clone 18 | WT gEgI | 6.79 × 10⁻⁹ | 57.10 × 10⁵ | 38.72 × 10⁻³ | 33 |
| mAb | gEgI mutant batch 1 | 26.97 × 10⁻⁹ | 18.98 × 10⁵ | 51.20 × 10⁻³ | 29 |
| | gEgI mutant batch 2 | 36.18 × 10⁻⁹ | 15.74 × 10⁵ | 60.08 × 10⁻³ | 32 |

The affinity constant (KD) measured for the clone 12 mAb was similar for WT and mutated gEgI heterodimer. The association constant (ka) was slightly higher for gEgI mutant batch 1. The dissociation constant (kd) was slightly higher with a mutated gEgI compared to the wildtype protein, but not significantly so.
The affinity constant (KD) measured for clone 18 mAb with WT gEgI heteromider was similar to the KD measured for clone 12 mAb. However, the KD obtained for clone 18 mAb on mutated gEgI heteromider was significantly lower than the KD obtained for wildtype gEgI. The formation of complexes (clone 18 mAb-gEgI) was very fast with wildtype gEgI and this rate of complexes association decreased significantly for gEgI mutated at the position 317. Finally, the dissociation of complexes was very fast between clone 18 mAb and wildtype gEgI protein. This rate of dissociation was slightly higher for gEgI mutated at position 317, but not significantly so.

### Example 5—Hybridoma Sequencing

The 29 hybridomas clones that were sequenced either by Sanger (clones 12, 13, 18, 35 and 47) or NGS (all remaining clones). The sequences isolated by Sanger were used to benchmark the bioinformatic algorithm designed and used to identify the sequences by NGS. CDR1, CDR2 and CDR3 were defined using Kabat definitions and an internal algorithm. The mAb isotypes, light chain types and V(D)L regions for each clone are presented in table 7. The VH and VL amino acid and nucleotide sequences as well as the CDR1, CDR2 and CDR3, are presented in tables 8-11. A sequence phylogenetic tree was also generated (FIG. 15, FIG. 16). This type of representation helps to reconstruct evolutionary (clonal) development of antibodies and to visually identify identical antibody sequences.
The following clones were found to share identical sequences for both VH and VL domains, meaning they correspond to the same clone:
clones 12 and 13,
clones 35 and 36, and
clones 3 and 21.
   For hybridoma clone 42, two functional VL chains (L1 and L2) were identified.

TABLE 7
Heavy (H) and light (L) chain V(D)J regions,
Heavy chain isotype, Light chain class

| mAb | | | | |
|---|---|---|---|---|
| clone | H VDJ regions | Isotype | L VJ regions | Class (V) |
| 2 | IGHV1-54*01 | IgG1 | IGKV3-4*01 | Kappa |
| | IGHD4-1*01_5-3 | | IGKJ2*01 | |
| | IGHJ4*0 | | | |
| 3, 21 | IGHV1-22*01 | IgG1 | IGKV3-7*01 | Kappa |
| | IGHD1-1*01_5-3 | | IGKJ2*01 | |
| | IGHJ3*01 | | | |
| 5 | IGHV7-1*02 | IgG1 | IGKV5-39*01 | Kappa |
| | IGHD1-1*01_5-3 | | IGKJ2*01 | |
| | IGHJ3*01 | | | |
| 9 | IGHV1S135*01 | IgG1 | IGKV8-30*01 | Kappa |
| | IGHD1-1*01_5-3 | | IGKJ2*01 | |
| | IGHJ4*01 | | | |
| 10 | IGHV7-1*02 | IgG1 | IGKV3-4*01 | Kappa |
| | IGHD3-2_01_3-5 | | IGKJ2*01 | |
| | IGHJ4*01 | | | |
| 12, 13 | IGHV2-9-1*01 | IgG1 | IGKV3-1*01 | Kappa |
| | IGHD2-4*01_5-3 | | IGKJ1*01 | |
| | IGHJ4*01 | | | |
| 14 | IGHV1-9*01 | IgG1 | IGKV3-12*01 | Kappa |
| | IGHD6-1_01_3-5 | | IGKJ5*01 | |
| | IGHJ4*01 | | | |
| 15 | IGHV2-3*01 | IgG3 | IGKV12-41*01 | Kappa |
| | IGHD2-4*01_5-3 | | IGKJ1*01 | |
| | IGHJ4*01 | | | |
| 16 | IGHV4-1*02 | IgG1 | IGKV1-135*01 | Kappa |
| | IGHD2-5*01_5-3 | | IGKJ1*01 | |
| | IGHJ4*01 | | | |
| 17 | IGHV2-9-1*01 | IgG3 | IGKV2-137*01 | Kappa |
| | IGHD1-2*01_5-3 | | IGKJ5*01 | |
| | IGHJ4*01 | | | |
| 18 | IGHV1-63*02 | IgG1 | IGKV6-32*01 | Kappa |
| | IGHD1-1*01_5-3 | | IGKJ4*01 | |
| | IGHJ2*02 | | | |
| 19 | IGHV1S137*01 | IgG1 | IGKV4-59*01 | Kappa |
| | IGHD1-1*01_5-3 | | IGKJ1*01 | |
| | IGHJ4*01 | | | |
| 20 | IGHV1-54*01 | IgG1 | IGKV3-10*01 | Kappa |
| | IGHD2-11*02_5-3 | | IGKJ1*01 | |
| | IGHJ4*01 | | | |
| 24 | IGHV1-26*01 | IgG1 | IGKV8-27*01 | Kappa |
| | IGHD5-6_01_3-5 | | IGKJ2*01 | |
| | IGHJ4*01 | | | |
| 34 | IGHV5-12*02 | IgG1 | IGKV4-57-1*01 | Kappa |
| | IGHD2-11*02_5-3 | | IGKJ5*01 | |
| | IGHJ3*01 | | | |
| 35, 36 | IGHV2-9-1*01 | IgG1 | IGKV2-137*01 | Kappa |
| | IGHD2-3*01_5-3 | | IGKJ4*01 | |
| | IGHJ4*01 | | | |
| 37 | IGHV1S29*02 | IgG2a | IGKV3-12*01 | Kappa |
| | IGHD4-1*01_5-3 | | IGKJ1*01 | |
| | IGHJ4*01 | | | |
| 39 | IGHV5-12-2*01 | IgG1 | IGKV1-88*01 | Kappa |
| | IGHD2-13*01_5-3 | | IGKJ1*01 | |
| | IGHJ2*01 | | | |
| 40 | IGHV9-3-1*01 | IgG1 | IGKV12-41*01 | Kappa |
| | IGHD2-9*01_5-3 | | IGKJ2*01 | |
| | IGHJ1*01 | | | |
| 41 | IGHV1-54*01 | IgG1 | IGKV3-4*01 | Kappa |
| | IGHD2-9*02_5-3 | | IGKJ1*01 | |
| | IGHJ4*01 | | | |
| 42 | IGHV2-9-1*01 | IgG1 | Light chain 1 (L1) | Kappa |
| | IGHD4-1*01_5-3 | | IGKV1-135*01 | |
| | | | IGKJ1*01 | |
| | IGHJ2*01 | | Light chain 2 (L2) | Kappa |
| | | | IGKV5-45*01 | |
| | | | IGKJ4*01 | |
| 43 | IGHV5-6-4*01 | IgG1 | IGKV8-21*01 | Kappa |
| | IGHD1-1*01_5-3 | | IGKJ5*01 | |
| | IGHJ2*01 | | | |
| 44 | IGHV1S135*01 | IgG2a | IGKV4-70*01 | Kappa |
| | IGHD5-7_01_3-5 | | IGKJ2*01 | |
| | IGHJ4*01 | | | |
| 45 | IGHV5-6-4*01 | IgG1 | IGKV3-12*01 | Kappa |
| | IGHD1-1*01_5-3 | | IGKJ2*01 | |
| | IGHJ4*01 | | | |
| 47 | IGHV7-3*02 | IgG1 | IGKV3-10*01 | Kappa |
| | IGHD3-1*01_5-3 | | IGKJ1*01 | |
| | IGHJ2*01 | | | |
| 48 | IGHV8-12*01 | IgG1 | IGKV5-48*01 | Kappa |
| | IGHD2-14*01_5-3 | | IGKJ5*01 | |
| | IGHJ4*01 | | | |

TABLE 8
VH amino acid sequences (CRDs: italic underlined) and HCDR1, HCDR2, HCDR3

| clone | VH | HCDR1 | HCDR2 | HCDR3 |
|---|---|---|---|---|
| 2 | QVQLQQSGAELVRPGTSVKVSCKASGYA | NYLIE | VINPGSGGTN | SRGYPYAM |
| | FTNYLIEWVKQRPGQGLEWIGVINPGSG | (SEQ ID | YNEKFKG | DY |
| | GTNYNEKFKGKATLTADKSSSTAYMQLS | NO: 10) | (SEQ ID | (SEQ ID |
| | SLTSDDSAVYFCARSRGYPYAMDYWGQG | | NO: 11) | NO: 12) |
| | TSVTVSS | | | |
| | (SEQ ID NO: 9) | | | |
| 3, 21 | EVQLQQSGPELVKPGASVKISCKTSGYT | EYTMH | GINPNNGGTS | SLVEGDFA |
| | FTEYTMHWVKQSHGKSLEWIGGINPNNG | (SEQ ID | YNQKFKG | Y |
| | GTSYNQKFKGKATLTVDKSSSTAYMELR | NO: 14) | (SEQ ID | (SEQ ID |
| | SLTSEDSAVYYCARSLVEGDFAYWGQGT | | NO: 15) | NO: 16) |
| | LVTVSA | | | |
| | (SEQ ID NO: 13) | | | |
| 5 | EVKLVESGGGLVQPGGSLRLSCATSGFT | DFYME | ASRNKANDYT | DFLPYYGS |
| | FSDFYMEWVRQPPGKRLEWIAASRNKAN | (SEQ ID | TEYSASVKG | PWFAY |
| | DYTTEYSASVKGRFIVSRDTSQSILYLQ | NO: 18) | (SEQ ID | (SEQ ID |
| | MNALRAEDTAIYYCARDFLPYYGSPWFA | | NO: 19) | NO: 20) |
| | YWGQGTLVTVSA | | | |
| | (SEQ ID NO: 17) | | | |
| 9 | EVQLQQSGPELEKPGASVKISCKASGYS | GYNMN | NIDPYYGGTS | SRFITTVV |
| | FTGYNMNWVKQSNGKSLEWIGNIDPYYG | (SEQ ID | YNQKFKG | ATDYYAMD |
| | GTSYNQKFKGKATLTLDKSSSTAYMQLK | NO: 22) | (SEQ ID | Y |
| | SLTSEDSAVYYCARSRFITTVVATDYYA | | NO: 23) | (SEQ ID |
| | MDYWGQGTSVTVSS | | | NO: 24) |
| | (SEQ ID NO: 21) | | | |
| 10 | EVKLVESGGGLVQPGGSLRLSCATSGFT | DFYME | ASRNKANDYT | ENYGNPAM |
| | FSDFYMEWVRQPPGKRLEWIAASRNKAN | (SEQ ID | TEYSASVKG | DY |
| | DYTTEYSASVKGRFIVSRDTSQSILYLQ | NO: 26) | (SEQ ID | (SEQ ID |
| | MNALRAEDTAIYYCARENYGNPAMDYWG | | NO: 27) | NO: 28) |
| | QGTSVTVSS | | | |
| | (SEQ ID NO: 25) | | | |
| 12, 13 | QVQLKESGPGLVAPSQSLSITCTVSGFS | SYGVH | VIWAGGSTNY | GGITSLHN |
| | LTSYGVHWVRQPPGKGLEWLGVIWAGGS | (SEQ ID | NSALMS | YVMDY |
| | TNYNSALMSRLSISKDNSKSQVFLKMNS | NO: 30) | (SEQ ID | (SEQ ID |
| | LQTDDTAMYFCARGGITSLHNYVMDYWG | | NO: 31) | NO: 32) |
| | QGTSVTVSS | | | |
| | (SEQ ID NO: 29) | | | |
| 14 | QVQLQQSGAELMKPGASVKISCKATGYT | SYWIE | EILPGSGSTN | GSAGNYYG |
| | FSSYWIEWVKQRPGHGLEWIGEILPGSG | (SEQ ID | YNEKFKG | SSYEAMDY |
| | STNYNEKFKGKATFTADTSSNTAYMQLS | NO: 34) | (SEQ ID | (SEQ ID |
| | SLTSEDSAVYYCASGSAGNYYGSSYEAM | | NO: 35) | NO: 36) |
| | DYWGQGTSVTVSS | | | |
| | (SEQ ID NO: 33) | | | |
| 15 | QVQLKESGPGLVAPSQSLSITCTVSGFS | SYGVS | VIWGDGSTNY | PDLYYDSP |
| | LTSYGVSWVRQPPGKGLEWLGVIWGDGS | (SEQ ID | HSALIS | YAMDY |
| | TNYHSALISRLSISKDNSKSQVFLKLNS | NO: 38) | (SEQ ID | (SEQ ID |
| | LQTDDTATYYCAKPDLYYDSPYAMDYWG | | NO: 39) | NO: 40) |
| | QGTSVTVSS | | | |
| | (SEQ ID NO: 37) | | | |
| 16 | EVKLLESGGGLVQPGGSLKLSCAASGFD | RYWMS | EINPDSSTIN | PLMDY |
| | FSRYWMSWVRQAPGKGLEWIGEINPDSS | (SEQ ID | YTPSLKD | (SEQ ID |
| | TINYTPSLKDKFIISRDNAKNTLYLQMS | NO: 42) | (SEQ ID | NO: 44) |
| | KVRSEDTALYYCARPLMDYWGQGTSVTV | | NO: 43) | |
| | SS | | | |
| | (SEQ ID NO: 41) | | | |
| 17 | QVQLKESGPGLVAPSQSLSITCTVSGFS | SYGVH | VIWAGGSSNY | DGYGYVYY |
| | LTSYGVHWVRQPPGKGLEWLGVIWAGGS | (SEQ ID | NSALMS | AMDY |
| | SNYNSALMSRLSISKDNSKSQVFLKMNS | NO: 46) | (SEQ ID | (SEQ ID |
| | LQTDDTAMYYCARDGYGYVYYAMDYWGQ | | NO: 47) | NO: 48) |
| | GTSVTVSS | | | |
| | (SEQ ID NO: 45) | | | |
| 18 | QVQLQQSGAELVRPGTSVKMSCKAAGYT | NYWIG | DIYPGGGYTK | DGYSPY |
| | FTNYWIGWVKQRPGHGLEWIGDIYPGGG | (SEQ ID | YNEKFKG | (SEQ ID |
| | YTKYNEKFKGKATLTADTSSSTAYMQLS | NO: 50) | (SEQ ID | NO: 52) |
| | SLTSEDSAIYYCARDGYSPYWGQGTSLT | | NO: 51) | |
| | VSS | | | |
| | (SEQ ID NO: 49) | | | |
| 19 | QVQLQQSGAELVRPGVSVKISCKGSGYT | DYAMH | VISTYYGDAR | RGITVVAD |
| | FTDYAMHWVKQSHAKSLEWIGVISTYYG | (SEQ ID | YNQKFKG | YAMDY |
| | DARYNQKFKGKATMTVDKSSSTAYMELA | NO: 54) | (SEQ ID | (SEQ ID |
| | RLTSEDSAIYYCARRGITVVADYAMDYW | | NO: 55) | NO: 56) |
| | GQGTSVTVSS | | | |
| | (SEQ ID NO: 53) | | | |
| 20 | QVQLQQSGAELVRPGTSVKVSCKASGYA | NYLIE | VINPGSGGTN | SHYGNYYA |
| | FTNYLIEWVKQRPGQGLEWIGVINPGSG | (SEQ ID | YNEKFKG | MDY |
| | GTNYNEKFKGKATLTADKSSSTAYMQLS | NO: 58) | (SEQ ID | (SEQ ID |
| | SLTSDDSAVYFCARSHYGNYYAMDYWGQ | | NO: 59) | NO: 60) |
| | GTSVTVSS | | | |
| | (SEQ ID NO: 57) | | | |
| 24 | EVQLQQSGPELVKPGASVKMSCKASGYT | DYYMK | DINPNNGDTF | RGFYYYGS |
| | FTDYYMKWVKQSHGKSLEWIGDINPNNG | (SEQ ID | YNQKFKG | RGGIMDY |
| | DTFYNQKFKGKATLTVDKSSSTAYMQLN | NO: 62) | (SEQ ID | (SEQ ID |
| | SLTSEDSAVYYCARRGFYYYGSRGGIMD | | NO: 63) | NO: 64) |
| | YWGQGTSVTVSS | | | |
| | (SEQ ID NO: 61) | | | |
| 34 | EVKLVESGGGLVQPGGSLKLSCATSGFT | DYYMY | YISNGGDSTY | SYYGNPFP |
| | FSDYYMYWVRQTPEKRLEWVAYISNGGD | (SEQ ID | YPDTVKG | Y |
| | STYYPDTVKGRFTISRDNAKNTLYLQMS | NO: 66) | (SEQ ID | (SEQ ID |
| | RLKSEDTAMYYCARSYYGNPFPYWGQGT | | NO: 67) | NO: 68) |
| | LVTVSA | | | |
| | (SEQ ID NO: 65) | | | |
| 35, 36 | QVQLKESGPGLVAPSQSLSITCTVSGFS | SYGVH | VIWAGGSTNY | DGYKFFYY |
| | LTSYGVHWVRQPPGKGLEWLGVIWAGGS | (SEQ ID | NSALMS | AMDY |
| | TNYNSALMSRLSISKDNSKSQVFLKMNS | NO: 70) | (SEQ ID | (SEQ ID |
| | LQTDDTAMYYCATDGYKFFYYAMDYWGQ | | NO: 71) | NO: 72) |
| | GTSVTVSS | | | |
| | (SEQ ID NO: 69) | | | |
| 37 | EVQLQQSGPELVKPGASVKISCKASGYT | DYNMH | YIYPYNGGTG | PGYYYAMD |
| | FTDYNMHWVKQSHGKSLEWIGYIYPYNG | (SEQ ID | YNQKFKT | Y |
| | GTGYNQKFKTKATLTVDNSSSTAYMELR | NO: 74) | (SEQ ID | (SEQ ID |
| | SLTSEDSAVYYCAIPGYYYAMDYWGQGT | | NO: 75) | NO: 76) |
| | SVTVSS | | | |
| | (SEQ ID NO: 73) | | | |
| 39 | EVKLVESGGGLVQPGGSLKLSCAASGFT | SYTMS | YISNGGGSTY | GVSYYGDL |
| | FSSYTMSWVRQTPEKRLEWVAYISNGGG | (SEQ ID | YPDTVKG | YYFDY |
| | STYYPDTVKGRFTISRDNAKNTLYLQMS | NO: 78) | (SEQ ID | (SEQ ID |
| | SLKSEDTAMYYCARGVSYYGDLYYFDYW | | NO: 79) | NO: 80) |
| | GQGTTLTVSS | | | |
| | (SEQ ID NO: 77) | | | |
| 40 | QIQLVQSGPELKKPGETVKISCKASGYT | NYGMN | WINTYTGEPT | SLYGYDEG |
| | FTNYGMNWVKQAPGKGLKWMGWINTYTG | (SEQ ID | YADDFKG | YFDV |
| | EPTYADDFKGRFAFSLETSASTAYLQIN | NO: 82) | (SEQ ID | (SEQ ID |
| | NLKNEDTATYFCARSLYGYDEGYFDVWG | | NO: 83) | NO: 84) |
| | AGTTVTVSS | | | |
| | (SEQ ID NO: 81) | | | |
| 41 | QVQLQQSGAELVRPGTSVKVSCKASGYA | NYLIE | VINPGSGGTN | SGLRRDYY |
| | FTNYLIEWVKQRPGQGLEWIGVINPGSG | (SEQ ID | YNEKFKG | AMDY |
| | GTNYNEKFKGKATLTADKSSSTAYMQLS | NO: 86) | (SEQ ID | (SEQ ID |
| | SLTSDDSAVYFCARSGLRRDYYAMDYWG | | NO: 87) | NO: 88) |
| | QGTSVTVSS | | | |
| | (SEQ ID NO: 85) | | | |
| 42 | QVQLKESGPGLVAPSQSLSITCTVSGFS | SYGVH | VIWAGGSTNY | EGNWDDFD |
| | LTSYGVHWVRQPPGKGLEWLGVIWAGGS | (SEQ ID | NSALMS | Y |
| | TNYNSALMSRLSISKDNSKSQVFLKMNS | NO: 90) | (SEQ ID | (SEQ ID |
| | LQTDDTAMYYCAREGNWDDFDYWGQGTT | | NO: 91) | NO: 92) |
| | LTVSS | | | |
| | (SEQ ID NO: 89) | | | |
| 43 | DVKLVESGGGLVKPGGSLKLSCAASGFT | SYTMS | TISSGGSYTY | GGYGSSFE |
| | FSSYTMSWVRQTPEKRLEWVATISSGGS | (SEQ ID | YPDSVKG | NYFDY |
| | YTYYPDSVKGRFTISRDNAKNTLYLQMS | NO: 94) | (SEQ ID | (SEQ ID |
| | SLKAEDTAMYYCTRGGYGSSFENYFDYW | | NO: 95) | NO: 96) |
| | GQGTTLTVSS | | | |
| | (SEQ ID NO: 93) | | | |
| 44 | EVQLQQSGPELEKPGASVKISCKASGYS | GYNMN | GNIDPYYGGI | EDRYLVHY |
| | FTGYNMNWVKQSNGKSLEWIGNIDPYYG | (SEQ ID | RYNQKFKG | YAMDY |
| | GIRYNQKFKGKATLTVDKSSSTAYMQLK | NO: 98) | (SEQ ID | (SEQ ID |
| | SLTSEDSAVYYCAREDRYLVHYYAMDYW | | NO: 99) | NO: 100) |
| | GQGTSVTVSS | | | |
| | (SEQ ID NO: 97) | | | |
| 45 | DVKLVESGGGLVKPGGSLKLSCAASGFT | SYTMS | TISSGGSYTY | NYGSRPPY |
| | FSSYTMSWVRQTPEKRLEWVATISSGGS | (SEQ ID | YPDSVKG | YAMDY |
| | YTYYPDSVKGRFTISRDNAKNTLYLQMS | NO: 102) | (SEQ ID | (SEQ ID |
| | SLKSEDTAMYYCTRNYGSRPPYYAMDYW | | NO: 103) | NO: 104) |
| | GQGTSVTVSS | | | |
| | (SEQ ID NO: 101) | | | |
| 47 | EVKLVESGGGLVQPGGSLRLSCATSGFT | DYYMS | FIRNKANGYT | DRPGFDY |
| | FTDYYMSWVRQPPGKALEWLGFIRNKAN | (SEQ ID | TEYSASVKG | (SEQ ID |
| | GYTTEYSASVKGRFTISRDNSQSILYLQ | NO: 106) | (SEQ ID | NO: 108) |
| | MNTLRAEDSATYYCARDRPGFDYWGQGT | | NO: 107) | |
| | TLTVSS | | | |
| | (SEQ ID NO: 105) | | | |
| 48 | QVTLKESGPGILQPSQTLSLTCSFSGFS | TSGMGVS | HIYWDDDKRY | RGFYYRYD |
| | LSTSGMGVSWIRQPSGKGLEWLAHIYWD | (SEQ ID | NPSLKS | GGRRYYYA |
| | DDKRYNPSLKSRLTISKDTSSNQVFLKI | NO: 110) | (SEQ ID | MDY |
| | TSVDTADTATYYCARRGFYYRYDGGRRY | | NO: 111) | (SEQ ID |
| | YYAMDYWGQGTSVTVSS | | | NO: 112) |
| | (SEQ ID NO: 109) | | | |

TABLE 9
VL amino acid sequences (CRDs: italic underlined) and LCRD1, LCDR2, LCDR3

| Clone | VL | LCDR1 | LCDR2 | LCDR3 |
|---|---|---|---|---|
| 2 | DIVLTQSPASLAVSLGQRATISCKASQS | KASQSVDY | AASNLES | QQSNEDPY |
| | VDYDGDSYMNWYQQKPGQPPKLLIYAAS | DGDSYMN | (SEQ ID | T |
| | NLESGIPARFSGSGSGTDFTLNIHPVEE | (SEQ ID | NO: 115) | (SEQ ID |
| | EDAATYYCQQSNEDPYTFGGGTKLEIK | NO: 114) | | NO: 116) |
| | (SEQ ID NO: 113) | | | |
| 3, 21 | DIVLTQSPASLAVSLGQRATISCRASQS | RASQSVST | YASNLES | QHSWEIPY |
| | VSTSSYSYMHWYQQKPGQPPKLLIKYAS | SSYSYMH | (SEQ ID | T |
| | NLESGVPARFSGSGSGTDFTLNIHPVEE | (SEQ ID | NO: 119) | (SEQ ID |
| | EDTATYYCQHSWEIPYTFGGGTKLEIK | NO: 118) | | NO: 120) |
| | (SEQ ID NO: 117) | | | |
| 5 | DIVMTQSPATLSVTPGDRVSLSCRASQS | RASQSISD | YASQSIS | QNGHSFPY |
| | ISDYLHWYQQKSHESPRLLIKYASQSIS | YLH | (SEQ ID | T |
| | GIPSRFSGSGSGSDFTLSINSVEPEDVG | (SEQ ID | NO: 123) | (SEQ ID |
| | VYYCQNGHSFPYTFGGGTKLEIK | NO: 122) | | NO: 124) |
| | (SEQ ID NO: 121) | | | |
| 9 | DIVMSQSPSSLAVSVGEKVTMSCKSSQS | KSSQSLLY | WASTRES | QQYYSYPP |
| | LLYSSNQKNYLAWYQQKPGQSPKLLIYW | SSNQKNYL | (SEQ ID | T |
| | ASTRESGVPDRFTGSGSGTDFTLTISSV | A | NO: 127) | (SEQ ID |
| | KAEDLAVYYCQQYYSYPPTFGGGTKLEI | (SEQ ID | | NO: 128) |
| | K | NO: 126) | | |
| | (SEQ ID NO: 125) | | | |
| 10 | DIVLTQSPASLAVSLGQRATISCKASQS | KASQSVDY | AASNLES | QQSNEDPY |
| | VDYDGDSYMNWYQQKPGQPPKLLIYAAS | DGDSYMN | (SEQ ID | T |
| | NLESGIPARFSGSGSGTDFTLNIHPVEE | (SEQ ID | NO: 131) | (SEQ ID |
| | EDAATYYCQQSNEDPYTFGGGTKLEIK | NO: 130) | | NO: 132) |
| | (SEQ ID NO: 129) | | | |
| 12, 13 | DIVLTQSPASLAVSLGQRATISCRASES | RASESVEY | AASNVES | QQSRKVPS |
| | VEYYGTSLMQWYQQKPGQPPKLLIYAAS | YGTSLMQ | (SEQ ID | T |
| | NVESGVPARFSGSGSGTHFSLNIHPVEE | (SEQ ID | NO: 135) | (SEQ ID |
| | DDIAMYFCQQSRKVPSTFGGGTKLEIK | NO: 134) | | NO: 136) |
| | (SEQ ID NO: 133) | | | |
| 14 | DIVLTQSPASLAVSLGQRATISCRASKS | RASKSVST | LASNLES | QHSRELPL |
| | VSTSGYSYMHWYQQKPGQPPKLLIYLAS | SGYSYMH | (SEQ ID | T |
| | NLESGVPARFSGSGSGTDFTLNIHPVEE | (SEQ ID | NO: 139) | (SEQ ID |
| | EDAATYYCQHSRELPLTFGAGTKLELK | NO: 138) | | NO: 140) |
| | (SEQ ID NO: 137) | | | |
| 15 | DIQMTQSPASLSASVGETVTITCRASGN | RASGNIHN | NAKTLAD | QHFWSTPW |
| | IHNYLAWYQQKQGKSPQLLVYNAKTLAD | YLA | (SEQ ID | T |
| | GVPSRFSGSGSGTQYSLKINSLQPEDFG | (SEQ ID | NO: 143) | (SEQ ID |
| | SYYCQHFWSTPWTFGGGTKLEIK | NO: 142) | | NO: 144) |
| | (SEQ ID NO: 141) | | | |
| 16 | DVVMTQTPLTLSVTIGQPASISCKSSQS | KSSQSLLD | LVSKLDS | WQGTHFPW |
| | LLDSDGKTYLKWLLQRPGQSPKRLIYLV | SDGKTYLK | (SEQ ID | T |
| | SKLDSGVPDRFTGSGSGTDFTLKISRVE | (SEQ ID | NO: 147) | (SEQ ID |
| | AEDLGVYYCWQGTHFPWTFGGGTKLEIK | NO: 146) | | NO: 148) |
| | (SEQ ID NO: 145) | | | |
| 17 | DIVMTQAAPSVPVTPGESVSISCRSSKS | RSSKSLLH | RMSNLAS | MQHLEYPF |
| | LLHSNGNTYLYWFLQRPGQSPQLLIYRM | SNGNTYLY | (SEQ ID | T |
| | SNLASGVPDRFSGSGSGTAFTLRISRVE | (SEQ ID | NO: 151) | (SEQ ID |
| | AEDVGVYYCMQHLEYPFTFGAGTKLELK | NO: 150) | | NO: 152) |
| | (SEQ ID NO: 149) | | | |
| 18 | TIVMTQTPKFLLVSAGDRVTITCKASQS | KASQSVSN | SASNRYT | QQDYTSPF |
| | VSNAVAWYQQKPGQSPKLLIYSASNRYT | AVA | (SEQ ID | T |
| | GVPDRFTGSGYGTDFTFTISSVQAEDLA | (SEQ ID | NO: 155) | (SEQ ID |
| | VYFCQQDYTSPFTFGSGTKLEIK | NO: 154) | | NO: 156) |
| | (SEQ ID NO: 153) | | | |
| 19 | QIVLTQSPAIMSASPGEKVTMSCSASSS | SASSSVTY | DTSKLAS | QQWSSNPP |
| | VTYMHWYQQKSGTSPKRWIYDTSKLASG | MH | (SEQ ID | T |
| | VPARFSGSGSGTSYSLTISSMEAEDAAT | (SEQ ID | NO: 159) | (SEQ ID |
| | YYCQQWSSNPPTFGGGTKLEIK | NO: 158) | | NO: 160) |
| | (SEQ ID NO: 157) | | | |
| 20 | NIVLTQSPASLAVSLGQRATISCRASES | RASESVDS | LASNLES | QQNNEDPW |
| | VDSYGNSFMHWYQQKPGQPPKLLIYLAS | YGNSFMH | (SEQ ID | T |
| | NLESGVPARFSGSGSRTDFTLTIDPVEA | (SEQ ID | NO: 163) | (SEQ ID |
| | DDAATYYCQQNNEDPWTFGGGTKLEIK | NO: 162) | | NO: 164) |
| | (SEQ ID NO: 161) | | | |
| 24 | NIMMTQSPSSLAVSAGEKVTMSCKSSQS | KSSQSVLY | WASTRES | HQYLSSY |
| | VLYSSNQKNYLAWYQQKPGQSPKLLIYW | SSNQKNYL | (SEQ ID | (SEQ ID |
| | ASTRESGVPDRFTGSGSGTDFTLTISSV | A | NO: 167) | NO: 168) |
| | QAEDLAVYYCHQYLSSYTFGGGTKLEIK | (SEQ ID | | |
| | (SEQ ID NO: 165) | NO: 166) | | |
| 34 | ENVLTQSPAIMSASPGEKVTMTCRASSS | RASSSVSS | STSNLAS | QQYSGYPL |
| | VSSSYLHWYQQKSGASPKLWIYSTSNLA | SYLH | (SEQ ID | T |
| | SGVPARFSGSGSGTSYSLTISSVEAEDA | (SEQ ID | NO: 171) | (SEQ ID |
| | ATYYCQQYSGYPLTFGAGTKLELK | NO: 170) | | NO: 172) |
| | (SEQ ID NO: 169) | | | |
| 35, 36 | DIVMTQAAPSVPVTPGESVSISCRSSKS | RSSKSLLH | RMSNLAS | MQHLEYPF |
| | LLHSNGNTYLYWFLQRPGQSPQLLIYRM | SNGNTYLY | (SEQ ID | T |
| | SNLASGVPDRFSGSGSGTAFTLRISRVE | (SEQ ID | NO: 175) | (SEQ ID |
| | AEDVGVYYCMQHLEYPFTFGSGTKLEIK | NO: 174) | | NO: 176) |
| | (SEQ ID NO: 173) | | | |
| 37 | DIVLTQSPASLAVSLGQRATISCRASKS | RASKSVST | LASNLES | HHSRELPR |
| | VSTSGYSYMHWYQQKPGQPPKLLIYLAS | SGYSYMH | (SEQ ID | T |
| | NLESGVPARFSGSGSGTDFTLNIHPVEE | (SEQ ID | NO: 179) | (SEQ ID |
| | EDASTYYCHHSRELPRTFGGGTKLEIK | NO: 178) | | NO: 180) |
| | (SEQ ID NO: 177) | | | |
| 39 | DVVVTQTPLSLPVSFGDQVSISCRSSQS | RSSQSLAN | GISNRFS | LQGTHQPW |
| | LANSYGNTYLSWYLHKPGQSPQLLIYGI | SYGNTYLS | (SEQ ID | T |
| | SNRFSGVPDRFSGSGSGTDFTLKISTIK | (SEQ ID | NO: 183) | (SEQ ID |
| | PEDLGMYYCLQGTHQPWTFGGGTKLEIK | NO: 182) | | NO: 184) |
| | (SEQ ID NO: 181) | | | |
| 40 | DIQMTQSPASLSASVGETVTITCRASGN | RASGNIHN | NAKTLAD | QHFWSTPY |
| | IHNYLAWYQQKQGKSPQLLVYNAKTLAD | YLA | (SEQ ID | T |
| | GVPSRFSGSGSGTQYSLKINSLQPEDFG | (SEQ ID | NO: 187) | (SEQ ID |
| | SYYCQHFWSTPYTFGGGTKLEIK | NO: 186) | | NO: 188) |
| | (SEQ ID NO: 185) | | | |
| 41 | DIVLTQSPASLAVSLGQRATISCKASQS | KASQSVDY | AASNLES | QQSNEDPW |
| | VDYDGDSYMNWYQQKPGQPPKLLIYAAS | DGDSYMN | (SEQ ID | T |
| | NLESGIPARFSGSGSGTDFTLNIHPVEE | (SEQ ID | NO: 191) | (SEQ ID |
| | EDAATYYCQQSNEDPWTFGGGTKLEIK | NO: 190) | | NO: 192) |
| | (SEQ ID NO: 189) | | | |
| 42-L1 | DVVMTQTPLTLSVTIGQPASISCKSSQS | KSSQSLLD | LVSKLDS | WQGTHFPW |
| | LLDSDGKTYLNWLLQRPGQSPKRLIYLV | SDGKTYLN | (SEQ ID | T |
| | SKLDSGVPDRFTGSGSGTDFTLKISRVE | (SEQ ID | NO: 195) | (SEQ ID |
| | AEDLGVYYCWQGTHFPWTFGGGTKLEIK | NO: 194) | | NO: 196) |
| | (SEQ ID NO: 193) | | | |
| 42-L2 | DIVLTQSPATLSVTPGDRVSLSCRASQS | RASQSISN | YASQSIS | QQSNSWPF |
| | ISNYLHWYQQKSHESPRLLIKYASQSIS | YLH | (SEQ ID | T |
| | GIPSRFSGSGSGTDFTLSINSVETEDFG | (SEQ ID | NO: 199) | (SEQ ID |
| | MYFCQQSNSWPFTFGSGTKLEIK | NO: 198) | | NO: 200) |
| | (SEQ ID NO: 197) | | | |
| 43 | DIVMSQSPSSLAVSAGEKVTMSCKSSQS | KSSQSLLN | WASTRES | KQSYNLPL |
| | LLNSRTRKNYLAWYQQKPGQSPKLLIYW | SRTRKNYL | (SEQ ID | T |
| | ASTRESGVPDRFTGSGSGTDFTLTISSV | A | NO: 203) | (SEQ ID |
| | QAEDLAVYSCKQSYNLPLTFGAGTKLEL | SEQ ID | | NO: 204) |
| | K | NO: 202) | | |
| | (SEQ ID NO: 201) | | | |
| 44 | QIVLTQSPAIMSASPGEKVTMTCSASSS | SASSSISY | DTSKLAS | HQRSSYPY |
| | ISYMHWYQQKPGTSPKRWIYDTSKLASG | MH | (SEQ ID | T |
| | VPARFSGSGSGTSYSLTISSMEAEDAAT | (SEQ ID | NO: 207) | (SEQ ID |
| | YYCHQRSSYPYTFGGGTKLEIK | NO: 206) | | NO: 208) |
| | (SEQ ID NO: 205) | | | |
| 45 | DIVLTQSPASLAVSLGQRATISCRASKS | RASKSVST | LASNLES | QHSRELPY |
| | VSTSGYSYMHWYQQKPGQPPKLLIYLAS | SGYSYMH | (SEQ ID | T |
| | NLESGVPARFSGSGSGTDFTLNIHPVEE | (SEQ ID | NO: 211) | (SEQ ID |
| | EDAATYYCQHSRELPYTFGGGTKLEIK | NO: 210) | | NO: 212) |
| | (SEQ ID NO: 209) | | | |
| 47 | NIVLTQSPASLAVSLGQRATISCRASES | RASESVDS | LASNLES | QQNNEDPP |
| | VDSFGNSFMHWYQQKPGQPPKLLIYLAS | FGNSFMH | (SEQ ID | WT |
| | NLESGVPARFSGSGSRTDFTLTIDPVEA | (SEQ ID | NO: 215) | (SEQ ID |
| | DDAATYYCQQNNEDPPWTFGGGTKLEIK | NO: 214) | | NO: 216) |
| | (SEQ ID NO: 213) | | | |
| 48 | DILLTQSPAILSVSPGERVSFSCRASQS | RASQSIGT | FASESIS | QQSNSWPL |
| | IGTSIHWYQQGTNGSPRLLIKFASESIS | SIH | (SEQ ID | T |
| | GIPSRFSGSGSGTDFTLSINSVESEDIA | (SEQ ID | NO: 219) | (SEQ ID |
| | DYYCQQSNSWPLTFGAGTKLELK | NO: 218) | | NO: 220) |
| | (SEQ ID NO: 217) | | | |

TABLE 10
VH DNA sequences (CRDs: italic underlined) and HCDR1, HCDR2, HCDR3

| clone | VH | HCDR1 | HCDR2 | HCDR3 |
|---|---|---|---|---|
| 2 | caggtccagctgcagcagtctggagctgagct | aattact | gtgatta | tcacgggg |
| | ggtaaggcctgggacttcagtgaaggtgtcct | tgataga | atcctgg | ttacccct |
| | gcaaggcttctggatacgccttcactaattac | g | aagtggt | atgctatg |
| | ttgatagagtgggtaaagcagaggcctggaca | (SEQ ID | ggtacta | gactac |
| | gggccttgagtggattggagtgattaatcctg | NO: | actacaa | (SEQ ID |
| | gaagtggtggtactaactacaatgagaagttc | 222) | tgagaag | NO: 224) |
| | aagggcaaggcaacactgactgcagacaaatc | | ttcaagg | |
| | ctccagcactgcctacatgcagctcagcagcc | | gc | |
| | tgacatctgatgactctgcggtctatttctgt | | (SEQ ID | |
| | gcgagatcacggggttacccctatgctatgga | | NO: | |
| | ctactggggtcaaggaacctcagtcaccgtct | | 223) | |
| | cctca | | | |
| | (SEQ ID NO: 221) | | | |
| 3, 21 | Gaggtccagctgcaacagtctggacctgagct | gaataca | ggtatta | tcgttagt |
| | ggtgaagcctggggcttcagtgaagatatcct | ccatgca | atcctaa | agaggggg |
| | gcaagacctctggatacacattcactgaatac | (SEQ ID | caatggt | actttgct |
| | accatgcactgggtgaagcagagccatggaaa | NO : | ggtacta | tac |
| | gagccttgagtggattggaggtattaatccta | 226) | gctacaa | (SEQ ID |
| | acaatggtggtactagctacaaccagaagttc | | ccagaag | NO: 228) |
| | aagggcaaggccacattgactgtagacaagtc | | ttcaagg | |
| | ctccagcacagcctacatggagctccgcagcc | | gc | |
| | tgacatctgaggattctgcagtctattactgt | | (SEQ ID | |
| | gcaagatcgttagtagagggggactttgctta | | NO: | |
| | ctggggccaagggactctggtcactgtcctg | | 227) | |
| | ca | | | |
| | (SEQ ID NO: 225) | | | |
| 5 | gaggtgaagctggtggaatctggaggaggctt | gatttct | gcaagta | gatttcct |
| | ggtacagcctgggggttctctgagactctcct | acatgga | gaaacaa | cccttact |
| | gtgcaacttctgggttcaccttcagtgatttc | g | agctaat | acggtagt |
| | tacatggagtgggtccgccagcctccagggaa | (SEQ ID | gattata | ccctggtt |
| | gagactggagtggattgctgcaagtagaaaca | NO: | caacaga | tgcttac |
| | aagctaatgattatacaacagagtacagtgca | 230) | gtacagt | (SEQ ID |
| | tctgtgaagggtcggttcatcgtctccagaga | | gcatctg | NO: 232) |
| | cacttcccaaagcatcctctaccttcagatga | | tgaaggg | |
| | atgccctgagagctgaggacactgccatttat | | t | |
| | tactgtgcaagagatttcctcccttactacgg | | (SEQ ID | |
| | tagtccctggtttgcttactggggccaaggga | | NO: 231) | |
| | ctctggtcactgtctctgca | | | |
| | (SEQ ID NO: 229) | | | |
| 9 | Gaggtccagctgcagcagtctggacctgagct | ggctaca | aatattg | tcgcggtt |
| | ggagaagcctggcgcttcagtgaagatatcct | acatgaa | atcctta | tattacta |
| | gcaaggcttctggttactcattcactggctac | c | ctatggt | cggtagta |
| | aacatgaactgggtgaagcagagcaatggaaa | (SEQ ID | ggtacta | gctaccga |
| | gagccttgagtggattggaaatattgatcctt | NO: | gctacaa | ttactatg |
| | actatggtggtactagctacaaccagaagttc | 234) | ccagaag | ctatggac |
| | aagggcaaggccacattgactctagacaaatc | | ttcaagg | tac |
| | ctccagcacagcctacatgcagctcaagagcc | | gc | (SEQ ID |
| | tgacatctgaggactctgcagtctattactgt | | (SEQ ID | NO: 236) |
| | gcaagatcgcggtttattactacggtagtagc | | NO: | |
| | taccgattactatgctatggactactggggtc | | 235) | |
| | aaggaacctcagtcaccgtctcctca | | | |
| | (SEQ ID NO: 233) | | | |
| 10 | gaggtgaagctggtggaatctggaggaggctt | gatttct | gcaagta | gagaacta |
| | ggtacagcctgggggttctctgagactctcct | acatgga | gaaacaa | tggtaacc |
| | gtgcaacttctgggttcaccttcagtgatttc | g | agctaat | ccgctatg |
| | tacatggagtgggtccgccagcctccagggaa | (SEQ ID | gattata | gactac |
| | gagactggagtggattgctgcaagtagaaaca | NO: | caacaga | (SEQ ID |
| | aagctaatgattatacaacagagtacagtgca | 238) | gtacagt | NO: 240) |
| | tctgtgaagggtcggttcatcgtctccagaga | | gcatctg | |
| | cacttcccaaagcatcctctaccttcagatga | | tgaaggg | |
| | atgccctgagagctgaggacactgccatttat | | t | |
| | tactgtgcaagagagaactatggtaaccccgc | | (SEQ ID | |
| | tatggactactggggtcaaggaacctcagtca | | NO: | |
| | ccgtctcctca | | 239) | |
| | (SEQ ID NO: 237) | | | |
| 12, 13 | caggtgcagctgaaggagtcaggacctggcct | agctatg | gtaatat | ggggggat |
| | ggtggcgccctcacagagcctgtccatcactt | gtgtaca | gggctgg | tacgtccc |
| | gcactgtctctgggttttcattaaccagctat | c | tggaagc | ttcataac |
| | ggtgtacactgggttcgccagcctccaggaaa | (SEQ ID | acaaatt | tatgttat |
| | gggtctggagtggctgggagtaatatgggctg | NO: | ataattc | ggactac |
| | gtggaagcacaaattataattcggctctcatg | 242) | ggctctc | (SEQ ID |
| | tccagactgagcatcagcaaagacaactccaa | | atgtcc | NO: 244) |
| | gagccaagttttcttaaaaatgaacagtctgc | | (SEQ ID | |
| | aaactgatgacacagccatgtacttctgtgcc | | NO: | |
| | agaggggggattacgtcccttcataactatgt | | 243) | |
| | tatggactactggggtcaaggaacctcagtca | | | |
| | ccgtctcctca | | | |
| | (SEQ ID NO: 241) | | | |
| 14 | Caggttcagctgcagcagtctggagctgagct | Agctact | gagattt | gggagtgc |
| | gatgaagcctggggcctcagtgaagatatcct | ggataga | tacctgg | aggtaatt |
| | gcaaggctactggctacacattcagtagctac | g | aagtggt | actacggt |
| | tggatagagtgggtaaagcagaggcctggaca | (SEQ ID | agtacta | agtagcta |
| | tggccttgagtggattggagagattttacctg | NO: | actacaa | cgaggcta |
| | gaagtggtagtactaactacaatgagaagttc | 246) | tgagaag | tggactac |
| | aagggcaaggccacattcactgcagatacatc | | ttcaagg | (SEQ ID |
| | ctccaacacagcctacatgcaactcagcagcc | | gc | NO: 248) |
| | tgacatctgaggactctgccgtctattactgt | | (SEQ ID | |
| | gcaagtgggagtgcaggtaattactacggtag | | NO: | |
| | tagctacgaggctatggactactggggtcaag | | 247) | |
| | gaacctcagtcaccgtctcctca (SEQ ID | | | |
| | NO: 245) | | | |
| 15 | Caggtgcagctgaaggagtcaggacctggcct | agctatg | gtaatat | ccggatct |
| | ggtggcgccctcacagagcctgtccatcacat | gtgtaag | ggggtga | ctactatg |
| | gcactgtctcagggttctcattaaccagctat | c | cgggagc | attctccc |
| | ggtgtaagctgggttcgccagcctccaggaaa | (SEQ ID | acaaatt | tatgctat |
| | gggtctggagtggctgggagtaatatggggtg | NO: | atcattc | ggactac |
| | acgggagcacaaattatcattcagctctcata | 250) | agctctc | (SEQ ID |
| | tccagactgagcatcagcaaggataactccaa | | atatcc | NO: 252) |
| | gagccaagttttcttaaaactgaacagtctgc | | (SEQ ID | |
| | aaactgatgacacagccacgtactactgtgcc | | NO: | |
| | aaaccggatctctactatgattctccctatgc | | 251) | |
| | tatggactactggggtcaaggaacctcagtca | | | |
| | ccgtctcctca | | | |
| | (SEQ ID NO: 249) | | | |
| 16 | Gaggtgaagcttctcgagtctggaggtggcct | agatact | gaaatta | ccccttat |
| | ggtgcagcctggaggatccctgaaactctcct | ggatgag | atccaga | ggactac |
| | gtgcagcctcaggattcgattttagtagatac | t | tagcagt | (SEQ ID |
| | tggatgagttgggtccggcaggctccagggaa | (SEQ ID | acgataa | NO: 256) |
| | agggctagaatggattggagaaattaatccag | NO: | actatac | |
| | atagcagtacgataaactatacgccatctcta | 254) | gccatct | |
| | aaggataaattcatcatctccagagacaacgc | | ctaaagg | |
| | caaaaatacgctgtacctgcaaatgagcaaag | | at | |
| | tgagatctgaggacacagccctttattactgt | | (SEQ ID | |
| | gcaagaccccttatggactactggggtcaagg | | NO: | |
| | aacctcagtcaccgtctcctca | | 255) | |
| | (SEQ ID NO: 253) | | | |
| 17 | Caggtgcagctgaaggagtcaggacctggcct | agctatg | gtaatat | gatggcta |
| | ggtggcgccctcacagagcctgtccatcactt | gtgtaca | gggctgg | cggctacg |
| | gcactgtctctgggttttcattaaccagctat | c | tggaagc | tttactat |
| | ggtgtacactgggttcgccagcctccaggaaa | (SEQ ID | tcaaatt | gctatgga |
| | gggtctggagtggctgggagtaatatgggctg | NO: | ataattc | ctac |
| | gtggaagctcaaattataattcggctctcatg | 258) | ggctctc | (SEQ ID |
| | tccagactgagcatcagcaaagacaactccaa | | atgtcc | NO: 260) |
| | gagccaagttttcttaaaaatgaacagtctgc | | (SEQ ID | |
| | aaactgatgacacagccatgtactactgtgcc | | NO: | |
| | agagatggctacggctacgtttactatgctat | | 259) | |
| | ggactactggggtcaaggaacctcagtcaccg | | | |
| | tctcctca | | | |
| | (SEQ ID NO: 257) | | | |
| 18 | caggtccagctgcagcagtctggagctgagct | aactatt | gatattt | gacggtta |
| | ggtaaggcctgggacttcagtgaagatgtcct | ggatagg | accctgg | ctccccct |
| | gcaaggctgctggatacaccttcactaactat | t | aggtggt | ac |
| | tggataggttgggtaaagcagaggcctggaca | (SEQ ID | tatacta | (SEQ ID |
| | tggccttgagtggattggagatatttaccctg | NO: | agtacaa | NO: 264) |
| | gaggtggttatactaagtacaatgagaagttc | 262) | tgagaag | |
| | aagggcaaggccacactgactgcagacacatc | | ttcaagg | |
| | ctccagtacagcctacatgcagctcagcagcc | | gc | |
| | tgacatctgaggactctgccatctattactgt | | (SEQ ID | |
| | gcaagagacggttactccccctactggggcca | | NO: | |
| | aggcacctctctcacagtctcctca | | 263) | |
| | (SEQ ID NO: 261) | | | |
| 19 | caggtccagctgcagcagtctggggctgagct | gattatg | gttatta | cgggggat |
| | ggtgaggcctggggtctcagtgaagatttcct | ctatgca | gtactta | tacggtag |
| | gcaagggttctggctacacattcactgattat | c | ctatggt | tagccgac |
| | gctatgcactgggtgaagcagagtcatgcaaa | (SEQ ID | gatgcta | tatgctat |
| | gagtctagagtggattggagttattagtactt | NO: | ggtacaa | ggactac |
| | actatggtgatgctaggtacaaccagaagttc | 266) | ccagaag | (SEQ ID |
| | aagggcaaggccacaatgactgtagacaaatc | | ttcaagg | NO: 268) |
| | ctccagcacagcctatatggaacttgccagac | | gc | |
| | tgacatctgaggattctgccatctattactgt | | (SEQ ID | |
| | gcaagacgggggattacggtagtagccgacta | | NO: | |
| | tgctatggactactggggtcaaggaacctcag | | 267) | |
| | tcaccgtctcctca | | | |
| | (SEQ ID NO: 265) | | | |
| 20 | Caggtccagctgcagcagtctggagctgagct | aattact | gtgatta | tcgcacta |
| | ggtaaggcctgggacttcagtgaaggtgtcct | tgataga | atcctgg | tggtaact |
| | gcaaggcttctggatacgccttcactaattac | g | aagtggt | actatgct |
| | ttgatagagtgggtaaagcagaggcctggaca | (SEQ ID | ggtacta | atggacta |
| | gggccttgagtggattggagtgattaatcctg | NO: | actacaa | c |
| | gaagtggtggtactaactacaatgagaagttc | 270) | tgagaag | (SEQ ID |
| | aagggcaaggcaacactgactgcagacaaatc | | ttcaagg | NO: 272) |
| | ctccagcactgcctacatgcagctcagcagcc | | gc | |
| | tgacatctgatgactctgcggtttatttctgt | | (SEQ ID | |
| | gcaagatcgcactatggtaactactatgctat | | NO: | |
| | ggactactggggtcaaggaacctcagtcaccg | | 271) | |
| | tctcctca | | | |
| | (SEQ ID NO: 269) | | | |
| 24 | Gaggtccagctgcaacagtctggacctgagct | gactact | gatatta | agaggttt |
| | ggtgaagcctggggcttcagtgaagatgtcct | acatgaa | atcctaa | ttattact |
| | gcaaggcttctggatacaccttcactgactac | g | caatggt | acggtagt |
| | tacatgaagtgggtgaagcagagccatggaaa | (SEQ ID | gatactt | agaggggg |
| | gagccttgagtggattggagatattaatccta | NO: | tctacaa | tattatgg |
| | acaatggtgatactttctacaaccagaagttc | 274) | ccagaag | actac |
| | aagggcaaggccacattgactgtagacaaatc | | ttcaagg | (SEQ ID |
| | ctccagcacagcctacatgcagctcaacagcc | | qc | NO: 276) |
| | tgacatctgaggactctgcagtctattactgt | | (SEQ ID | |
| | gcaagaagaggtttttattactacggtagtag | | NO: | |
| | agggggtattatggactactggggtcaaggaa | | 275) | |
| | cctcagtcaccgtctcctca | | | |
| | (SEQ ID NO: 273) | | | |
| 34 | Gaagtgaagctggtggagtctgggggaggctt | gactatt | tacatta | tcctacta |
| | agtgcagcctggagggtccctgaaactctcct | acatgta | gtaatgg | tggtaacc |
| | gtgcaacctctggattcactttcagtgactat | t | tggtgat | cttttcct |
| | tacatgtattgggttcgccagactccagagaa | (SEQ ID | agcacct | tac |
| | gaggctggagtgggtcgcatacattagtaatg | NO: | attatcc | (SEQ ID |
| | gtggtgatagcacctattatccagacactgta | 278) | agacact | NO: 280) |
| | aagggccgattcaccatctccagagacaatgc | | gtaaagg | |
| | caagaacaccctgtacctgcaaatgagccgtc | | gc | |
| | tgaagtctgaggacacagccatgtattactgt | | (SEQ ID | |
| | gcaagatcctactatggtaacccttttcctta | | NO: 279) | |
| | ctggggccaggggactctggtcactgtcctg | | | |
| | ca | | | |
| | (SEQ ID NO: 277) | | | |
| 35, 36 | caggtgcagctgaaggagtcaggacctggcct | agctatg | gtaatat | gatggtta |
| | ggtggcgccctcacagagcctgtccatcactt | gtgtaca | gggctgg | caagtttt |
| | gcactgtctctgggttttcattaaccagctat | c | tggaagc | tttactat |
| | ggtgtacactgggttcgccagcctccaggaaa | (SEQ ID | acaaatt | gctatgga |
| | gggtctggagtggctgggagtaatatgggctg | NO: | ataattc | ctac |
| | gtggaagcacaaattataattcggctctcatg | 282) | ggctctc | (SEQ ID |
| | tccagactgagcatcagcaaagacaactccaa | | atgtcc | NO: 284) |
| | gagccaagttttcttaaaaatgaacagtctgc | | (SEQ ID | |
| | aaactgatgacacagccatgtactactgtgcc | | NO: | |
| | actgatggttacaagtttttttactatgctat | | 283) | |
| | ggactactggggtcaaggaacctcagtcaccg | | | |
| | tctcctca | | | |
| | (SEQ ID NO: 281) | | | |
| 37 | Gaggtccagcttcagcagtcaggacctgagct | gactaca | tatattt | cctgggta |
| | ggtgaaacctggggcctcagtgaagatatcct | acatgca | atcctta | ttactatg |
| | gcaaggcttctggatacacattcactgactac | c | caatggt | ctatggac |
| | aacatgcactgggtgaagcagagccatggaaa | (SEQ ID | ggtactg | tac |
| | gagccttgagtggattggatatatttatcctt | NO: | gctacaa | (SEQ ID |
| | acaatggtggtactggctacaaccagaaattc | 286) | ccagaaa | NO: 288) |
| | aagaccaaggccacattgactgtagacaattc | | ttcaaga | |
| | ctccagcacagcctacatggagctccgcagcc | | CC | |
| | tgacatctgaggactctgcagtctattactgt | | (SEQ ID | |
| | gcaatccctgggtattactatgctatggacta | | NO: | |
| | ctggggtcaaggaacctcagtcaccgtctcct | | 287) | |
| | ca | | | |
| | (SEQ ID NO: 285) | | | |
| 39 | Gaagtgaagctggtggagtctgggggaggttt | agctata | tacatta | ggggtatc |
| | agtgcagcctggagggtccctgaaactctcct | ccatgtc | gtaatgg | ctactatg |
| | gtgcagcctctggattcactttcagtagctat | t | tggtggt | gtgacctg |
| | accatgtcttgggttcgccagactccagagaa | (SEQ ID | agcacct | tactactt |
| | gaggctggagtgggtcgcatacattagtaatg | NO: | actatcc | tgactac |
| | gtggtggtagcacctactatccagacactgta | 290) | agacact | (SEQ ID |
| | aagggccgattcaccatctccagagacaatgc | | gtaaagg | NO: 292) |
| | caagaacaccctgtacctgcaaatgagcagtc | | gc | |
| | tgaagtctgaggacacggccatgtattactgt | | (SEQ ID | |
| | gcaagaggggtatcctactatggtgacctgta | | NO: | |
| | ctactttgactactggggccaaggcaccactc | | 291) | |
| | tcacagtctcctca | | | |
| | (SEQ ID NO: 289) | | | |
| 40 | Cagatccagttggtgcagtctggacctgagct | aactatg | tggataa | tccctcta |
| | gaagaagcctggagagacagtcaagatctcct | gaatgaa | acaccta | tggttacg |
| | gcaaggcttctgggtataccttcacaaactat | c | cactgga | acgagggg |
| | ggaatgaactgggtgaagcaggctccaggaaa | (SEQ ID | gagccaa | tacttcga |
| | gggtttaaagtggatgggctggataaacacct | NO: | catatgc | tgtc |
| | acactggagagccaacatatgctgatgacttc | 294) | tgatgac | (SEQ ID |
| | aagggacggtttgccttctctttggaaacctc | | ttcaagg | NO: 296) |
| | tgccagcactgcctatttgcagatcaacaacc | | ga | |
| | tcaaaaatgaggacacggctacatatttctgt | | (SEQ ID | |
| | gcaagatccctctatggttacgacgaggggta | | NO: | |
| | cttcgatgtctggggcgcagggaccacggtca | | 295) | |
| | ccgtctcctca | | | |
| | (SEQ ID NO: 293) | | | |
| 41 | Caggtccagctgcagcagtctggagctgagct | aattact | gtgatta | tcgggatt |
| | ggtaaggcctgggacttcagtgaaggtgtcct | tgataga | atcctgg | acgacggg |
| | gcaaggcttctggatacgccttcactaattac | g | aagtggt | attactat |
| | ttgatagagtgggtaaagcagaggcctggaca | (SEQ ID | ggtacta | gctatgga |
| | gggccttgagtggattggagtgattaatcctg | NO: | actacaa | ctac |
| | gaagtggtggtactaactacaatgagaagttc | 298) | tgagaag | (SEQ ID |
| | aagggcaaggcaacactgactgcagacaaatc | | ttcaagg | NO: 300) |
| | ctccagcactgcctacatgcagctcagcagcc | | gc | |
| | tgacatctgatgactctgcggtctatttctgt | | (SEQ ID | |
| | gcaagatcgggattacgacgggattactatgc | | NO: | |
| | tatggactactggggtcaaggaacctcagtca | | 299) | |
| | ccgtctcctca | | | |
| | (SEQ ID NO: 297) | | | |
| 42 | Caggtgcagctgaaggagtcaggacctggcct | agctatg | gtaatat | gagggtaa |
| | ggtggcgccctcacagagcctgtccatcactt | gtgtaca | gggctgg | ctgggacg |
| | gcactgtctctgggttttcattaaccagctat | c | tggaagc | attttgac |
| | ggtgtacactgggttcgccagcctccaggaaa | (SEQ ID | acaaatt | tac |
| | gggtctggagtggctgggagtaatatgggctg | NO: | ataattc | (SEQ ID |
| | gtggaagcacaaattataattcggctctcatg | 302) | ggctctc | NO: 304) |
| | tccagactgagcatcagcaaagacaactccaa | | atgtcc | |
| | gagccaagttttcttaaaaatgaacagtctgc | | (SEQ ID | |
| | aaactgatgacacagccatgtactactgtgcc | | NO: | |
| | agagagggtaactgggacgattttgactactg | | 303) | |
| | gggccaaggcaccactctcacagtctcctca | | | |
| | (SEQ ID NO: 301) | | | |
| 43 | Gacgtgaagttggtggagtctgggggaggctt | agctata | accatta | gggggtta |
| | agtgaagcctggagggtccctgaaactctcct | ccatgtc | gtagtgg | cggtagta |
| | gtgcagcctctggattcactttcagtagctat | t | tggtagt | gcttcgag |
| | accatgtcttgggttcgccagactccggagaa | (SEQ ID | tacacct | aactactt |
| | gaggctggagtgggtcgcaaccattagtagtg | NO: | actatcc | tgactac |
| | gtggtagttacacctactatccagacagtgtg | 306) | agacagt | (SEQ ID |
| | aagggccgattcaccatctccagagacaatgc | | gtgaagg | NO: 308) |
| | caagaacaccctgtacctgcaaatgagcagtc | | gc | |
| | tgaaggctgaggacacagccatgtattactgt | | (SEQ ID | |
| | acaagagggggttacggtagtagcttcgagaa | | NO: | |
| | ctactttgactactggggccaaggcaccactc | | 307) | |
| | tcacagtctcctca | | | |
| | (SEQ ID NO: 305) | | | |
| 44 | Gaggtccagctgcagcagtctggacctgagct | ggctaca | aatattg | gaggatag |
| | ggagaagcctggcgcttcagtgaagatatcct | acatgaa | accctta | gtacctag |
| | gcaaggcttctggttactcattcactggctac | c | ctatggt | ttcattac |
| | aacatgaactgggtgaagcagagcaatggaaa | (SEQ ID | ggtatta | tatgctat |
| | gagccttgagtggattggaaatattgaccctt | NO: | ggtacaa | ggactac |
| | actatggtggtattaggtacaaccagaagttc | 310) | ccagaag | (SEQ ID |
| | aagggcaaggccacattgactgtagacaaatc | | ttcaagg | NO: 312) |
| | ctccagcacagcctacatgcagctcaagagcc | | gc | |
| | tgacatctgaggactctgcagtctattactgt | | (SEQ ID | |
| | gcaagagaggataggtacctagttcattacta | | NO: | |
| | tgctatggactactggggtcaaggaacctcag | | 311) | |
| | tcaccgtctcctca | | | |
| | (SEQ ID NO: 309) | | | |
| 45 | Gacgtgaagctggtggagtctgggggaggctt | agctata | accatta | aactacgg |
| | agtgaagcctggagggtccctgaaactctcct | ccatgtc | gtagtgg | tagtcgac |
| | gtgcagcctctggattcactttcagtagctat | t | tggtagt | ccccttac |
| | accatgtcttgggttcgccagactccggagaa | (SEQ ID | tacacct | tatgctat |
| | gaggctggagtgggtcgcaaccattagtagtg | NO: | actatcc | ggactac |
| | gtggtagttacacctactatccagacagtgtg | 314) | agacagt | (SEQ ID |
| | aagggccgattcaccatctccagagacaatgc | | gtgaagg | NO: 316) |
| | caagaacaccctgtacctgcaaatgagcagtc | | gc | |
| | tgaagtctgaggacacagccatgtattactgt | | (SEQ ID | |
| | acaagaaactacggtagtcgacccccttacta | | NO: | |
| | tgctatggactactggggtcaaggaacctcag | | 315) | |
| | tcaccgtctcctca | | | |
| | (SEQ ID NO: 313) | | | |
| 47 | gaggtgaagctggtggagtctggaggaggctt | gattact | tttatta | gatcgccc |
| | ggtacagcctgggggttctctgagactctcct | acatgag | gaaacaa | gggctttg |
| | gtgcaacttctgggttcaccttcactgattac | c | agctaat | actac |
| | tacatgagctgggtccgccagcctccaggaaa | (SEQ ID | ggttaca | (SEQ ID |
| | ggcacttgagtggttgggttttattagaaaca | NO: | caacaga | NO: ) 320 |
| | aagctaatggttacacaacagagtacagtgca | 318) | gtacagt | |
| | tctgtgaagggtcggttcaccatctccagaga | | gcatctg | |
| | taattcccaaagcatcctctatcttcaaatga | | tgaaggg | |
| | acaccctgagagctgaggacagtgccacttat | | t | |
| | tactgtgcaagagatcgcccgggctttgacta | | (SEQ ID | |
| | ctggggccaaggcaccactctcacagtctcct | | NO: | |
| | ca | | 319) | |
| | (SEQ ID NO: 317) | | | |
| 48 | Caggttactctgaaagagtctggccctgggat | acttctg | cacattt | agggggtt |
| | attgcagccctcccagaccctcagtctgactt | gtatggg | actggga | ctactata |
| | gttctttctctgggttttcactgagcacttct | tgtgagc | tgatgac | ggtacgac |
| | ggtatgggtgtgagctggattcgtcagccttc | (SEQ ID | aagcgct | ggggggcg |
| | aggaaagggtctggagtggctggcacacattt | NO: | ataaccc | ccgctatt |
| | actgggatgatgacaagcgctataacccatcc | 322) | atccctg | actatgct |
| | ctgaagagccggctcacaatctccaaggatac | | aagagc | atggacta |
| | ctccagcaaccaggtattcctcaagatcacca | | (SEQ ID | c |
| | gtgtggacactgcagatactgccacatactac | | NO: | (SEQ ID |
| | tgtgctcgaagggggttctactataggtacga | | 323) | NO: 324) |
| | cggggggcgccgctattactatgctatggact | | | |
| | actggggtcaaggaacctcagtcaccgtctcc | | | |
| | tca | | | |
| | (SEQ ID NO: 321) | | | |

TABLE 11
VL DNA sequences (CRDs: italic underlined) and LCDR1, LCDR2, LCDR3

| mAb | VL | LCDR1 | LCDR2 | LCDR3 |
|---|---|---|---|---|
| 2 | gacattgtgctgacccaatctccagcttcttt | aaggccag | gctgcat | cagcaaa |
| | ggctgtgtctctagggcagagggccaccatct | ccaaagtg | ccaatct | gtaatga |
| | cctgcaaggccagccaaagtgttgattatgat | ttgattat | agaa | ggatccg |
| | ggtgatagttatatgaactggtaccaacagaa | gatggtga | (SEQ ID | tacacg |
| | accaggacagccacccaaactcctcatctatg | tagttata | NO: | (SEQ ID |
| | ctgcatccaatctagaatctgggatcccagcc | tgaac | 327) | NO: |
| | aggtttagtggcagtgggtctgggacagactt | (SEQ ID | | 328) |
| | caccctcaacatccatcctgtggaggaggagg | NO: 326) | | |
| | atgctgcaacctattactgtcagcaaagtaat | | | |
| | gaggatccgtacacgttcggaggggggaccaa | | | |
| | gctggaaataaaa | | | |
| | (SEQ ID NO: 325) | | | |
| 3, 21 | gacattgtgctgacacagtctcctgcttcctt | agggccag | tatgcat | cagcaca |
| | agctgtatctctggggcagagggccaccatct | ccaaagtg | ccaacct | gttggga |
| | catgcagggccagccaaagtgtcagcacatct | tcagcaca | agaatct | gattccg |
| | agctatagttatatgcactggtaccaacagaa | tctagcta | (SEQ ID | tacacg |
| | accaggacagccacccaaactcctcataaagt | tagttata | NO: | (SEQ ID |
| | atgcatccaacctagaatctggggtccctgcc | tgcac | 331) | NO: |
| | aggttcagtggcagtgggtctgggacagactt | (SEQ ID | | 332) |
| | caccctcaacatccatcctgtggaggaggagg | NO: 330) | | |
| | atactgcaacatattactgtcagcacagttgg | | | |
| | gagattccgtacacgttcggaggggggaccaa | | | |
| | gctggaaataaaa | | | |
| | (SEQ ID NO: 329) | | | |
| 5 | gacattgtgatgactcagtctccagccaccct | agggccag | tatgctt | caaaatg |
| | gtctgtgactccaggagatagagtctctcttt | ccagagta | cccaatc | gtcacag |
| | cctgcagggccagccagagtattagcgactac | ttagcgac | catctct | ctttccg |
| | ttacactggtatcaacaaaaatcacatgagtc | tacttaca | (SEQ ID | tacacg |
| | tccaaggcttctcatcaaatatgcttcccaat | c | NO: | (SEQ ID |
| | ccatctctgggatcccctccaggttcagtggc | (SEQ ID | 335) | NO: |
| | agtggatcagggtcagatttcactctcagtat | NO: 334) | | 336) |
| | caacagtgtggaacctgaagatgttggagtgt | | | |
| | attactgtcaaaatggtcacagctttccgtac | | | |
| | acgttcggaggggggaccaagctggaaataaa | | | |
| | a | | | |
| | (SEQ ID NO: 333) | | | |
| 9 | Gacattgtgatgtcacagtctccatcctccct | aagtccag | tgggcat | cagcaat |
| | agctgtgtcagttggagagaaggttactatga | tcagagcc | ccactag | attatag |
| | gctgcaagtccagtcagagccttttatatagt | ttttatat | ggaatct | ctatcct |
| | agcaatcaaaagaactacttggcctggtacca | agtagcaa | (SEQ ID | cccacg |
| | gcagaaaccagggcagtctcctaaactgctga | tcaaaaga | NO: | (SEQ ID |
| | tttactgggcatccactagggaatctggggtc | actacttg | 339) | NO: |
| | cctgatcgcttcacaggcagtggatctgggac | (SEQ ID | | 340) |
| | agatttcactctcaccatcagcagtgtgaagg | NO: 338) | | |
| | ctgaagacctggcagtttattactgtcagcaa | | | |
| | tattatagctatcctcccacgttcggaggggg | | | |
| | gaccaagctggaaataaaa | | | |
| | (SEQ ID NO: 337) | | | |
| 10 | Gacattgtgctgacccaatctccagcttcttt | aaggccag | gctgcat | cagcaaa |
| | ggctgtgtctctagggcagagggccaccatct | ccaaagtg | ccaatct | gtaatga |
| | cctgcaaggccagccaaagtgttgattatgat | ttgattat | agaatct | ggatccg |
| | ggtgatagttatatgaactggtaccaacagaa | gatggtga | (SEQ ID | tacacg |
| | accaggacagccacccaaactcctcatctatg | tagttata | NO: | (SEQ ID |
| | ctgcatccaatctagaatctgggatcccagcc | tgaac | 343) | NO: |
| | aggtttagtggcagtgggtctgggacagactt | (SEQ ID | | 344) |
| | caccctcaacatccatcctgtggaggaggagg | NO: 342) | | |
| | atgctgcaacctattactgtcagcaaagtaat | | | |
| | gaggatccgtacacgttcggaggggggaccaa | | | |
| | gctggaaataaaa | | | |
| | (SEQ ID NO: 341) | | | |
| 12, 13 | gacattgtgctcacccaatctccagcttcttt | agagccag | gctgcat | cagcaaa |
| | ggctgtgtctctagggcagagagccaccatct | tgaaagtg | ccaacgt | gtaggaa |
| | cctgcagagccagtgaaagtgttgaatattat | ttgaatat | agaatct | ggttcct |
| | ggcacaagtttaatgcagtggtaccaacagaa | tatggcac | (SEQ ID | tcgacg |
| | accaggacagccacccaaactcctcatctatg | aagtttaa | NO: | (SEQ ID |
| | ctgcatccaacgtagaatctggggtccctgcc | tgcag | 347) | NO: |
| | aggtttagtggcagtgggtctgggacacactt | (SEQ ID | | 348) |
| | cagcctcaacatccatcctgtggaggaggatg | NO: 346) | | |
| | atattgcaatgtatttctgtcagcaaagtagg | | | |
| | aaggttccttcgacgttcggtggaggcaccaa | | | |
| | gctggaaatcaaa | | | |
| | (SEQ ID NO: 345) | | | |
| 14 | Gacattgtgctgacacagtctcctgcttcctt | agggccag | cttgcat | cagcaca |
| | agctgtatctctggggcagagggccaccatct | caaaagtg | ccaacct | gtaggga |
| | catgcagggccagcaaaagtgtcagtacatct | tcagtaca | agaatct | gcttcct |
| | ggctatagttatatgcactggtaccaacagaa | tctggcta | (SEQ ID | ctcacg |
| | accaggacagccacccaaactcctcatctatc | tagttata | NO: | (SEQ ID |
| | ttgcatccaacctagaatctggggtccctgcc | tgcac | 351) | NO: |
| | aggttcagtggcagtgggtctgggacagactt | (SEQ ID | | 352) |
| | caccctcaacatccatcctgtggaggaggagg | NO: 350) | | |
| | atgctgcaacctattactgtcagcacagtagg | | | |
| | gagcttcctctcacgttcggtgctgggaccaa | | | |
| | gctggagctgaaa | | | |
| | (SEQ ID NO: 349) | | | |
| 15 | Caggtcctggcgttgctgctgctgtggcttac | cgagcaag | aatgcaa | caacatt |
| | aggtgccagatgtgacatccagatgactcagt | tgggaata | aaacctt | tttggag |
| | ctccagcctccctatctgcatctgtgggagaa | ttcacaat | agcagat | tactccg |
| | actgtcaccatcacatgtcgagcaagtgggaa | tatttagc | (SEQ ID | tggacg |
| | tattcacaattatttagcatggtatcagcaga | a | NO: | (SEQ ID |
| | aacagggaaaatctcctcagctcctggtctat | (SEQ ID | 355) | NO: |
| | aatgcaaaaaccttagcagatggtgtgccatc | NO: 354) | | 356) |
| | aaggttcagtggcagtggatcaggaacacaat | | | |
| | attctctcaagatcaacagcctgcagcctgaa | | | |
| | gattttgggagttattactgtcaacatttttg | | | |
| | gagtactccgtggacgttcggtggaggcacca | | | |
| | agctggaaatcaaa | | | |
| | (SEQ ID NO: 353) | | | |
| 16 | Gatgttgtgatgacccagactccactcacttt | aagtcaag | ctggtgt | tggcaag |
| | gtcggttaccattggacaaccagcctccatct | tcagagcc | ctaaact | gtacaca |
| | cttgcaagtcaagtcagagcctcttagatagt | tcttagat | ggactct | ttttccg |
| | gatggaaagacatatttgaagtggttgttaca | agtgatgg | (SEQ ID | tggacg |
| | gaggccaggccagtctccaaagcgcctaatct | aaagacat | NO: | (SEQ ID |
| | atctggtgtctaaactggactctggagtccct | atttgaag | 359) | NO: |
| | gacaggttcactggcagtggatcagggacaga | (SEQ ID | | 360) |
| | tttcacactgaaaatcagcagagtggaggctg | NO: 358) | | |
| | aggatttgggagtttattattgctggcaaggt | | | |
| | acacattttccgtggacgttcggtggaggcac | | | |
| | caagctggaaatcaaa | | | |
| | (SEQ ID NO: 357) | | | |
| 17 | Gatattgtgatgactcaggctgcaccctctgt | aggtctag | cggatgt | atgcaac |
| | acctgtcactcctggagagtcagtatccatct | taagagtc | ccaacct | atctaga |
| | cctgcaggtctagtaagagtctcctgcatagt | tcctgcat | tgcctca | atatcct |
| | aatggcaacacttacttgtattggttcctgca | agtaatgg | (SEQ ID | ttcacg |
| | gaggccaggccagtctcctcagctcctgatat | caacactt | NO: | (SEQ ID |
| | atcggatgtccaaccttgcctcaggagtccca | acttgtat | 363) | NO: |
| | gacaggttcagtggcagtgggtcaggaactgc | (SEQ ID | | 364) |
| | tttcacactgagaatcagtagagtggaggctg | NO: 362) | | |
| | aggatgtgggtgtttattactgtatgcaacat | | | |
| | ctagaatatcctttcacgttcggtgctgggac | | | |
| | caagctggagctgaaa | | | |
| | (SEQ ID NO: 361) | | | |
| 18 | actattgtgatgacccagactcccaaattcct | aaggccag | tctgcat | cagcagg |
| | gcttgtatcagcaggagacagggttaccataa | tcagagtg | ccaatcg | attatac |
| | cctgcaaggccagtcagagtgtgagtaatgct | tgagtaat | ctacact | ctctcca |
| | gtagcttggtaccaacagaagccaggccagtc | gctgtagc | (SEQ ID | ttcacg |
| | tcctaaactgctgatatactctgcatccaatc | t | NO: | (SEQ ID |
| | gctacactggagtccctgatcgcttcactggc | (SEQ ID | 367) | NO: |
| | agtggatatgggacggatttcactttcaccat | NO: 366) | | 368) |
| | cagctctgtgcaggctgaagacctggcagttt | | | |
| | atttctgtcagcaggattatacctctccattc | | | |
| | acgttcggctcggggacaaagttggaaataaa | | | |
| | a | | | |
| | (SEQ ID NO: 365) | | | |
| 19 | Caaattgttctcacccagtctccagcaatcat | agtgccag | gacacat | cagcagt |
| | gtctgcttctccaggggagaaggtcaccatgt | ctcaagtg | ccaaact | ggagtag |
| | cctgcagtgccagctcaagtgtaacttacatg | taacttac | ggcttct | taaccca |
| | cactggtaccagcagaagtcaggcacctcccc | atgcac | (SEQ ID | ccgacg |
| | caaaagatggatttatgacacatccaaactgg | (SEQ ID | NO: | (SEQ ID |
| | cttctggagtccctgctcgcttcagtggcagt | NO: 370) | 371) | NO: |
| | gggtctgggacctcttactctctcacaatcag | | | 372) |
| | cagcatggaggctgaagatgctgccacttatt | | | |
| | actgccagcagtggagtagtaacccaccgacg | | | |
| | ttcggtggaggcaccaagctggaaatcaaa | | | |
| | (SEQ ID NO: 369) | | | |
| 20 | aacattgtgctgacccaatctccagcttcttt | agagccag | cttgcat | cagcaaa |
| | ggctgtgtctctagggcagagggccaccatat | tgaaagtg | ccaacct | ataatga |
| | cctgcagagccagtgaaagtgttgatagttat | ttgatagt | agaatct | ggatccg |
| | ggcaatagttttatgcactggtaccagcagaa | tatggcaa | (SEQ ID | tggacg |
| | accaggacagccacccaaactcctcatctatc | tagtttta | NO: | (SEQ ID |
| | ttgcatccaacctagaatctggggtccctgcc | tgcac | 375) | NO: |
| | aggttcagtggcagtgggtctaggacagactt | (SEQ ID | | 376) |
| | caccctcaccattgatcctgtggaggctgatg | NO: 374) | | |
| | atgctgcaacctattactgtcagcaaaataat | | | |
| | gaggatccgtggacgttcggtggaggcaccaa | | | |
| | gctggaaatcaaa | | | |
| | (SEQ ID NO: 373) | | | |
| 24 | Aacattatgatgacacagtcgccatcatctct | aagtccag | tgggcat | catcaat |
| | ggctgtgtctgcaggagaaaaggtcactatga | tcaaagtg | ccactag | acctctc |
| | gctgtaagtccagtcaaagtgttttatacagt | ttttatac | ggaatct | ctcgtac |
| | tcaaatcagaagaactacttggcctggtacca | agttcaaa | (SEQ ID | acg |
| | gcagaaaccagggcagtctcctaaactgctga | tcagaaga | NO: | (SEQ ID |
| | tctactgggcatccactagggaatctggtgtc | actacttg | 379) | NO: |
| | cctgatcgcttcacaggcagtggatctgggac | gc | | 380) |
| | agattttactcttaccatcagcagtgtacaag | (SEQ ID | | |
| | ctgaagacctggcagtttattactgtcatcaa | NO: 378) | | |
| | tacctctcctcgtacacgttcggaggggggac | | | |
| | caagctggaaataaaa | | | |
| | (SEQ ID NO: 377) | | | |
| 34 | Gaaaatgtgctcacccagtctccagcaatcat | agggccag | agcacat | cagcagt |
| | gtctgcatctccaggggaaaaggtcaccatga | ctcaagtg | ccaactt | acagtgg |
| | cctgcagggccagctcaagtgtaagttccagt | taagttcc | ggcttct | ttaccca |
| | tacttgcactggtaccagcagaagtcaggtgc | agttactt | (SEQ ID | ctcacg |
| | ctcccccaaactctggatttatagcacatcca | gcac | NO: | (SEQ ID |
| | acttggcttctggagtccctgctcgcttcagt | (SEQ ID | 383) | NO: |
| | ggcagtgggtctgggacctcttactctctcac | NO: 382) | | 384) |
| | aatcagcagtgtggaggctgaagatgctgcca | | | |
| | cttattactgccagcagtacagtggttaccca | | | |
| | ctcacgttcggtgctgggaccaagctggagct | | | |
| | gaaa | | | |
| | (SEQ ID NO: 381) | | | |
| 35, 36 | gatattgtgatgactcaggctgcaccctctgt | aggtctag | cggatgt | atgcaac |
| | acctgtcactcctggagagtcagtatccatct | taagagtc | ccaacct | atctaga |
| | cctgcaggtctagtaagagtctcctgcatagt | tcctgcat | tgcctca | atatcca |
| | aatggcaacacttacttgtattggttcctgca | agtaatgg | (SEQ ID | ttcacg |
| | gaggccaggccagtctcctcagctcctgatat | caacactt | NO: | (SEQ ID |
| | atcggatgtccaaccttgcctcaggagtccca | acttgtat | 387) | NO: |
| | gacaggttcagtggcagtgggtcaggaactgc | (SEQ ID | | 388) |
| | tttcacactgagaatcagtagagtggaggctg | NO: 386) | | |
| | aggatgtgggtgtttattactgtatgcaacat | | | |
| | ctagaatatccattcacgttcggctcggggac | | | |
| | aaagttggaaataaaa | | | |
| | (SEQ ID NO: 385) | | | |
| 37 | Gacattgtgctgacacagtctcctgcttcctt | agggccag | cttgcat | catcaca |
| | agctgtatctctggggcagagggccaccatct | caaaagtg | ccaacct | gtaggga |
| | catgcagggccagcaaaagtgtcagtacatct | tcagtaca | agaatct | acttcct |
| | ggctatagttatatgcactggtaccaacagaa | tctggcta | (SEQ ID | cggacg |
| | accaggacagccacccaaactcctcatctatc | tagttata | NO: | (SEQ ID |
| | ttgcatccaacctagaatctggggtccctgcc | tgcac | 391) | NO: |
| | aggttcagtggcagtgggtctgggacagactt | (SEQ ID | | 392) |
| | caccctcaacatccatcctgtggaggaggagg | NO: 390) | | |
| | atgcttcaacctattactgtcatcacagtagg | | | |
| | gaacttcctcggacgttcggtggaggcaccaa | | | |
| | gctggaaatcaaa | | | |
| | (SEQ ID NO: 389) | | | |
| 39 | Gatgttgtggtgactcaaactccactctccct | aggtctag | gggattt | ttacaag |
| | gcctgtcagctttggagatcaagtttctatct | tcagagtc | ccaacag | gtacaca |
| | cttgcaggtctagtcagagtcttgcaaacagt | ttgcaaac | attttct | tcagccg |
| | tatgggaacacctatttgtcttggtacctgca | agttatgg | (SEQ ID | tggacg |
| | caagcctggccagtctccacagctcctcatct | gaacacct | NO: | (SEQ ID |
| | atgggatttccaacagattttctggggtgcca | atttgtct | 395) | NO: |
| | gacaggttcagtggcagtggttcagggacaga | (SEQ ID | | 396) |
| | tttcacactcaagatcagcacaataaagcctg | NO: 394) | | |
| | aggacttgggaatgtattactgcttacaaggt | | | |
| | acacatcagccgtggacgttcggtggaggcac | | | |
| | caagctggaaatcaaa | | | |
| | (SEQ ID NO: 393) | | | |
| 40 | Gacatccagatgactcagtctccagcctccct | cgagcaag | aatgcaa | caacatt |
| | atctgcatctgtgggagaaactgtcaccatca | tgggaata | aaacctt | tttggag |
| | catgtcgagcaagtgggaatattcacaattat | ttcacaat | agcagat | tactccg |
| | ttagcatggtatcagcagaaacagggaaaatc | tatttagc | (SEQ ID | tacacg |
| | tcctcagctcctggtctataatgcaaaaacct | a | NO: | (SEQ ID |
| | tagcagatggtgtgccatcaaggttcagtggc | (SEQ ID | 399) | NO: |
| | agtggatcaggaacacaatattctctcaagat | NO: 398) | | 400) |
| | caacagcctgcagcctgaagattttgggagtt | | | |
| | attactgtcaacatttttggagtactccgtac | | | |
| | acgttcggaggggggaccaagctggaaataaa | | | |
| | a | | | |
| | (SEQ ID NO: 397) | | | |
| 41 | Gacattgtgctgacccaatctccagcttcttt | aaggccag | gctgcat | cagcaaa |
| | ggctgtgtctctagggcagagggccaccatct | ccaaagtg | ccaatct | gtaatga |
| | cctgcaaggccagccaaagtgttgattatgat | ttgattat | agaatct | ggatccg |
| | ggtgatagttatatgaactggtaccaacagaa | gatggtga | (SEQ ID | tggacg |
| | accaggacagccacccaaactcctcatctatg | tagttata | NO: | (SEQ ID |
| | ctgcatccaatctagaatctgggatcccagcc | tgaac | 403) | NO: |
| | aggtttagtggcagtgggtctgggacagactt | (SEQ ID | | 404) |
| | caccctcaacatccatcctgtggaggaggagg | NO: 402) | | |
| | atgctgcaacctattactgtcagcaaagtaat | | | |
| | gaggatccgtggacgttcggtggaggcaccaa | | | |
| | gctggaaatcaaa | | | |
| | (SEQ ID NO: 401) | | | |
| 42-L1 | Gatgttgtgatgacccagactccactcacttt | aagtcaag | ctggtgt | tggcaag |
| | gtcggttaccattggacaaccagcctccatct | tcagagcc | ctaaact | gtacaca |
| | cttgcaagtcaagtcagagcctcttagatagt | tcttagat | ggactct | ttttccg |
| | gatggaaagacatatttgaattggttgttaca | agtgatgg | (SEQ ID | tggacg |
| | gaggccaggccagtctccaaagcgcctaatct | aaagacat | NO: | (SEQ ID |
| | atctggtgtctaaactggactctggagtccct | atttgaat | 407) | NO: |
| | gacaggttcactggcagtggatcagggacaga | (SEQ ID | | 408) |
| | tttcacactgaaaatcagcagagtggaggctg | NO: 406) | | |
| | aggatttgggagtttattattgctggcaaggt | | | |
| | acacattttccgtggacgttcggtggaggcac | | | |
| | caagctggaaatcaaa | | | |
| | (SEQ ID NO: 405) | | | |
| 42-L2 | Gatattgtgctaactcagtctccagccaccct | agggccag | tatgctt | caacaga |
| | gtctgtgactccaggagatagagtcagtcttt | tcaaagta | cccagtc | gtaacag |
| | cctgcagggccagtcaaagtattagcaactac | ttagcaac | catctct | ctggcca |
| | ctacactggtatcaacaaaaatcacatgagtc | tacctaca | (SEQ ID | ttcacg |
| | tccaaggcttctcatcaagtatgcttcccagt | c | NO: | (SEQ ID |
| | ccatctctgggatcccctccaggttcagtggc | (SEQ ID | 411) | NO: |
| | agtggatcagggacagatttcactctcagtat | NO: 410) | | 412) |
| | caacagtgtggagactgaagattttggaatgt | | | |
| | atttctgtcaacagagtaacagctggccattc | | | |
| | acgttcggctcggggacaaagttggaaataaa | | | |
| | a | | | |
| | (SEQ ID NO: 409) | | | |
| 43 | Gacattgtgatgtcacagtctccatcctccct | aaatccag | tgggcat | aagcaat |
| | ggctgtgtcagcaggagagaaggtcactatga | tcagagtc | ccactag | cttataa |
| | gctgcaaatccagtcagagtctgctcaacagt | tgctcaac | ggaatct | tcttccg |
| | agaacccgaaagaactacttggcttggtacca | agtagaac | (SEQ ID | ctcacg |
| | gcagaaaccagggcagtctcccaaactgctga | ccgaaaga | NO: | (SEQ ID |
| | tttactgggcatccactagggaatctggggtc | actacttg | 415) | NO: |
| | cctgatcgcttcacaggcagtggatctgggac | gct | | 416) |
| | agatttcactctcaccatcagcagtgtgcagg | (SEQ ID | | |
| | ctgaagacctggcagtttattcctgcaagcaa | NO: 414) | | |
| | tcttataatcttccgctcacgttcggtgctgg | | | |
| | gaccaagctggagctgaaa | | | |
| | (SEQ ID NO: 413) | | | |
| 44 | Caaattgttctcacccagtctccagcaatcat | agtgccag | gacacat | catcagc |
| | gtctgcatctccaggggagaaggtcaccatga | ctcaagta | ccaaact | ggagtag |
| | cctgcagtgccagctcaagtataagttacatg | taagttac | ggcttct | ttacccg |
| | cactggtaccagcagaagccaggcacctcccc | atgcac | (SEQ ID | tacacg |
| | caaaagatggatttatgacacatccaaactgg | (SEQ ID | NO: | (SEQ ID |
| | cttctggagtccctgctcgcttcagtggcagt | NO: 418) | 419) | NO: |
| | gggtctgggacctcttattctctcacaatcag | | | 420) |
| | cagcatggaggctgaagatgctgccacttatt | | | |
| | actgccatcagcggagtagttacccgtacacg | | | |
| | ttcggaggggggaccaagctggaaataaaa | | | |
| | (SEQ ID NO: 417) | | | |
| 45 | Gacattgtgctgacacagtctcctgcttcctt | agggccag | cttgcat | cagcaca |
| | agctgtatctctggggcagagggccaccatct | caaaagtg | ccaacct | gtaggga |
| | catgcagggccagcaaaagtgtcagtacatct | tcagtaca | agaatct | gcttccg |
| | ggctatagttatatgcactggtaccaacagaa | tctggcta | (SEQ ID | tacacg |
| | accaggacagccacccaaactcctcatctatc | tagttata | NO: | (SEQ ID |
| | ttgcatccaacctagaatctggggtccctgcc | tgcac | 423) | NO: |
| | aggttcagtggcagtgggtctgggacagactt | (SEQ ID | | 424) |
| | caccctcaacatccatcctgtggaggaggagg | NO: 422) | | |
| | atgctgcaacctattactgtcagcacagtagg | | | |
| | gagcttccgtacacgttcggaggggggaccaa | | | |
| | gctggaaataaaa | | | |
| | (SEQ ID NO: 421) | | | |
| 47 | aacattgtgctgacccaatctccagcttcttt | agagccag | cttgcat | cagcaaa |
| | ggctgtgtctctagggcagagggccaccatat | tgaaagtg | ccaacct | ataatga |
| | cctgcagagccagtgaaagtgttgatagtttt | ttgatagt | agaatct | ggatcct |
| | ggcaatagttttatgcactggtaccagcagaa | tttggcaa | (SEQ ID | ccgtgga |
| | accaggacagccacccaaactcctcatctatc | tagtttta | NO: | CO |
| | ttgcatccaacctagaatctggggtccctgcc | tgcac | 427) | (SEQ ID |
| | aggttcagtggcagtgggtctaggacagactt | (SEQ ID | | NO: |
| | caccctcaccattgatcctgtggaggctgatg | NO: 426) | | 428) |
| | atgctgcaacctattactgtcagcaaaataat | | | |
| | gaggatcctccgtggacgttcggtggaggcac | | | |
| | caagctggaaatcaaa | | | |
| | (SEQ ID NO: 425) | | | |
| 48 | Gacatcttgctgactcagtctccagccatcct | agggccag | tttgctt | caacaaa |
| | gtctgtgagtccaggagaaagagtcagtttct | tcagagca | ctgagtc | gtaatag |
| | cctgcagggccagtcagagcattggcacaagc | ttggcaca | tatctct | ctggccg |
| | atacactggtatcagcaaggaacaaatggttc | agcataca | (SEQ ID | ctcacg |
| | tccaaggcttctcataaagtttgcttctgagt | c | NO: | (SEQ ID |
| | ctatctctgggatcccttccaggtttagtggc | (SEQ ID | 431) | NO: |
| | agtggatcagggacagattttactcttagcat | NO: 430) | | 432) |
| | caacagtgtggagtctgaagatattgcagatt | | | |
| | attactgtcaacaaagtaatagctggccgctc | | | |
| | acgttcggtgctgggaccaagctggagctgaa | | | |
| | a | | | |
| | (SEQ ID NO: 429) | | | |

### Example 6—Gyrolab Assay to Evaluate the Functionality of Antibodies

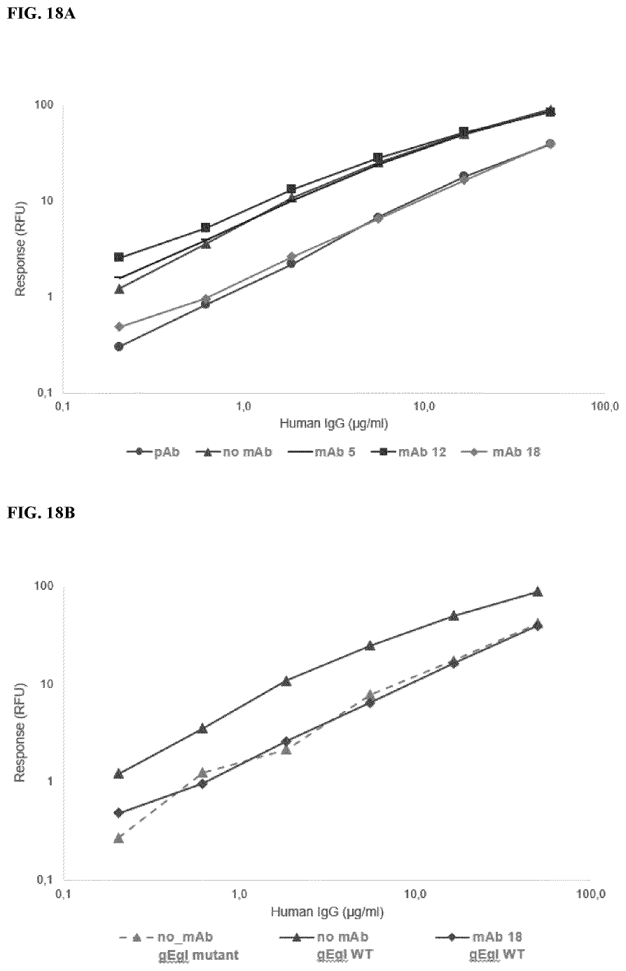

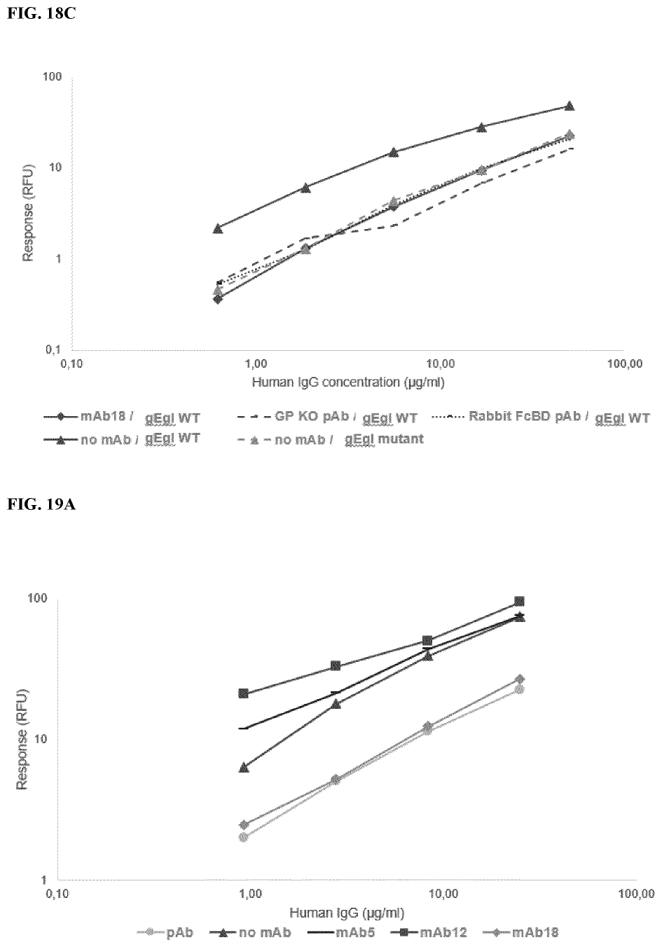

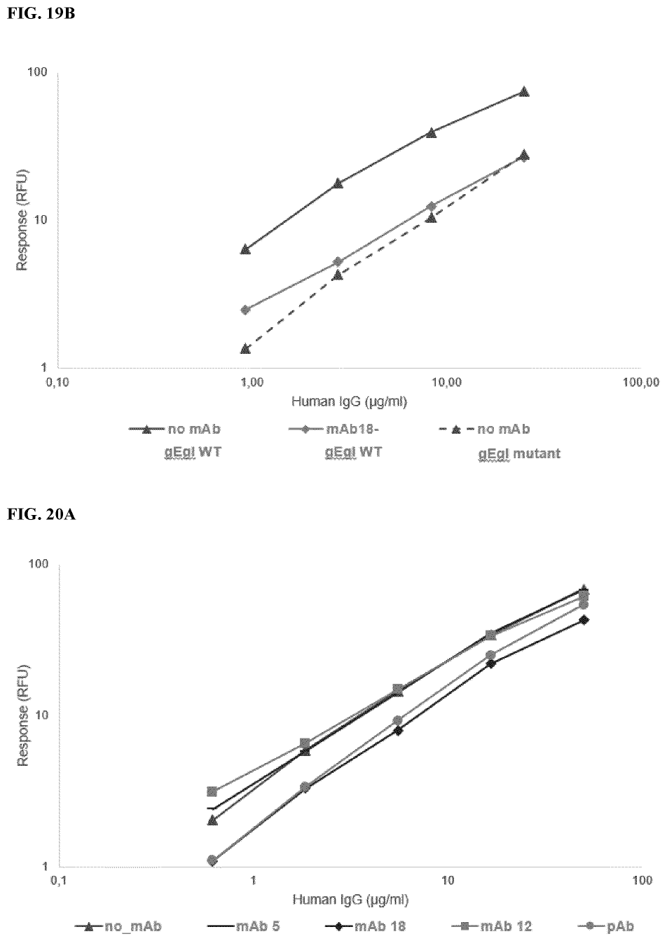

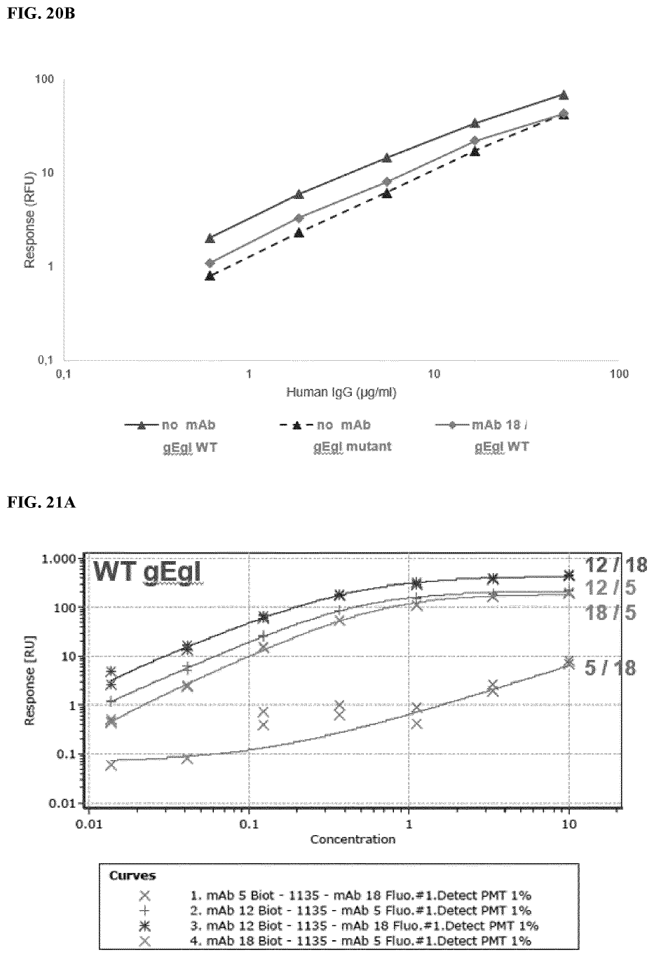

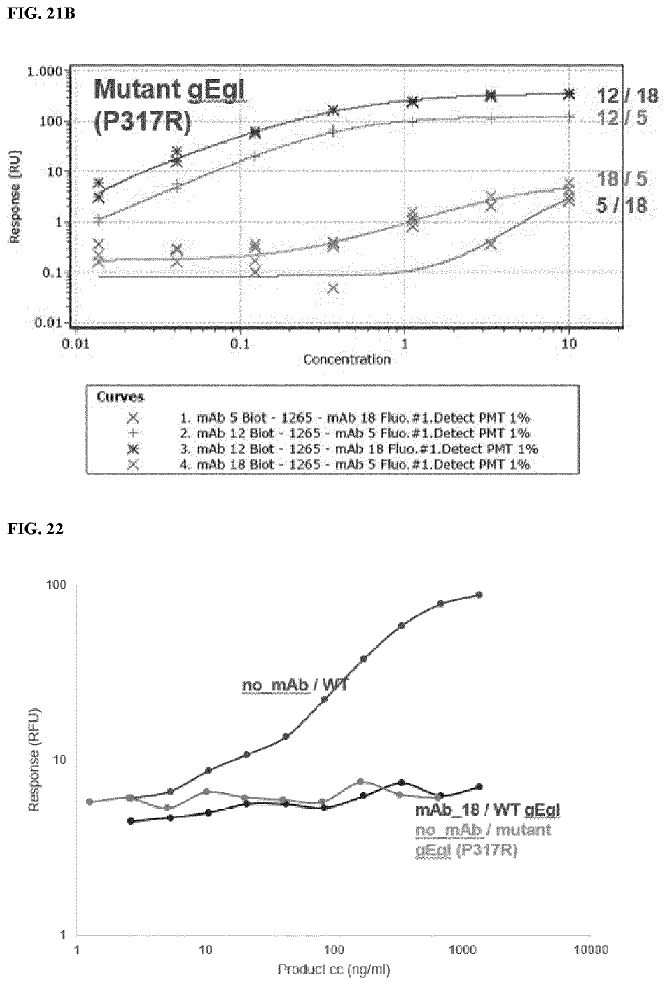

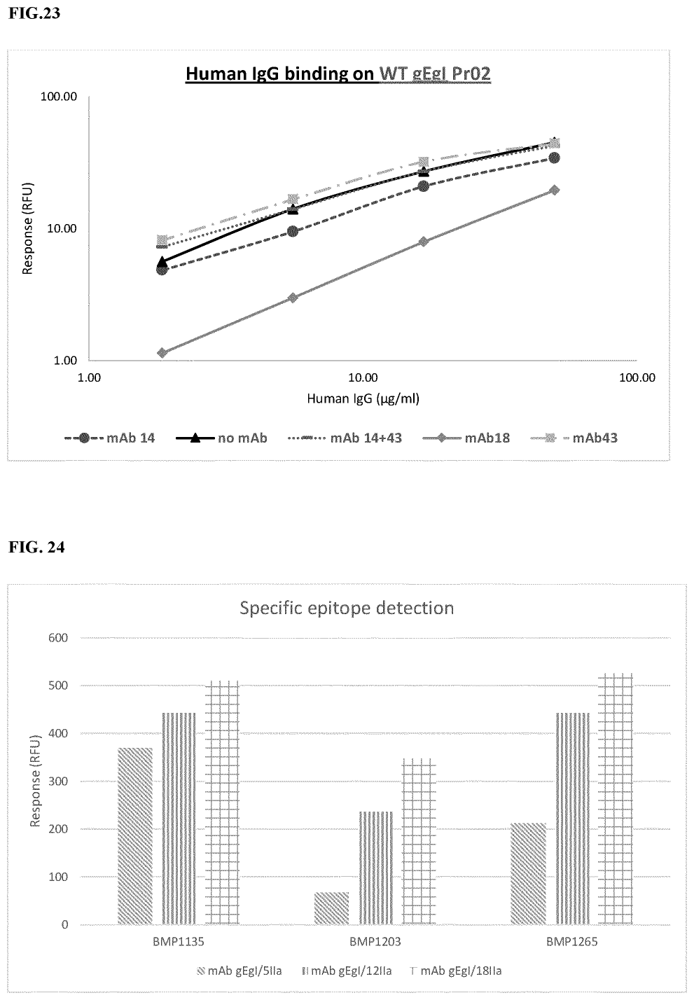

The binding of the Fe domain of human IgGs to the Fc binding domain (FCBD) of the gE protein forming part of the Fc HSV gEgI heterodimer triggers an immune-evasion mechanism. A functional antibody specific to the gE FCBD has the ability to block binding of the Fe domain of human IgGs and avoid the immune-evasion mechanism (FIG. 14).
Three different designs of the gyrolab assay were perfomed to evaluate the functionality of antibodies (FIG. 17). Two forms of the gEgI heterodimer were used, a wildtype (WT) form and a mutant form comprising a mutation in the gE FCBD (P317R) which abolished the binding to human IgGs.
In the first design, the test antibody the was added first, followed by human IgGs. This design allows to assess the ability of the test antibody to block accessibility of the human IgG epitope (FIG. 17). The polyclonal antibodies and the mAb from clone 18 were able to block the accessibility of the human-IgG epitope on WT gEgI, and the drop in human IgG binding is similar to one observed with the mutant gEgI (P317R) in absence of antibodies (FIG. 18A, FIG. 18B, FIG. 18C).
In the second design, the test antibody and the human IgGs were added at the same time. This design allows to assess the ability of the test antibody to compete with the human IgG for binding to the gE FCBD (FIG. 17). The polyclonal antibodies and the mAb from clone 18 were able to compete with the human-IgG epitope for binding to WT gEgI, and the drop in human IgG binding is similar to one observed with the mutant gEgI (P317R) in absence of antibodies (FIG. 19A, FIG. 19B).
In the third design, the human IgGs were added first, followed by test antibody. This design allows to assess the ability of the test antibody to displace the human IgGs bound to the gE FCBD (FIG. 17). The polyclonal antibodies and the mAb from clone 18 were able to compete with the human-IgG epitope for binding to WT gEgI, and the drop in human IgG binding is similar to one observed with the mutant gEgI (P317R) in absence of antibodies (FIG. 20A, FIG. 20B).
The mAb from clone 18 can thus be described as functional in the sense that it is able to block the binding of human IgGs to the FCBD of gE.
Two additional mAb sandwich designs (Design 4) were tested in the gyrolab assay (FIG. 17).
In the first mAb sandwich design, a first biotinylated mAb antibody was captured on the streptaviding bead, then WT or mutated gEgI was added, and finally a second fluorescent mAb was added. This design allows to assess the ability of the first and second mAb to act as capture and detection antibody respectively in a sandwich assay. In this assay:
mAb 5 was not able to capture the WT gEgI, mAb 18 and 12 were able to capture the WT gEgI, and mAb 5, 12 and 18 were able to detect the WT gEgI (FIG. 21A);
mAbs 5 and 18 were not able to capture mutant gEgI (P317R), mAb 12 was able to capture mutant gEgI (P317R)WT gEgI, and mAb 5, 12 and 18 were able to detect mutant gEgI (P317R) (FIG. 21B).
   In the second mAb sandwich design (competition), a biotinylated mAb 12 was captured on the streptaviding bead, then WT or mutated gEgI was added, and finally fluorescent mAb 18 (or no mAbs) and human IgGs were added together. This design allowed to confirm mAb18 is functional (FIG. 22).
   Two other mAbs, GEGI/14IIa (=mAb clone 14) and GEGI/43IIa (=mAb clone 43), specific for the FcBD have also been assessed and as illustrated in FIG. 23 these two mAbs separately or mixed together, did not show a significant impact on human IgG binding compared to mAb GEGI/18IIa.

### Example 7—IVRP (In-Vitro Relative Potency) Assays Design

Based on the results presented herein:
mAb 5 recognises a conformational epitope and is specific to the heterodimer
mAb 12 is specific to the gE FCBD and can be used as a capture antibody
mAb 18 is functional (ability to block binding of human IgGs to gE FCBD) and specific to the gE FCBD.
   Therefore, the following two in-vitro relative potency (IVRP) sandwich assays, for example using the gyrolab assay, can be performed to confirm the heterodimeric form, the conformation and functionality (ability to bind to human gGs) of a HSV gEgI heterodimer:
1^{st} assay: mAb 12 as capture antibody and mAb 5 as detection antibody (to confirm conformation and heterodimer form), and
2^{nd} assay: mAb 12 as capture antibody and mAb 18 as detection antibody (to confirm functionality).

### Example 8—Epitope Accessibility

### Antigen:

| Antigen | | | | |
|---|---|---|---|---|
| name | Antigen type | Host | Produced in | Information |
| BMP1135 | HSV-2 gEgI WT | HEK293 | Ag design | |
| BMP1203 | HSV-2 gEgI WT | CHO | Ag design | Potential |
| | | | | unstable aliquot |
| BMP1265 | HSV-2 gEgI KO | CHO | Ag design | |
| HSV Pr02 | HSV-2 gEgI WT | CHO | MDS | |
| HSV Pr05 | HSV-2 gEgI KO | CHO | MDS | |

Ag produced by Ag design team contains a HIS-TAG
WT = Wild type − unmutated Ag
KO = knock-out construct/mutation P317-R

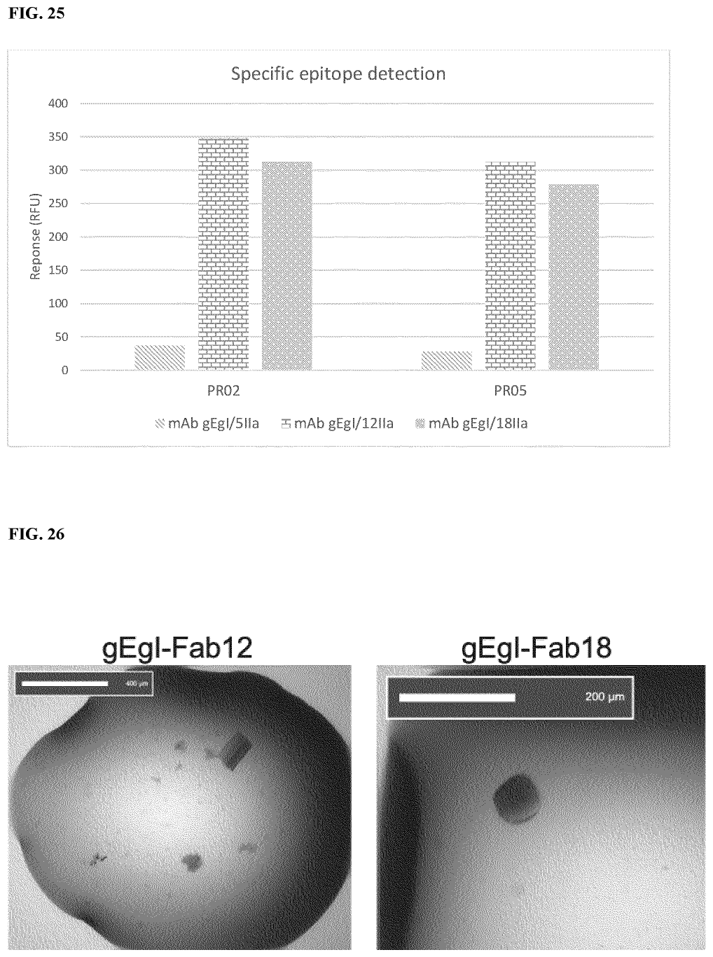

The accessibility of the specific epitopes of interest were assessed by direct detection, for different HSV-2 gEgI constructs. Direct detection was carried out using a 2 step Gyros immunoassay. The different mAbs were labelled with fluorochrome. FIGS. 24 and 25 show the detection of these epitopes present in HSV-2 gEgI.
Of note, the data generated on BMP1203 cannot be considered as the stability of the aliquot can be compromised by inadequate storage.
This data shows that antigenic site specific of the mAb GEGI/5IIa, 12IIa and 18IIa are present in each type of HSV-2 gEgI constructs or production.

### Example 9—Structural Characterization and Epitope Mapping for HSV-2 Anti-gE mAbs 12 and 18

### Aim

The aim of this experiment was to provide high resolution structural information on the HSV-2 antigen and the epitopes targeted by mAb12 and mAb18 by solving structures of gE-Fab (antigen binding fragment) complexes for each antibody. These antibodies can be used as immunotools for use in analytical technologies especially antigenicity and in vitro potency assays. Specifically, mAb 12 is selected as a capture mAb and mAb 18 as a detection mAb. In this report, the X-ray structures for the complexes between gE_FcBD (the Fc binding domain of gE) and Fab 12 and gE_FcBD and Fab18 were determined, and the paratope-epitope contacts mapped.

### Materials

HSV2-gEgI expressed in GnTi-cells—eNB N74940-2
Fab 12—eNB N74940-1
Fab 18—eNB N74940-1

### Principle

X-ray crystallography can be used for determining the 3D structure of macromolecules at high resolution and allow the detailed description of antibody-antigen interactions. Such information can be used to rationally guide vaccine design efforts (i.e introduction of stabilizing mutations, removing/masking of unwanted epitopes, increase thermostability), verification of protein state/conformation, and mapping of antibody-antigen interactions.
X-ray crystallography relies on the formation of protein crystals which are obtained after sparse matrix screening of protein that is greater than 95% pure with 1000's of possible buffer conditions in a high through-put format. Screens are monitored for the formation of protein crystals, which can form as early as Day 1 or up to over 1 year in some extreme cases. There is no guarantee that protein crystals will ever form and is highly dependent on the protein sample. Characteristics such as high flexibility of the protein often hinder the formation of protein crystals and can require modification of the protein construct used for X-ray crystallography studies.
Protein crystals are protected from radiation by soaking in a cryogenic buffer, flash frozen in liquid nitrogen, and exposed to a high intensity x-ray beam, often from a synchrotron. If diffraction of the x-rays extends to high enough resolution (generally greater than 4 Angstroms), then a data set is collected by rotating the crystal while exposing it to a constant beam of x-rays and collecting the diffraction images. These images are then used for solving of the 3D protein structure.
In this report, the X-ray structures for gE_FcBD-Fab 12 and gE_FcBD-Fab18 were each solved from data collected from individual protein crystals at the Advanced Photon Source (APS) located at Argonne National Labs (SER-CAT, beamline 22-ID).

### Method

### Crystal Screening

HSV-2 gEgI expressed in GnTi-cells was incubated with either excess Fab 12 or excess Fab 18 overnight at 4 degrees Celsius. Size exclusion chromatography (Superdex 200 10/300 GL) in buffer 10 mM Tris pH 7.5, 50 mM NaCl was used to isolate the gEgI-Fab complexes and remove the excess Fab. Each complex was concentrated to −7 mg/mL using Amicon spin filters with a kDa MW cutoff. Six sparse matrix crystallization screens were setup using an STP Labtech Mosquito liquid handler at a protein to buffer ratio of 1:1 on sitting drop vapor diffusion trays and the trays stored in a room temperature Rock Imager for monitoring of crystal growth.
A single crystal for the gEgI-Fab12 complex was found at Day 60 in buffer containing 0.02 M Sodium/potassium phosphate 20% w/v PEG 3350.
A single crystal was also identified for the gEgI-Fab18 complex at Day 70 in buffer 25% PEG 3350 (FIG. 26).
The crystals from each complex were cryoprotected in the respective mother liquor supplemented with 20% ethylene glycol, flash cooled in liquid nitrogen and shipped to APS for data screening/collection.

### Data Collection

The crystal of gEgI-Fab12 diffracted x-rays to 2.3 Å and a data set was collected and processed/scaled into space group P2₁2₁2₁ using HKL3000 software.
The crystal of gEgI-Fab18 diffracted x-rays to 2.75 Å and a data set was collected and processed/scaled into space group P2₁2₁2₁ using HKL3000 software.

### Structure Determination

Molecular replacement was used to solve the structure of gEgI-Fab12 with a homology model of the gE Fc Binding domain (gE_FcBD) used as one search model (SWISS-MODEL) and a homology model of Fab 12 built in MOE used as the second search model.
Molecular replacement was also used to solve the structure of gEgI-Fab18 with the gE_FcBD from the Fab12 bound complex used as one search model and a homology model of Fab 18 built in MOE used as the second search model.

### Results

### Crystal Structure of the 2E FcBD-Fab12 Complex

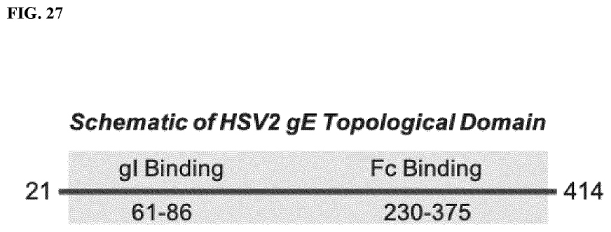

The x-ray structure of the gE_FcBD-Fab12 complex was solved to 2.3 Å resolution. Analysis of the x-ray data revealed two copies of Fab 12 and two copies of gE_FcBD in the asymmetric unit of the crystal, leaving an approximate solvent content of 55%. No additional portions of gE or any of gI were found to be present in the crystal data. Since the crystal screens were set up using the full length gEgI antigen (Full length gE schematic shown in FIG. 27), the x-ray structure implies that the gE portion bound to gI (gI binding domain of gE) was cleaved in situ and the gE_FcBD-Fab12 then formed crystals. The x-ray structure of gE_FcBD-Fab12 was refined to R_{work}/R_{free} values of 23/27 with good Ramachandran and stereochemistry (Table 12).

TABLE 12
X-ray data collection and refinement statistics

| | gE_FcBD-Fab12 | | gE_FcBD-Fab18 | |
|---|---|---|---|---|
| Data Collection | | | | |
| Wavelength (λ) | 1 | | 1 | |
| Resolution range | 42.18-2.31 | (2.39-2.31) * | 38.12-2.75 | (2.84-2.75) |
| Space group | P 2₁2₁2₁ | | P 2₁2₁2₁ | |
| Cell dimensions | | | | |
| a, b, c (Å) | 61.6, 90.7, 289.2 | | 51.7, 78.5, 158.4 | |
| α, β, γ (°) | 90, 90, 90 | | 90, 90, 90 | |
| Total reflections | 428687 | (36796) | 110390 | (9132) |
| Unique reflections | 70345 | (5585) | 17220 | (1254) |
| Multiplicity | 6.1 | (5.6) | 6.4 | (5.8) |
| Completeness (%) | 94.2 | (78.1) | 94.7 | (73.4) |
| I/σI | 19.9 | (4.6) | 8.3 | (3.1) |
| Wilson B-factor | 30.25 | | 44.75 | |
| R-merge | 0.07672 | (0.24) | 0.300 | (1.412) |
| CC_{1/2} | 0.996 | (0.982) | 0.979 | (0.579) |
| Refinement | | | | |
| Resolution (Å) | 42.18-2.31 | | 38.12-2.75 | |
| No. reflections | 68170 | (5586) | 16515 | (1254) |

| | | |
|---|---|---|
| R_{work}/R_{free} (%) | 22/28 | 20/27 |
| No. atoms | | |
| Protein | 9168 | 4533 |
| solvent | 562 | 53 |
| R.m.s. deviations: | | |
| Bond lengths (Å) | 0.009 | 0.012 |
| Bond angles (°) | 1.15 | 1.35 |
| Ramachandran plot^{#} | | |
| Favored (%) | 93.88 | 90.27 |
| Allowed (%) | 5.20 | 8.19 |
| Outliers (%) | 0.92 | 1.54 |
| Average B-factor | 46.95 | 37.42 |
| macromolecules | 47.15 | 37.46 |
| solvent | 43.19 | 34.10 |
| Number of | 33 | N/A |
| TLS groups | | |
| PDB ID | N/A | N/A |

R.m.s. deviation, root-mean square deviation.
* Values in parentheses are for the highest resolution shell.
^{#}Measured using Molprobity

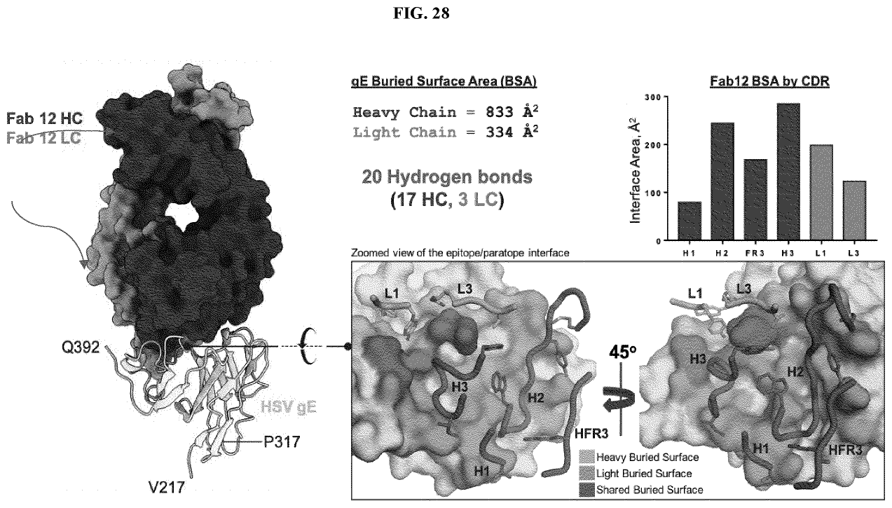

The structure reveals that Fab 12 binds a large, conformation epitope on gE covering a total buried surface area (BSA) of 1167 Å², centered around gE residue Tyr302, and distal from gE residue Pro317 (mutated to an arginine in the drug product), consistent with binding data that the P317R mutation does not affect binding of mAb 12 (FIG. 28). Heavy chain complementarity determining regions (CDRs) H1, H2, H3, and heavy framework region 3 all interact with gE and form a total of 17 hydrogen bonds (Table 13). The heavy chain H3, which buries ˜300 Å², consists mainly of van der Waals interactions (vdWs) with the exception of Ser102_{HCDR3} which forms a hydrogen bond with Ser299 of gE.

TABLE 13
Fab 12 Heavy chain hydrogen-bond interactions with gE_FcBD.

| HSV2-gE | Fab HC |
|---|---|
| Residue, atom | Residue, atom |
| Arg 262, NH2 | Thr57, O |
| Ser267, N | Thr30, O |
| Ser267, OG | Ala53, O |
| Ser268, N | Thr30, O |
| Ser268, OG | Thr31, OG |
| Ser268, OG | Thr30, O |
| Ser299, OG | Ser102, OG |
| Gln378, NE2 | Gly54, O |
| Asp264, OD1 | Ser56, OG |
| Asp264, OD1 | Ser56, N |
| Asp264, OD1 | Gly55, N |
| Asp264, OD2 | Ala53, N |
| Asp264, OD2 | Gly54, N |
| Ser267, OG | Asn73, ND2 |
| Gln350, O | Thr57, N |
| Gln350, OE1 | Thr57, OG1 |
| Gln350, OE1 | Tyr59, OH |

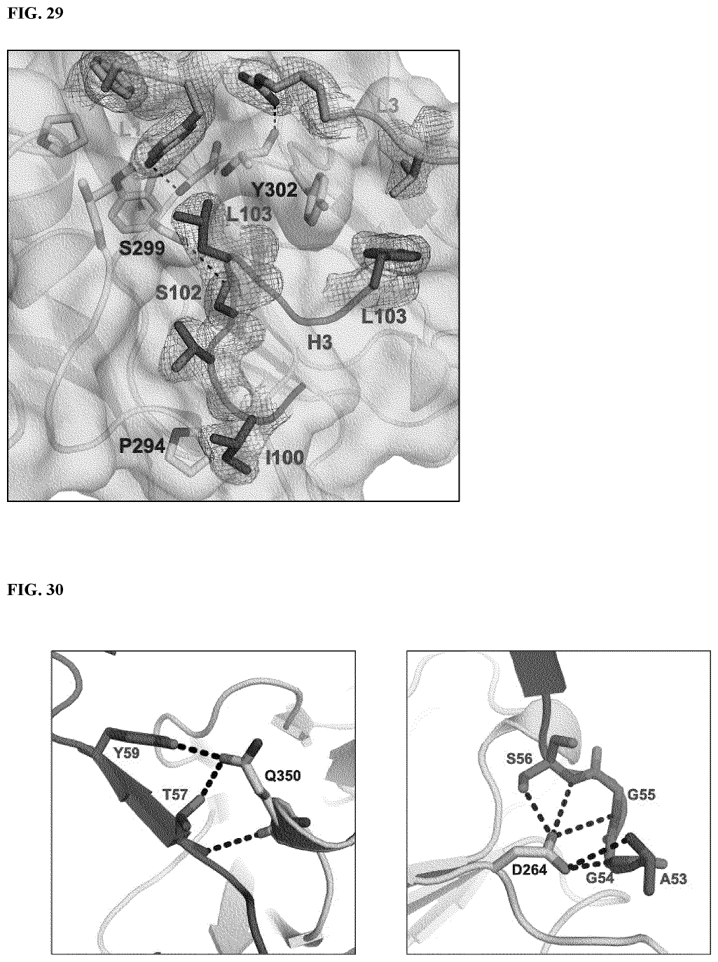

Specific vdWs interactions include Ile100_{HCDR3} which stacks against Pro294, Leu103_{HCDR3} which interacts with Leu288, and Tyr106_{HCDR3} with Tyr302 (FIG. 29). The Fab12 CDR H2 interactions are also quite extensive (˜250 Å² of BSA) and account for eleven of the heavy chain hydrogen bonds with gE. Two residues on gE, Asp264 and Gln350, account for eight of those hydrogen bonds (FIG. 30).
The Fab 12 CDR L1 and L3 bury a total of 334 Å² on gE, forming a single hydrogen bond with L1 (Tyr31_{HCDR1} with the main chain nitrogen of Trp300) and two hydrogen bonds with L3 (the main chain oxygen of Ser95_{LCDR3} with the hydroxyl group oxygen of Tyr302, and the Arg96_{LCDR3} NH1 nitrogen with the main chain oxygen of Ala301) (FIG. 29).
In conclusion, the 2.3 Å x-ray structure of the gE_FcBD in complex with Fab 12 provides an atomic view of the conformational epitope targeted by mAb 12. The structure provides evidence that the epitope is not located near the Pro317Arg mutation and supports the use of mAb 12 as a capture antibody for TRD antigenicity and in vitro potency assays.

### Crystal Structure of the 2E FcBD-Fab18 Complex

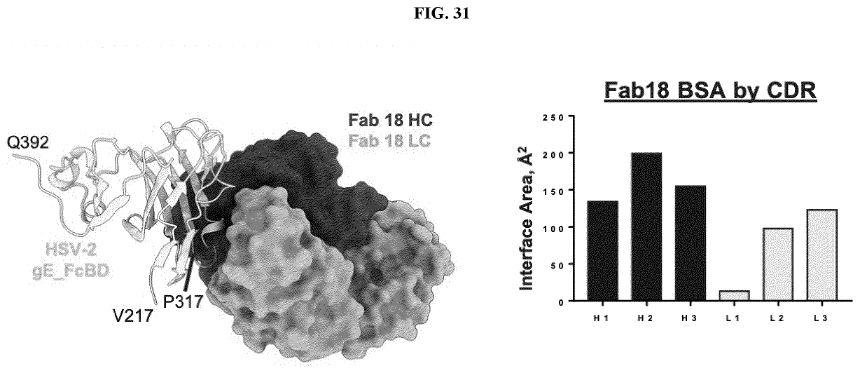

The x-ray structure of the gE_FcBD-Fab18 complex was solved at 2.75 Å resolution. One copy of Fab 18 and one copy of gE_FcBD were found to be present in the asymmetric unit of the crystal, leaving an approximate solvent content of 55%. No additional portions of gE or any of gI were found to be present in the crystal data. Since the crystal screens were set up using the full length gEgI antigen (Full length gE schematic shown in FIG. 27), the x-ray structure implies that the gE portion bound to gI (gI binding domain of gE) was cleaved in situ and the gE_FcBD-Fab18 then formed crystals, as also seen with the Fab 12 bound structure described above. The x-ray structure was refined to R_{work}/R_{free} values of 20/28 with good Ramachandran and stereochemistry (Table 12).
The structure reveals that Fab 18 binds a conformation epitope on gE covering a total BSA of 786 Å² utilizing all three-heavy chain CDRs (489 Å²) and all three-light chain CDRs (234 Å²) and located distant from the Fab 12 binding site and in the proximity of Pro317 (FIG. 31). The location of the epitope is consistent with binding studies showing no competition with mAb 12 and reduced binding to the Pro317Arg mutant.

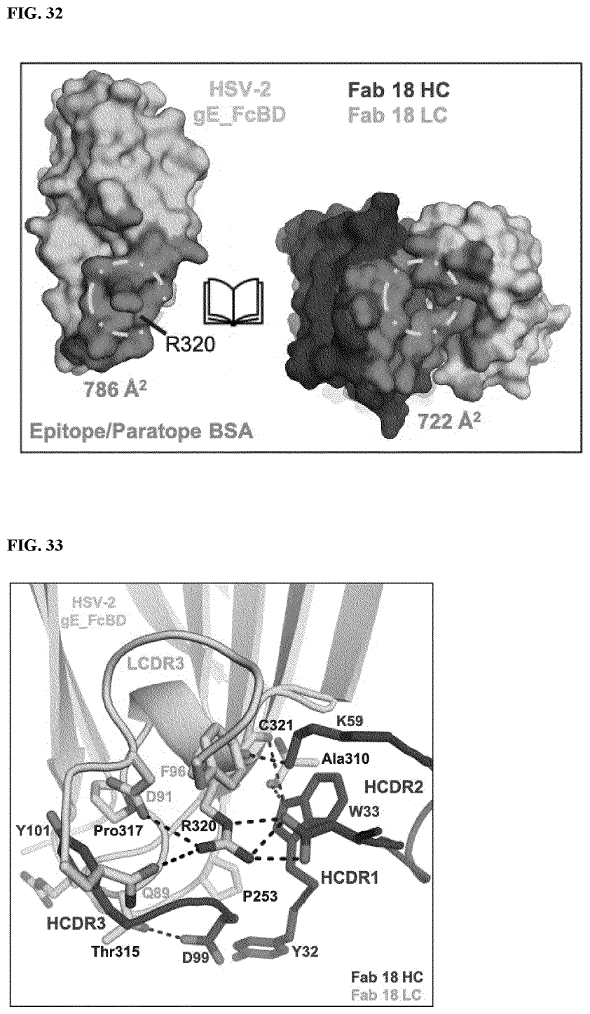

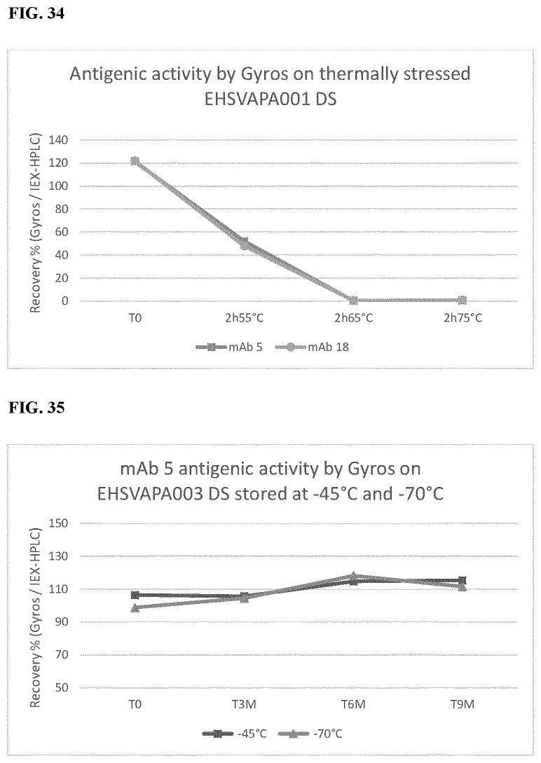

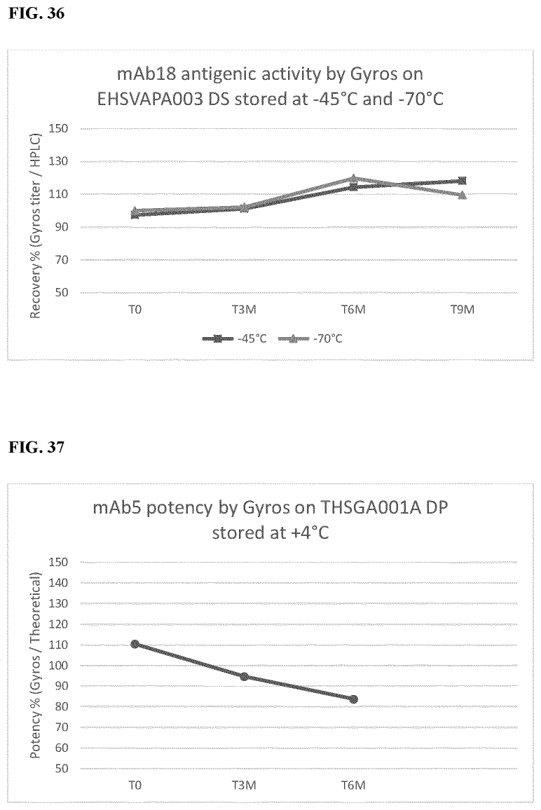

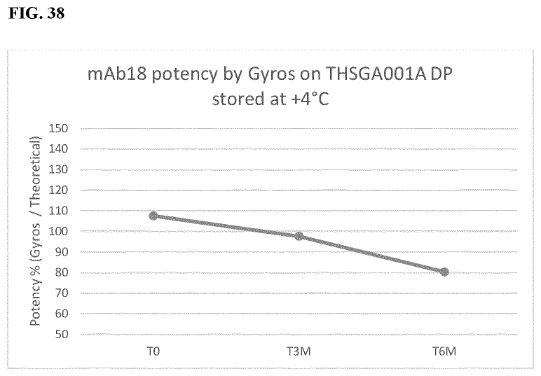

The Fab 18 epitope on gE centers around contacts with Arg320, which protrudes into a pocket formed by the Fab 18 heavy and light chains (FIG. 32). Of the nine total hydrogen bonds formed between gE and Fab 18, five of them are from interactions with Arg320: heavy chain residues Lys59 (one hydrogen bond), and Asp50 (2 hydrogen bonds), and light chain residues Asp9l (one hydrogen bond) and Gln89 (one hydrogen bond) (FIG. 33). The Tyr101_{HCDR3} phenyl ring is positioned 4.4 Å from the Pro317 side chain. This distance is favorable for vdWs contacts, but also explains the reduced binding affinity seen with the gE Pro317Arg mutant because the arginine side chain would be too large to be easily accommodated with causing as steric clash with the Tyr101_{HCDR3} side chain (FIG. 33).

In conclusion, the 2.75 Å x-ray structure of the gE_FcBD in complex with Fab 18 provides an atomic view of the conformational epitope targeted by mAb 18. This structure provides precise epitope mapping of the mAb 18 interactions with gE, and provides a rational for the reduced binding affinity of mAb 18 to the gE P317R mutant. This work supports the use of mAb 18 as the detection antibody for TRD antigenicity and in vitro potency assays.

1. A monoclonal antibody (mAb) which binds to an epitope on a HSV gEgI heterodimer.

2. The mAb of claim 1, wherein the dissociation constant (K_{D}) between said mAb and the HSV gEgI heterodimer is lower than 5×10⁻⁷ M.

3. The mAb of claim 1, wherein said mAb binds to an epitope located on the HSV gE Fc binding domain (FCBD).

4. The mAb of claim 3, wherein said mAb comprises any one or a combination of CDRs selected from SEQ ID NOs: 50-52, 30-32, 34-36, 94-96, 48-50, 154-156, 134-136, 138-140, 202-204 and 214-216, or variants thereof, wherein the variant has 1, 2, or 3 amino acid deletions, substitutions or insertions.

5. The mAb of claim 3, comprising a heavy chain and a light chain, wherein the heavy chain variable region has a sequence selected from SEQ ID NO: 49, SEQ ID NO: 29, SEQ ID NO: 33, SEQ ID NO: 93 and SEQ ID NO: 105, or a sequence which is at least 80%, 85%, 90%, 95%, 96%, 97%, 98% or 99% identical thereto, and wherein the light chain variable region has a sequence selected from SEQ ID NO: 153, SEQ ID NO: 133, SEQ ID NO: 137, SEQ ID NO: 201 and SEQ ID NO: 213, or a sequence which is at least 80%, 85%, 90%, 95%, 96%, 97%, 98% or 99% identical thereto.

6. The mAb of claim 1, wherein said mAb binds to a gE epitope located outside of the HSV gE FCBD.

7. The mAb of claim 6, wherein said mAb comprises any one or a combination of CDRs selected from SEQ ID NOs: 14-16, 74-76, 98-100,110-112, 118-120, 178-180, 206-208 and 218-220, or variants thereof, wherein the variant has 1, 2, or 3 amino acid deletions, substitutions or insertions.

8. The mAb of claim 6, comprising a heavy chain and a light chain, wherein the heavy chain variable region has a sequence selected from SEQ ID NO: 13, SEQ ID NO: 73, SEQ ID NO: 97 and SEQ ID NO: 109, or a sequence which is at least 80%, 85%, 90%, 95%, 96%, 97%, 98% or 99% identical thereto, and wherein the light chain variable region has a sequence selected from SEQ ID NO: 117, SEQ ID NO: 177, SEQ ID NO: 205 and SEQ ID NO: 217, or a sequence which is at least 80%, 85%, 90%, 95%, 96%, 97%, 98% or 99% identical thereto.

9. A binding assay for in vitro analysis of a sample comprising a HSV gE antigen or gEgI heterodimer comprising the steps of:
i) contacting the sample with a detection antibody directed against a HSV gE or gEgI epitope under conditions sufficient to form an immune complex; and
ii) measuring the interaction between the HSV gE antigen or gEgI heterodimer and detection antibody from step (i).

10. The assay of claim 9, wherein said mAb is a functional mAb which is specific to the gE FCBD and has the ability to block binding of the Fc domain of human IgGs.

11. The assay of claim 10, wherein said functional mAb comprises a heavy chain and a light chain, wherein the heavy chain variable region comprises the HCDR1 shown in SEQ ID NO: 50, the HCDR2 shown in SEQ ID NO: 51 and the HCDR3 shown in SEQ ID NO: 52, and the light chain variable region comprises the LCDR1 shown in SEQ ID NO: 154, the LCDR2 shown in SEQ ID NO: 155 and the LCDR3 shown in SEQ ID NO: 156.

12. (canceled)

13. (canceled)

14. (canceled)

15. (canceled)

16. The mAb of claim 1, wherein the HSV gEgI heterodimer is a HSV2 gEgI heterodimer.

17. A method for treatment of recurrent herpes infection, or prevention or reduction of the frequency of recurrent herpes virus infection, in a subject comprising administering an immunologically effective amount of the mAb of claim 1 to the subject.

18. The method of claim 17, wherein the subject is a human subject.

19. The method of claim 17, wherein said mAb is a functional mAb which is specific to the gE FCBD and has the ability to block binding of the Fc domain of human IgGs.
